(19) United States
(12) Patent Application Publication
(10) Pub. No.: US 2013/0040935 A1
(43) Pub. Date: Feb. 14, 2013

(54) CYCLIC KETO-ENOLS FOR THERAPY

(71) Applicants: Ningshu Liu, Berlin (DE); Kai Thede, Berlin (DE); Philip Lienau, Berlin (DE); Arne Scholz, Berlin (DE); Christoph-Stephan Hilger, Berlin (DE); Ulf Bömer, Glienicke (DE); Maher Najjar, Nurnberg (DE); Knut Eis, Berlin (DE); Reiner Fischer, Monheim (DE); Wahed Ahmed Moradi, Monheim (DE)

(72) Inventors: Ningshu Liu, Berlin (DE); Kai Thede, Berlin (DE); Philip Lienau, Berlin (DE); Arne Scholz, Berlin (DE); Christoph-Stephan Hilger, Berlin (DE); Ulf Bömer, Glienicke (DE); Maher Najjar, Nurnberg (DE); Knut Eis, Berlin (DE); Reiner Fischer, Monheim (DE); Wahed Ahmed Moradi, Monheim (DE)

(73) Assignee: BAYER INTELLECTUAL PROPERTY GMBH, Monheim (DE)

(21) Appl. No.: 13/578,914

(22) PCT Filed: Feb. 8, 2011
(86) PCT No.: PCT/EP11/51771

§ 371 Date: Oct. 26, 2012

(30) Foreign Application Priority Data
Feb. 15, 2010 (DE) 10 2010 008 644.4

(51) Int. Cl. A61K 31/4015; C07D 209/54; C07D 207/38; C07D 513/04; C07D 209/96; C07D 491/113; C07D 307/94; C07D 307/33; C07D 498/04; A61K 31/407; A61K 31/403; A61K 31/542; A61K 31/365; A61K 31/382; A61K 31/553; A61P 35/00; C07D 491/107

(52) U.S. Cl. 514/211.08; 548/410; 548/408; 548/544; 544/47; 548/411; 549/265; 549/319; 540/545; 514/409; 514/425; 514/224.2; 514/462; 514/473; 514/460; 514/432

(57) ABSTRACT

The invention relates to 5′-biphenyl-substituted cyclic ketoenols for therapeutic purposes, to pharmaceutical compositions and to their use in therapy, in particular for the prophylaxis and therapy of tumour disorders.

The present invention relates to 5′-biphenyl-substituted cyclic ketoenols for therapeutic purposes, to pharmaceutical compositions and to their use in therapy, in particular for the prophylaxis and/or therapy of tumour disorders.

Acetyl-CoA carboxylases (ACCs) play a key role in cellular fatty acid homeostasis. ACCs are biotin-containing enzymes which catalyze the carboxylation of acetyl-CoA to malonyl-CoA in an ATP-dependent manner (Kim, 1997; Harwood, 2005; Tong, 2005). This reaction, which proceeds as two semi-reactions, a biotin carboxylase (BC) reaction and a carboxyltransferase (CT) reaction, is the first initial step in the fatty acid biosynthesis and is the rate-determining step of the pathway. Two human ACC isoforms, ACC1 and ACC2, are known, which are encoded by two different genes (LuTFI ABU-ELHEIGA et al., 1995, Jane WIDMER, et al. 1996). ACC1 is expressed in lipogenic tissue (liver, fatty tissue), is localized in the cytosol and fills the malonyl-CoA pool which serves as C2 unit donor for the de novo synthesis of long-chain fatty acids by FASN and subsequent chain elongation. ACC2 is expressed in particular in oxidative tissues (liver, heart, skeletal muscle) (Bianchi et al., 1990; Kim, 1997), is associated with the mitochondria, and regulates a second pool of malonyl-CoA. This regulates the fatty acid oxidation by inhibiting carnitine palmitoyl transferase I, the enzyme which facilitates the import of long-chain fatty acids into the mitochondria for β-oxidation (Milgraum L Z, et al., 1997, Widmer J. et al., 1996). Both enzymes have very high sequence homology and are regulated in a similar manner by a combination of transcriptional, translational and posttranslational mechanisms. In humans as well as in animals, the ACC activity is under the strict control of a number of dietary, hormonal and other physiological mechanisms which act through forward allosteric activation by citrate, feedback inhibition by long-chain fatty acids, reversible phosphorylation and/or inactivation or modulation of the enzyme production by modified gene expression, for example.

ACC1 knockout mice are embryonally lethal (Swinnen, et al., 2006, Abu-Elheiga, et al. 2005). ACC2 knockout mice show reduced malonyl-CoA concentrations in skeletal and heart muscle, increased fatty acid oxidation in the muscle, reduced liver fat levels, reduced amounts of total body fat, increased levels of UCP3 in skeletal muscle (as a sign of increased energy output), reduced body weight, lower plasma concentrations of free fatty acids, reduced plasma glucose levels, reduced amounts of tissue glycogen, and they are protected against diet-induced diabetes and obesity (Abu-Elheiga et al., 2001, 2003; Oh et al., 2005).

In addition to being involved in the fatty acid synthesis in lipogenic tissues and the fatty acid oxidation in oxidative tissues, an upregulation of ACC and an increased lipogenesis was observed in many tumour cells (Swinnen, et al., 2004, Heemers, et al., 2000, Swinnen, et al., 2002, Rossi, et al., 2003, Milgraum, et al., 1997, Yahagi, et al., 2005). With high probability, this phenotype contributes to the development and progression of tumours; however, the associated regulatory mechanisms still have to be elucidated.

EP0454782 and U.S. Pat. No. 5,759,837 claim the use of inhibitors of fatty acid synthesis for inhibiting tumor cell growth. Cyclic ketoenols are not disclosed.

A number of substances capable of inhibiting plant- and/or insect-ACC have been found.

PCT patent application PCT/EPP99/01787, published as WO 99/48869, which corresponds to the European patent EP 1 066 258 B1, relates to novel arylphenyl-substituted cyclic ketoenols, to a plurality of processes for their preparation and to their use as pesticides and herbicides.

Pharmaceutical properties of 3-acylpyrrolidine-2,4-diones have been described in the prior art (S. Suzuki et al. Chem. Pharm. Bull. 15 1120 (1967)). Furthermore, N-phenylpyrrolidine-2,4-diones have been synthesized by R. Schmierer and H. Mildenberger (Liebigs Ann. Chem. 1985, 1095). A biological activity of these compounds has not been described.

EP-A-0 262 399 and GB-A-2 266 888 disclose compounds of a similar structure (3-arylpyrrolidine-2,4-diones); however, these compounds have not been known to have any herbicidal, insecticidal or acaricidal activity. Known to have herbicidal, insecticidal or acaricidal activity are unsubstituted bicyclic 3-arylpyrrolidine-2,4-dione derivatives (EP-A-355 599, EP-A-415 211 and JP A 12 053 670), and also substituted monocyclic 3-arylpyrrolidine-2,4-dione derivatives (EP-A-377 893 and EP-A-442 077).

Also known are polycyclic 3-arylpyrrolidine-2,4-dione derivatives (EP-A-442 073) and also 1H-arylpyrrolidinedione derivatives (EP-A-456 063, EP-A-521 334, EP-A-596 298, EP-A-613 884, EP-A-613 885, WO 95/01 971, WO 95/26 954, WO 95/20 572, EP-A-0 668 267, WO 96/25 395, WO 96/35 664, WO 97/01 535, WO 97/02 243, WO 97/36 868, WO 97/43275, WO 98/05638, WO 98/06721, WO 98/25928, WO 99/24437, WO 99/43649, WO 99/48869, WO 99/55673, WO 01/17972, WO 01/23354, WO 01/74770, WO 03/013249, WO 03/062244, WO 2004/007448, WO 2004/024 688, WO 04/065366, WO 04/080962, WO 04/111042, WO 05/044791, WO 05/044796, WO 05/048710, WO 05/049569, WO 05/066125, WO 05/092897, WO 06/000355, WO 06/029799, WO 06/056281, WO 06/056282, WO 06/089633, WO 07/048,545, DEA 102 00505 9892, WO 07/073,856, WO 07/096,058, WO 07/121,868, WO 07/140,881, WO 08/067,873, WO 08/067,910, WO 08/067,911, WO 08/138,551, WO 09/015,801, WO 09/039,975, WO 09/049,851, WO 09/115,262, WO10/052,161, WO 10/063,378, WO 10/063,670, WO10/063,380, WO10/066,780 and WO10/102,758.

Moreover, ketal-substituted 1-H-arylpyrrolidine-2,4-diones are known from WO 99/16748 and (spiro)-ketal-substituted N-alkoxyalkoxy-substituted arylpyrrolidinediones are known from JP-A-14 205 984 and Ito M. et al. Bioscience, Biotechnology and Biochemistry 67, 1230-1238, (2003). Moreover, WO 06/024411 discloses herbicidal compositions which comprise ketoenols.

It is known that certain substituted Δ³-dihydrofuran-2-one derivatives have herbicidal properties (cf. DE-A-4 014 420). The synthesis of the tetronic acid derivatives used as starting materials (such as, for example, 3-(2-methylphenyl)-4-hydroxy-5 (4 fluorophenyl)-Δ³-dihydrofuranone-(2)) is likewise described in DE-A-4 014 420. Compounds of a similar structure are known from the publication Campbell et al., J. Chem. Soc., Perkin Trans. 1, 1985 (8) 1567-76, without any insecticidal and/or acaricidal activity being stated. 3-Aryl-Δ³-dihydrofuranone derivatives having herbicidal, acaricidal and insecticidal properties are furthermore known from: EP-A-528 156, EP-A-647 637, WO 95/26 954, WO 96/20 196, WO 96/25 395, WO 96/35 664, WO 97/01 535, WO 97/02 243, WO 97/36 868, WO 98/05 638, WO 98/06 721, WO 99/16 748, WO 98/25 928, WO 99/43 649, WO 99/48 869, WO 99/55 673, WO 01/23354, WO 01/74 770, WO 01/17 972, WO 04/024 688, WO 04/080 962, WO 04/111 042, WO 05/092 897, WO 06/000 355, WO 06/029 799, WO 07/048,545, WO 07/073,856, WO 07/096,058, WO 07/121,868, WO 07/140,881, WO 08/067,911, WO 08/083,950, WO 09/015,801, WO 09/039,975 and PCT/EP2010/003020.

3-Aryl-Δ³-dihydrothiophenone derivatives. too, are known from WO 95/26 345, 96/25 395, WO 97/01 535, WO 97/02 243, WO 97/36 868, WO 98/05638, WO 98/25928, WO 99/16748, WO 99/43649, WO 99/48869, WO 99/55673, WO 01/17972, WO 01/23354, WO 01/74770, WO 03/013249, WO 04/080 962, WO 04/111 042, WO 05/092897, WO 06/029799 and WO 07/096,058.

Certain phenylpyrone derivatives which are unsubstituted in the phenyl ring are already known (cf. A. M. Chirazi, T. Kappe and E. Ziegler, Arch. Pharm. 309, 558 (1976) and K.-H. Boltze and K. Heidenbluth, Chem. Ber. 91, 2849). Phenylpyrone derivatives which are substituted in the phenyl ring and have herbicidal, acaricidal and insecticidal properties are described in EP-A-588 137, WO 96/25 395, WO 96/35 664, WO 97/01 535, WO 97/02 243, WO 97/16 436, WO 97/19 941, WO 97/36 868, WO 98/05638, WO 99/43649, WO 99/48869, WO 99/55673, WO 01/17972, WO 01/74770, WO 03/013249, WO 04/080 962, WO 04/111 042, WO 05/092897, WO 06/029799 and WO 07/096,058.

Certain 5-phenyl-1,3-thiazine derivatives which are unsubstituted in the phenyl ring are already known (cf. E. Ziegler and E. Steiner, Monatsh. 95, 147 (1964), R. Ketcham, T. Kappe and E. Ziegler, J. Heterocycl. Chem. 10, 223 (1973)). 5-Phenyl-1,3-thiazine derivatives which are substituted in the phenyl ring and have herbicidal, acaricidal and insecticidal properties are described in WO 94/14 785, WO 96/25 395, WO 96/35 664, WO 97/01 535, WO 97/02 243, WO 97/02 243, WO 97/36 868, WO 99/43649, WO 99/48869, WO 99/55673, WO 01/17972, WO 01/74770, WO 03/013249, WO 04/080 962, WO 04/111 042, WO 05/092897, WO 06/029799 and WO 07/096,058.

It is known that certain substituted 2-arylcyclopentanediones have herbicidal, insecticidal and acaricidal properties (cf., for example, U.S. Pat. Nos. 4,283,348; 4,338,122; 4,436,666; 4,526,723; 4,551,547; 4,632,698; WO 96/01 798; WO 96/03 366, WO 97/14 667 and also WO 98/39281, WO 99/43649, WO99/48869, WO 99/55673, WO 01/17972, WO 01/74770, WO 03/062244, WO 04/080962, WO04/111042, WO05/092897, WO06/029799, WO07/080,066, WO07/096,058, WO09/019,005, WO09/019,015, WO09/049,851, WO 10/069,834, WO10/000,773, WO10/057,880, WO10/081,894, WO10/089,210, WO10/102,848 and WO10/133,232. Compounds having similar substitutions are also known; 3-hydroxy-5,5-dimethyl-2-phenylcyclopent 2 en 1 one from the publication Micklefield et al., Tetrahedron, (1992), 7519-26 and also the natural compound involutin (−)cis 5 (3,4-dihydroxyphenyl)-3,4-dihydroxy 2 (4 hydroxyphenyl)cyclopent-2-enone from the publication Edwards et al., J. Chem. Soc. S, (1967), 405-9. An insecticidal or acaricidal action is not described. Moreover, 2-(2,4,6-trimethylphenyl)-1,3-indanedione is known from the publication J. Economic Entomology, 66, (1973), 584 and the laid-open application DE-A 2 361 084, with herbicidal and acaricidal activities being stated.

It is known that certain substituted 2-arylcyclohexanediones have herbicidal, insecticidal and acaricidal properties (U.S. Pat. Nos. 4,175,135, 4,256,657, 4,256,658, 4,256,659, 4,257,858, 4,283,348, 4,303,669, 4,351,666, 4,409,153, 4,436,666, 4,526,723, 4,613,617, 4,659,372, DE-A 2 813 341, and also Wheeler, T. N., J. Org. Chem. 44, 4906 (1979)), WO 99/43649, WO 99/48869, WO 99/55673, WO 01/17972, WO 01/74770, WO 03/013249, WO 04/080 962, WO 04/111 042, WO 05/092897, WO 06/029799, WO 07/096,058, WO 08/071,405, WO 08/110,307, WO 08/110,308, WO 09/074,314, WO 08/145,336, WO 09/015,887, WO09/074,314, WO10/046,194, WO10/081,755 and WO10/089,211.

It is known that certain substituted 4-arylpyrazolidine-3,5-diones have acaricidal, insecticidal and herbicidal properties (cf., for example, WO 92/16 510, EP-A-508 126, WO 96/11 574, WO 96/21 652, WO 99/47525, WO 01/17 351, WO 01/17 352, WO 01/17 353, WO 01/17 972, WO 01/17 973, WO 03/028 466, WO 03/062 244, WO 04/080 962, WO 04/111 042, WO 05/005428, WO 05/016873, WO 05/092897, WO 06/029799 and WO 07/096,058).

It is known that certain tetrahydropyridones have herbicidal properties (JP 0832530). Specific 4-hydroxytetrahydropyridones having acaricidal, insecticidal and herbicidal properties are also known (JP 11152273). Furthermore, 4-hydroxytetrahydropyridones have been disclosed as pesticides and herbicides in WO 01/79204 and WO 07/096,058. 4-Hydroxyquinolones are disclosed in WO 03/01045.

It is known that certain 5,6-dihydropyrone derivatives as protease inhibitors have antiviral properties (WO 95/14012). Furthermore, 4-phenyl 6 (2 phenethyl)-5,6-dihydropyrone is known from the synthesis of kawalactone derivatives (Kappe et al., Arch. Pharm. 309, 558-564 (1976)). Moreover, 5,6-dihydropyrone derivatives are known as intermediates (White, J. D., Brenner, J. B., Deinsdale, M. J., J. Amer. Chem. Soc. 93, 281-282 (1971)). 3-Phenyl-5,6-dihydropyrone derivatives with applications in crop protection are described in WO 01/98288 and WO 07/09658.

4′-Biphenyl-substituted tetronic acid derivatives for the therapy of viral disorders are disclosed in WO 2008/022725.

WO 2005/089118 and WO2007/039286 disclose, in a general manner, nitrogenous bicyclic structures for therapy, 5′-biphenyl-substituted cyclic ketoenols not being specifically mentioned.

4-Phenyl-substituted [1.2]-oxazine-3,5-diones as herbicides were initially described in WO 01/17972. Furthermore, 4-acyl-substituted [1.2]-oxazine-3,5-diones as pesticides, but especially as herbicides and growth regulators, are described, for example, in EP-A-39 48 89; WO 92/07837, U.S. Pat. No. 5,728,831, and as herbicides and pesticides in WO 03/048138.

Based on this prior art, it was an object of the present invention to provide novel structures for the therapy of disorders. The structures according to the invention should be suitable in particular for the prophylaxis and therapy of tumour disorders and have advantages compared to the structures known from the prior art.

Surprisingly, it has now been found that a specific sub-group of the aryl-substituted cyclic ketoenols described in the prior art also inhibits human ACC and is suitable for the therapy of disorders.

Here, it was unforeseeable whether and which of the structures known as insecticides or herbicides would achieve the object of the invention, that is to say to provide structures which can be used in the therapy of human disorders.

The applicant is unaware of any 5′-biphenyl-substituted cyclic ketoenols in accordance with the present invention being described in the prior art for the therapy of disorders, in particular not for the therapy of tumour disorders.

The object is achieved by compounds of the formula (I).

It has now been found that compounds of the formula (I)

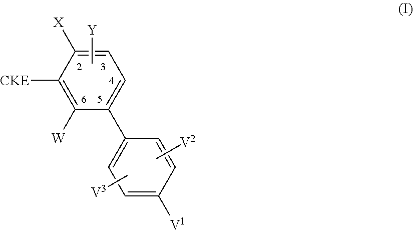

in which
X represents halogen, nitro or cyano or
   represents an optionally monohalogen- or polyhalogen-substituted C₁-C₆-alkyl, C₁-C₆-alkoxy, C₁-C₆-alkoxy-C₁-C₆-alkoxy, C₃-C₇-cycloalkyl or a C₃-C₇-cycloalkyl-C₁-C₆-alkoxy radical, and
W and Y independently of one another represent hydrogen, nitro, cyano or halogen or represent an optionally monohalogen- or polyhalogen-substituted C₁-C₆-alkyl, C₁-C₆-alkoxy or C₃-C₇-cycloalkyl radical, and
V¹, V² and V³ independently of one another represent hydrogen, halogen, nitro or cyano or represent a C₁-C₆-alkyl, halo-C₁-C₆-alkyl, C₁-C₆-alkoxy, halo-C₁-C₆-alkoxy, C₁-C₆-alkylthio, C₁-C₆-alkylsulphinyl, C₁-C₆-alkylsulphonyl, C₁-C₆-alkoxy-C₁-C₆-alkyl, C₃-C₁₀-cycloalkyl radical or represent a monocyclic heterocycloalkyl radical, and/or
V¹ and V² together with the carbon atoms to which they are attached form a saturated or unsaturated cycle T¹ which optionally contains at least one further heteroatom and has 4 to 7 ring atoms and whose ring-forming atoms may be mono- or polysubstituted by identical or different substituents selected from the group consisting of halogen and a C₁-C₆-alkyl radical,
CKE represents one of the groups

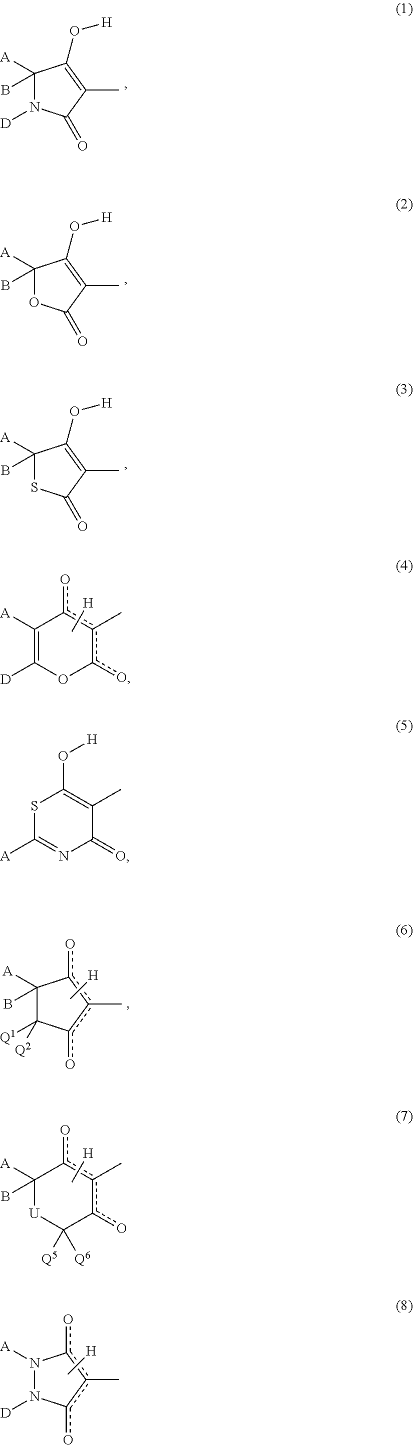

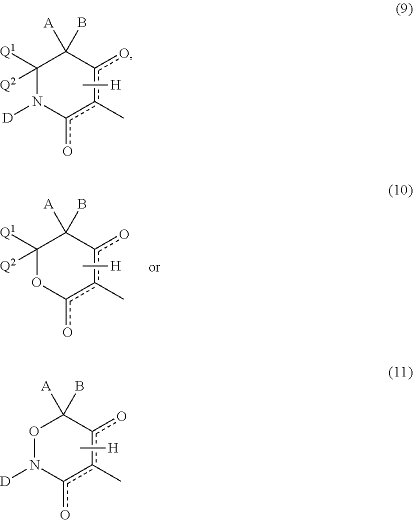

in which
U represents —S—, —S(O)—, —S(O)₂—, —O—,

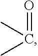

a substituted

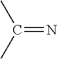

group
or represents a C₁-C₄-alkylene group which is optionally substituted by Q³ and Q⁴, and
A represents hydrogen or
   represents an optionally monohalogen- or polyhalogen-substituted C₁-C₆-alkyl, C₂-C₆-alkenyl, C₁-C₆-alkoxy-C₁-C₆-alkyl or C₁-C₆-alkylthio-C₁-C₆-alkyl radical or
   represents a C₃-C₇-cycloalkyl, C₃-C₇-cycloalkyl-C₁-C₄-alkyl or monocyclic heterocyclyl or heterocyclyl-C₁-C₄-alkyl radical,
   each of which may be mono- or polysubstituted by identical or different substituents selected from the group consisting of halogen and a C₁-C₆-alkyl radical or
   represents an aryl, aryl-C₁-C₆-alkyl or heteroaryl radical, each of which may optionally be mono- or polysubstituted by identical or different substituents selected from the group consisting of halogen, cyano, nitro and C₁-C₆-alkyl, halo-C₁-C₆-alkyl, C₁-C₆-alkoxy and halo-C₁-C₆-alkoxy radicals and
B represents hydrogen or represents a C₁-C₆-alkyl or C₁-C₆-alkoxy-C₁-C₆-alkyl radical, or
A and B together with the carbon atom to which they are attached form a saturated or unsaturated cycle T² which optionally contains at least one heteroatom and has 3 to 8 ring atoms and whose ring-forming atoms may be mono- or polysubstituted by identical or different substituents selected from the group consisting of the radicals R¹, R² and R³,
   where R¹, R² and R³ independently of one another
   a) represent halogen, hydroxyl or cyano or
   b) represent a C₁-C₆-alkyl, C₁-C₆-alkoxy, C₁-C₆-alkoxy-C₁-C₆-alkyl, C₁-C₆-alkylcarbonyl,
      C₁-C₆-alkoxycarbonyl, C₁-C₆-alkylaminocarbonyl, C₁-C₆-alkylthio, C₁-C₆-alkylsulphinyl, C₁-C₆-alkylsulphonyl, C₁-C₆-alkylaminosulphonyl, C₁-C₆-alkoxy-C₁-C₆-alkoxy, halo-C₁-C₆-alkyl or halo-C₁-C₆-alkoxy radical which is optionally hydroxyl-substituted in the alkyl moiety, or
   c) represent an aryl, arylcarbonyl, arylsulphonyl, arylamino, heteroaryl, heteroarylcarbonyl, heteroarylsulphonyl or heteroarylamino radical, or
   d) represent a C₃-C₇-cycloalkyl, C₃-C₇-cycloalkylcarbonyl, C₃-C₇-cycloalkylsulphonyl, heterocyclyl, heterocyclylcarbonyl or heterocyclylsulphonyl radical,
      where the radicals mentioned under c) and d) may optionally be mono- or polysubstituted at the ring system by identical or different substituents selected from the group consisting of halogen, hydroxyl, cyano, nitro and C₁-C₆-alkyl, C₁-C₆-alkoxy, halo-C₁-C₆-alkyl, halo-C₁-C₆-alkoxy, C₁-C₆-alkoxy-C₁-C₆-alkyl, C₃-C₁₀-cycloalkyl and 3- to 6-membered heterocycloalkyl radicals, and/or
   e) two of the radicals R¹, R² and R³ together with the ring atom(s) of the cycle T² to which they are attached may form a further saturated or unsaturated cycle T³ which optionally contains at least one heteroatom and has 3 to 7 ring atoms and may be mono- or polysubstituted by identical or different substituents selected from the group consisting of the radicals R⁴, R⁵ and R⁶, where R⁴, R⁵ and R⁶ independently of one another represent a C₁-C₆-alkyl or C₁-C₆-alkoxy radical, and
D represents hydrogen or
   represents a C₁-C₆-alkyl, C₂-C₆-alkenyl, C₂-C₆-alkynyl or C₁-C₆-alkoxy-C₁-C₆-alkyl radical or
   represents a C₃-C₇-cycloalkyl, C₃-C₇-cycloalkyl-C₁-C₄-alkyl or monocyclic heterocyclyl or heterocyclyl-C₁-C₄-alkyl radical or
   represents an aryl, aryl-C₁-C₆-alkyl, heteroaryl or heteroaryl-C₁-C₆-alkyl radical, where the radicals mentioned may optionally be mono- or polysubstituted by identical or different substituents selected from the group consisting of halogen, hydroxyl, cyano, nitro and C₁-C₆-alkyl, halo-C₁-C₆-alkyl, C₁-C₆-alkoxy, halo-C₁-C₆-alkoxy, C₁-C₆-alkoxy-C₁-C₆-alkyl, C₃-C₁₀-cycloalkyl and monocyclic heterocycloalkyl radicals, or
A and D together with the atoms to which they are attached form a saturated or unsaturated cycle T⁴ which optionally contains at least one further heteroatom and has 3 to 7 ring atoms, which may be bridged and whose ring-forming atoms may be mono- or polysubstituted by identical or different substituents selected from the group consisting of the radicals R⁷, R⁸ and R⁹,
   where R⁷, R⁸ and R⁹ independently of one another represent hydroxyl, halogen or represent a C₁-C₆-alkyl or C₁-C₆-alkoxy radical, and
A and Q¹ together with the atoms to which they are attached form a saturated or unsaturated cycle T⁵ which optionally contains at least one further heteroatom and has 5 to 7 ring atoms and whose ring-forming atoms may be mono- or polysubstituted by identical or different substituents selected from the group consisting of halogen, hydroxyl, cyano, nitro and C₁-C₆-alkyl, halo-C₁-C₆-alkyl, C₁-C₆-alkoxy, halo-C₁-C₆-alkoxy, C₁-C₆-alkoxy-C₁-C₆-alkyl and C₃-C₁₀-cycloalkyl radicals,
   with the proviso that B and Q² represent a bond if the cycle T⁵ formed by A and Q¹ is aromatic,
Q¹ represents hydrogen or represents a C₁-C₆-alkyl or C₁-C₆-alkoxy radical which is optionally mono- or polysubstituted by identical or different substituents selected from the group consisting of halogen, hydroxyl and a C₁-C₆-alkoxy radical or
   represents a C₃-C₇-cycloalkyl, C₃-C₇-cycloalkyl-C₁-C₄-alkyl or monocyclic heterocyclyl or heterocyclyl-C₁-C₄-alkyl radical,
   each of which may optionally be mono- or polysubstituted by identical or different substituents selected from the group consisting of halogen, hydroxyl and C₁-C₆-alkyl, halo-C₁-C₆-alkyl, C₁-C₆-alkoxy, C₁-C₆-alkoxy-C₁-C₆-alkoxy and halo-C₁-C₆-alkoxy radicals or
   represents a phenyl radical which may optionally be mono- or polysubstituted by identical or different substituents selected from the group consisting of halogen, hydroxyl, cyano, nitro and C₁-C₆-alkyl, halo-C₁-C₆-alkyl, C₁-C₆-alkoxy, halo-C₁-C₆-alkoxy, C₁-C₆-alkoxy-C₁-C₆-alkyl and C₃-C₁₀-cycloalkyl radicals, and
Q², Q⁴, Q⁵ and Q⁶ independently of one another represent hydrogen or represent a C₁-C₆-alkyl radical, and
Q³ represents hydrogen or
   represents a C₁-C₆-alkyl or C₁-C₆-alkoxy radical which is optionally mono- or polysubstituted by identical or different substituents selected from the group consisting of halogen, hydroxyl and a C₁-C₆-alkoxy radical or
   represents a C₃-C₇-cycloalkyl, C₃-C₇-cycloalkyl-C₁-C₄-alkyl or a monocyclic heterocyclyl or heterocyclyl-C₁-C₄-alkyl radical,
   each of which may optionally be mono- or polysubstituted by identical or different substituents selected from the group consisting of halogen, hydroxyl and C₁-C₆-alkyl, halo-C₁-C₆-alkyl, C₁-C₆-alkoxy, C₁-C₆-alkoxy-C₁-C₆-alkoxy and halo-C₁-C₆-alkoxy radicals or
   represents a phenyl radical which may optionally be mono- or polysubstituted by identical or different substituents selected from the group consisting of halogen, hydroxyl, cyano, nitro and C₁-C₆-alkyl, halo-C₁-C₆-alkyl, C₁-C₆-alkoxy, halo-C₁-C₆-alkoxy, C₁-C₆-alkoxy-C₁-C₆-alkyl and C₃-C₁₀-cycloalkyl radicals, or
Q¹ and Q² together with the carbon atom to which they are attached form a saturated or unsaturated cycle T⁶ which optionally contains at least one further heteroatom having 3 to 7 ring atoms,
   whose ring-forming atoms may be mono- or polysubstituted by identical or different substituents selected from the group consisting of halogen, hydroxyl, cyano, nitro and C₁-C₆-alkyl, halo-C₁-C₆-alkyl, C₁-C₆-alkoxy, halo-C₁-C₆-alkoxy, C₁-C₆-alkoxy-C₁-C₆-alkyl and C₃-C₁₀-cycloalkyl radicals, or
Q³ and Q⁴ together with the carbon atom to which they are attached form a saturated or unsaturated cycle T⁷ which optionally contains at least one heteroatom and has 3 to 7 ring atoms and whose ring-forming atoms may be mono- or polysubstituted by identical or different substituents selected from the group consisting of halogen, hydroxyl, cyano, nitro and C₁-C₆-alkyl, halo-C₁-C₆-alkyl, C₁-C₆-alkoxy, halo-C₁-C₆-alkoxy, C₁-C₆-alkoxy-C₁-C₆-alkyl and C₃-C₁₀-cycloalkyl radicals,
   are suitable for use as a medicament.

The medicaments are suitable for the prophylaxis and/or therapy of human or animal disorders, in particular for the prophylaxis and/or therapy of tumour disorders.

The compounds according to the invention are particularly suitable for the prophylaxis and/or therapy of cancer.

Accordingly, the present invention provides 5′-biphenyl-substituted ketoenols of the formula (I) for therapeutic purposes, pharmaceutical compositions and their use in therapy, in particular for the prophylaxis and/or therapy of tumour disorders.

The therapy of disorders is preferred.

The invention is based on the following definitions:

### Alkyl:

Alkyl represents a straight-chain or branched saturated monovalent hydrocarbon radical having generally 1 to 6 (C₁-C₆-alkyl), preferably 1 to 4 (C₁-C₄-alkyl), and particularly preferably 1 to 3 carbon atoms (C₁-C₃-alkyl).

The following may be mentioned by way of example and by way of preference:

methyl, ethyl, propyl, butyl, pentyl, hexyl, isopropyl, isobutyl, sec-butyl, ten-butyl, isopentyl, 2-methylbutyl, 1-methylbutyl, 1-ethylpropyl, 1,2-dimethylpropyl, neo-pentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl, 1,2-dimethylbutyl.

Particular preference is given to a methyl, ethyl, propyl or isopropyl radical.

Alkylene=alkanediyl:

Alkylene represents a straight-chain or branched saturated divalent hydrocarbon radical having generally 1 to 6 (C₁-C₆-alkylene), preferably 1 to 4 (C₁-C₄-alkylene), and particularly preferably 1 to 3 (C₁-C₃-alkylene) carbon atoms.

The following may be mentioned by way of example and by way of preference:

methylene, ethylene, propylene, butylene, pentylene, hexylene, isopropylene, isobutylene, sec-butylene, tert-butylene, isopentylene, 2-methylbutylene, 1-methylbutylene, 1-ethylpropylene, 1,2-dimethylpropylene, neo-pentylene, 1,1-dimethylpropylene, 4-methylpentylene, 3-methylpentylene, 2-methylpentylene, 1-methylpentylene, 2-ethylbutylene, 1-ethylbutylene, 3,3-dimethylbutylene, 2,2-dimethylbutylene, 1,1-dimethylbutylene, 2,3-dimethylbutylene, 1,3-dimethylbutylene, 1,2-dimethylbutylene.

Particular preference is given to methylene, ethylene or propylene.

### Alkenyl:

Alkenyl represents a straight-chain or branched monovalent hydrocarbon radical having at least one double bond and generally 2 to 6 (C₂-C₆-alkenyl), preferably 2 to 4 (C₂-C₄-alkenyl), and particularly preferably 2 or 3 (C₂-C₃-alkenyl) carbon atoms.

The following may be mentioned by way of example and by way of preference:

vinyl, allyl, (E)-2-methylvinyl, (Z)-2-methylvinyl, homoallyl, (E)-but-2-enyl, (Z) but 2 enyl, (E)-but-1-enyl, (Z)-but-1-enyl, pent-4-enyl, (E)-pent-3-enyl, (Z)-pent-3-enyl, (E)-pent-2-enyl, (Z)-pent-2-enyl, (E)-pent-1-enyl, (Z)-pent-1-enyl, hex-5-enyl, (E)-hex-4-enyl, (Z)-hex-4-enyl, (E)-hex-3-enyl, (Z)-hex-3-enyl, (E)-hex-2-enyl, (Z)-hex-2-enyl, (E)-hex-1-enyl, (Z)-hex-1-enyl, isopropenyl, 2-methylprop-2-enyl, 1-methylprop-2-enyl, 2-methylprop-1-enyl, (E)-1-methylprop-1-enyl, (Z)-1-methylprop-1-enyl, 3-methylbut-3-enyl, 2-methylbut-3-enyl, 1-methylbut-3-enyl, 3-methylbut-2-enyl, (E)-2-methylbut-2-enyl, (Z)-2-methylbut-2-enyl, (E)-1-methylbut-2-enyl, (Z)-1-methylbut-2-enyl, (E)-3-methylbut-1-enyl, (Z)-3-methylbut-1-enyl, (E)-2-methylbut-1-enyl, (Z)-2-methylbut-1-enyl, (E)-1-methylbut-1-enyl, (Z)-1-methylbut-1-enyl, 1,1-dimethylprop-2-enyl, 1-ethylprop-1-enyl, 1-propylvinyl, 1-isopropylvinyl, 4-methylpent-4-enyl, 3-methylpent-4-enyl, 2-methylpent-4-enyl, 1-methylpent-4-enyl, 4-methylpent-3-enyl, (E)-3-methylpent-3-enyl, (Z)-3-methylpent-3-enyl, (E)-2-methylpent-3-enyl, (Z)-2-methylpent-3-enyl, (E)-1-methylpent-3-enyl, (Z)-1-methylpent-3-enyl, (E)-4-methylpent-2-enyl, (Z)-4-methylpent-2-enyl, (E)-3-methylpent-2-enyl, (Z)-3-methylpent-2-enyl, (E)-2-methylpent-2-enyl, (Z)-2-methylpent-2-enyl, (E)-1-methylpent-2-enyl, (Z)-1-methylpent-2-enyl, (E)-4-methylpent-1-enyl, (Z)-4-methylpent-1-enyl, (E)-3-methylpent-1-enyl, (Z)-3-methylpent-1-enyl, (E)-2-methylpent-1-enyl, (Z)-2-methylpent-1-enyl, (E)-1-methylpent-1-enyl, (Z)-1-methylpent-1-enyl, 3-ethylbut-3-enyl, 2-ethylbut-3-enyl, 1-ethylbut-3-enyl, (E)-3-ethylbut-2-enyl, (Z)-3-ethylbut-2-enyl, (E)-2-ethylbut-2-enyl, (Z)-2-ethylbut-2-enyl, (E)-1-ethylbut-2-enyl, (Z)-1-ethylbut-2-enyl, (E)-3-ethylbut-1-enyl, (Z)-3-ethylbut-1-enyl, 2-ethylbut-1-enyl, (E)-1-ethylbut-1-enyl, (Z)-1-ethylbut-1-enyl, 2-propylprop-2-enyl, 1-propylprop-2-enyl, 2-isopropylprop-2-enyl, 1-isopropylprop-2-enyl, (E)-2-propylprop-1-enyl, (Z)-2-propylprop-1-enyl, (E)-1-propylprop-1-enyl, (Z)-1-propylprop-1-enyl, (E)-2-isopropylprop-1-enyl, (Z)-2-isopropylprop-1-enyl, (E)-1-isopropylprop-1-enyl, (Z)-1-isopropylprop-1-enyl, (E)-3,3-dimethylprop-1-enyl, (Z)-3,3-dimethylprop-1-enyl, 1-(1,1-dimethylethyl)ethenyl, buta-1,3-dienyl, penta-1,4-dienyl, hexa-1,5-dienyl, methylhexadienyl. Particular preference is given to vinyl or allyl.
Alkenylene=alkenediyl:

Alkenylene represents a straight-chain or branched divalent hydrocarbon radical having at least one double bond and generally 2 to 6 (C₂-C₆-alkenylene), preferably 2 to 4 (C₂-C₄-alkenylene), and particularly preferably 2 or 3 (C₂-C₃-alkenylene) carbon atoms.

### Alkynyl:

Alkynyl represents a straight-chain or branched monovalent hydrocarbon radical having at least one triple bond and generally 2 to 6 (C₂-C₆-alkynyl), preferably 2 to 4 (C₂-C₄-alkynyl), and particularly preferably 2 or 3 (C₂-C₃-alkynyl) carbon atoms.

The following may be mentioned by way of example and by way of preference:

ethynyl, prop-1-ynyl, prop-2-ynyl, but-1-ynyl, but-2-ynyl, but-3-ynyl, pent-1-ynyl, pent-2-ynyl, pent-3-ynyl, pent-4-ynyl, hex-1-ynyl, hex-2-ynyl, hex-3-ynyl, hex-4-ynyl, hex-5-ynyl, 1-methylprop-2-ynyl, 2-methylbut-3-ynyl, 1-methylbut-3-ynyl, 1-methylbut-2-ynyl, 3-methylbut-1-ynyl, 1-ethylprop-2-ynyl, 3-methylpent-4-ynyl, 2-methylpent-4-ynyl, 1-methylpent-4-ynyl, 2-methylpent-3-ynyl, 1-methylpent-3-ynyl, 4-methylpent-2-ynyl, 1-methylpent-2-ynyl, 4-methylpent-1-ynyl, 3-methylpent-1-ynyl, 2-ethylbut-3-ynyl, 1-ethylbut-3-ynyl, 1-ethylbut-2-ynyl, 1-propylprop-2-ynyl, 1-isopropylprop-2-ynyl, 2,2-dimethylbut-3-ynyl, 1,1-dimethylbut-3-ynyl, 1,1-dimethylbut-2-ynyl or 3,3-dimethylbut-1-ynyl.

Particular preference is given to ethynyl, prop-1-ynyl or prop-2-ynyl.

### Cycloalkyl:

Cycloalkyl represents a mono- or bicyclic saturated monovalent hydrocarbon radical having generally 3 to 10 (C₃-C₁₀-cycloalkyl), preferably 3 to 8 (C₃-C₈-cycloalkyl), and particularly preferably 3 to 7 (C₃-C₇-cycloalkyl) carbon atoms.

The following may be mentioned by way of example and by way of preference for monocyclic cycloalkyl radicals:

cyclopropyl, cyclobutyl, cyclopentyl, cyclohexyl and cycloheptyl.

Particular preference is given to a cyclopropyl, a cyclopentyl or a cyclohexyl radical. The following may be mentioned by way of example for bicyclic cycloalkyl radicals:

perhydropentalenyl, decalinyl.

### Cycloalkylcarbonyl

Cycloalkylcarbonyl represents the group C(O)-cycloalkyl.

### Cycloalkylsulphonyl

Cycloalkylsulphonyl represents the group S(O)₂-cycloalkyl.

### Cycloalkylalkyl:

Cycloalkylalkyl represents an alkyl radical which is substituted by a cyclic saturated hydrocarbon ring.

Here, Cₙ-cycloalkyl-Cₘ-alkyl means that the alkyl moiety has m carbon atoms and the cycloalkyl moiety has n carbon atoms.

The following may be mentioned by way of example and by way of preference:

cyclopropylmethyl, cyclobutylethyl, cyclopentylethyl.

### Cycloalkylalkoxy:

Cycloalkylalkoxy represents an alkoxy radical which is substituted by a cyclic saturated hydrocarbon ring.

Here, Cₙ-cycloalkyl-Cₘ-alkoxy means that the alkoxy moiety has m carbon atoms and the cycloalkyl moiety has n carbon atoms.

The following may be mentioned by way of example and by way of preference:

cyclopropylmethoxy, cyclobutylethoxy, cyclopentylethoxy.

### Alkoxy:

Alkoxy represents a straight-chain or branched saturated alkyl ether radical of the formula O-alkyl having generally 1 to 6 (C₁-C₆-alkoxy), preferably 1 to 4 (C₁-C₄-alkoxy), and particularly preferably 1 to 3 (C₁-C₃-alkoxy) carbon atoms.

The following may be mentioned by way of example and by way of preference:

methoxy, ethoxy, n-propoxy, isopropoxy, tert-butoxy, n-pentoxy and n-hexoxy.

### Alkylthio

Alkylthio represents a straight-chain or branched saturated alkylthio ether radical of the formula —S-alkyl having generally 1 to 6 (C₁-C₆-alkylthio), preferably 1 to 4 (C₁-C₄-alkylthio), and particularly preferably 1 to 3 (C₁-C₃-alkylthio) carbon atoms.

### Alkoxyalkyl

Alkoxyalkyl represents an alkyl radical substituted by alkoxy.

Here, Cₙ-alkoxy-Cₘ-alkyl means that the alkoxy moiety has n carbon atoms and the alkyl moiety through which the radical is attached has m carbon atoms.

The following may be mentioned by way of example and by way of preference:

methoxymethyl, methoxyethyl, ethoxymethyl and ethoxyethyl.

### Alkylthioalkyl

Alkylthioalkyl represents an alkyl radical substituted by alkylthio.

Here, Cₙ-alkylthio-Cₘ-alkyl means that the alkylthio moiety has n carbon atoms and the alkyl moiety through which the radical is attached has m carbon atoms.

### Alkoxyalkoxy

Alkoxyalkoxy represents an alkoxy radical substituted by alkoxy.

Here, Cₙ-alkoxy-Cₘ-alkoxy means that the outer alkoxy moiety has n carbon atoms and the alkoxy moiety through whose oxygen function the radical is attached has m carbon atoms.

The following may be mentioned by way of example and by way of preference:

methoxyethoxy and ethoxyethoxy.

### Alkylamino

Alkylamino represents an amino radical having one or two alkyl substituents (selected independently of one another) having generally 1 to 6, preferably 1 to 3, carbon atoms.

(C₁-C₃)-Alkylamino, for example, represents a monoalkylamino radical having 1 to 3 carbon atoms or represents a dialkylamino radical having in each case 1 to 3 carbon atoms per alkyl substituent.

The following may be mentioned by way of example:

methylamino, ethylamino, n-propylamino, isopropylamino, tert-butylamino, n-pentylamino, n-hexylamino, N,N-dimethylamino, N,N-diethylamino, N-ethyl-N-methylamino, N-methyl-N-n-propylamino, N-isopropyl-N-n-propylamino, N-t-butyl-N-methylamino, N-ethyl-N-n-pentylamino and N-n-hexyl-N-methylamino.

### Alkylcarbonyl

Alkylcarbonyl represents the group C(O)-alkyl having generally 1 to 6, preferably 1 to 4, and particularly preferably 1 to 3 carbon atoms in the alkyl moiety.

The following may be mentioned by way of example:

acetyl and propanoyl.

### Alkoxycarbonyl

Alkoxycarbonyl represents the group C(O)—O-alkyl having generally 1 to 6, preferably 1 to 4, and particularly preferably 1 to 3 carbon atoms in the alkyl moiety.

The following may be mentioned by way of example:

methoxycarbonyl, ethoxycarbonyl, propoxycarbonyl, isopropoxycarbonyl, tert-butoxycarbonyl, n-pentoxycarbonyl and n-hexoxycarbonyl.

### Alkylaminocarbonyl

Alkylaminocarbonyl represents the group C(O)-alkylamino having one or two alkyl substituents (selected independently of one another) having generally 1 to 6, preferably 1 to 3, carbon atoms. (C₁-C₃)-Alkylaminocarbonyl, for example, represents a monoalkylaminocarbonyl radical having 1 to 3 carbon atoms or represents a dialkylaminocarbonyl radical having in each case 1 to 3 carbon atoms per alkyl substituent.

The following may be mentioned by way of example:

methylaminocarbonyl, ethylaminocarbonyl, n-propylaminocarbonyl, isopropylaminocarbonyl, tert-butylaminocarbonyl, n-pentylaminocarbonyl, n-hexylaminocarbonyl, N,N-dimethylaminocarbonyl, N,N-diethylaminocarbonyl, N-ethyl-N-methylaminocarbonyl, N-methyl-N-n-propylaminocarbonyl, N-isopropyl-N-n-propylaminocarbonyl, N-t-butyl-N-methylaminocarbonyl, N-ethyl-N-n-pentylamino carbonyl and N-n-hexyl-N-methylaminocarbonyl.

### Alkylsulphinyl

Alkylsulphinyl represents a straight-chain or branched saturated radical of the formula S(O)-alkyl having generally 1 to 6 (C₁-C₆-alkylsulphinyl), preferably 1 to 4 (C₁-C₄-alkylsulphinyl), and particularly preferably 1 to 3 (C₁-C₃-alkylsulphinyl) carbon atoms.

The following may be mentioned by way of example and by way of preference:

methylsulphinyl, ethylsulphinyl, propylsulphinyl.

### Alkylsulphonyl

Alkylsulphonyl represents a straight-chain or branched saturated radical of the formula S(O)₂-alkyl having generally 1 to 6 (C₁-C₆-alkylsulphonyl), preferably 1 to 4 (C₁-C₄-alkylsulphonyl), and particularly preferably 1 to 3 (C₁-C₃-alkylsulphonyl) carbon atoms.

The following may be mentioned by way of example and by way of preference:

methylsulphonyl, ethylsulphonyl, propylsulphonyl.

### Alkylaminosulphonyl

Alkylaminosulphonyl represents the group S(O)₂-alkylamino having one or two alkyl substituents (selected independently of one another) having generally 1 to 6, preferably 1 to 3, carbon atoms. (C₁-C₃)-Alkylaminosulphonyl, for example, represents a monoalkylaminosulphonyl radical having 1 to 3 carbon atoms or represents a dialkylaminosulphonyl radical having in each case 1 to 3 carbon atoms per alkyl substituent.

The following may be mentioned by way of example:

methylaminosulphonyl, ethylaminosulphonyl, n-propylaminosulphonyl, isopropylaminosulphonyl, tert-butylaminosulphonyl, n-pentylaminosulphonyl, n-hexylaminosulphonyl, N,N-dimethyl-aminosulphonyl, N,N-diethylaminosulphonyl, N-ethyl-N-methylaminosulphonyl, N-methyl-N-n-propylaminosulphonyl, N-isopropyl-N-n-propylaminosulphonyl, N-t-butyl-N-methylaminosulphonyl, N-ethyl-N-n-pentylamino-sulphonyl and N-n-hexyl-N-methylaminosulphonyl.

### Aryl

Aryl is a monovalent aromatic mono- or bicyclic ring system without any heteroatoms having 6 or carbon atoms.

The following may be mentioned by way of example and by way of preference:

phenyl (C₆-aryl), naphthyl (C₁₀-aryl).

Particular preference is given to phenyl.

### Arylcarbonyl

Arylcarbonyl represents the group C(O)-aryl.

### Arylsulphonyl

Arylsulphonyl represents the group S(O)₂-aryl.

### Arylamino

Arylamino represents the group NR-aryl, where R represents hydrogen or alkyl.

### Arylalkyl

Arylalkyl represents a straight-chain or branched saturated monovalent alkyl group substituted by an aromatic mono- or bicyclic ring system without any heteroatoms.

Here, Cₙ-aryl-Cₘ-alkyl means that the alkyl moiety has m carbon atoms and the aryl moiety has n carbon atoms.

The following may be mentioned by way of example and by way of preference:

phenylmethyl, phenylethyl.

### Heteroatoms

Heteroatoms are to be understood as meaning oxygen, nitrogen or sulphur atoms.

### Heteroaryl

Heteroaryl is a monovalent mono- or bicyclic ring system having at least one heteroatom and at least one aromatic ring. The heteroatoms present may be nitrogen atoms, oxygen atoms and/or sulphur atoms. The binding valency may be at any aromatic carbon atom or at a nitrogen atom.

A monocyclic heteroaryl radical in accordance with the present invention has 5 or 6 ring atoms.

Heteroaryl radicals having 5 ring atoms include, for example, the rings:

thienyl, thiazolyl, furyl, pyrrolyl, oxazolyl, imidazolyl, pyrazolyl, isoxazolyl, isothiazolyl, oxadiazolyl, triazolyl, tetrazolyl and thiadiazolyl.

Heteroaryl radicals having 6 ring atoms include, for example, the rings:

pyridyl, pyridazinyl, pyrimidinyl, pyrazinyl and triazinyl.

A bicyclic heteroaryl radical in accordance with the present invention has 9 or 10 ring atoms.

Heteroaryl radicals having 9 ring atoms include, for example, the rings:

phthalidyl, thiophthalidyl, indolyl, isoindolyl, indazolyl, benzothiazolyl, benzofuryl, benzothienyl, benzimidazolyl, benzoxazolyl, azocinyl, indolizinyl, purinyl, indolinyl.

Heteroaryl radicals having 10 ring atoms include, for example, the rings:

isoquinolinyl, quinolinyl, quinolizinyl, quinazolinyl, quinoxalinyl, cinnolinyl, phthalazinyl, 1,7- or 1,8-naphthyridinyl, pteridinyl, chromanyl.

Monocyclic heteroaryl rings having 5 or 6 ring atoms are preferred.

### Heteroarylcarbonyl

Heteroarylcarbonyl represents the group C(O)-heteroaryl.

### Heteroarylsulphonyl

Heteroarylsulphonyl represents the group S(O)₂-heteroaryl.

### Heteroarylamino

Heteroarylamino represents the group NR-heteroaryl, where R represents hydrogen or alkyl.

### Heteroarylalkyl

Heteroarylalkyl represents a straight-chain or branched saturated monovalent alkyl group substituted by an aromatic mono- or bicyclic ring system having at least one heteroatom different from carbon.

Here, monocyclic heteroaryl-Cₘ-alkyl means that the alkyl moiety has m carbon atoms and the heteroaryl moiety is monocyclic and therefore has 5 or 6 ring atoms.

### Heterocyclyl

Heterocyclyl for the purpose of the invention is a non-aromatic mono- or bicyclic ring system having at least one heteroatom or a hetero group. The heteroatoms present may be nitrogen atoms, oxygen atoms and/or sulphur atoms. The hetero groups present may be S(O)—, —S(O)₂— or —N⁺(O⁻)—. A monocyclic heterocyclyl ring in accordance with the present invention may have 3 to 8, preferably 5 to 8, particularly preferably 5 or 6, ring atoms.

The following may be mentioned by way of example and by way of preference for monocyclic heterocyclyl radicals having 3 ring atoms:

aziridinyl.

The following may be mentioned by way of example and by way of preference for monocyclic heterocyclyl radicals having 4 ring atoms:

azetidinyl, oxetanyl.

The following may be mentioned by way of example and by way of preference for monocyclic heterocyclyl radicals having 5 ring atoms:

pyrrolidinyl, imidazolidinyl, pyrazolidinyl, pyrrolinyl, dioxolanyl and tetrahydrofuranyl.

The following may be mentioned by way of example and by way of preference for monocyclic heterocyclyl radicals having 6 ring atoms:

piperidinyl, piperazinyl, morpholinyl, dioxanyl, tetrahydropyranyl and thiomorpholinyl.

The following may be mentioned by way of example and by way of preference for monocyclic heterocyclyl radicals having 7 ring atoms:

azepanyl, oxepanyl, [1,3]-diazepanyl, [1,4]-diazepanyl.

The following may be mentioned by way of example and by way of preference for monocyclic heterocyclyl radicals having 8 ring atoms:

oxocanyl, azocanyl.

A bicyclic heterocyclyl radical in accordance with the present invention may have 5 to 12, preferably 8 to 10, ring atoms.

Preference is given to 5- to 8-membered monocyclic saturated heterocyclyl radicals having up to two heteroatoms from the group consisting of O, N and S.

Particular preference is given to morpholinyl, piperidinyl and pyrrolidinyl.

### Heterocylylalkyl

Heterocyclylalkyl represents an alkyl radical which is substituted by a heterocyclyl radical. Here, monocyclic heterocyclyl-Cₘ-alkyl radical means, for example, that the alkyl moiety has m carbon atoms and the heterocyclyl moiety has 3 to 8 ring atoms.

### Heterocyclylcarbonyl

Heterocyclylcarbonyl represents the group C(O)-heterocyclyl.

### Heterocyclylsulphonyl

Heterocyclylsulphonyl represents the group S(O)₂-heterocyclyl.

### Halogen

The term halogen includes fluorine, chlorine, bromine and iodine.

Preference is given to fluorine and chlorine.

### Haloalkyl:

Haloalkyl represents an alkyl radical having at least one halogen substituent.

The following may be mentioned by way of example and by way of preference:

difluoromethyl, trifluoromethyl, 2,2,2-trifluoroethyl, pentafluoroethyl, 5,5,5,4,4-pentafluoropentyl or 5,5,5,4,4,3,3-heptafluoropentyl.

Preference is given to perfluorinated alkyl radicals such as trifluoromethyl or pentafluoroethyl.

### Haloalkoxy

Haloalkoxy represents an alkoxy radical having at least one halogen substituent.

Preference is given to fluoroalkoxy radicals.

The following may be mentioned by way of example and by way of preference:

difluoroethoxy, trifluoromethoxy or 2,2,2-trifluoroethoxy.

### Cycle

Cycle includes all ring systems.

### Unsaturated Cycle

An unsaturated cycle includes ring systems having at least one double bond in the ring and aromatic ring systems.

A radical which may be mono-, di- or polysubstituted is a radical without any substituents or with one, two or more than two substituents.

In formula (I), X may represent:

halogen, nitro or cyano or
an optionally monohalogen- or polyhalogen-substituted C₁-C₆-alkyl, C₁-C₆-alkoxy, C₁-C₆-alkoxy-C₁-C₆-alkoxy, C₃-C₇-cycloalkyl or a C₃-C₇-cycloalkyl-C₁-C₆-alkoxy radical.

In formula (I), X may preferably represent:

halogen or an optionally monohalogen- or polyhalogen-substituted C₁-C₃-alkyl or C₁-C₃-alkoxy radical.

In formula (I), W and Y independently of one another may represent:

hydrogen, nitro, cyano or halogen or
an optionally monohalogen- or polyhalogen-substituted C₁-C₆-alkyl, C₁-C₆-alkoxy or C₃-C₇-cycloalkyl radical.

In formula (I), W and Y independently of one another may preferably represent:

hydrogen, cyano or halogen or
an optionally monohalogen- or polyhalogen-substituted C₁-C₃-alkyl or C₁-C₃-alkoxy radical.

In formula (I), W and Y independently of one another may more preferably represent:

hydrogen or halogen or
an optionally monohalogen- or polyhalogen-substituted C₁-C₃-alkyl or C₁-C₃-alkoxy radical.

In formula (I), W and Y independently of one another may particularly preferably represent:

hydrogen or an optionally monohalogen- or polyhalogen-substituted C₁-C₃-alkyl radical.

In formula (I), V¹, V² and V³ independently of one another may represent:

hydrogen, halogen, nitro or cyano or a C₁-C₆-alkyl, halo-C₁-C₆-alkyl, C₁-C₆-alkoxy, halo-C₁-C₆-alkoxy, C₁-C₆-alkylthio, C₁-C₆-alkylsulphinyl, C₁-C₆-alkylsulphonyl, C₁-C₆-alkoxy-C₁-C₆-alkyl, C₃-C₁₀-cycloalkyl or a monocyclic heterocycloalkyl radical, and/or
V¹ and V² together with the carbon atoms to which they are attached form a saturated or unsaturated cycle T¹ which optionally contains at least one further heteroatom and has 4 to 7 ring atoms and whose ring-forming atoms may be mono- or polysubstituted by identical or different substituents selected from the group consisting of halogen and a C₁-C₆-alkyl radical.

In formula (I), V¹, V² and V³ independently of one another may preferably represent:

hydrogen, halogen or cyano or a C₁-C₃-alkyl, C₁-C₃-haloalkyl, C₁-C₃-alkoxy, C₁-C₃-haloalkoxy or C₁-C₃-alkoxy-C₁-C₃-alkyl radical, and/or
V¹ and V² together with the carbon atoms to which they are attached form a saturated or unsaturated cycle T¹ which optionally contains at least one further heteroatom and has 5 or 6 ring atoms and whose ring-forming atoms may be mono- or polysubstituted by identical or different substituents selected from the group consisting of halogen and a C₁-C₃-alkyl radical.

In formula (I), V¹, V² and V³ independently of one another may more preferably represent:

hydrogen or halogen or represent a C₁-C₃-alkyl, C₁-C₃-haloalkyl, C₁-C₃-alkoxy, C₁-C₃-haloalkoxy or C₁-C₃-alkoxy-C₁-C₃-alkyl radical, and/or
V¹ and V² together with the carbon atoms to which they are attached form a saturated or unsaturated cycle T¹ which optionally contains at least one further heteroatom and has 5 or 6 ring atoms and whose ring-forming atoms may be mono- or polysubstituted by identical or different substituents selected from the group consisting of halogen and a C₁-C₃-alkyl radical.

In formula (I), V¹, V² and V³ independently of one another may particularly preferably represent:

hydrogen, halogen or a C₁-C₃-alkyl or C₁-C₃-haloalkyl radical.

The group CKE may represent one of the following groups:

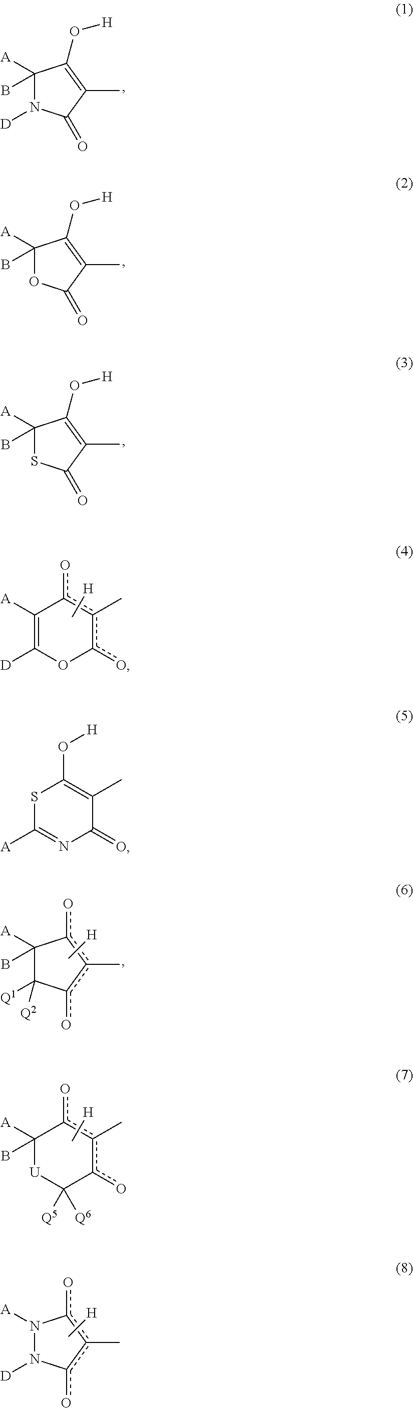

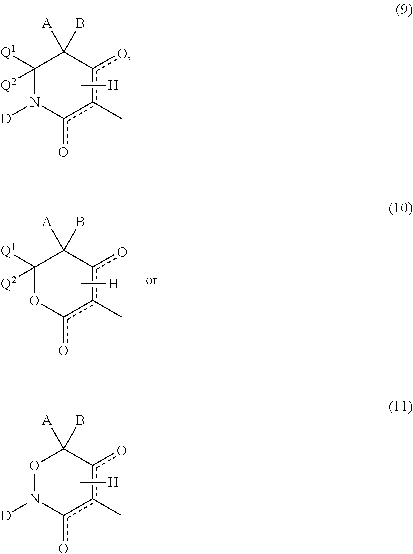

The group CKE may preferably represent one of the following groups:

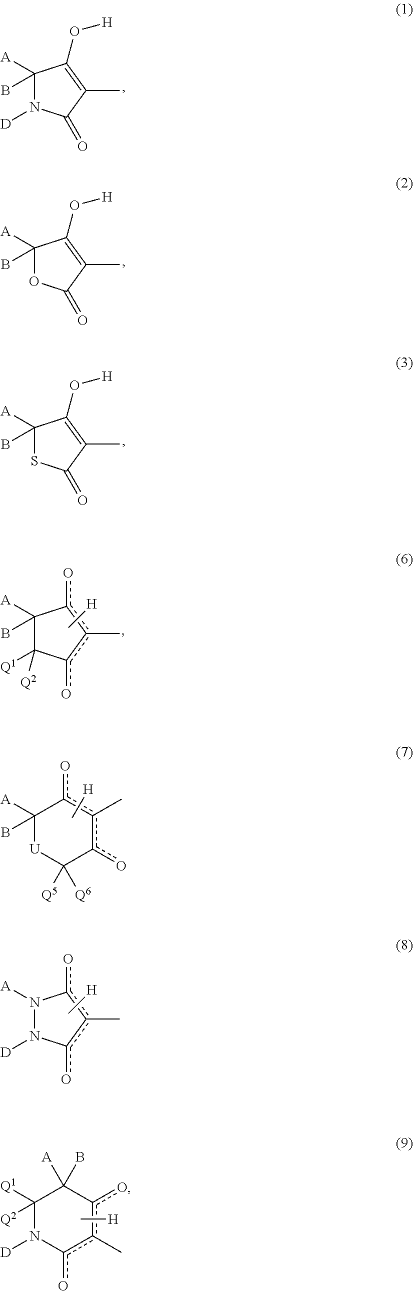

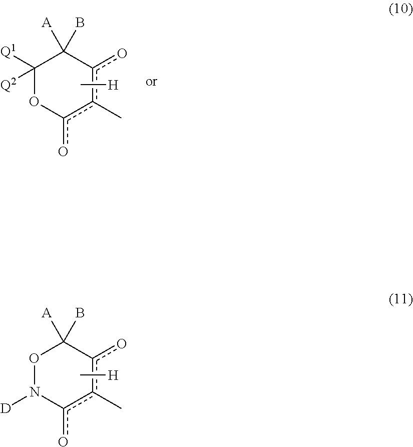

The group CKE may more preferably represent one of the following groups:

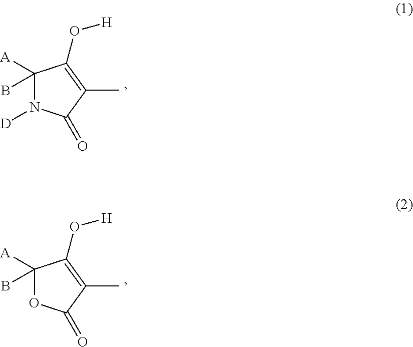

In the group CKE of the formula (I), U may represent:

### —S—, —S(O)—, —S(O)₂—, —O—,

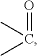

a substituted

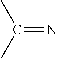

group
or an optionally Q³- and Q⁴-substituted C₁-C₄-alkylene group.

In the group CKE of the formula (I), U may preferably represent an optionally Q³- and Q⁴-substituted methylene group.

In the group CKE of the formula (I), A may represent:

hydrogen or
an optionally monohalogen- or polyhalogen-substituted C₁-C₆-alkyl, C₂-C₆-alkenyl, C₁-C₆-alkoxy-C₁-C₆-alkyl or C₁-C₆-alkylthio-C₁-C₆-alkyl radical or
a C₃-C₇-cycloalkyl, C₃-C₇-cycloalkyl-C₁-C₄-alkyl or monocyclic heterocyclyl or heterocyclyl-C₁-C₄-alkyl radical, each of which may be mono- or polysubstituted by identical or different substituents selected from the group consisting of halogen and a C₁-C₆-alkyl radical or an aryl, aryl-C₁-C₆-alkyl or heteroaryl radical, each of which may optionally be mono- or polysubstituted by identical or different substituents selected from the group consisting of halogen, cyano, nitro and C₁-C₆-alkyl, halo-C₁-C₆-alkyl, C₁-C₆-alkoxy and halo-C₁-C₆-alkoxy radicals.

In the group CKE of the formula (I), A may preferably represent:

hydrogen or
an optionally monohalogen- or polyhalogen-substituted C₁-C₆-alkyl or C₁-C₆-alkoxy-C₁-C₆-alkyl radical or
a C₃-C₇-cycloalkyl radical or 4- to 7-membered monocyclic heterocyclyl radical, each of which may be mono- or polysubstituted by identical or different substituents selected from the group consisting of halogen and a C₁-C₃-alkyl radical or
a phenyl, phenyl-C₁-C₃-alkyl or monocyclic heteroaryl radical, each of which may optionally be mono- or polysubstituted by identical or different substituents selected from the group consisting of halogen, cyano and C₁-C₃-alkyl, halo-C₁-C₃-alkyl, C₁-C₃-alkoxy and halo-C₁-C₃-alkoxy radicals.

In the group CKE of the formula (I), A may more preferably represent:

hydrogen or
an optionally monohalogen- or polyhalogen-substituted C₁-C₆-alkyl or C₁-C₆-alkoxy-C₁-C₆-alkyl radical or
a C₃-C₇-cycloalkyl radical or 4- to 7-membered monocyclic heterocyclyl radical, each of which may be mono- or polysubstituted by identical or different substituents selected from the group consisting of halogen and a C₁-C₃-alkyl radical.

In the group CKE of the formula (I), A may particularly preferably represent:

hydrogen or
an optionally monohalogen- or polyhalogen-substituted C₁-C₆-alkyl or C₁-C₆-alkoxy-C₁-C₆-alkyl radical or
a C₃-C₆-cycloalkyl radical which may be mono- or polysubstituted by identical or different substituents selected from the group consisting of halogen and a C₁-C₃-alkyl radical.

In the group CKE of the formula (I), B may represent:

hydrogen or a C₁-C₆-alkyl or C₁-C₆-alkoxy-C₁-C₆-alkyl radical.

In the group CKE of the formula (I), B may preferably represent:

hydrogen or a C₁-C₆-alkyl or C₁-C₃-alkoxy-C₁-C₃-alkyl radical.

In the group CKE of the formula (I), A and B together with the carbon atom to which they are attached may form:

a saturated or unsaturated cycle T² which optionally contains at least one heteroatom and has 3 to 8 ring atoms and whose ring-forming atoms may be mono- or polysubstituted by identical or different substituents selected from the group consisting of the radicals R¹, R² and R³,
where R¹, R² and R³ independently of one another
a) represent halogen, hydroxyl or cyano or
b) represent a C₁-C₆-alkyl, C₁-C₆-alkoxy, C₁-C₆-alkoxy-C₁-C₆-alkyl, C₁-C₆-alkylcarbonyl, C₁-C₆-alkoxycarbonyl, C₁-C₆-alkylaminocarbonyl, C₁-C₆-alkylthio, C₁-C₆-alkylsulphinyl, C₁-C₆-alkylsulphonyl, C₁-C₆-alkylaminosulphonyl, C₁-C₆-alkoxy-C₁-C₆-alkoxy, halo-C₁-C₆-alkyl or halo-C₁-C₆-alkoxy radical which is optionally hydroxyl-substituted in the alkyl moiety, or
c) represent an aryl, arylcarbonyl, arylsulphonyl, arylamino, heteroaryl, heteroarylcarbonyl, heteroarylsulphonyl or heteroarylamino radical, or
d) represent a C₃-C₇-cycloalkyl, C₃-C₇-cycloalkylcarbonyl, C₃-C₇-cycloalkylsulphonyl, heterocyclyl, heterocyclylcarbonyl or heterocyclylsulphonyl radical, where the radicals mentioned under c) and d) may optionally be mono- or polysubstituted at the ring system by identical or different substituents selected from the group consisting of halogen, hydroxyl, cyano, nitro and C₁-C₆-alkyl, C₁-C₆-alkoxy, halo-C₁-C₆-alkyl, halo-C₁-C₆-alkoxy, C₁-C₆-alkoxy-C₁-C₆-alkyl, C₃-C₁₀-cycloalkyl and 3- to 6-membered heterocycloalkyl radicals, and/or
e) two of the radicals R¹, R² and R³ together with the ring atom(s) of the cycle T² to which they are attached may form a further saturated or unsaturated cycle T³ which optionally contains at least one heteroatom and has 3 to 7 ring atoms and which may be mono- or polysubstituted by identical or different substituents selected from the group consisting of the radicals R⁴, R⁵ and R⁶, where R⁴, R⁵ and R⁶ independently of one another represent a C₁-C₆-alkyl or C₁-C₆-alkoxy radical.

In the group CKE of the formula (I), A and B together with the carbon atom to which they are attached may preferably form:

a saturated or unsaturated cycle T² which optionally contains one or two heteroatoms and has 3 to 8 ring atoms and whose ring-forming atoms may be mono- or polysubstituted by identical or different substituents selected from the group consisting of the radicals R¹, R² and R³,
where R¹, R² and R³ independently of one another
a) represent halogen or hydroxyl or
b) represent a C₁-C₅-alkyl, C₁-C₅-alkoxy, C₁-C₃-alkoxy-C₁-C₃-alkyl, C₁-C₃-alkylcarbonyl, C₁-C₃-alkoxycarbonyl, C₁-C₃-alkylaminocarbonyl, C₁-C₃-alkylthio, C₁-C₃-alkylsulphinyl, C₁-C₃-alkylsulphonyl, C₁-C₃-alkylaminosulphonyl, C₁-C₃-alkoxy-C₁-C₃-alkoxy, halo-C₁-C₃-alkyl or halo-C₁-C₃-alkoxy radical which is optionally hydroxyl-substituted in the alkyl moiety, or
c) represent a phenyl, phenylcarbonyl, phenylsulphonyl or phenylamino radical, or
d) represent a C₃-C₇-cycloalkyl, C₃-C₇-cycloalkylcarbonyl, C₃-C₇-cycloalkylsulphonyl or in each case 4- to 7-membered monocyclic heterocyclyl, heterocyclylcarbonyl or heterocyclylsulphonyl radical,
   where the radicals mentioned under c) and d) may optionally be mono- or polysubstituted at the ring system by identical or different substituents selected from the group consisting of halogen, hydroxyl and C₁-C₃-alkyl, halo-C₁-C₃-alkyl, C₁-C₃-alkoxy, halo-C₁-C₃-alkoxy and C₁-C₃-alkoxy-C₁-C₃-alkyl radicals, and/or
e) two of the radicals R¹, R² and R³ together with the ring atom(s) of the cycle T² to which they are attached may form a further saturated or aromatic cycle T³ which optionally contains one or two heteroatoms and has 5 to 7 ring atoms and may be mono- or polysubstituted by identical or different substituents selected from the group consisting of the radicals R⁴, R⁵ and R⁶, where R⁴, R⁵ and R⁶ independently of one another represent a C₁-C₃-alkyl or C₁-C₃-alkoxy radical.

In the group CKE of the formula (I), A and B together with the carbon atom to which they are attached may more preferably form:

a saturated or unsaturated cycle T² which optionally contains one or two heteroatoms and has 3 to 8 ring atoms and whose ring-forming atoms may be mono- or polysubstituted by identical or different substituents selected from the group consisting of the radicals R¹, R² and R³,
where R¹, R² and R³ independently of one another
a) represent halogen or hydroxyl or
b) represent a C₁-C₄-alkyl, C₁-C₄-alkoxy, C₁-C₃-alkoxy-C₁-C₃-alkyl, C₁-C₃-alkoxy-C₁-C₃-alkoxy, halo-C₁-C₃-alkyl or halo-C₁-C₃-alkoxy radical which is optionally hydroxyl-substituted in the alkyl moiety, or
c) represent a C₃-C₇-cycloalkyl or 4- to 7-membered monocyclic heterocyclyl radical, each of which is optionally mono- or polysubstituted at the ring system by identical or different substituents selected from the group consisting of halogen, hydroxyl and C₁-C₃-alkyl, halo-C₁-C₃-alkyl, C₁-C₃-alkoxy, halo-C₁-C₃-alkoxy and C₁-C₃-alkoxy-C₁-C₃-alkyl radicals, and/or
d) two of the radicals R¹, R² and R³ together with the ring atom(s) of the cycle T² to which they are attached may form a further saturated or aromatic cycle T³ which optionally contains one or two heteroatoms and has 5 to 7 ring atoms and which may be mono- or polysubstituted by identical or different substituents selected from the group consisting of the radicals R⁴, R⁵ and R⁶
   where R⁴, R⁵ and R⁶ independently of one another represent a C₁-C₃-alkyl or C₁-C₃-alkoxy radical.

In the group CKE of the formula (I), A and B together with the carbon atom to which they are attached may likewise more preferably form:

a saturated cycle T² which optionally contains one heteroatom and has 3 to 8 ring atoms and whose ring-forming atoms may be mono- or disubstituted by identical or different substituents selected from the group consisting of the radicals R¹ and R²,
where R¹ and R² independently of one another
a) represent hydroxyl or
b) represent a C₁-C₃-alkyl, C₁-C₃-alkoxy, C₁-C₂-alkoxy-C₁-C₃-alkyl, C₁-C₃-alkoxy-C₁-C₃-alkoxy, halo-C₁-C₃-alkyl or halo-C₁-C₃-alkoxy radical which is optionally hydroxyl-substituted in the alkyl moiety.

In the group CKE of the formula (I), A and B together with the carbon atom to which they are attached may particularly preferably form:

a saturated cycle T² which optionally contains one or two heteroatoms and has 3 to 8 ring atoms and whose ring-forming atoms may be mono- or polysubstituted by identical or different substituents selected from the group consisting of the radicals R¹, R² and R³,
where R¹, R² and R³ independently of one another
a) represent a C₁-C₃-alkyl, C₁-C₃-alkoxy, C₁-C₃-alkoxy-C₁-C₃-alkyl, C₁-C₃-alkoxy-C₁-C₃-alkoxy or halo-C₁-C₃-alkoxy radical which is optionally hydroxyl-substituted in the alkyl moiety, and/or
b) two of the radicals R¹, R² and R³ together with the ring atom(s) of the cycle T² to which they are attached may form a further saturated or aromatic cycle T³ which optionally contains at least one oxygen atom and has 5 to 7 ring atoms and which may be mono- or polysubstituted by a C₁-C₃-alkyl radical.

In the group CKE of the formula (I), A and B together with the carbon atom to which they are attached may likewise particularly preferably form:

a saturated cycle T² which optionally contains an oxygen atom and has 5 to 6 ring atoms and whose ring-forming atoms may be mono- or disubstituted by identical or different substituents selected from the group consisting of the radicals R¹ and R²,
where R¹ and R² independently of one another
a) represent hydroxyl or
b) represent a C₁-C₃-alkyl, hydroxymethyl, C₁-C₂-alkoxy, methoxy-C₁-C₂-alkyl, trifluoromethyl, pentafluoroethyl or 2,2,2-trifluoroethoxy radical.

In the group CKE of the formula (I), D may represent:

hydrogen or
a C₁-C₆-alkyl, C₂-C₆-alkenyl, C₂-C₆-alkynyl or C₁-C₆-alkoxy-C₁-C₆-alkyl radical or
a C₃-C₇-cycloalkyl, C₃-C₇-cycloalkyl-C₁-C₄-alkyl or monocyclic heterocyclyl or heterocyclyl-C₁-C₄-alkyl radical, or
an aryl, aryl-C₁-C₆-alkyl, heteroaryl or heteroaryl-C₁-C₆-alkyl radical,
where the radicals mentioned may optionally be mono- or polysubstituted by identical or different substituents selected from the group consisting of halogen, hydroxyl, cyano, nitro and C₁-C₆-alkyl, halo-C₁-C₆-alkyl, C₁-C₆-alkoxy, halo-C₁-C₆-alkoxy, C₁-C₆-alkoxy-C₁-C₆-alkyl, C₃-C₁₀-cycloalkyl and monocyclic heterocycloalkyl radicals.

In the group CKE of the formula (I), D may preferably represent:

hydrogen or
a C₁-C₆-alkyl or C₁-C₆-alkoxy-C₁-C₆-alkyl radical or
a C₃-C₇-cycloalkyl or 4- to 7-membered monocyclic heterocyclyl radical or
a phenyl or phenyl-C₁-C₃-alkyl radical,
where the radicals mentioned may optionally be mono- or polysubstituted by identical or different substituents selected from the group consisting of halogen, hydroxyl, cyano and C₁-C₃-alkyl, halo-C₁-C₃-alkyl, C₁-C₃-alkoxy, halo-C₁-C₃-alkoxy, C₁-C₃-alkoxy-C₁-C₃-alkyl and C₃-C₇-cycloalkyl radicals.

In the group CKE of the formula (I), D may more preferably represent:

hydrogen or
a C₁-C₆-alkyl or C₁-C₆-alkoxy-C₁-C₆-alkyl radical or
a C₃-C₇-cycloalkyl or 4- to 7-membered monocyclic heterocyclyl radical,
where the radicals mentioned may optionally be mono- or polysubstituted by identical or different substituents selected from the group consisting of halogen and hydroxyl and C₁-C₃-alkyl, halo-C₁-C₃-alkyl, C₁-C₃-alkoxy, halo-C₁-C₃-alkoxy and C₁-C₃-alkoxy-C₁-C₃-alkyl radicals.

In the group CKE of the formula (I), D may particularly preferably represent:

hydrogen or
a C₁-C₆-alkyl or C₃-C₇-cycloalkyl radical,
where the radicals mentioned may be mono- or polysubstituted by identical or different substituents selected from the group consisting of halogen, hydroxyl and a C₁-C₃-alkyl radical.

In the group CKE of the formula (I), A and D together with the atoms to which they are attached may alternatively form:

a saturated or unsaturated cycle T⁴ which optionally contains at least one further heteroatom and has 3 to 7 ring atoms, which may be bridged and whose ring-forming atoms may be mono- or polysubstituted by identical or different substituents selected from the group consisting of the radicals R⁷, R⁸ and R⁹,
where R⁷, R⁸ and R⁹ independently of one another represent hydroxyl, halogen or a C₁-C₆-alkyl or C₁-C₆-alkoxy radical.

If the group CKE of the formula (I) is the group 8, A and D together with the atoms to which they are attached may alternatively preferably form:

a saturated or unsaturated cycle T⁴ which optionally contains a further heteroatom and has 5 to 7 ring atoms, which may be bridged and whose ring-forming atoms may be mono- or polysubstituted by identical or different substituents selected from the group consisting of the radicals R⁷, R⁸ and R⁹,
where R⁷, R⁸ and R⁹ independently of one another represent halogen or a C₁-C₃-alkyl or C₁-C₃-alkoxy radical.

If the group CKE of the formula (I) is the group 8, A and D together with the atoms to which they are attached may alternatively more preferably form:

a saturated cycle T⁴ which optionally contains a further heteroatom and has 5 to 7 ring atoms, which may be bridged and whose ring-forming atoms may be mono- or polysubstituted by identical or different substituents selected from the group consisting of the radicals R⁷, R⁸ and R⁹,
where R⁷, R⁸ and R⁹ independently of one another represent halogen or a C₁-C₃-alkyl radical.

In the group CKE of the formula (I), A and Q¹ together with the atoms to which they are attached may form:

a saturated or unsaturated cycle T⁵ which optionally contains at least one further heteroatom and has 5 to 7 ring atoms and whose ring-forming atoms may be mono- or polysubstituted by identical or different substituents selected from the group consisting of halogen, hydroxyl, cyano, nitro and C₁-C₆-alkyl, halo-C₁-C₆-alkyl, C₁-C₆-alkoxy, halo-C₁-C₆-alkoxy, C₁-C₆-alkoxy-C₁-C₆-alkyl and C₃-C₁₀-cycloalkyl radicals,
with the proviso that B and Q² represent a bond if the cycle T⁵ formed by A and Q¹ is aromatic.

In the group CKE of the formula (I), A and Q¹ together with the atoms to which they are attached may preferably form:

an unsaturated cycle T⁵ which optionally contains at least one further heteroatom and has 5 to 7 ring atoms and whose ring-forming atoms may be mono- or polysubstituted by identical or different substituents selected from the group consisting of halogen and C₁-C₃-alkyl, halo-C₁-C₃-alkyl, C₁-C₃-alkoxy, halo-C₁-C₃-alkoxy and C₁-C₃-alkoxy-C₁-C₃-alkyl radicals,
with the proviso that B and Q² represent a bond if the cycle T⁵ formed by A and Q¹ is aromatic.

In the group CKE of the formula (I), A and Q¹ together with the atoms to which they are attached may particularly preferably form:

an aromatic cycle T⁵ which has 6 ring atoms and whose ring-forming atoms may be mono- or polysubstituted by halogen,
with the proviso that in this case B and Q² represent a bond.

In the group CKE of the formula (I), Q¹ may represent:

hydrogen or
a C₁-C₆-alkyl or C₁-C₆-alkoxy radical which is optionally mono- or polysubstituted by identical or different substituents selected from the group consisting of halogen, hydroxyl and a C₁-C₆-alkoxy radical, or
a C₃-C₇-cycloalkyl, C₃-C₇-cycloalkyl-C₁-C₄-alkyl or monocyclic heterocyclyl or heterocyclyl-C₁-C₄-alkyl radical, each of which may optionally be mono- or polysubstituted by identical or different substituents selected from the group consisting of halogen, hydroxyl and C₁-C₆-alkyl, halo-C₁-C₆-alkyl, C₁-C₆-alkoxy, C₁-C₆-alkoxy-C₁-C₆-alkoxy and halo-C₁-C₆-alkoxy radicals, or a phenyl radical which may optionally be mono- or polysubstituted by identical or different substituents selected from the group consisting of halogen, hydroxyl, cyano, nitro and C₁-C₆-alkyl, halo-C₁-C₆-alkyl, C₁-C₆-alkoxy, halo-C₁-C₆-alkoxy, C₁-C₆-alkoxy-C₁-C₆-alkyl and C₃-C₁₀-cycloalkyl radicals.

In the group CKE of the formula (I), Q¹ may preferably represent:

hydrogen or
a C₁-C₃-alkyl or C₁-C₃-alkoxy radical which is optionally mono- or polysubstituted by identical or different substituents selected from the group consisting of halogen, hydroxyl and a C₁-C₆-alkoxy radical, or
a C₃-C₇-cycloalkyl or 4- to 7-membered monocyclic heterocyclyl radical, each of which may optionally be mono- or polysubstituted by identical or different substituents selected from the group consisting of halogen, hydroxyl and C₁-C₃-alkyl, halo-C₁-C₃-alkyl, C₁-C₃-alkoxy, C₁-C₃-alkoxy-C₁-C₃-alkoxy, and halo-C₁-C₃-alkoxy radicals.

In the group CKE of the formula (I), Q¹ may particularly preferably represent:

hydrogen or a C₁-C₃-alkyl radical.

In the group CKE of the formula (I), Q², Q⁴, Q⁵ and Q⁶ independently of one another may represent hydrogen or a C₁-C₆-alkyl radical.

In the group CKE of the formula (I), Q², Q⁴, Q⁵ and Q⁶ independently of one another may preferably represent hydrogen or a C₁-C₃-alkyl radical.

In the group CKE of the formula (I), Q³ may represent:

hydrogen or
a C₁-C₆-alkyl or C₁-C₆-alkoxy radical which is optionally mono- or polysubstituted by identical or different substituents selected from the group consisting of halogen, hydroxyl and a C₁-C₆-alkoxy radical, or
a C₃-C₇-cycloalkyl, C₃-C₇-cycloalkyl-C₁-C₄-alkyl or monocyclic heterocyclyl or heterocyclyl-C₁-C₄-alkyl radical, each of which may optionally be mono- or polysubstituted by identical or different substituents selected from the group consisting of halogen, hydroxyl and C₁-C₆-alkyl, halo-C₁-C₆-alkyl, C₁-C₆-alkoxy, C₁-C₆-alkoxy-C₁-C₆-alkoxy and halo-C₁-C₆-alkoxy radicals, or represent a phenyl radical which may optionally be mono- or polysubstituted by identical or different substituents selected from the group consisting of halogen, hydroxyl, cyano, nitro and C₁-C₆-alkyl, halo-C₁-C₆-alkyl, C₁-C₆-alkoxy, halo-C₁-C₆-alkoxy, C₁-C₆-alkoxy-C₁-C₆-alkyl and C₃-C₁₀-cycloalkyl radicals.

In the group CKE of the formula (I), Q³ may preferably represent:

hydrogen or
a C₁-C₃-alkyl or C₁-C₃-alkoxy radical which is optionally mono- or polysubstituted by identical or different substituents selected from the group consisting of halogen, hydroxyl and a C₁-C₆-alkoxy radical.

In the group CKE of the formula (I), Q³ may particularly preferably represent:

hydrogen or a C₁-C₃-alkyl radical.

In the group CKE of the formula (I), Q¹ and Q² together with the carbon atom to which they are attached may form a saturated or unsaturated cycle T⁶ which optionally contains at least one further heteroatom and has 3 to 7 ring atoms and whose ring-forming atoms may be mono- or polysubstituted by identical or different substituents selected from the group consisting of halogen, hydroxyl, cyano, nitro and C₁-C₆-alkyl, halo-C₁-C₆-alkyl, C₁-C₆-alkoxy, halo-C₁-C₆-alkoxy, C₁-C₆-alkoxy-C₁-C₆-alkyl and C₃-C₁₀-cycloalkyl radicals.

In the group CKE of the formula (I), Q³ and Q⁴ together with the carbon atom to which they are attached may form:

a saturated or unsaturated cycle T⁷ which optionally contains at least one heteroatom and has 3 to 7 ring atoms and whose ring-forming atoms may be mono- or polysubstituted by identical or different substituents selected from the group consisting of halogen, hydroxyl, cyano, nitro and C₁-C₆-alkyl, halo-C₁-C₆-alkyl, C₁-C₆-alkoxy, halo-C₁-C₆-alkoxy, C₁-C₆-alkoxy-C₁-C₆-alkyl and C₃-C₁₀-cycloalkyl radicals.

In the group CKE of the formula (I), Q³ and Q⁴ together with the carbon atom to which they are attached may preferably form:

a saturated cycle T⁷ which optionally contains at least one heteroatom and has 5 to 7 ring atoms and whose ring-forming atoms may be mono- or polysubstituted by identical or different substituents selected from the group consisting of halogen, hydroxyl and C₁-C₃-alkyl, halo-C₁-C₃-alkyl, C₁-C₆-alkoxy, halo-C₁-C₃-alkoxy and C₁-C₃-alkoxy-C₁-C₃-alkyl radicals.

In the group CKE of the formula (I), Q³ and Q⁴ together with the carbon atom to which they are attached may particularly preferably form:

a saturated cycle T⁷ which has 6 ring atoms and whose ring-forming atoms may be mono- or polysubstituted by identical or different substituents selected from the group consisting of halogen and a C₁-C₆-alkyl radical.

A preferred group of compounds of the general formula (I) for use as medicaments is formed by compounds of the general formula (I)

in which
X represents halogen or
   an optionally monohalogen- or polyhalogen-substituted C₁-C₃-alkyl or C₁-C₃-alkoxy radical, and
W and Y independently of one another represent hydrogen, cyano or halogen or
   an optionally monohalogen- or polyhalogen-substituted C₁-C₃-alkyl or C₁-C₃-alkoxy radical, and
V¹, V² and V³ independently of one another represent hydrogen, halogen or cyano or a C₁-C₃-alkyl, C₁-C₃-haloalkyl, C₁-C₃-alkoxy, C₁-C₃-haloalkoxy or C₁-C₃-alkoxy-C₁-C₃-alkyl radical, and/or
V¹ and V² together with the carbon atoms to which they are attached form a saturated or unsaturated cycle T¹ which optionally contains at least one further heteroatom and has 5 or 6 ring atoms and whose ring-forming atoms may be mono- or polysubstituted by identical or different substituents selected from the group consisting of halogen and a C₁-C₃-alkyl radical,
CKE represents one of the groups

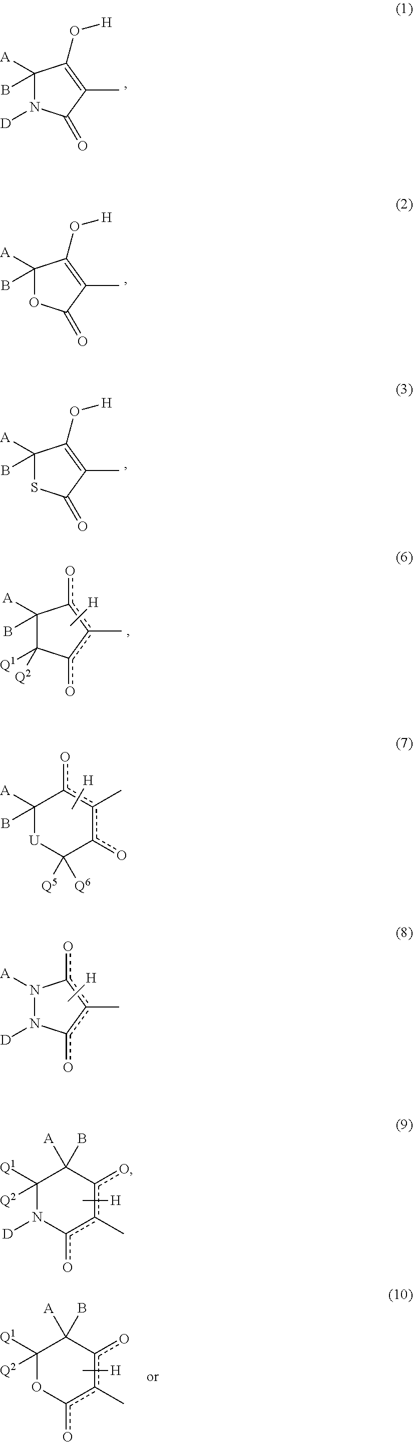

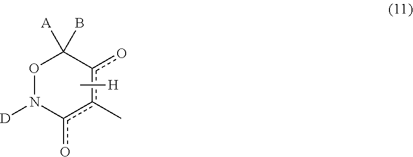

in which
U represents an optionally Q³- and Q⁴-substituted methylene group, and
A represents hydrogen or
   an optionally monohalogen- or polyhalogen-substituted C₁-C₆-alkyl or C₁-C₆-alkoxy-C₁-C₆-alkyl radical or
   a C₃-C₇-cycloalkyl radical or 4- to 7-membered monocyclic heterocyclyl radical, each of which may be mono- or polysubstituted by identical or different substituents selected from the group consisting of halogen and a C₁-C₃-alkyl radical, or
   a phenyl, phenyl-C₁-C₃-alkyl or monocyclic heteroaryl radical, each of which may be mono- or polysubstituted by identical or different substituents selected from the group consisting of halogen, cyano and C₁-C₃-alkyl, halo-C₁-C₃-alkyl, C₁-C₃-alkoxy and halo-C₁-C₃-alkoxy radicals, and
   B represents hydrogen or represents a C₁-C₆-alkyl or C₁-C₆-alkoxy-C₁-C₆-alkyl radical, or
   A and B together with the carbon atom to which they are attached form a saturated or unsaturated cycle T² which optionally contains one or two heteroatoms and has 3 to 8 ring atoms and whose ring-forming atoms may be mono- or polysubstituted by identical or different substituents selected from the group consisting of the radicals R¹, R² and R³,
      where R¹, R² and R³ independently of one another
      a) represent halogen or hydroxyl or
      b) represent a C₁-C₅-alkyl, C₁-C₅-alkoxy, C₁-C₃-alkoxy-C₁-C₃-alkyl, C₁-C₃-alkylcarbonyl, C₁-C₃-alkoxycarbonyl, C₁-C₃-alkylaminocarbonyl, C₁-C₃-alkylthio, C₁-C₃-alkylsulphinyl, C₁-C₃-alkylsulphonyl, C₁-C₃-alkylaminosulphonyl, C₁-C₃-alkoxy-C₁-C₃-alkoxy, halo-C₁-C₃-alkyl or halo-C₁-C₃-alkoxy radical which is optionally hydroxyl-substituted in the alkyl moiety and/or
      c) represent a phenyl, phenylcarbonyl, phenylsulphonyl or phenylamino radical, or
      d) represent a C₃-C₇-cycloalkyl, C₃-C₇-cycloalkylcarbonyl, C₃-C₇-cycloalkylsulphonyl or in each case 4- to 7-membered monocyclic heterocyclyl, heterocyclylcarbonyl or heterocyclylsulphonyl radical, where the radicals mentioned under c) and d) may optionally be mono- or polysubstituted at the ring system by identical or different substituents selected from the group consisting of halogen, hydroxyl and C₁-C₃-alkyl, halo-C₁-C₃-alkyl, C₁-C₃-alkoxy, halo-C₁-C₃-alkoxy and C₁-C₃-alkoxy-C₁-C₃-alkyl radicals, and/or
      e) two of the radicals R¹, R² and R³ together with the ring atom(s) of the cycle T² to which they are attached may form a further saturated or aromatic cycle T³ which optionally contains one or two heteroatoms and has 5 to 7 ring atoms and may be mono- or polysubstituted by identical or different substituents selected from the group consisting of the radicals R⁴, R⁵ and R⁶,
         where R⁴, R⁵ and R⁶ independently of one another represent a C₁-C₃-alkyl or C₁-C₃-alkoxy radical, and
   D represents hydrogen or
      represents a C₁-C₆-alkyl or C₁-C₆-alkoxy-C₁-C₆-alkyl radical or
      represents a C₃-C₇-cycloalkyl or 4- to 7-membered monocyclic heterocyclyl radical or
      represents a phenyl or phenyl-C₁-C₃-alkyl radical,
      where the radicals mentioned may optionally be mono- or polysubstituted by identical or different substituents selected from the group consisting of halogen, hydroxyl, cyano and C₁-C₃-alkyl, halo-C₁-C₃-alkyl, C₁-C₃-alkoxy, halo-C₁-C₃-alkoxy, C₁-C₃-alkoxy-C₁-C₃-alkyl and C₃-C₇-cycloalkyl radicals, or
   if CKE is the group 8,
   A and D alternatively together with the atoms to which they are attached form a saturated or unsaturated cycle T⁴ which optionally contains a further heteroatom and has 5 to 7 ring atoms, which may be bridged and whose ring-forming atoms may be mono- or polysubstituted by identical or different substituents selected from the group consisting of the radicals R⁷, R⁸ and R⁹,
      where R⁷, R⁸ and R⁹ independently of one another represent halogen or a C₁-C₃-alkyl or C₁-C₃-alkoxy radical, and
   A and Q¹ together with the atoms to which they are attached form an unsaturated cycle T⁵ which optionally contains at least one further heteroatom and has 5 to 7 ring atoms and whose ring-forming atoms may be mono- or polysubstituted by identical or different substituents selected from the group consisting of halogen and C₁-C₃-alkyl, halo-C₁-C₃-alkyl, C₁-C₃-alkoxy, halo-C₁-C₃-alkoxy and C₁-C₃-alkoxy-C₁-C₃-alkyl radicals,
      with the proviso that B and Q² represent a bond if the cycle T⁵ formed by A and Q¹ is aromatic, and
   Q¹ represents hydrogen or
      represents a C₁-C₃-alkyl or C₁-C₃-alkoxy radical which may optionally be mono- or polysubstituted by identical or different substituents selected from the group consisting of halogen, hydroxyl and a C₁-C₆-alkoxy radical or
      represents a C₃-C₇-cycloalkyl or 4- to 7-membered monocyclic heterocyclyl radical, each of which may optionally be mono- or polysubstituted by identical or different substituents selected from the group consisting of halogen, hydroxyl and C₁-C₃-alkyl, halo-C₁-C₃-alkyl, C₁-C₃-alkoxy, C₁-C₃-alkoxy-C₁-C₃-alkoxy and halo-C₁-C₃-alkoxy radicals, and
   Q², Q⁴, Q⁵ and Q⁶ independently of one another represent hydrogen or represent a C₁-C₃-alkyl radical, and
   Q³ represents hydrogen or
      represents a C₁-C₃-alkyl or C₁-C₃-alkoxy radical which may optionally be mono- or polysubstituted by identical or different substituents selected from the group consisting of halogen, hydroxyl and a C₁-C₆-alkoxy radical, or
   Q³ and Q⁴ together with the carbon atom to which they are attached form a saturated cycle T⁷ which optionally contains at least one heteroatom and has 5 to 7 ring atoms and whose ring-forming atoms may be mono- or polysubstituted by identical or different substituents selected from the group consisting of halogen, hydroxyl and C₁-C₃-alkyl, halo-C₁-C₃-alkyl, C₁-C₆-alkoxy, halo-C₁-C₃-alkoxy and C₁-C₃-alkoxy-C₁-C₃-alkyl radicals.

A more preferred group of compounds of the general formula (I) for use as medicaments is formed by compounds of the general formula (I),

in which
X represents halogen or
   represents an optionally monohalogen- or polyhalogen-substituted C₁-C₃-alkyl or C₁-C₃-alkoxy radical and
W and Y independently of one another represent hydrogen or halogen or
   represent an optionally monohalogen- or polyhalogen-substituted C₁-C₃-alkyl or C₁-C₃-alkoxy radical, and
V¹, V² and V³ independently of one another represent hydrogen or halogen or represent a C₁-C₃-alkyl, C₁-C₃-haloalkyl, C₁-C₃-alkoxy, C₁-C₃-haloalkoxy or C₁-C₃-alkoxy-C₁-C₃-alkyl radical, and/or
V¹ and V² together with the carbon atoms to which they are attached form a saturated or unsaturated cycle T¹ which optionally contains at least one further heteroatom and has 5 or 6 ring atoms and whose ring-forming atoms may be mono- or polysubstituted by identical or different substituents selected from the group consisting of halogen and a C₁-C₃-alkyl radical,
CKE represents one of the groups

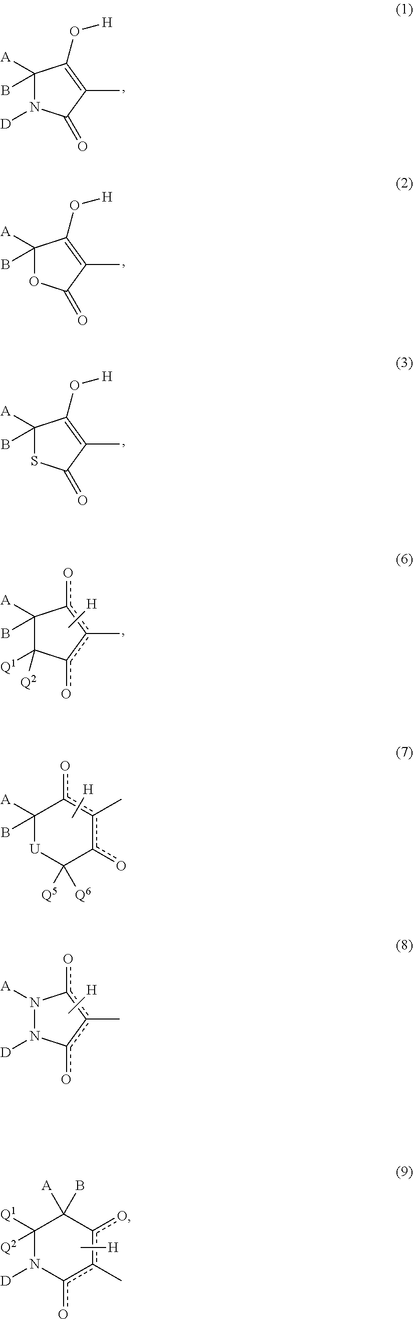

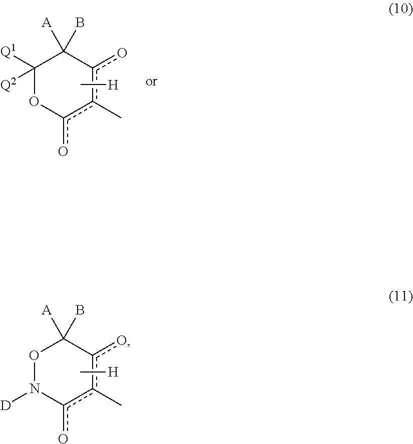

in which
U represents an optionally Q³- and Q⁴-substituted methylene group, and
A represents hydrogen or
   represents an optionally monohalogen- or polyhalogen-substituted C₁-C₆-alkyl or C₁-C₆-alkoxy-C₁-C₆-alkyl radical or
   represents a C₃-C₇-cycloalkyl radical or 4- to 7-membered monocyclic heterocyclyl radical, each of which may be mono- or polysubstituted by identical or different substituents selected from the group consisting of halogen and a C₁-C₃-alkyl radical, and
B represents hydrogen or represents a C₁-C₆-alkyl or C₁-C₃-alkoxy-C₁-C₃-alkyl radical, or
A and B together with the carbon atom to which they are attached form a saturated or unsaturated cycle T² which optionally contains one or two heteroatoms and has 3 to 8 ring atoms and whose ring-forming atoms may be mono- or polysubstituted by identical or different substituents selected from the group consisting of the radicals R¹, R² and R³,
   where R¹, R² and R³ independently of one another
   a) represent halogen or hydroxyl or
   b) represent a C₁-C₄-alkyl, C₁-C₄-alkoxy, C₁-C₃-alkoxy-C₁-C₃-alkyl, C₁-C₃-alkoxy-C₁-C₃-alkoxy, halo-C₁-C₃-alkyl or halo-C₁-C₃-alkoxy radical which is optionally hydroxyl-substituted in the alkyl moiety or
   c) represent a C₃-C₇-cycloalkyl or 4- to 7-membered monocyclic heterocyclyl radical, each of which may optionally be mono- or polysubstituted in the ring system by identical or different substituents selected from the group consisting of halogen, hydroxyl and C₁-C₃-alkyl, halo-C₁-C₃-alkyl, C₁-C₃-alkoxy, halo-C₁-C₃-alkoxy and C₁-C₃-alkoxy-C₁-C₃-alkyl radicals, and/or
   d) two of the radicals R¹, R² and R³ together with the ring atom(s) of the cycle T² to which they are attached may form a further saturated or aromatic cycle T³ which optionally contains one or two heteroatoms and has 5 to 7 ring atoms and which may be mono- or polysubstituted by identical or different substituents selected from the group consisting of the radicals R⁴, R⁵ and R⁶,
      where R⁴, R⁵ and R⁶ independently of one another represent a C₁-C₃-alkyl or C₁-C₃-alkoxy radical, and
   D represents hydrogen or
      represents a C₁-C₆-alkyl or C₁-C₆-alkoxy-C₁-C₆-alkyl radical or represents a C₃-C₇-cycloalkyl or 4- to 7-membered monocyclic heterocyclyl radical,
      where the radicals mentioned may optionally be mono- or polysubstituted by identical or different substituents selected from the group consisting of halogen and hydroxyl and C₁-C₃-alkyl, halo-C₁-C₃-alkyl, C₁-C₃-alkoxy, halo-C₁-C₃-alkoxy and C₁-C₃-alkoxy-C₁-C₃-alkyl radicals, or
   if CKE is the group 8,
   A and D alternatively together with the atoms to which they are attached form a saturated or unsaturated cycle T⁴ which optionally contains a further heteroatom and has 5 to 7 ring atoms, which may be bridged and whose ring-forming atoms may be mono- or polysubstituted by identical or different substituents selected from the group consisting of the radicals R⁷, R⁸ and R⁹,
      where R⁷, R⁸ and R⁹ independently of one another represent halogen or a C₁-C₃-alkyl or C₁-C₃-alkoxy radical, and
   A and Q¹ together with the atoms to which they are attached form an unsaturated cycle T⁵ which optionally contains at least one further heteroatom and has 5 to 7 ring atoms and whose ring-forming atoms may be mono- or polysubstituted by identical or different substituents selected from the group consisting of halogen and C₁-C₃-alkyl, halo-C₁-C₃-alkyl, C₁-C₃-alkoxy, halo-C₁-C₃-alkoxy and C₁-C₃-alkoxy-C₁-C₃-alkyl radicals,
      with the proviso that B and Q² represent a bond if the cycle T⁵ formed by A and Q¹ is aromatic, and
   Q¹ represents hydrogen or
      represents a C₁-C₃-alkyl or C₁-C₃-alkoxy radical which may optionally be mono- or polysubstituted by identical or different substituents selected from the group consisting of halogen, hydroxyl and a C₁-C₆-alkoxy radical or
      represents a C₃-C₇-cycloalkyl or 4- to 7-membered monocyclic heterocyclyl radical, each of which may optionally be mono- or polysubstituted by identical or different substituents selected from the group consisting of halogen, hydroxyl and C₁-C₃-alkyl, halo-C₁-C₃-alkyl, C₁-C₃-alkoxy, C₁-C₃-alkoxy-C₁-C₃-alkoxy and halo-C₁-C₃-alkoxy radicals, and
   Q², Q⁴, Q⁵ and Q⁶ independently of one another represent hydrogen or represent a C₁-C₃-alkyl radical, and
   Q³ represents hydrogen or represents a C₁-C₃-alkyl or C₁-C₃-alkoxy radical which may optionally be mono- or polysubstituted by identical or different substituents selected from the group consisting of halogen, hydroxyl and a C₁-C₆-alkoxy radical, or
   Q³ and Q⁴ together with the carbon atom to which they are attached form a saturated cycle T⁷ which optionally contains at least one heteroatom and has 5 to 7 ring atoms and whose ring-forming atoms may be mono- or polysubstituted by identical or different substituents selected from the group consisting of halogen, hydroxyl and C₁-C₃-alkyl, halo-C₁-C₃-alkyl, C₁-C₆-alkoxy, halo-C₁-C₃-alkoxy and C₁-C₃-alkoxy-C₁-C₃-alkyl radicals.

A particularly preferred group of compounds of the general formula (I) for use as medicaments is formed by compounds of the general formula (I)

in which
X represents halogen or an optionally monohalogen- or polyhalogen-substituted C₁-C₃-alkyl radical, and
W and Y independently of one another represent hydrogen or an optionally monohalogen- or polyhalogen-substituted C₁-C₃-alkyl radical,
V¹, V² and V³ independently of one another represent hydrogen, halogen or a C₁-C₃-alkyl or C₁-C₃-haloalkyl radical, and
CKE represents one of the groups

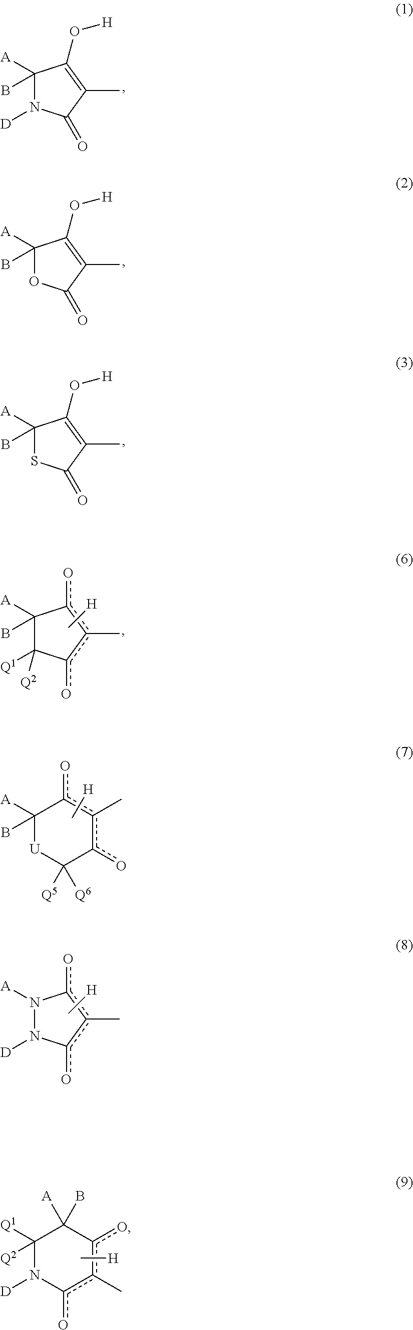

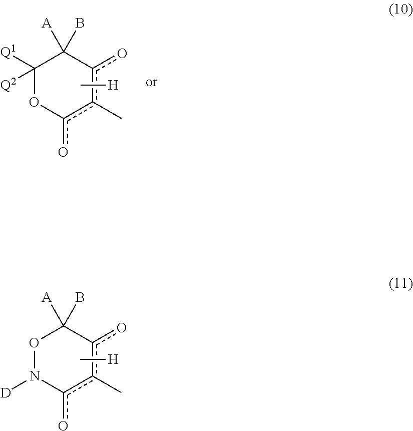

in which
U represents an optionally Q³- and Q⁴-substituted methylene group,
A represents hydrogen or
   represents an optionally monohalogen- or polyhalogen-substituted C₁-C₆-alkyl or C₁-C₆-alkoxy-C₁-C₆-alkyl radical or
   represents a C₃-C₆-cycloalkyl radical which may be mono- or polysubstituted by identical or different substituents selected from the group consisting of halogen and a C₁-C₃-alkyl radical, and
B represents hydrogen or a C₁-C₆-alkyl or C₁-C₃-alkoxy-C₁-C₃-alkyl radical, or
A and B together with the carbon atom to which they are attached form a saturated cycle T² which optionally contains one or two heteroatoms and has 3 to 8 ring atoms and whose ring-forming atoms may be mono- or polysubstituted by identical or different substituents selected from the group consisting of the radicals R¹, R² and R³,
   where R¹, R² and R³ independently of one another
   a) represent a C₁-C₃-alkyl, C₁-C₃-alkoxy, C₁-C₃-alkoxy-C₁-C₃-alkyl, C₁-C₃-alkoxy-C₁-C₃-alkoxy or halo-C₁-C₃-alkoxy radical which is optionally substituted in the alkyl moiety by hydroxyl, and/or
   b) two of the radicals R¹, R² and R³ together with the ring atom(s) of the cycle T² to which they are attached may form a further saturated or aromatic cycle T³ which optionally contains at least one oxygen atom and has 5 to 7 ring atoms and which may be mono- or polysubstituted by a C₁-C₃-alkyl radical, and
D represents hydrogen or a C₁-C₆-alkyl or C₃-C₇-cycloalkyl radical, where the radicals mentioned may be mono- or polysubstituted by identical or different substituents selected from the group consisting of halogen, hydroxyl and a C₁-C₃-alkyl radical, or
if CKE is the group 8,
A and D alternatively together with the atoms to which they are attached form a saturated cycle T⁴ which optionally contains a further heteroatom and has 5 to 7 ring atoms, which may be bridged and whose ring-forming atoms may be mono- or polysubstituted by identical or different substituents selected from the group consisting of the radicals R⁷, R⁸ and R⁹,
   where R⁷, R⁸ and R⁹ independently of one another represent halogen or a C₁-C₃-alkyl radical, or
A and Q¹ together with the atoms to which they are attached form an aromatic cycle T⁵ which has 6 ring atoms and whose ring-forming atoms may be mono- or polysubstituted by halogen,
   with the proviso that in this case B and Q² represent a bond, and
Q¹ represents hydrogen or a C₁-C₃-alkyl radical, and
Q², Q⁴, Q⁵ and Q⁶ independently of one another represent hydrogen or represent a C₁-C₃-alkyl radical, and
Q³ represents hydrogen or represents a C₁-C₃-alkyl radical, or
Q³ and Q⁴ together with the carbon atom to which they are attached form a saturated cycle T⁷ which has 6 ring atoms and whose ring-forming atoms may be mono- or polysubstituted by identical or different substituents selected from the group consisting of halogen and a C₁-C₆-alkyl radical.

Within the group of compounds of the general formula (I), depending on the CKE group, the following sub-groups result:

compounds of the general formula (I-1)

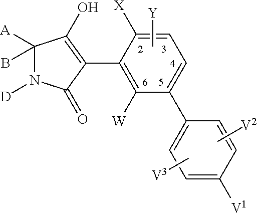

compounds of the general formula (I-2)

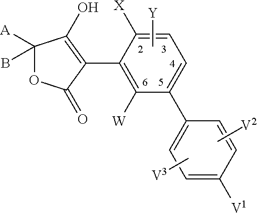

compounds of the general formula (I-3)

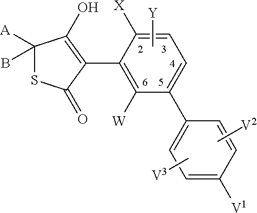

compounds of the general formula (I-4)

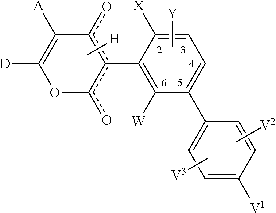

compounds of the general formula (I-5)

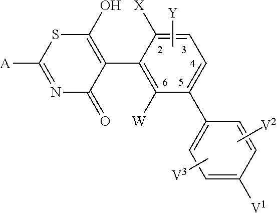

compounds of the general formula (I-6)

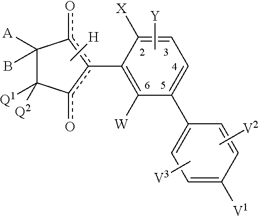

compounds of the general formula (I-7)

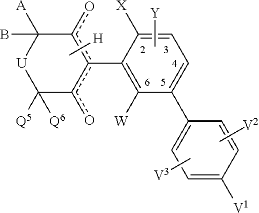

compounds of the general formula (I-8)

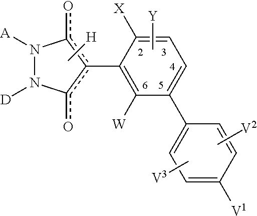

compounds of the general formula (I-9)

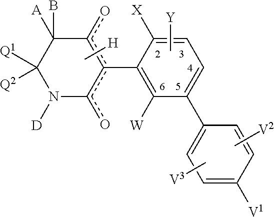

compounds of the general formula (I-10)

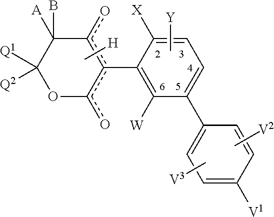

compounds of the general formula (1-11)

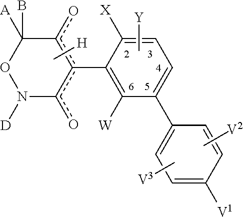

A preferred sub-group of compounds of the general formula (I) for use as medicaments is formed by compounds of the general formula (I-1)

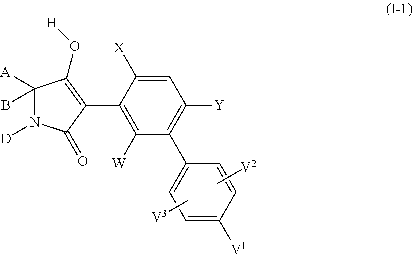

in which
X represents halogen or
   represents an optionally monohalogen- or polyhalogen-substituted C₁-C₃-alkyl or C₁-C₃-alkoxy radical, and
W and Y independently of one another represent hydrogen or halogen or
   represent an optionally monohalogen- or polyhalogen-substituted C₁-C₃-alkyl radical, and
V¹, V² and V³ independently of one another represent hydrogen or halogen or represent a C₁-C₃-alkyl, C₁-C₃-haloalkyl, C₁-C₃-alkoxy, C₁-C₃-haloalkoxy or C₁-C₃-alkoxy-C₁-C₃-alkyl radical, and/or
V¹ and V² together with the carbon atoms to which they are attached form a saturated or unsaturated cycle T¹ which optionally contains at least one further heteroatom and has 5 or 6 ring atoms and whose ring-forming atoms may be mono- or polysubstituted by identical or different substituents selected from the group consisting of halogen and a C₁-C₃-alkyl radical,
A represents hydrogen or
   represents an optionally monohalogen- or polyhalogen-substituted C₁-C₆-alkyl or C₁-C₆-alkoxy-C₁-C₆-alkyl radical or
   represents a C₃-C₇-cycloalkyl radical or 4- to 7-membered monocyclic heterocyclyl radical, each of which may be mono- or polysubstituted by identical or different substituents selected from the group consisting of halogen and a C₁-C₃-alkyl radical, and
B represents hydrogen or represents a C₁-C₆-alkyl or C₁-C₃-alkoxy-C₁-C₃-alkyl radical, or
A and B together with the carbon atom to which they are attached form a saturated or unsaturated cycle T² which optionally contains one or two heteroatoms and has 3 to 8 ring atoms and whose ring-forming atoms may be mono- or polysubstituted by identical or different substituents selected from the group consisting of the radicals R¹, R² and R³,
   where R¹, R² and R³ independently of one another
   a) represent halogen or hydroxyl or
   b) represent a C₁-C₄-alkyl, C₁-C₄-alkoxy, C₁-C₃-alkoxy-C₁-C₃-alkyl, C₁-C₃-alkoxy-C₁-C₃-alkoxy, halo-C₁-C₃-alkyl or halo-C₁-C₃-alkoxy radical which is optionally hydroxyl-substituted in the alkyl moiety and/or
   c) two of the radicals R¹, R² and R³ together with the ring atom(s) of the cycle T² to which they are attached may form a further saturated or aromatic cycle T³ which optionally contains one or two heteroatoms and has 5 to 7 ring atoms and which may be mono- or polysubstituted by identical or different substituents selected from the group consisting of the radicals R⁴, R⁵ and R⁶,
      where R⁴, R⁵ and R⁶ independently of one another represent a C₁-C₃-alkyl or C₁-C₃-alkoxy radical, and
D represents hydrogen or
   represents a C₁-C₆-alkyl or C₁-C₆-alkoxy-C₁-C₆-alkyl radical or
   represents a C₃-C₇-cycloalkyl or 4- to 7-membered monocyclic heterocyclyl radical, where the radicals mentioned may optionally be mono- or polysubstituted by identical or different substituents selected from the group consisting of halogen and hydroxyl and C₁-C₃-alkyl, halo-C₁-C₃-alkyl, C₁-C₃-alkoxy, halo-C₁-C₃-alkoxy and C₁-C₃-alkoxy-C₁-C₃-alkyl radicals, or
A and D together with the atoms to which they are attached form a saturated or unsaturated cycle T⁴ which optionally contains a further heteroatom and has 5 to 7 ring atoms and whose ring-forming atoms may be mono- or polysubstituted by identical or different substituents selected from the group consisting of the radicals R⁷, R⁸ and R⁹,
   where R⁷, R⁸ and R⁹ independently of one another represent halogen or a C₁-C₃-alkyl or C₁-C₃-alkoxy radical.

In formula (I-1), X may represent:

halogen or an optionally monohalogen- or polyhalogen-substituted C₁-C₃-alkyl or C₁-C₃-alkoxy radical.

In formula (I-1), X may preferably represent:

chlorine, bromine or a methyl, ethyl, trifluoromethyl, difluoromethoxy or trifluoromethoxy radical.

In formula (I-1), X may particularly preferably represent:

chlorine or a methyl radical.

In formula (I-1), W and Y independently of one another may represent:

hydrogen or halogen or an optionally monohalogen- or polyhalogen-substituted C₁-C₃-alkyl radical.

In formula (I-1), W and Y independently of one another may preferably represent:

hydrogen, fluorine, chlorine or a methyl, ethyl or trifluoromethyl radical.

In formula (I-1), W and Y independently of one another may more preferably represent:

hydrogen or a methyl radical.

In formula (I-1), X, W and Y dependently of one another may represent:

X represents chlorine or represents a methyl radical, W represents hydrogen or represents a methyl radical and Y represents hydrogen, fluorine, chlorine or represents a methyl radical, or
X and W represent methyl and Y represents hydrogen or
X and Y represent methyl and W represents hydrogen, or
X represents methyl, W represents hydrogen and Y represents chlorine or fluorine.

In formula (I-1), X, W and Y dependently of one another may preferably represent:

X represents a methyl radical and W and Y represent hydrogen, or
X and W represent a methyl radical and Y represents hydrogen, or
X and Y represent a methyl radical and W represents hydrogen.

In formula (I-1), V¹, V² and V³ independently of one another may represent:

hydrogen or halogen or
a C₁-C₃-alkyl, C₁-C₃-haloalkyl, C₁-C₃-alkoxy, C₁-C₃-haloalkoxy or C₁-C₃-alkoxy-C₁-C₃-alkyl radical, and/or
V¹ and V² together with the carbon atoms to which they are attached form a saturated or unsaturated cycle T¹ which optionally contains at least one further heteroatom and has 5 or 6 ring atoms and whose ring-forming atoms may be mono- or polysubstituted by identical or different substituents selected from the group consisting of halogen and a C₁-C₃-alkyl radical.

In formula (I-1), V¹, V² and V³ independently of one another may preferably represent:

hydrogen or fluorine, chlorine, bromine or
a methyl, ethyl, trifluoromethyl, methoxy, ethoxy, difluoromethoxy, trifluoromethoxy or a C₁-C₃-alkoxy-C₁-C₃-alkyl radical and/or
V¹ and V² together with the carbon atoms to which they are attached form a saturated or unsaturated cycle T¹ which optionally contains at least one further heteroatom and has 5 or 6 ring atoms and whose ring-forming atoms may be mono- or polysubstituted by identical or different substituents selected from the group consisting of fluorine, chlorine and a methyl or ethyl radical.

In formula (I-1), V′, V² and V³ independently of one another may preferably represent:

hydrogen, chlorine or fluorine or a methyl or a trifluoromethyl radical.

In formula (I-1), V¹, V² and V³ may more preferably represent:

V¹ represents hydrogen, chlorine or fluorine or represents a methyl or a trifluoromethyl radical, and
V² and V³ independently of one another represent hydrogen, chlorine or fluorine.

In formula (I-1), V¹, V² and V³ may particularly preferably represent:

V¹ represents chlorine, fluorine or a methyl radical
and
V² and V³ independently of one another represent hydrogen, chlorine or fluorine.

In formula (I-1), V¹ may with extraordinary preference represent chlorine or fluorine, in particular chlorine.

In formula (I-1), A may represent:

hydrogen or
an optionally monohalogen- or polyhalogen-substituted C₁-C₆-alkyl or C₁-C₆-alkoxy-C₁-C₆-alkyl radical or
a C₃-C₇-cycloalkyl radical or 4- to 7-membered monocyclic heterocyclyl radical, each of which may be mono- or polysubstituted by identical or different substituents selected from the group consisting of halogen and a C₁-C₃-alkyl radical.

In formula (I-1), A may preferably represent:

hydrogen or
an optionally halogen-substituted C₁-C₄-alkyl or C₁-C₃-alkoxy-C₁-C₃-alkyl radical or
a C₃-C₆-cycloalkyl radical which may be mono- or polysubstituted by identical or different substituents selected from the group consisting of halogen and a C₁-C₃-alkyl radical.

In formula (I-1), A may more preferably represent:

hydrogen or
an optionally halogen-substituted C₁-C₄-alkyl or C₁-C₃-alkoxy-C₁-C₃-alkyl radical or a C₃-C₆-cycloalkyl radical.

In formula (I-1), A may particularly preferably represent:

hydrogen or a C₁-C₄-alkyl, methoxy-C₁-C₂-alkyl or a C₃-C₆-cycloalkyl radical, in particular a methyl, ethyl, isopropyl, n-propyl, isobutyl, sec-butyl, methoxymethyl, methoxyethyl, cyclopropyl, cyclopentyl or cyclohexyl radical.

In formula (I-1), B may represent:

hydrogen or a C₁-C₆-alkyl or C₁-C₃-alkoxy-C₁-C₃-alkyl radical.

In formula (I-1), B may preferably represent:

hydrogen or a C₁-C₃-alkyl radical, in particular a methyl, ethyl or n-propyl radical.

In formula (I-1), B may more preferably represent:

hydrogen or a methyl radical.

In formula (I-1), A and B may together with the carbon atom to which they are attached form:

a saturated or unsaturated cycle T² which optionally contains one or two heteroatoms and has 3 to 8 ring atoms and whose ring-forming atoms may be mono- or polysubstituted by identical or different substituents selected from the group consisting of the radicals R¹, R² and R³,
where R¹, R² and R³ independently of one another
a) represent halogen or hydroxyl or
b) represent a C₁-C₄-alkyl, C₁-C₄-alkoxy, C₁-C₃-alkoxy-C₁-C₃-alkyl, C₁-C₃-alkoxy-C₁-C₃-alkoxy, halo-C₁-C₃-alkyl or halo-C₁-C₃-alkoxy radical which is optionally hydroxyl-substituted in the alkyl moiety and/or
c) two of the radicals R¹, R² and R³ together with the ring atom(s) of the cycle T² to which they are attached may form a further saturated or aromatic cycle T³ which optionally contains one or two heteroatoms and has 5 to 7 ring atoms and may be mono- or polysubstituted by identical or different substituents selected from the group consisting of the radicals R⁴, R⁵ and R⁶, where R⁴, R⁵ and R⁶ independently of one another represent a C₁-C₃-alkyl or C₁-C₃-alkoxy radical.

In formula (I-1), A and B together with the carbon atom to which they are attached may preferably form:

a saturated cycle T² which optionally contains one or two heteroatoms and has 3 to 8 ring atoms and whose ring-forming atoms may be mono- or disubstituted by identical or different substituents selected from the group consisting of the radicals R¹ and R², where R¹ and R² independently of one another
a) represent a C₁-C₄-alkyl, C₁-C₄-alkoxy, C₁-C₃-alkoxy-C₁-C₃-alkyl, C₁-C₃-alkoxy-C₁-C₃-alkoxy or halo-C₁-C₃-alkoxy radical which is optionally hydroxyl-substituted in the alkyl moiety, and/or
b₁) the radicals R¹ and R³ together with the ring atom of the cycle T² to which they are attached may form a further saturated cycle T³ which optionally contains one or two oxygen atoms and has 5 to 7 ring atoms and may be mono- or disubstituted by a C₁-C₃-alkyl radical, or
b₂) the radicals R¹ and R² together with the adjacent ring atoms of the cycle T² to which they are attached may form a further aromatic cycle T³ which has 6 ring atoms and may be mono- or disubstituted by a C₁-C₃-alkyl radical.

In formula (I-1), A and B together with the carbon atom to which they are attached may likewise preferably form:

a saturated cycle T² which optionally contains one or two heteroatoms and has 3 to 8 ring atoms and whose ring-forming atoms may be mono- or disubstituted by identical or different substituents selected from the group consisting of the radicals R¹ and R², where R¹ and R² independently of one another
a) represent hydroxyl or
b) represent a C₁-C₄-alkyl, C₁-C₄-alkoxy, C₁-C₃-alkoxy-C₁-C₃-alkyl, C₁-C₃-alkoxy-C₁-C₃-alkoxy, halo-C₁-C₃-alkyl or halo-C₁-C₃-alkoxy radical which is optionally substituted in the alkyl moiety by hydroxyl.

In formula (I-1), A and B together with the carbon atom to which they are attached may more preferably form:

a saturated cycle T² which optionally contains one heteroatom and has 3 to 8 ring atoms and whose ring-forming atoms may be mono- or disubstituted by identical or different substituents selected from the group consisting of the radicals R¹ and R², where R¹ and R² independently of one another
a) represent a C₁-C₄-alkyl, C₁-C₄-alkoxy, C₁-C₃-alkoxy-C₁-C₃-alkyl, C₁-C₃-alkoxy-C₁-C₃-alkoxy or halo-C₁-C₃-alkoxy radical, and/or
b₁) the radicals R¹ and R² together with the ring atom of the cycle T² to which they are attached may form a further saturated cycle T³ which optionally contains one or two oxygen atoms and has 5 to 7 ring atoms and may be mono- or disubstituted by a C₁-C₃-alkyl radical, or
b₂) the radicals R¹ and R² together with the adjacent ring atoms of the cycle T² to which they are attached may form a further aromatic cycle T³ which has 6 ring atoms.

In formula (I-1), A and B together with the carbon atom to which they are attached may likewise more preferably form:

a saturated cycle T² which optionally contains one heteroatom and has 3 to 8 ring atoms and whose ring-forming atoms may be mono- or disubstituted by identical or different substituents selected from the group consisting of the radicals R¹ and R², where R¹ and R² independently of one another
a) represent hydroxyl or
b) represent a C₁-C₃-alkyl, C₁-C₃-alkoxy, C₁-C₂-alkoxy-C₁-C₃-alkyl, C₁-C₃-alkoxy-C₁-C₃-alkoxy, halo-C₁-C₃-alkyl or halo-C₁-C₃-alkoxy radical which is optionally substituted in the alkyl moiety by hydroxyl.

In formula (I-1), A and B together with the carbon atom to which they are attached may likewise more preferably form:

a saturated cycle T² which optionally contains one oxygen atom and has 3 to 8 ring atoms and whose ring-forming atoms may be mono- or disubstituted by identical or different substituents selected from the group consisting of the radicals R¹ and R², where R¹ and R² independently of one another
a) represent a C₁-C₃-alkyl, C₁-C₄-alkoxy, methoxy-C₁-C₃-alkyl, C₁-C₂-alkoxyethoxy or 2,2,2-trifluoroethoxy radical and/or
b₁) the radicals R¹ and R² together with the ring atom of the cycle T² to which they are attached may form a further saturated cycle T³ which optionally contains one or two oxygen atoms and has 5 or 6 ring atoms and may be mono- or disubstituted by a methyl radical, or
b₂) the radicals R¹ and R² together with the adjacent ring atoms of the cycle T² to which they are attached may form a further aromatic cycle T³ which has 6 ring atoms.

In formula (I-1), A and B together with the carbon atom to which they are attached may particularly preferably form:

a saturated cycle T² which optionally contains one oxygen atom and has 5 to 6 ring atoms and whose ring-forming atoms may be mono- or disubstituted by identical or different substituents selected from the group consisting of the radicals R¹ and R², where R¹ and R² independently of one another
a) represent a C₁-C₃-alkyl, C₁-C₄-alkoxy, methoxy-C₁-C₃-alkyl or 2,2,2-trifluoroethoxy radical and/or
b) the radicals R¹ and R² together with the ring atom of the cycle T² to which they are attached may form a further saturated cycle T³ which optionally contains one or two oxygen atoms and has 5 or 6 ring atoms and may be mono- or disubstituted by a methyl radical.

In formula (I-1), A and B together with the carbon atom to which they are attached may likewise particularly preferably form:

a saturated cycle T² which optionally contains one oxygen atom and has 5 to 6 ring atoms and whose ring-forming atoms may be mono- or disubstituted by identical or different substituents selected from the group consisting of the radicals R¹ and R², where R¹ and R² independently of one another
a) represent hydroxyl or
b) represent a C₁-C₃-alkyl, hydroxymethyl, C₁-C₂-alkoxy, methoxy-C₁-C₂-alkyl, trifluoromethyl, pentafluoroethyl or 2,2,2-trifluoroethoxy radical.

In formula (I-1), A and B together with the carbon atom to which they are attached may with extraordinary preference form:

a saturated cycle T² which optionally contains one oxygen atom and has 6 ring atoms and whose ring-forming atoms may be mono- or disubstituted by identical or different substituents selected from the group consisting of the radicals R¹ and R²,
where R¹ and R² independently of one another represent a C₁-C₃-alkyl, C₁-C₄-alkoxy, methoxy-C₁-C₂-alkyl, 2,2,2-trifluoroethoxy radical.

In formula (I-1), A and B together with the carbon atom to which they are attached may very preferably form a cyclohexane ring or tetrahydropyran ring.

If A and B together with the carbon atom to which they are attached form a cyclohexane ring, the optional substituents R¹, R² and R³ of the cycle T² formed by A and B preferably independently of one another represent hydroxyl or represent a C₁-C₃-alkyl, C₁-C₂-alkoxy or methoxy-C₁-C₂-alkyl radical which is substituted in the alkyl moiety by hydroxyl or represent a pentafluoroethyl, trifluoromethyl or 2,2,2-trifluoroethoxy radical.

If A and B together with the carbon atom to which they are attached form a cyclohexane ring, the optional substituents R¹ and R² of the cycle T² formed by A and B more preferably independently of one another represent hydroxyl or represent a C₁-C₃-alkyl, hydroxymethyl, C₁-C₂-alkoxy, methoxy-C₁-C₂-alkyl, trifluoromethyl, pentafluoroethyl or 2,2,2-trifluoroethoxy radical.

If A and B together with the carbon atom to which they are attached form a cyclohexane ring, a single substituent is particularly preferred, where R¹ represents a C₁-C₃-alkyl, C₁-C₂-alkoxy, methoxy-C₁-C₂-alkyl, trifluoromethyl or 2,2,2-trifluoroethoxy radical.

In formula (I-1), D may represent:

hydrogen or a C₁-C₆-alkyl or C₁-C₆-alkoxy-C₁-C₆-alkyl radical or
a C₃-C₇-cycloalkyl or 4- to 7-membered monocyclic heterocyclyl radical,
where the radicals mentioned may optionally be mono- or polysubstituted by identical or different substituents selected from the group consisting of halogen and/or hydroxyl and C₁-C₃-alkyl, halo-C₁-C₃-alkyl, C₁-C₃-alkoxy, halo-C₁-C₃-alkoxy or C₁-C₃-alkoxy-C₁-C₃-alkyl radicals.

In formula (I-1), D may preferably represent:

hydrogen or a C₁-C₆-alkyl or C₃-C₇-cycloalkyl radical.

In formula (I-1), D may more preferably represent:

hydrogen or a C₁-C₄-alkyl or C₃-C₆-cycloalkyl radical, in particular a methyl, ethyl, n-propyl, isopropyl, cyclopropyl, cyclopentyl or cyclohexyl radical.

In formula (I-1), D may particularly preferably represent:

hydrogen.

In formula (I-1), A and D together with the atoms to which they are attached may form:

a saturated or unsaturated cycle T⁴ which optionally contains a further heteroatom and has 5 to 7 ring atoms and whose ring-forming atoms may be mono- or polysubstituted by identical or different substituents selected from the group consisting of the radicals R⁷, R⁸ and R⁹,
where R⁷, R⁸ and R⁹ independently of one another represent halogen or a C₁-C₃-alkyl or C₁-C₃-alkoxy radical.

In formula (I-1), A and D together with the atoms to which they are attached may preferably form:

a saturated cycle T⁴ which optionally contains a further heteroatom and has 5 to 7 ring atoms.

In formula (I-1), A and D together with the atoms to which they are attached may more preferably form:

a saturated cycle T⁴ which optionally contains sulphur as a further heteroatom and has 5 to 7 ring atoms.

In formula (I-1), A and D together with the atoms to which they are attached may particularly preferably form:

a saturated cycle T⁴ which optionally contains sulphur as a further heteroatom and has 6 ring atoms.

A preferred sub-group of compounds of the general formula (I-1) for use as medicaments is formed by compounds of the general formula (I-1)

in which
X represents chlorine or represents a methyl radical, and
W and Y independently of one another represent hydrogen or represent a methyl radical,
V¹, V² and V³ independently of one another represent hydrogen, chlorine or fluorine or represent a methyl or a trifluoromethyl radical, and
A represents hydrogen or represents an optionally halogen-substituted C₁-C₄-alkyl or C₁-C₃-alkoxy-C₁-C₃-alkyl radical or
   represents a C₃-C₆-cycloalkyl radical which may be mono- or polysubstituted by identical or different substituents selected from the group consisting of halogen and a C₁-C₃-alkyl radical, and
B represents hydrogen or represents a C₁-C₃-alkyl radical, or
A and B together with the carbon atom to which they are attached form a saturated cycle T² which optionally contains one or two heteroatoms and has 3 to 8 ring atoms and whose ring-forming atoms may be mono- or disubstituted by identical or different substituents selected from the group consisting of the radicals R¹ and R², where R¹ and R² independently of one another
   a) represent a C₁-C₄-alkyl, C₁-C₄-alkoxy, C₁-C₃-alkoxy-C₁-C₃-alkyl, C₁-C₃-alkoxy-C₁-C₃-alkoxy or halo-C₁-C₃-alkoxy radical which is optionally hydroxyl-substituted in the alkyl moiety, and/or
   b₁) the radicals R¹ and R² together with the ring atom of the cycle T² to which they are attached may form a further saturated cycle T³ which optionally contains one or two oxygen atoms and has 5 to 7 ring atoms and may be mono- or disubstituted by a C₁-C₃-alkyl radical, or
   b₂) the radicals R¹ and R² together with the adjacent ring atoms of the cycle T² to which they are attached may form a further aromatic cycle T³ which has 6 ring atoms and may be mono- or disubstituted by a C₁-C₃-alkyl radical, and
D represents hydrogen or represents a C₁-C₆-alkyl or C₃-C₇-cycloalkyl radical.

A more preferred sub-group of compounds of the general formula (I-1) for use as medicaments is formed by compounds of the general formula (I-1)

in which
X represents chlorine or represents a methyl radical, and
W and Y independently of one another represent hydrogen or represent a methyl radical,
V¹, V² and V³ independently of one another represent hydrogen, chlorine or fluorine or represent a methyl or a trifluoromethyl radical, and
A represents hydrogen or represents an optionally halogen-substituted C₁-C₄-alkyl or C₁-C₃-alkoxy-C₁-C₃-alkyl radical or represents a C₃-C₆-cycloalkyl radical, and
B represents hydrogen or represents a C₁-C₃-alkyl radical, or
A and B together with the carbon atom to which they are attached form a saturated cycle T² which optionally contains one heteroatom and has 3 to 8 ring atoms and whose ring-forming atoms may be mono- or disubstituted by identical or different substituents selected from the group consisting of the radicals R¹ and R², where R¹ and R² independently of one another
   a) represent a C₁-C₄-alkyl, C₁-C₄-alkoxy, C₁-C₃-alkoxy-C₁-C₃-alkyl, C₁-C₃-alkoxy-C₁-C₃-alkoxy or halo-C₁-C₃-alkoxy radical, and/or
   b₁) the radicals R¹ and R² together with the ring atom of the cycle T² to which they are attached may form a further saturated cycle T³ which optionally contains one or two oxygen atoms and has 5 to 7 ring atoms and may be mono- or disubstituted by a C₁-C₃-alkyl radical, or
   b₂) the radicals R¹ and R² together with the adjacent ring atoms of the cycle T² to which they are attached may form a further aromatic cycle T³ which has 6 ring atoms, and
D represents hydrogen or represents a C₁-C₆-alkyl or C₃-C₇-cycloalkyl radical.

A likewise more preferred sub-group of compounds of the general formula (I-1) for use as medicaments is formed by compounds of the general formula (I-1)

in which
X represents chlorine or represents a methyl radical, and
W and Y independently of one another represent hydrogen or represent a methyl radical,
V¹ represents hydrogen, chlorine or fluorine or represents a methyl or a trifluoromethyl radical, and
V² and V³ independently of one another represent hydrogen, chlorine or fluorine,
A represents hydrogen or represents a C₁-C₄-alkyl, methoxy-C₁-C₂-alkyl or C₃-C₆-cycloalkyl radical,
B represents hydrogen or represents a methyl radical, or
A and B together with the carbon atom to which they are attached form a saturated cycle T² which optionally contains one oxygen atom and has 3 to 8 ring atoms and whose ring-forming atoms may be mono- or disubstituted by identical or different substituents selected from the group consisting of the radicals R¹ and R², where R¹ and R² independently of one another
   a) represent a C₁-C₃-alkyl, C₁-C₄-alkoxy, methoxy-C₁-C₃-alkyl, C₁-C₂-alkoxyethoxy or 2,2,2-trifluoroethoxy radical and/or
   b₁) the radicals R¹ and R² together with the ring atom of the cycle T² to which they are attached may form a further saturated cycle T³ which optionally contains one or two oxygen atoms and has 5 or 6 ring atoms and may be mono- or disubstituted by a methyl radical, or
   b₁) the radicals R¹ and R² together with the adjacent ring atoms of the cycle T² to which they are attached may form a further aromatic cycle T³ which has 6 ring atoms, and
D represents hydrogen or represents a C₁-C₄-alkyl or C₃-C₆-cycloalkyl radical.
   A particularly preferred sub-group of compounds of the general formula (I-1) for use as medicaments is formed by compounds of the general formula (I-1)
   in which
X represents chlorine or represents a methyl radical, and
W and Y independently of one another represent hydrogen or represent a methyl radical,
V¹ represents chlorine, fluorine or a methyl radical, and
V² and V³ independently of one another represent hydrogen, chlorine or fluorine,
A and B together with the carbon atom to which they are attached form a saturated cycle T² which optionally contains one oxygen atom and has 5 to 6 ring atoms and whose ring-forming atoms may be mono- or disubstituted by identical or different substituents selected from the group consisting of the radicals R¹ and R²,
   where R¹ and R² independently of one another
   a) represent a C₁-C₃-alkyl, C₁-C₄-alkoxy, methoxy-C₁-C₂-alkyl or 2,2,2-trifluoroethoxy radical and/or
   b) the radicals R¹ and R² together with the ring atom of the cycle T² to which they are attached may form a further saturated cycle T³ which optionally contains one or two oxygen atoms and has 5 or 6 ring atoms and may be mono- or disubstituted by a methyl radical, and
D represents hydrogen.

A likewise particularly preferred sub-group of compounds of the general formula (I-1) for use as medicaments is formed by compounds of the general formula (I-1)

in which
X represents chlorine or represents a methyl radical, and
W and Y independently of one another represent hydrogen or represent a methyl radical,
V¹ represents chlorine, fluorine or a methyl radical, and
V² and V³ independently of one another represent hydrogen, chlorine or fluorine,
A and B together with the carbon atom to which they are attached form a saturated cycle T² which optionally contains one oxygen atom and has 6 ring atoms and whose ring-forming atoms may be mono- or disubstituted by identical or different substituents selected from the group consisting of the radicals R¹ and R², where R¹ and R² independently of one another represent hydroxyl or represent a C₁-C₃-alkyl, hydroxymethyl, C₁-C₂-alkoxy, methoxy-C₁-C₂-alkyl, trifluoromethyl, pentafluoroethyl or 2,2,2-trifluoroethoxy radical, and
D represents hydrogen.

A preferred sub-group of compounds of the general formula (I) for use as medicaments is also formed by compounds of the general formula (I-2)

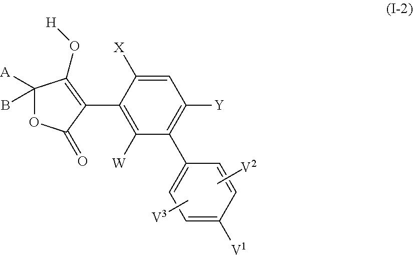

in which
X represents halogen or
   represents an optionally monohalogen- or polyhalogen-substituted C₁-C₃-alkyl or C₁-C₃-alkoxy radical, and
W and Y independently of one another represent hydrogen or halogen or
   represent an optionally monohalogen- or polyhalogen-substituted C₁-C₃-alkyl radical, and
V¹, V² and V³ independently of one another represent hydrogen or halogen or represent a C₁-C₃-alkyl, C₁-C₃-haloalkyl, C₁-C₃-alkoxy, C₁-C₃-haloalkoxy or C₁-C₃-alkoxy-C₁-C₃-alkyl radical, and/or
V¹ and V² together with the carbon atoms to which they are attached form a saturated or unsaturated cycle T¹ which optionally contains at least one further heteroatom and has 5 or 6 ring atoms and whose ring-forming atoms may be mono- or polysubstituted by identical or different substituents selected from the group consisting of halogen and a C₁-C₃-alkyl radical,
A represents hydrogen or
   represents an optionally monohalogen- or polyhalogen-substituted C₁-C₆-alkyl or C₁-C₆-alkoxy-C₁-C₆-alkyl radical or
   represents a C₃-C₇-cycloalkyl radical or 4- to 7-membered monocyclic heterocyclyl radical, each of which may be mono- or polysubstituted by identical or different substituents selected from the group consisting of halogen and a C₁-C₃-alkyl radical, and
B represents hydrogen or represents a C₁-C₆-alkyl or C₁-C₃-alkoxy-C₁-C₃-alkyl radical, or
A and B together with the carbon atom to which they are attached form a saturated or unsaturated cycle T² which optionally contains one or two heteroatoms and has 3 to 8 ring atoms and whose ring-forming atoms may be mono- or polysubstituted by identical or different substituents selected from the group consisting of the radicals R¹, R² and R³,
   where R¹, R² and R³ independently of one another
   a) represent halogen or hydroxyl or
   b) represent a C₁-C₄-alkyl, C₁-C₄-alkoxy, C₁-C₃-alkoxy-C₁-C₃-alkyl, C₁-C₃-alkoxy-C₁-C₃-alkoxy, halo-C₁-C₃-alkyl or halo-C₁-C₃-alkoxy radical which is optionally hydroxyl-substituted in the alkyl moiety and/or
   c) two of the radicals R¹, R² and R³ together with the ring atom(s) of the cycle T² to which they are attached may form a further saturated or aromatic cycle T³ which optionally contains one or two heteroatoms and has 5 to 7 ring atoms and may be mono- or polysubstituted by identical or different substituents selected from the group consisting of R⁴, R⁵ and R⁶,
      where R⁴, R⁵ and R⁶ independently of one another represent a C₁-C₃-alkyl or C₁-C₃-alkoxy radical.

In formula (I-2), X may represent:

halogen or an optionally monohalogen- or polyhalogen-substituted C₁-C₃-alkyl or C₁-C₃-alkoxy radical.

In formula (I-2), X may preferably represent:

chlorine, bromine or a methyl, ethyl, trifluoromethyl, difluoromethoxy or trifluoromethoxy radical.

In formula (I-2), X may preferably represent:

chlorine or a methyl radical.

In formula (I-2), W and Y may independently of one another represent:

hydrogen or halogen or an optionally monohalogen- or polyhalogen-substituted C₁-C₃-alkyl radical.

In formula (I-2), W and Y independently of one another may preferably represent:

hydrogen, fluorine, chlorine or a methyl, ethyl or trifluoromethyl radical.

In formula (I-2), W and Y independently of one another may more preferably represent:

hydrogen or a methyl radical.

In formula (I-2), X, W and Y independently of one another may represent:

X represents chlorine or a methyl radical, W represents hydrogen or a methyl radical and Y represents hydrogen, fluorine, chlorine or a methyl radical or
X and W represent methyl and Y represents hydrogen or
X and Y represent methyl and W represents hydrogen or
X represents methyl, W represents hydrogen and Y represents chlorine or fluorine.

In formula (I-2), X, W and Y independently of one another may preferably represent:

X represents a methyl radical and W and Y represent hydrogen or
X and W represent a methyl radical and Y represents hydrogen or
X and Y represent a methyl radical and W represents hydrogen.

In formula (I-2), V¹, V² and V³ independently of one another may represent:

hydrogen or halogen or a C₁-C₃-alkyl, C₁-C₃-haloalkyl, C₁-C₃-alkoxy, C₁-C₃-haloalkoxy or C₁-C₃-alkoxy-C₁-C₃-alkyl radical, and/or
V¹ and V² together with the carbon atoms to which they are attached form a saturated or unsaturated cycle T¹ which optionally contains at least one further heteroatom and has 5 or 6 ring atoms and whose ring-forming atoms may be mono- or polysubstituted by identical or different substituents selected from the group consisting of halogen and a C₁-C₃-alkyl radical.

In formula (I-2), V¹, V² and V³ independently of one another may preferably represent:

hydrogen or fluorine, chlorine, bromine or
a methyl, ethyl, trifluoromethyl, methoxy, ethoxy, difluoromethoxy, trifluoromethoxy or a C₁-C₃-alkoxy-C₁-C₃-alkyl radical and/or
V¹ and V² together with the carbon atoms to which they are attached form a saturated or unsaturated cycle T¹ which optionally contains at least one further heteroatom and has 5 or 6 ring atoms and whose ring-forming atoms may be mono- or polysubstituted by identical or different substituents selected from the group consisting of fluorine, chlorine and/or a methyl or ethyl radical.

In formula (I-2), V¹, V² and V³ independently of one another may preferably represent:

hydrogen, chlorine or fluorine or a methyl or a trifluoromethyl radical.

In formula (I-2), V¹, V² and V³ may more preferably represent:

V¹ represents hydrogen, chlorine or fluorine or a methyl or a trifluoromethyl radical, and
V² and V³ independently of one another represent hydrogen, chlorine or fluorine.

In formula (I-2), V¹, V² and V³ may particularly preferably represent:

V¹ chlorine, fluorine or a methyl radical
and
V² and V³ independently of one another represent hydrogen, chlorine or fluorine.

In formula (I-2), V¹ may with extraordinary preference represent chlorine or fluorine, in particular chlorine.

In formula (I-2), A may preferably represent:

hydrogen or
an optionally halogen-substituted C₁-C₄-alkyl or C₁-C₃-alkoxy-C₁-C₃-alkyl radical or
a C₃-C₆-cycloalkyl radical which may be mono- or polysubstituted by identical or different substituents selected from the group consisting of halogen and a C₁-C₃-alkyl radical.

In formula (I-2), A may more preferably represent:

hydrogen or
an optionally halogen-substituted C₁-C₄-alkyl or C₁-C₃-alkoxy-C₁-C₃-alkyl radical or a C₃-C₆-cycloalkyl radical.

In formula (I-2), A may particularly preferably represent:

hydrogen or
a C₁-C₄-alkyl, methoxy-C₁-C₂-alkyl or a C₃-C₆-cycloalkyl radical, in particular a methyl, ethyl, isopropyl, n-propyl, isobutyl, sec-butyl, methoxymethyl, methoxyethyl, cyclopropyl, cyclopentyl or cyclohexyl radical.

In formula (I-2), B may represent:

hydrogen or a C₁-C₆-alkyl or C₁-C₃-alkoxy-C₁-C₃-alkyl radical.

In formula (I-2), B may preferably represent:

hydrogen or a C₁-C₃-alkyl radical, in particular a methyl, ethyl or
n-propyl radical.

In formula (I-2), B may preferably represent:

hydrogen or a C₁-C₃-alkyl radical.

In formula (I-2), B may more preferably represent:

hydrogen or a methyl radical.

In formula (I-2), A and B together with the carbon atom to which they are attached may form:

a saturated or unsaturated cycle T² which optionally contains one or two heteroatoms and has 3 to 8 ring atoms and whose ring-forming atoms may be mono- or polysubstituted by identical or different substituents selected from the group consisting of the radicals R¹, R² and R³,
where R¹, R² and R³ independently of one another
a) represent halogen or hydroxyl or
b) represent a C₁-C₄-alkyl, C₁-C₄-alkoxy, C₁-C₃-alkoxy-C₁-C₃-alkyl, C₁-C₃-alkoxy-C₁-C₃-alkoxy, halo-C₁-C₃-alkyl or halo-C₁-C₃-alkoxy radical which is optionally hydroxyl-substituted in the alkyl moiety and/or
c) two of the radicals R¹, R² and R³ together with the ring atom(s) of the cycle T² to which they are attached may form a further saturated or aromatic cycle T³ which optionally contains one or two heteroatoms and has 5 to 7 ring atoms and may be mono- or polysubstituted by identical or different substituents selected from the group consisting of the radicals R⁴, R⁵ and R⁶,
   where R⁴, R⁵ and R⁶ independently of one another represent a C₁-C₃-alkyl or C₁-C₃-alkoxy radical.

In formula (I-2), A and B together with the carbon atom to which they are attached may preferably form:

a saturated cycle T² which optionally contains one oxygen atom and has 3 to 8 ring atoms and whose ring-forming atoms may be mono- or disubstituted by identical or different substituents selected from the group consisting of the radicals R¹ and R², where R¹ and R² independently of one another
a) represent a C₁-C₃-alkyl, C₁-C₃-alkoxy, C₁-C₃-alkoxy-C₁-C₃-alkyl, C₁-C₃-alkoxy-C₁-C₃-alkoxy or halo-C₁-C₃-alkoxy radical which is optionally hydroxyl-substituted in the alkyl moiety, and/or
b₁) the radicals R¹ and R² together with the ring atom of the cycle T² to which they are attached may form a further saturated cycle T³ which optionally contains one or two oxygen atoms and has 5 to 7 ring atoms and may be mono- or disubstituted by a C₁-C₃-alkyl radical, or
b₂) the radicals R¹ and R² together with the adjacent ring atoms of the cycle T² to which they are attached may form a further aromatic cycle T³ which has 6 ring atoms and may be mono- or disubstituted by a C₁-C₃-alkyl radical.

In formula (I-2), A and B together with the carbon atom to which they are attached may likewise preferably form:

a saturated cycle T² which optionally contains one or two heteroatoms and has 3 to 8 ring atoms and whose ring-forming atoms may be mono- or disubstituted by identical or different substituents selected from the group consisting of the radicals R¹ and R², where R¹ and R² independently of one another
a) represent hydroxyl or
b) represent a C₁-C₄-alkyl, C₁-C₄-alkoxy-, C₁-C₃-alkoxy-C₁-C₃-alkyl, C₁-C₃-alkoxy-C₁-C₃-alkoxy-, halo-C₁-C₃-alkyl or halo-C₁-C₃-alkoxy radical which is optionally hydroxyl-substituted in the alkyl moiety.

In formula (I-2), A and B together with the carbon atom to which they are attached may more preferably form:

a saturated cycle T² which optionally contains one oxygen or sulphur atom and has 5 to 7 ring atoms and whose ring-forming atoms may be mono- or disubstituted by identical or different substituents selected from the group consisting of the radicals R¹ and R²,
where R¹ and R² independently of one another
a) represent a C₁-C₃-alkyl, C₁-C₃-alkoxy, C₁-C₃-alkoxy-C₁-C₃-alkyl or halo-C₁-C₃-alkoxy radical and/or
b) the radicals R¹ and R² together with the ring atom of the cycle T² to which they are attached may form a further saturated cycle T³ which optionally contains one or two oxygen atoms and has 5 or 6 ring atoms.

In formula (I-2), A and B together with the carbon atom to which they are attached may likewise more preferably form:

a saturated cycle T² which optionally contains one heteroatom and has 3 to 8 ring atoms and whose ring-forming atoms may be mono- or polysubstituted by identical or different substituents selected from the group consisting of the radicals R¹ and R², where R¹ and R² independently of one another
a) represent hydroxyl or
b) represent a C₁-C₃-alkyl, C₁-C₃-alkoxy, C₁-C₂-alkoxy-C₁-C₃-alkyl, C₁-C₃-alkoxy-C₁-C₃-alkoxy, halo-C₁-C₃-alkyl or halo-C₁-C₃-alkoxy radical which is optionally hydroxyl-substituted in the alkyl moiety.

In formula (I-2), A and B together with the carbon atom to which they are attached may particularly preferably form:

a saturated cycle T² which optionally contains one oxygen atom and has 5 to 6 ring atoms and whose ring-forming atoms may be mono- or disubstituted by identical or different substituents selected from the group consisting of the radicals R¹ and R², where R¹ and R² independently of one another
a) represent a C₁-C₃-alkyl, C₁-C₄-alkoxy, methoxy-C₁-C₂-alkyl, 2,2,2-trifluoroethoxy radical and/or
b) the radicals R¹ and R² together with the ring atom of the cycle T² to which they are attached may form a further saturated cycle T³ which optionally contains one or two oxygen atoms and has 5 or 6 ring atoms and may be mono- or disubstituted by a methyl radical.

In formula (I-I), A and B together with the carbon atom to which they are attached may likewise particularly preferably form:

a saturated cycle T² which optionally contains one oxygen atom and has 5 to 6 ring atoms and whose ring-forming atoms may be mono- or disubstituted by identical or different substituents selected from the group consisting of the radicals R¹ and R², where R¹ and R² independently of one another
a) represent hydroxyl or
b) represent a C₁-C₃-alkyl, hydroxymethyl, C₁-C₂-alkoxy, methoxy-C₁-C₂-alkyl, trifluoromethyl, pentafluoroethyl or 2,2,2-trifluoroethoxy radical.

In formula (I-2), A and B together with the carbon atom to which they are attached may with extraordinary preference form:

a saturated cycle T² which optionally contains one oxygen atom and has 6 ring atoms and whose ring-forming atoms may be mono- or disubstituted by identical or different substituents selected from the group consisting of the radicals R¹ and R², where R¹ and R² independently of one another represent a C₁-C₃-alkyl, C₁-C₄-alkoxy, methoxy-C₁-C₂-alkyl or 2,2,2-trifluoroethoxy radical.

In formula (I-2), A and B together with the carbon atom to which they are attached may very preferably form a cyclohexane ring or tetrahydropyran ring.

If A and B together with the carbon atom to which they are attached form a cyclohexane ring, the optional substituents R¹, R² and R³ of the cycle T² formed by A and B preferably independently of one another represent hydroxyl or represent a C₁-C₃-alkyl, C₁-C₂-alkoxy or methoxy-C₁-C₂-alkyl radical which is substituted in the alkyl moiety by hydroxyl or represent a pentafluoroethyl, trifluoromethyl or 2,2,2-trifluoroethoxy radical.

If A and B together with the carbon atom to which they are attached form a cyclohexane ring, the optional substituents R¹ and R² of the cycle T² formed by A and B more preferably independently of one another represent hydroxyl or represent a C₁-C₃-alkyl, hydroxymethyl, C₁-C₂-alkoxy, methoxy-C₁-C₂-alkyl, trifluoromethyl, pentafluoroethyl or 2,2,2-trifluoroethoxy radical.

If A and B together with the carbon atom to which they are attached form a cyclohexane ring, a single substituent is particularly preferred, where R¹ represents a C₁-C₃-alkyl, C₁-C₂-alkoxy, methoxy-C₁-C₂-alkyl, trifluoromethyl or 2,2,2-trifluoroethoxy radical.

A preferred sub-group of compounds of the general formula (I-2) for use as medicaments is formed by compounds of the general formula (I-2)

in which
X represents chlorine or represents a methyl radical, and
W and Y independently of one another represent hydrogen or represent a methyl radical,
V¹, V² and V³ independently of one another represent hydrogen, chlorine or fluorine or represent a methyl or a trifluoromethyl radical, and
A represents hydrogen or represents an optionally halogen-substituted
   C₁-C₃-alkyl or C₁-C₃-alkoxy-C₁-C₃-alkyl radical or
   represents a C₃-C₆-cycloalkyl radical which may be mono- or polysubstituted by identical or different substituents selected from the group consisting of halogen and a C₁-C₃-alkyl radical, and
B represents hydrogen or represents a C₁-C₃-alkyl radical, or
A and B together with the carbon atom to which they are attached form a saturated cycle T² which optionally contains one oxygen atom and has 3 to 8 ring atoms and whose ring-forming atoms may be mono- or disubstituted by identical or different substituents selected from the group consisting of the radicals R¹ and R², where R¹ and R² independently of one another
   a) represent a C₁-C₃-alkyl, C₁-C₃-alkoxy, C₁-C₃-alkoxy-C₁-C₃-alkyl, C₁-C₃-alkoxy-C₁-C₃-alkoxy or halo-C₁-C₃-alkoxy radical which is optionally hydroxyl-substituted in the alkyl moiety, and/or
   b₁) the radicals R¹ and R² together with the ring atom of the cycle T² to which they are attached may form a further saturated cycle T³ which optionally contains one or two oxygen atoms and has 5 to 7 ring atoms and may be mono- or disubstituted by a C₁-C₃-alkyl radical, or
   b₂) the radicals R¹ and R² together with the adjacent ring atoms of the cycle T² to which they are attached may form a further aromatic cycle T³ which has 6 ring atoms and may be mono- or disubstituted by a C₁-C₃-alkyl radical.

A more preferred sub-group of compounds of the general formula (I-2) for use as medicaments is formed by compounds of the general formula (I-2)

in which
X represents chlorine or represents a methyl radical, and
W and Y independently of one another represent hydrogen or represent a methyl radical,
V¹ represents hydrogen, chlorine or fluorine, and
V² and V³ independently of one another represent hydrogen, chlorine or fluorine,
A represents hydrogen or represents a C₁-C₃-alkyl or C₃-C₆-cycloalkyl radical, and
B represents hydrogen or represents a methyl radical, or
A and B together with the carbon atom to which they are attached form a saturated cycle T² which optionally contains one oxygen or sulphur atom and has 5 to 7 ring atoms and whose ring-forming atoms may be mono- or disubstituted by identical or different substituents selected from the group consisting of the radicals R¹ and R²,
   where R¹ and R² independently of one another
   a) represent a C₁-C₃-alkyl, C₁-C₃-alkoxy, C₁-C₃-alkoxy-C₁-C₃-alkyl or halo-C₁-C₃-alkoxy radical and/or
   b) the radicals R¹ and R² together with the ring atom of the cycle T² to which they are attached may form a further saturated cycle T³ which optionally contains one or two oxygen atoms and has 5 or 6 ring atoms.

A first group of the compounds of the formula (I) described in the experimental part is formed by compounds which, as a result of a specific disclosure, belong to the prior art. If known, these compounds are marked in the present application by a reference to the example number or table in the publication in which they are disclosed.

A second group of the compounds of the formula (I) described in the experimental part is formed by compounds which are embraced by a generic disclosure of the prior art. These compounds are marked by the reference “compound according to”.

A third group of the compounds of the formula (I) described in the experimental part is formed by compounds which are neither part of the prior art owing to a specific disclosure nor embraced by a generic disclosure of the prior art.

The present application provides the compounds of the second and third group of the compounds of the formula (I-I) described in the experimental section:
(5s,8s)-3-(4′-chloro-3′-fluoro-4-methylbiphenyl-3-yl)-4-hydroxy-8-(trifluoromethyl)-1-azaspiro[4.5]dec-3-en-2-one
(5s,8s)-3-(4′-chloro-5-fluoro-4-methylbiphenyl-3-yl)-4-hydroxy-8-(trifluoromethyl)-1-azaspiro[4.5]dec-3-en-2-one
(5s,8s)-3-(4,4′-dichloro-3′-fluorobiphenyl-3-yl)-4-hydroxy-8-(trifluoromethyl)-1-azaspiro[4.5]dec-3-en-2-one
(5s,8s)-3-(4′-chloro-3′-fluoro-2,4-dimethylbiphenyl-3-yl)-4-hydroxy-8-(trifluoromethyl)-1-azaspiro[4.5]dec-3-en-2-one
3-(4′-chloro-3′,6-difluoro-4-methylbiphenyl-3-yl)-4-hydroxy-8-(trifluoromethyl)-1-azaspiro[4.5]dec-3-en-2-one
(5s,8s)-3-(4′-chloro-2,4-dimethylbiphenyl-3-yl)-4-hydroxy-8-(trifluoromethyl)-1-azaspiro[4.5]dec-3-en-2-one
(5s,8s)-4-hydroxy-8-(trifluoromethyl)-3-(3′,4′,5-trifluoro-4-methylbiphenyl-3-yl)-1-azaspiro[4.5]dec-3-en-2-one
3-(4′-chloro-4,6-dimethylbiphenyl-3-yl)-4-hydroxy-8 (trifluoromethyl)-1-azaspiro[4.5]dec-3-en-2-one
(5s,8s)-3-(4′-chloro-4,6-dimethylbiphenyl-3-yl)-4-hydroxy-8-(trifluoromethyl)-1-azaspiro[4.5]dec-3-en-2-one
(5s,8s)-3-(4-chloro-3′,4′-difluorobiphenyl-3-yl)-4-hydroxy-8-(trifluoromethyl)-1-azaspiro[4.5]dec-3-en-2-one
3-(4′-chloro-6-fluoro-4-methylbiphenyl-3-yl)-4-hydroxy-8-(trifluoromethyl)-1-azaspiro[4.5]dec-3-en-2-one
(5s,8s)-3-(3′,4′-difluoro-2,4-dimethylbiphenyl-3-yl)-4-hydroxy-8-(trifluoromethyl)-1-azaspiro[4.5]dec-3-en-2-one
(5s,8s)-3-(4′-chloro-3′,5-difluoro-4-methylbiphenyl-3-yl)-4-hydroxy-8-(trifluoromethyl)-1-azaspiro[4.5]dec-3-en-2-one
(5s,8s)-3-(4′-chloro-3′,5-difluoro-4-methylbiphenyl-3-yl)-4-hydroxy-8-methoxy-1-azaspiro[4.5]dec-3-en-2-one
(5s,8s)-3-(4′-chloro-5-fluoro-4-methylbiphenyl-3-yl)-4-hydroxy-8-methoxy-1-azaspiro[4.5]dec-3-en-2-one
(5s,8s)-4-hydroxy-8-methoxy-3-(3′,4′,5-trifluoro-4-methylbiphenyl-3-yl)-1-azaspiro[4.5]dec-3-en 2-one
(5s,8s)-3-(4′,6-dichloro-3′-fluoro-4-methylbiphenyl-3-yl)-4-hydroxy-8-methoxy-1-azaspiro[4.5]dec-3-en-2-one
(5s,8s)-3-(4′,6-dichloro-4-methylbiphenyl-3-yl)-4-hydroxy-8-methoxy-1-azaspiro[4.5]dec-3-en-2-one
(5s,8s)-3-(4′-chloro-4-methylbiphenyl-3-yl)-4-hydroxy-8-(hydroxymethyl)-1-azaspiro[4.5]dec-3-en-2-one
(5s,8s)-3-(4′-chloro-4-methylbiphenyl-3-yl)-4-hydroxy-8-(trifluoromethyl)-1-azaspiro[4.5]dec-3-en-2-one
(5s,8s)-3-(4,4′-dichlorobiphenyl-3-yl)-4-hydroxy-8-methoxy-1-azaspiro[4.5]dec-3-en-2-one
(5s,8s)-3-(4′-chloro-2,4-dimethylbiphenyl-3-yl)-4-hydroxy-8-methoxy-1-azaspiro[4.5]dec-3-en-2-one
(5r,8r)-3-(4′-chloro-4-methylbiphenyl-3-yl)-4,8-dihydroxy-8-(trifluoromethyl)-1-azaspiro[4.5]dec-3-en-2-one
(5r,8r)-3-(4′-chloro-3′-fluoro-4-methylbiphenyl-3-yl)-4,8-dihydroxy-8-(trifluoromethyl)-1-azaspiro[4.5]dec-3-en-2-one
(5r,8r)-3-(3′,4′-difluoro-4-methylbiphenyl-3-yl)-4,8-dihydroxy-8-(trifluoromethyl)-1-azaspiro[4.5]dec-3-en-2-one
(5r,8r)-3-(4′-chloro-2,4-dimethylbiphenyl-3-yl)-4,8-dihydroxy-8-(trifluoromethyl)-1-azaspiro[4.5]dec-3-en-2-one
(5r,8r)-3-(4′-chloro-3′-fluoro-2,4-dimethylbiphenyl-3-yl)-4,8-dihydroxy-8-(trifluoromethyl)-1-azaspiro[4.5]dec-3-en-2-one
(5r,8r)-3-(4′-chloro-6-fluoro-4-methylbiphenyl-3-yl)-4,8-dihydroxy-8-(trifluoromethyl)-1-azaspiro[4.5]dec-3-en-2-one
(5s,8s)-3-(3′,4′-difluoro-4-methylbiphenyl-3-yl)-4-hydroxy-8-(trifluoromethyl)-1-azaspiro[4.5]dec-3-en-2-one
(5r,8r)-3-(3′,4′-difluoro-4-methylbiphenyl-3-yl)-4-hydroxy-8-(trifluoromethyl)-1-azaspiro[4.5]dec-3-en-2-one
(5r,8r)-3-(4′-chloro-3′-fluoro-4-methylbiphenyl-3-yl)-4,8-dihydroxy-8-(pentafluoroethyl)-1-azaspiro[4.5]dec-3-en-2-one)
(5r,8r)-3-(4′-chloro-6-fluoro-4-methylbiphenyl-3-yl)-4,8-dihydroxy-8-(pentafluoroethyl)-1-azaspiro[4.5]dec-3-en-2-one
(5S,7S)-3-(4′-chloro-4-methylbiphenyl-3-yl)-4-hydroxy-7-(trifluoromethyl)-1-azaspiro[4.5]dec-3-en-2-one
(5s,8s)-3-(4,4′-dichlorobiphenyl-3-yl)-4-hydroxy-8 (trifluoromethyl)-1-azaspiro[4.5]dec-3-en-2-one
(5s,8s)-3-(4′-chloro-6-fluoro-4-methylbiphenyl-3-yl)-4-hydroxy-8-methoxy-1-azaspiro[4.5]dec-3-en-2-one
(5S,7S)-3-(4,4′-dichlorobiphenyl-3-yl)-4-hydroxy-7-(trifluoromethyl)-1-azaspiro[4.5]dec-3-en-2-one
3-(4,4′-dichlorobiphenyl-3-yl)-4-hydroxy-8,8-dimethyl-1-azaspiro[4.5]dec-3-en-2-one
3-(4,4′-dichlorobiphenyl-3-yl)-4-hydroxy-8-methyl-1-azaspiro[4.5]dec-3-en-2-one
3-(4,4′-dichlorobiphenyl-3-yl)-4-hydroxy-1-azaspiro[4.5]dec-3-en-2-one
(5s,8s)-3-(4,4′-dichlorobiphenyl-3-yl)-4-hydroxy-8-isopropyl-1-azaspiro[4.5]dec-3-en-2-one
(5s,8s)-3-(2′-chloro-4′-fluoro-4-methylbiphenyl-3-yl)-4-hydroxy-8-methoxy-1-azaspiro[4.5]dec-3-en-2-one

The present application furthermore provides the compounds, described in the experimental section, of the formula (I-1) for use as medicaments:
3-(4′-chloro-2,4-dimethylbiphenyl-3-yl)-4-hydroxy-8-oxa-1-azaspiro[4.5]dec-3-en-2-one (Ex. 1-1),
(5s,8s)-3-(4′-chloro-4-methylbiphenyl-3-yl)-4-hydroxy-8-methoxy-1-azaspiro[4.5]dec-3-en-2-one (Ex. 1-2),
3-(4′-chloro-4-methylbiphenyl-3-yl)-4-hydroxy-7-methyl-8-oxa-1-azaspiro[4.5]dec-3-en-2-one (Ex. 1-3),
3-(4′-chloro-2,4-dimethylbiphenyl-3-yl)-4-hydroxy-8-methyl-1-azaspiro[4.5]dec-3-en-2-one (Ex. 1-4),
3-(4′-chloro-4-methylbiphenyl-3-yl)-4-hydroxy-7-oxa-1-azaspiro[4.5]dec-3-en-2-one (Ex. 1-5),
3-(4′-chloro-4-methylbiphenyl-3-yl)-4-hydroxy-8-oxa-1-azaspiro[4.5]dec-3-en-2-one (Ex. 1-6),
3-(4′-chloro-4,6-dimethylbiphenyl-3-yl)-4-hydroxy-8-methoxy-1-azaspiro[4.5]dec-3-en-2-one (Ex. 1-7),
3-(4′-chloro-4,6-dimethylbiphenyl-3-yl)-4-hydroxy-7-oxa-1-azaspiro[4.5]dec-3-en-2-one (Ex. 1-8),
3-(4′-chloro-2,4,6-trimethylbiphenyl-3-yl)-4-hydroxy-8-methoxy-1-azaspiro[4.5]dec-3-en-2-one (Ex. 1-9),
3-(4′-chloro-2,4-dimethylbiphenyl-3-yl)-4-hydroxy-7-oxa-1-azaspiro[4.5]dec-3-en-2-one (Ex. 1-10),
3-(4,4′-dichlorobiphenyl-3-yl)-4-hydroxy-8-methoxy-1-azaspiro[4.5]dec-3-en-2-one (Ex. 1-11),
4-hydroxy-3-[4-methyl-4′-(trifluoromethyl)biphenyl-3-yl]-1-azaspiro[4.5]dec-3-en-2-one (Ex. 1-12),
3-(4,4′-dimethylbiphenyl-3-yl)-4-hydroxy-1-azaspiro[4.5]dec-3-en-2-one (Ex. 1-13),
3-(4,4′-dimethylbiphenyl-3-yl)-4-hydroxy-7-oxa-1-azaspiro[4.5]dec-3-en-2-one (Ex. 1-14),
3-(4,4′-dimethylbiphenyl-3-yl)-4-hydroxy-8-oxa-1-azaspiro[4.5]dec-3-en-2-one (Ex. 1-15),
3-(4′-chloro-4-methylbiphenyl-3-yl)-4-hydroxy-1-methyl-1,5-dihydro-2H-pyrrol-2-one (Ex. 1-16),
3-(4′-chloro-4-methylbiphenyl-3-yl)-4-hydroxy-1 (propan 2-yl)-1,5-dihydro-2H-pyrrol-2-one (Ex. 1-17),
3-(4′-chloro-4-methylbiphenyl-3-yl)-1 cyclopropyl-4-hydroxy-1,5-dihydro-2H-pyrrol-2-one (Ex. 1-18),
3-(4′-chloro-4-methylbiphenyl-3-yl)-4-hydroxy-5-methyl-1-(propan-2-yl)-1,5-dihydro-2H-pyrrol-2-one (Ex. 1-19),
3-(4′-chloro-4-methylbiphenyl-3-yl)-4-hydroxy-8,8-dimethyl-1-azaspiro[4.5]dec-3-en-2-one (Ex. 1-20),
7-(4′-chloro-4-methylbiphenyl-3-yl)-8-hydroxy-1,3,4,8a-tetrahydro-6H-pyrrolo[2,1-c][1,4]thiazin-6-one (Ex. 1-21),
3-(4′-chloro-4-methylbiphenyl-3-yl)-1 cyclohexyl-4-hydroxy-1,5-dihydro-2H-pyrrol-2-one (Ex. 1-22),
3-(4′-chloro-4-methylbiphenyl-3-yl)-4-hydroxy-1-azaspiro[4.7]dodec-3-en-2-one (Ex. 1-23),
3-(4′-chloro-4-methylbiphenyl-3-yl)-4-hydroxy-8 (propan 2-yl)-1-azaspiro[4.5]dec-3-en-2-one (Ex. 1-24),
4′-(4′-chloro-4-methylbiphenyl-3-yl)-3′-hydroxy-1,3-dihydrospiro[indene-2,2′-pyrrol]-5′(1′H)-one (Ex. 1-25),
3-(4′-chloro-4-methylbiphenyl-3-yl)-4-hydroxy-1-azaspiro[4.6]undec-3-en-2-one (Ex. 1-26),
3-(4′-chloro-4-methylbiphenyl-3-yl)-4-hydroxy-1-(2-methylpropyl)-1,5-dihydro-2H-pyrrol-2-one (Ex. 1-27),
3-(4′-chloro-4-methylbiphenyl-3-yl)-8-ethoxy-4-hydroxy-1-azaspiro[4.5]dec-3-en-2-one (Ex. 1-28),
(5s,8s)-3-(4,4′-dichlorobiphenyl-3-yl)-4-hydroxy-8-(2,2,2-trifluoroethoxy)-1-azaspiro[4.5]dec-3-en-2-one (Ex. 1-29),
(5s,8s)-3-(4′-chloro-2,4-dimethylbiphenyl-3-yl)-4-hydroxy-8-(2,2,2-trifluoroethoxy)-1-azaspiro[4.5]dec-3-en-2-one (Ex. 1-30),
3-(4′-fluoro-2,4-dimethylbiphenyl-3-yl)-4-hydroxy-8-(2,2,2-trifluoroethoxy)-1-azaspiro[4.5]dec-3-en-2-one (Ex. 1-31),
3-(4′-fluoro-4-methylbiphenyl-3-yl)-4-hydroxy-8-(2,2,2-trifluoroethoxy)-1-azaspiro[4.5]dec-3-en-2-one (Ex. 1-32),
3-(4′-chloro-4-methylbiphenyl-3-yl)-4-hydroxy-8-(2,2,2-trifluoroethoxy)-1-azaspiro[4.5]dec-3-en-2-one (Ex. 1-33),
11-(4′-chloro-2,4-dimethylbiphenyl-3-yl)-12-hydroxy-1-oxa-9-azadispiro[4.2.4.2]tetradec-11-en-10-one (Ex. 1-34),
11-(3′,4′-difluoro-4-methylbiphenyl-3-yl)-12-hydroxy-1-oxa-9-azadispiro[4.2.4.2]tetradec-11-en-10-one (Ex. 1-35),
4-hydroxy-3-(3′,4′,5′-trifluoro-4-methylbiphenyl-3-yl)-8 oxa 1 azaspiro[4.5]dec-3-en-2-one (Ex. 1-36),
3-(3′,4′-difluoro-4-methylbiphenyl-3-yl)-4-hydroxy-8-oxa-1-azaspiro[4.5]dec-3-en-2-one (Ex. 1-37),
3-(4-chloro-3′,4′,5′-trifluorobiphenyl-3-yl)-4-hydroxy-8-oxa-1-azaspiro[4.5]dec-3-en-2-one (Ex. 1-38),
3-(4-chloro-3′-fluoro-4′-methylbiphenyl-3-yl)-4-hydroxy-8-oxa-1-azaspiro[4.5]dec-3-en-2-one (Ex. 1-39),
3-(4′-fluoro-2,4-dimethylbiphenyl-3-yl)-4-hydroxy-1-azaspiro[4.5]dec-3-en-2-one (Ex. 1-40),
3-(4-chloro-4′-fluorobiphenyl-3-yl)-4-hydroxy-8-oxa-1-azaspiro[4.5]dec-3-en-2-one (Ex. 1-41),
(5s,8s)-3-(4′-fluoro-2,4,6-trimethylbiphenyl-3-yl)-4-hydroxy-8-methoxy-1-azaspiro[4.5]dec-3-en-2-one (Ex. 1-42),
(5s,8s)-3-(3′-chloro-4′-fluoro-4-methylbiphenyl-3-yl)-4-hydroxy-8-methoxy-1-azaspiro[4.5]dec-3-en-2-one (Ex. 1-43),
(5s,8s)-3-(4-chloro-4′-fluorobiphenyl-3-yl)-4-hydroxy-8-methoxy-1-azaspiro[4.5]dec-3-en-2-one (Ex. 1-44),
(5s,8s)-3-(4′-fluoro-2,4-dimethylbiphenyl-3-yl)-4-hydroxy-8-methoxy-1-azaspiro[4.5]dec-3-en-2-one (Ex. 1-45),
3-(4′-chloro-4-methylbiphenyl-3-yl)-4-hydroxy-5-(methoxymethyl)-5-methyl-1,5-dihydro-2H-pyrrol-2-one (Ex. 1-46),
3-(4′-chloro-4-methylbiphenyl-3-yl)-4-hydroxy-5 (2 methoxyethyl)-5-methyl-1,5-dihydro-2H-pyrrol-2-one (Ex. 1-47),
3-(4′-chloro-2,4-dimethylbiphenyl-3-yl)-4-hydroxy-7-methoxy-1-azaspiro[4.4]non-3-en-2-one (Ex. 1-48),
rel-(5R,7R)-3-(4′-chloro-4-methylbiphenyl-3-yl)-4-hydroxy-7-(2-methylpropoxy)-1-azaspiro[4.4]non-3-en-2-one (Ex. 1-49),
rel-(5R,7S)-3-(4′-chloro-4-methylbiphenyl-3-yl)-7 (2 ethoxyethoxy)-4-hydroxy-1-azaspiro[4.4]non-3-en-2-one (Ex. 1-50),
3-(4′-chloro-4-methylbiphenyl-3-yl)-4-hydroxy-7-(methoxymethyl)-1-azaspiro[4.5]dec-3-en-2-one (Ex. 1-51),
3-(4,4′-dichlorobiphenyl-3-yl)-4-hydroxy-7-(methoxymethyl)-1-azaspiro[4.5]dec-3-en-2-one (Ex. 1-52),
3-(4′-chloro-2,4-dimethylbiphenyl-3-yl)-4-hydroxy-8-(methoxymethyl)-1-azaspiro[4.5]dec-3-en 2-one (Ex. 1-53),
3-(4,4′-dichlorobiphenyl-3-yl)-4-hydroxy-8-(methoxymethyl)-1-azaspiro[4.5]dec-3-en-2-one (Ex. 1-54),
3-(4′-chloro-2,4-dimethylbiphenyl-3-yl)-4-hydroxy-7-(2-methoxyethyl)-1-azaspiro[4.5]dec-3-en 2-one (Ex. 1-55),
3-(4′-chloro-4-methylbiphenyl-3-yl)-4-hydroxy-8-(methoxymethyl)-1-azaspiro[4.5]dec-3-en-2-one (Ex. 1-56),
3-(4′-chloro-2,4-dimethylbiphenyl-3-yl)-4-hydroxy-8-(2-methoxyethyl)-1-azaspiro[4.5]dec-3-en 2-one (Ex. 1-57),
3-(4,4′-dichlorobiphenyl-3-yl)-4-hydroxy-9,13-dioxa-1-azadispiro[4.2.5.2]pentadec-3-en-2-one (Ex. 1-58),
3-(4′-chloro-4-methylbiphenyl-3-yl)-4-hydroxy-9,13-dioxa-1-azadispiro[4.2.5.2]pentadec-3-en 2-one (Ex. 1-59),
11-(4′-chloro-4-methylbiphenyl-3-yl)-12-hydroxy-1,4-dioxa-9-azadispiro[4.2.4.2]tetradec-11-en-10-one (Ex. 1-60),
11-(4,4′-dichlorobiphenyl-3-yl)-12-hydroxy-1,4-dioxa-9-azadispiro[4.2.4.2]tetradec-11-en-10-one (Ex. 1-61),
11-(4′-chloro-2,4-dimethylbiphenyl-3-yl)-12-hydroxy-2-methyl-1,4-dioxa-9-azadispiro[4.2.4.2]tetradec-11-en-10-one (Ex. 1-62),
11-(4′-chloro-4-methylbiphenyl-3-yl)-12-hydroxy-2-methyl-1,4-dioxa-9-azadispiro[4.2.4.2]tetradec-11-en-10-one (Ex. 1-63),
11-(4′-chloro-4-methylbiphenyl-3-yl)-12-hydroxy-2,3-dimethyl-1,4-dioxa-9-azadispiro[4.2.4.2]tetradec-11-en-10-one (Ex. 1-64),
3-(4,4′-dichlorobiphenyl-3-yl)-4-hydroxy-11,11-dimethyl-9,13-dioxa-1-azadispiro[4.2.5.2]pentadec-3-en-2-one (Ex. 1-65),
3-(4′-chloro-4-methylbiphenyl-3-yl)-4-hydroxy-11-methyl-9,13-dioxa-1-azadispiro[4.2.5.2]pentadec-3-en-2-one (Ex. 1-66),
3-(4′-chloro-4-methylbiphenyl-3-yl)-4-hydroxy-11,11-dimethyl-9,13-dioxa-1-azadispiro[4.2.5.2]pentadec-3-en-2-one (Ex. 1-67),
11-(4,4′-dichlorobiphenyl-3-yl)-12-hydroxy-2-methyl-1,4-dioxa-9-azadispiro[4.2.4.2]tetradec-11-en-10-one (Ex. 1-68),
3-(4,4′-dichlorobiphenyl-3-yl)-4-hydroxy-11-methyl-9,13-dioxa-1-azadispiro[4.2.5.2]pentadec-3-en-2-one (Ex. 1-69),
11-(4′-chloro-2,4-dimethylbiphenyl-3-yl)-12-hydroxy-1,4-dioxa-9-azadispiro[4.2.4.2]tetradec-11-en-10-one (Ex. 1-70),
11-(4,4′-dichlorobiphenyl-3-yl)-12-hydroxy-2,3-dimethyl-1,4-dioxa-9-azadispiro[4.2.4.2]tetradec-11-en-10-one (Ex. 1-71),
3-(4′-chloro-2,4-dimethylbiphenyl-3-yl)-4-hydroxy-7-methoxy-1-azaspiro[4.5]dec-3-en-2-one (Ex. 1-72),
3-(4′-chloro-2,4-dimethylbiphenyl-3-yl)-7-ethoxy-4-hydroxy-1-azaspiro[4.5]dec-3-en-2-one (Ex. 1-73),
rel-(5R,7R)-3-(4,4′-dichlorobiphenyl-3-yl)-4-hydroxy-7-(2-methoxyethoxy)-1-azaspiro[4.5]dec-3-en-2-one (Ex. 1-74),
3-(4′-chloro-2,4,6-trimethylbiphenyl-3-yl)-8-ethoxy-4-hydroxy-1-azaspiro[4.5]dec-3-en-2-one (Ex. 1-75),
3-(4′-chloro-4-methylbiphenyl-3-yl)-4-hydroxy-8-methyl-1-azaspiro[4.5]dec-3-en-2-one (Ex. 1-76),
3-(4′-chloro-4-methylbiphenyl-3-yl)-4-hydroxy-1-azaspiro[4.5]dec-3-en-2-one (Ex. 1-77),
3-(4′-chloro-4-methylbiphenyl-3-yl)-4-hydroxy-5-methyl-5-(propan-2-yl)-1,5-dihydro-2H-pyrrol-2-one (Ex. 1-78),
3-(3′-chloro-4-methylbiphenyl-3-yl)-8-ethoxy-4-hydroxy-1-azaspiro[4.5]dec-3-en-2-one (Ex. 1-79),
3-(2′,5′-difluoro-4-methylbiphenyl-3-yl)-4-hydroxy-8-methoxy-1-azaspiro[4.5]dec-3-en-2-one (Ex. 1-80),
3-(3′,4′-dichloro-4-methylbiphenyl-3-yl)-4-hydroxy-8-methoxy-1-azaspiro[4.5]dec-3-en-2-one (Ex. 1-81),
3-(4′-chloro-4-methylbiphenyl-3-yl)-4-hydroxy-7-methyl-1-azaspiro[4.5]dec-3-en-2-one (Ex. 1-82),
3-(4′-chloro-4-methylbiphenyl-3-yl)-4-hydroxy-8-propyl-1-azaspiro[4.5]dec-3-en-2-one (Ex. 1-83),
6-(4′-chloro-4-methylbiphenyl-3-yl)-7-hydroxy-4-azaspiro[2.4]hept-6-en-5-one (Ex. 1-84),
3-(4′-chloro-4-methylbiphenyl-3-yl)-4-hydroxy-1-azaspiro[4.4]non-3-en-2-one (Ex. 1-85),
3-(4′-chloro-4-methylbiphenyl-3-yl)-5-cyclopropyl-4-hydroxy-5-methyl-1,5-dihydro-2H-pyrrol-2-one (Ex. 1-86),
(5r,8r)-3-(4′-chloro-2,4-dimethylbiphenyl-3-yl)-4-hydroxy-8-methoxy-8-methyl-1-azaspiro[4.5]dec-3-en-2-one (Ex. 1-87),
(5r,8r)-3-(4′-chloro-2,4-dimethylbiphenyl-3-yl)-8-ethyl-4-hydroxy-8-methoxy-1-azaspiro[4.5]dec-3-en-2-one (Ex. 1-88),
(5r,8r)-8-ethyl-3-(4′-fluoro-2,4-dimethylbiphenyl-3-yl)-4-hydroxy-8-methoxy-1-azaspiro[4.5]dec-3-en-2-one (Ex. 1-89),
(5s,8s)-3-(4′-chloro-3′-fluoro-4-methylbiphenyl-3-yl)-4-hydroxy-8-(trifluoromethyl)-1-azaspiro[4.5]dec-3-en-2-one (Ex. 1-90)
(5s,8s)-3-(4′-chloro-5-fluoro-4-methylbiphenyl-3-yl)-4-hydroxy-8-(trifluoromethyl)-1-azaspiro[4.5]dec-3-en-2-one (Ex. 1-91)
(5s,8s)-3-(4,4′-dichloro-3′-fluorobiphenyl-3-yl)-4-hydroxy-8-(trifluoromethyl)-1-azaspiro[4.5]dec-3-en-2-one (Ex. 1-92)
(5s,8s)-3-(4′-chloro-3′-fluoro-2,4-dimethylbiphenyl-3-yl)-4-hydroxy-8-(trifluoromethyl)-1-azaspiro[4.5]dec-3-en-2-one (Ex. 1-93)
3-(4′-chloro-3′,6-difluoro-4-methylbiphenyl-3-yl)-4-hydroxy-8-(trifluoromethyl)-1-azaspiro[4.5]dec-3-en-2-one (Ex. 1-94)
(5s,8s)-3-(4′-chloro-2,4-dimethylbiphenyl-3-yl)-4-hydroxy-8-(trifluoromethyl)-1-azaspiro[4.5]dec-3-en-2-one (Ex. 1-95)
(5s,8s)-4-hydroxy-8-(trifluoromethyl)-3-(3′,4′,5-trifluoro-4-methylbiphenyl-3-yl)-1-azaspiro[4.5]dec-3-en-2-one (Ex. 1-96)
3-(4′-chloro-4,6-dimethylbiphenyl-3-yl)-4-hydroxy-8 (trifluoromethyl)-1-azaspiro[4.5]dec-3-en-2-one (Ex. 1-97)
(5s,8s)-3-(4′-chloro-4,6-dimethylbiphenyl-3-yl)-4-hydroxy-8-(trifluoromethyl)-1-azaspiro[4.5]dec-3-en-2-one (Ex. 1-98)
(5s,8s)-3-(4-chloro-3′,4′-difluorobiphenyl-3-yl)-4-hydroxy-8-(trifluoromethyl)-1-azaspiro[4.5]dec-3-en-2-one (Ex. 1-99)
3-(4′-chloro-6-fluoro-4-methylbiphenyl-3-yl)-4-hydroxy-8-(trifluoromethyl)-1-azaspiro[4.5]dec-3-en-2-one (Ex. 1-100)
(5s,8s)-3-(3′,4′-difluoro-2,4-dimethylbiphenyl-3-yl)-4-hydroxy-8-(trifluoromethyl)-1-azaspiro[4.5]dec-3-en-2-one (Ex. 1-101)
(5s,8s)-3-(4′-chloro-3′,5-difluoro-4-methylbiphenyl-3-yl)-4-hydroxy-8-(trifluoromethyl)-1-azaspiro[4.5]dec-3-en-2-one (Ex. 1-102)
(5s,8s)-3-(4,4′-dichloro-3′-fluorobiphenyl-3-yl)-4-hydroxy-8-methoxy-1-azaspiro[4.5]dec-3-en-2-one (Ex. 1-103)
(5s,8s)-3-(4-chloro-3′,4′-difluorobiphenyl-3-yl)-4-hydroxy-8-methoxy-1-azaspiro[4.5]dec-3-en-2-one (Ex. 1-104)
(5s,8s)-3-(4′-chloro-3′,5-difluoro-4-methylbiphenyl-3-yl)-4-hydroxy-8-methoxy-1-azaspiro[4.5]dec-3-en-2-one (Ex. 1-105)
(5s,8s)-3-(4′-chloro-5-fluoro-4-methylbiphenyl-3-yl)-4-hydroxy-8-methoxy-1-azaspiro[4.5]dec-3-en-2-one (Ex. 1-106)
(5s,8s)-4-hydroxy-8-methoxy-3-(3′,4′,5-trifluoro-4-methylbiphenyl-3-yl)-1-azaspiro[4.5]dec-3-en 2-one (Ex. 1-107)
(5s,8s)-3-(4′,6-dichloro-3′-fluoro-4-methylbiphenyl-3-yl)-4-hydroxy-8-methoxy-1-azaspiro[4.5]dec-3-en-2-one (Ex. 1-108)
(5s,8s)-3-(4′,6-dichloro-4-methylbiphenyl-3-yl)-4-hydroxy-8-methoxy-1-azaspiro[4.5]dec-3-en-2-one (Ex. 1-109)
(5s,8s)-3-(4′-chloro-3′-fluoro-4-methylbiphenyl-3-yl)-4-hydroxy-8-methyl-1-azaspiro[4.5]dec-3-en-2-one (Ex. 1-110)
(5s,8s)-3-(4′-chloro-3′-fluoro-4-methylbiphenyl-3-yl)-4-hydroxy-8-(methoxymethyl)-1-azaspiro[4.5]dec-3-en-2-one (Ex. 1-111)
(5s,8s)-3-(3′,4′-difluoro-4-methylbiphenyl-3-yl)-4-hydroxy-8-(methoxymethyl)-1-azaspiro[4.5]dec-3-en-2-one (Ex. 1-112)
(5s,8s)-3-(4,4′-dichloro-3′-fluorobiphenyl-3-yl)-4-hydroxy-8-(methoxymethyl)-1-azaspiro[4.5]dec-3-en-2-one (Ex. 1-113)
(5s,8s)-3-(4′-chloro-3′-fluoro-4-methylbiphenyl-3-yl)-4-hydroxy-8-(2-methoxyethyl)-1-azaspiro[4.5]dec-3-en-2-one (Ex. 1-114)
3-(3′,4′-difluoro-4-methylbiphenyl-3-yl)-4-hydroxy-1-azaspiro[4.5]dec-3-en-2-one (Ex. 1-115)
(5s,8s)-3-(4′-chloro-4-methylbiphenyl-3-yl)-4-hydroxy-8-(methoxymethyl)-1-azaspiro[4.5]dec-3-en-2-one (Ex. 1-116)
(5s,8s)-3-(4′-chloro-4-methylbiphenyl-3-yl)-4-hydroxy-8-(hydroxymethyl)-1-azaspiro[4.5]dec-3-en-2-one (Ex. 1-117)
(5s,8s)-3-(4′-chloro-3′-fluoro-4-methylbiphenyl-3-yl)-4-hydroxy-8-methoxy-1-azaspiro[4.5]dec-3-en-2-one (Ex. 1-118)
(5s,8s)-3-(4′-chloro-4-methylbiphenyl-3-yl)-4-hydroxy-8-(trifluoromethyl)-1-azaspiro[4.5]dec-3-en-2-one (Ex. 1-119)
(5s,8s)-3-(4,4′-dichlorobiphenyl-3-yl)-4-hydroxy-8-methoxy-1-azaspiro[4.5]dec-3-en-2-one (Ex. 1-120)
(5s,8s)-3-(4′-chloro-2,4-dimethylbiphenyl-3-yl)-4-hydroxy-8-methoxy-1-azaspiro[4.5]dec-3-en-2-one (Ex. 1-121)
(5r,8r)-3-(4′-chloro-4-methylbiphenyl-3-yl)-4,8-dihydroxy-8-(trifluoromethyl)-1-azaspiro[4.5]dec-3-en-2-one (Ex. 1-122)
(5r,8r)-3-(4′-chloro-3′-fluoro-4-methylbiphenyl-3-yl)-4,8-dihydroxy-8-(trifluoromethyl)-1-azaspiro[4.5]dec-3-en-2-one (Ex. 1-123)
(5r,8r)-3-(3′,4′-difluoro-4-methylbiphenyl-3-yl)-4,8-dihydroxy-8-(trifluoromethyl)-1-azaspiro[4.5]dec-3-en-2-one (Ex. 1-124)
(5r,8r)-3-(4′-chloro-2,4-dimethylbiphenyl-3-yl)-4,8-dihydroxy-8-(trifluoromethyl)-1-azaspiro[4.5]dec-3-en-2-one (Ex. 1-125)
(5r,8r)-3-(4′-chloro-3′-fluoro-2,4-dimethylbiphenyl-3-yl)-4,8-dihydroxy-8-(trifluoromethyl)-1-azaspiro[4.5]dec-3-en-2-one (Ex. 1-126)
(5r,8r)-3-(4′-chloro-6-fluoro-4-methylbiphenyl-3-yl)-4,8-dihydroxy-8-(trifluoromethyl)-1-azaspiro[4.5]dec-3-en-2-one (Ex. 1-127)
(5s,8s)-3-(3′,4′-difluoro-4-methylbiphenyl-3-yl)-4-hydroxy-8-(trifluoromethyl)-1-azaspiro[4.5]dec-3-en-2-one (Ex. 1-128)
(5r,8r)-3-(3′,4′-difluoro-4-methylbiphenyl-3-yl)-4-hydroxy-8-(trifluoromethyl)-1-azaspiro[4.5]dec-3-en-2-one (Ex. 1-129)
(5r,8r)-3-(4′-chloro-3′-fluoro-4-methylbiphenyl-3-yl)-4,8-dihydroxy-8-(pentafluoroethyl)-1-azaspiro[4.5]dec-3-en-2-one (Ex. 1-130)
(5r,8r)-3-(4′-chloro-6-fluoro-4-methylbiphenyl-3-yl)-4,8-dihydroxy-8-(pentafluoroethyl)-1-azaspiro[4.5]dec-3-en-2-one (Ex. 1-131)
(5s,8s)-3-(4′-chloro-4,6-dimethylbiphenyl-3-yl)-4-hydroxy-8-methoxy-1-azaspiro[4.5]dec-3-en-2-one (Ex. 1-132)
(5S,7S)3-(4′-chloro-4-methylbiphenyl-3-yl)-4-hydroxy-7-(trifluoromethyl)-1-azaspiro[4.5]dec-3-en-2-one (Ex. 1-133)
(5s,8s)-3-(4,4′-dichlorobiphenyl-3-yl)-4-hydroxy-8 (trifluoromethyl)-1-azaspiro[4.5]dec-3-en-2-one (Ex. 1-134)
(5s,8s)-3-(4′-chloro-6-fluoro-4-methylbiphenyl-3-yl)-4-hydroxy-8-methoxy-1-azaspiro[4.5]dec-3-en-2-one (Ex. 1-135)
(5s,8s)-3-(3′,4′-difluoro-4-methylbiphenyl-3-yl)-4-hydroxy-8-methoxy-1-azaspiro[4.5]dec-3-en-2-one (Ex. 1-136)
(5S,7S)-3-(4,4′-dichlorobiphenyl-3-yl)-4-hydroxy-7-(trifluoromethyl)-1-azaspiro[4.5]dec-3-en-2-one (Ex. 1-137)
3-(4,4′-dichlorobiphenyl-3-yl)-4-hydroxy-8,8-dimethyl-1-azaspiro[4.5]dec-3-en-2-one (Ex. 1-138)
3-(4,4′-dichlorobiphenyl-3-yl)-4-hydroxy-8-methyl-1-azaspiro[4.5]dec-3-en-2-one (Ex. 1-139)
3-(4,4′-dichlorobiphenyl-3-yl)-4-hydroxy-1-azaspiro[4.5]dec-3-en-2-one (Ex. 1-140)
(5s,8s)-3-(4,4′-dichlorobiphenyl-3-yl)-4-hydroxy-8-isopropyl-1-azaspiro[4.5]dec-3-en-2-one (Ex. 1-141)
(5s,8s)-3-(4′-chloro-2′-fluoro-4-methylbiphenyl-3-yl)-4-hydroxy-8-methoxy-1-azaspiro[4.5]dec-3-en-2-one (Ex. 1-142)
(5s,8s)-3-(2′,4′-dichloro-4-methylbiphenyl-3-yl)-4-hydroxy-8-methoxy-1-azaspiro[4.5]dec-3-en-2-one (Ex. 1-143)
(5s,8s)-3-(2′-chloro-4′-fluoro-4-methylbiphenyl-3-yl)-4-hydroxy-8-methoxy-1-azaspiro[4.5]dec-3-en-2-one (Ex. 1-144)
(5s,8s)-3-(3′,4′-difluoro-4-methylbiphenyl-3-yl)-4-hydroxy-8-methyl-1-azaspiro[4.5]dec-3-en-2-one
(5s,8s)-3-(4′-chloro-4-methylbiphenyl-3-yl)-8-ethoxy-4-hydroxy-1-azaspiro[4.5]dec-3-en-2-one (Ex. 1-146)
(5r,8r)-3-(4′-chloro-4-methylbiphenyl-3-yl)-8-ethoxy-4-hydroxy-1-azaspiro[4.5]dec-3-en-2-one (Ex. 1-147)

The present application furthermore provides the compounds, described in the experimental section, of the formula (I-2) for use as medicaments:
3-(4′-chloro-4-methylbiphenyl-3-yl)-8-ethyl-4-hydroxy 1 oxaspiro[4.5]dec-3-en-2-one (Ex. 2-1),
3-(4′-chloro-2,4-dimethylbiphenyl-3-yl)-4-hydroxy-1-oxaspiro[4.4]non-3-en-2-one (Ex. 2-2),
3-(4′-chloro-4-methylbiphenyl-3-yl)-4-hydroxy-5,5-dimethylfuran-2(5H)-one (Ex. 2-3),
3-(4′-chloro-2,4-dimethylbiphenyl-3-yl)-4-hydroxy-1-oxaspiro[4.5]dec-3-en-2-one (Ex. 2-4),
3-(4′-chloro-2,4,6-trimethylbiphenyl-3-yl)-4-hydroxy-5,5-dimethylfuran-2(5H)-one (Ex. 2-5),
3-(4′-chloro-2,4,6-trimethylbiphenyl-3-yl)-4-hydroxy-1-oxaspiro[4.5]dec-3-en-2-one (Ex. 2-6),
3-(2′-chloro-4-methylbiphenyl-3-yl)-4-hydroxy-8-methoxy-1-oxaspiro[4.5]dec-3-en-2-one (Ex. 2-7),
6-(4′-chloro-4-methylbiphenyl-3-yl)-7-hydroxy-4 oxaspiro[2.4]hept-6-en-5-one (Ex. 2-8),
3-(4′-chloro-4-methylbiphenyl-3-yl)-4-hydroxy-1,7-dioxaspiro[4.5]dec-3-en-2-one (Ex. 2-9),
3-(4′-chloro-4-methylbiphenyl-3-yl)-4-hydroxy-1-oxaspiro[4.5]dec-3-en-2-one (Ex. 2-10),
(4′-chloro-4,6-dimethylbiphenyl-3-yl)-4-hydroxy-7-methoxy-1-oxaspiro[4.5]dec-3-en-2-one (Ex. 2-11),
3-(4′-chloro-2,4,6-trimethylbiphenyl-3-yl)-4-hydroxy-7-methoxy-1-oxaspiro[4.5]dec-3-en-2-one (Ex. 2-12),
3-(4,4′-dichlorobiphenyl-3-yl)-4-hydroxy-7-methoxy-1-oxaspiro[4.5]dec-3-en-2-one (Ex. 2-13),
3-(4′-chloro-4-methylbiphenyl-3-yl)-4-hydroxy-7-methoxy-1-oxaspiro[4.5]dec-3-en-2-one (Ex. 2-14),
11-(4′-chloro-4-methylbiphenyl-3-yl)-12-hydroxy-1,4,9-trioxadispiro[4.2.4.2]tetradec-11-en-10-one (Ex. 2-15),
3-(4,4′-dichlorobiphenyl-3-yl)-4-hydroxy-8-(methoxymethyl)-1-oxaspiro[4.5]dec-3-en-2-one (Ex. 2-16),
3-(4′-chloro-2,4-dimethylbiphenyl-3-yl)-4-hydroxy-8-(methoxymethyl)-1-oxaspiro[4.5]dec-3-en 2-one (Ex. 2-17),
3-(4′-chloro-4-methylbiphenyl-3-yl)-4-hydroxy-7-(2-methoxyethyl)-1-oxaspiro[4.5]dec-3-en-2-one (Ex. 2-18),
3-(4,4′-dichlorobiphenyl-3-yl)-4-hydroxy-7-(2-methoxyethyl)-1-oxaspiro[4.5]dec-3-en-2-one (Ex. 2-19),
3-(4′-chloro-4-methylbiphenyl-3-yl)-4-hydroxy-8-(methoxymethyl)-1-oxaspiro[4.5]dec-3-en-2-one (Ex. 2-20),
3-(4′-fluoro-4-methylbiphenyl-3-yl)-4-hydroxy-1-oxaspiro[4.5]dec-3-en-2-one (Ex. 2-21),
3-(3′,4′-difluoro-4-methylbiphenyl-3-yl)-4-hydroxy-1-oxaspiro[4.4]non-3-en-2-one (Ex. 2-22),
3-(3′-chloro-4′-fluoro-4-methylbiphenyl-3-yl)-4-hydroxy-1-oxaspiro[4.4]non-3-en-2-one (Ex. 2-23),
3-(4-chloro-3′,4′,5′-trifluorobiphenyl-3-yl)-4-hydroxy-1-oxaspiro[4.4]non-3-en-2-one (Ex. 2-24),
3-(4-chloro-3′,4′-difluorobiphenyl-3-yl)-4-hydroxy-1-oxaspiro[4.5]dec-3-en-2-one (Ex. 2-25),
(5s,8r)-3-(4′-chloro-2,4-dimethylbiphenyl-3-yl)-4-hydroxy-1,9-dioxadispiro[4.2.4.2]tetradec-3-en-2-one (Ex. 2-26),
3-(4,4′-dichlorobiphenyl-3-yl)-4-hydroxy-1,9-dioxadispiro[4.2.4.2]tetradec-3-en-2-one (Ex. 2-27),
(5r,8s)-3-(4′-fluoro-4-methylbiphenyl-3-yl)-4-hydroxy-1,9-dioxadispiro[4.2.4.2]tetradec-3-en-2-one (Ex. 2-28),
3-(4′-chloro-4-methylbiphenyl-3-yl)-4-hydroxy-1,9-dioxadispiro[4.2.4.2]tetradec-3-en-2-one (Ex. 2-29),
(5r,8s)-3-(4′-chloro-4,6-dimethylbiphenyl-3-yl)-4-hydroxy-1,9-dioxadispiro[4.2.4.2]tetradec-3-en-2-one (Ex. 2-30),
(5r,8s)-3-(4′-chloro-2,4,6-trimethylbiphenyl-3-yl)-4-hydroxy-1,9-dioxadispiro[4.2.4.2]tetradec-3-en-2-one (Ex. 2-31),
3-(4′-chloro-2,4-dimethylbiphenyl-3-yl)-4-hydroxy-8-(2,2,2-trifluoroethoxy)-1-oxaspiro[4.5]dec-3-en-2-one (Ex. 2-32),
3-(4′-chloro-4-methylbiphenyl-3-yl)-4-hydroxy-8-(2,2,2-trifluoroethoxy)-1-oxaspiro[4.5]dec-3-en-2-one (Ex. 2-33),
3-(4′-chloro-2,4-dimethylbiphenyl-3-yl)-4-hydroxy-8-methoxy-1-oxaspiro[4.5]dec-3-en-2-one (Ex. 2-34),
5-tert-butyl-3-(4′-chloro-4-methylbiphenyl-3-yl)-4-hydroxyfuran-2(5H)-one (Ex. 2-35),
3-(4′-chloro-4-methylbiphenyl-3-yl)-4-hydroxy-8-propyl-1-oxaspiro[4.5]dec-3-en-2-one (Ex. 2-36),
3-(4′-chloro-4-methylbiphenyl-3-yl)-4-hydroxy-1,8-dioxaspiro[4.5]dec-3-en-2-one (Ex. 2-37),
3-(4′-chloro-4-methylbiphenyl-3-yl)-4-hydroxy-1-oxaspiro[4.6]undec-3-en-2-one (Ex. 2-38),
3-(4′-chloro-4-methylbiphenyl-3-yl)-4-hydroxy-8-methoxy-1-oxaspiro[4.5]dec-3-en-2-one (Ex. 2-39),
3-(4′-chloro-4-methylbiphenyl-3-yl)-5-cyclohexyl-4-hydroxy-5-methylfuran-2(5H)-one (Ex. 2-40),
3-(4′-chloro-4,6-dimethylbiphenyl-3-yl)-4-hydroxy-1,8-dioxaspiro[4.5]dec-3-en-2-one (Ex. 2-41),
3-(4′-chloro-4,6-dimethylbiphenyl-3-yl)-4-hydroxy-1-oxa-8-thiaspiro[4.5]dec-3-en-2-one (Ex. 2-42),
3-(4′-chloro-4,6-dimethylbiphenyl-3-yl)-4-hydroxy-1-oxaspiro[4.6]undec-3-en-2-one (Ex. 2-43),
3-(4′-chloro-4,6-dimethylbiphenyl-3-yl)-4-hydroxy-8-methoxy-1-oxaspiro[4.5]dec-3-en-2-one (Ex. 2-44),
3-(4′-chloro-4,6-dimethylbiphenyl-3-yl)-4-hydroxy-5-methylfuran-2(5H)-one (Ex. 2-45),
3-(4′-chloro-4,6-dimethylbiphenyl-3-yl)-4-hydroxy-1,7-dioxaspiro[4.5]dec-3-en-2-one (Ex. 2-46),
3-(4′-chloro-4,6-dimethylbiphenyl-3-yl)-4-hydroxy-8-propoxy-1-oxaspiro[4.5]dec-3-en-2-one (Ex. 2-47),
3-(4′-chloro-2,4,6-trimethylbiphenyl-3-yl)-4-hydroxy-1-oxaspiro[4.6]undec-3-en-2-one (Ex. 2-48),
3-(4′-chloro-2,4,6-trimethylbiphenyl-3-yl)-4-hydroxy-1-oxaspiro[4.4]non-3-en-2-one (Ex. 2-49),
3-(3′-chloro-4-methylbiphenyl-3-yl)-4-hydroxy-1-oxaspiro[4.6]undec-3-en-2-one (Ex. 2-50),

The present application furthermore provides the compounds, described in the experimental section, of the formula (I-3), (I-6), (I-7), (I-8), (I-9), (I-10) and (I-11) for use as medicaments:
3-(4′-chloro-4-methylbiphenyl-3-yl)-4-hydroxy-8-methoxy-1 thiaspiro[4.5]dec-3-en-2-one (Ex. 3-1)
3-(4′-chloro-4-methylbiphenyl-3-yl)-4-hydroxy-1 thiaspiro[4.5]dec-3-en-2-one (Ex. 3-2)
3-(4′-chloro-4,6-dimethylbiphenyl-3-yl)-4-hydroxy-1 thiaspiro[4.5]dec-3-en-2-one (Ex. 3-3)
3-(4′-chloro-2,4,6-trimethylbiphenyl-3-yl)-4-hydroxy-1 thiaspiro[4.5]dec-3-en-2-one (Ex. 3-4)
3-(4,4′-dimethylbiphenyl-3-yl)-4-hydroxyspiro[4.5]dec-3-en-2-one (Ex. 6-1)
3-(2′,4′-dichloro-4-methylbiphenyl-3-yl)-4-hydroxyspiro[4.5]dec-3-en-2-one (Ex. 6-2)
3-(3′-chloro-4-methylbiphenyl-3-yl)-4-hydroxyspiro[4.5]dec-3-en-2-one (Ex. 6-3)
3-(4′-chloro-4-methylbiphenyl-3-yl)-4-hydroxyspiro[4.4]non-3-en-2-one (Ex. 6-4)
3-(4′-chloro-4-methylbiphenyl-3-yl)-8-ethyl-4-hydroxyspiro[4.5]dec-3-en-2-one (Ex. 6-5)
3-(4′-chloro-4-methylbiphenyl-3-yl)-4-hydroxy-8-propylspiro[4.5]dec-3-en-2-one (Ex. 6-6)
3-(4′-chloro-4-methylbiphenyl-3-yl)-4-hydroxyspiro[4.6]undec-3-en-2-one (Ex. 6-7)
3-(4′-chloro-4,6-dimethylbiphenyl-3-yl)-4-hydroxyspiro[4.5]dec-3-en-2-one (Ex. 6-8)
3-(4′-chloro-2,4,6-trimethylbiphenyl-3-yl)-4-hydroxyspiro[4.5]dec-3-en-2-one (Ex. 6-9)
2-(4′-chloro-4-methylbiphenyl-3-yl)-3-hydroxy-5-methylcyclohex-2-en-1-one (Ex. 7-1)
3-(4′-chloro-4-methylbiphenyl-3-yl)-4-hydroxyspiro[5.5]undec-3-en-2-one (Ex. 7-2)
2-(4′-chloro-4,6-dimethylbiphenyl-3-yl)-3-hydroxy-5,5-dimethylcyclohex-2-en-1-one (Ex. 7-3)
2-(4′-chloro-4,6-dimethylbiphenyl-3-yl)-3-hydroxy-4,4-dimethylcyclohex-2-en-1-one (Ex. 7-4)
2-(4′-chloro-4-methylbiphenyl-3-yl)-3-hydroxy-5,5-dimethylcyclohex-2-en-1-one (Ex. 7-5)
2-(4′-fluoro-2,4-dimethylbiphenyl-3-yl)tetrahydro-1H-pyrazolo[1,2-a]pyridazine-1,3(2H)-dione (Ex. 8-1)
2-(4,4′-dichlorobiphenyl-3-yl)tetrahydro-1H-pyrazolo[1,2-a]pyridazine-1,3(2H)-dione (Ex. 8-2)
2-(4′-chloro-4-methylbiphenyl-3-yl)tetrahydro-1H-pyrazolo[1,2-a]pyridazine-1,3(2H)-dione (Ex. 8-3)
2-(3′,4-dichloro-4′-fluorobiphenyl-3-yl)tetrahydro-1H-pyrazolo[1,2-a]pyridazine-1,3(2H)-dione (Ex. 8-4)
8-(2′,4′-difluoro-4-methylbiphenyl-3-yl)tetrahydro-7H-pyrazolo[1,2-d][1,4,5]oxadiazepine-7,8(8H)-dione (Ex. 8-5)
8-(4′-chloro-4-methylbiphenyl-3-yl)tetrahydro-7H-pyrazolo[1,2-d][1,4,5]oxadiazepine-7,9(8H)-dione (Ex. 8-6)
8-(4′-fluoro-2,4-dimethylbiphenyl-3-yl)tetrahydro-7H-pyrazolo[1,2-d][1,4,5]oxadiazepine-7,9(8H)-dione (Ex. 8-7)
8-(4′-chloro-2,4-dimethylbiphenyl-3-yl)tetrahydro-7H-pyrazolo[1,2-d][1,4,5]oxadiazepine-7,9(8H)-dione (Ex. 8-8)
8-(2′,4,4′-trichlorobiphenyl-3-yl)tetrahydro-7H-pyrazolo[1,2-d][1,4,5]oxadiazepine-7,9(8H)-dione (Ex. 8-9)
8-(3′,4,4′-trichlorobiphenyl-3-yl)tetrahydro-7H-pyrazolo[1,2-d][1,4,5]oxadiazepine-7,9(8H)-dione (Ex. 8-10)
8-(4-chloro-2′,4′-difluorobiphenyl-3-yl)tetrahydro-7H-pyrazolo[1,2-d][1,4,5]oxadiazepine-7,9(8H)-dione (Ex. 8-11)
2-(4′-chloro-4-methylbiphenyl-3-yl)-6-fluoro-6-methyldihydro-1H,5H-pyrazolo[1,2-a]pyrazole-1,3(2H)-dione (Ex. 8-12)
2-(4,4′-dichlorobiphenyl-3-yl)-6-fluoro-6-methyldihydro-1H,5H-pyrazolo[1,2-a]pyrazole-1,3(2H)-dione (Ex. 8-13)
4-(4′-chloro-4-methylbiphenyl-3-yl)-1,2-dimethyl-1H-pyrazole-3,5(2H,4H)-dione (Ex. 8-14)
4-(4,4′-dichlorobiphenyl-3-yl)-1,2-dimethyl-1H-pyrazole-3,5(2H,4H)-dione (Ex. 8-15)
2-(4′-chloro-4-methylbiphenyl-3-yl)tetrahydro-1H-5,8-methanopyrazolo[1,2-a]pyridazine-1,3(2H)-dione (Ex. 8-16)
2-(4,4′-dichlorobiphenyl-3-yl)tetrahydro-1H-5,8-methanopyrazolo[1,2-a]pyridazine-1,3(2H)-dione (Ex. 8-17)
3-(4′-chloro-4-methylbiphenyl-3-yl)-4-hydroxy-6,6-dimethyl-5,6-dihydropyridin-2(1H)-one (Ex. 9-1)
3-(4′-chloro-4-methylbiphenyl-3-yl)-4-hydroxy-5,5-dimethyl-5,6-dihydropyridin-2(1H)-one (Ex. 9-2)
4-(4′-chloro-4-methylbiphenyl-3-yl)-5-hydroxy-2-azaspiro[5.5]undec-4-en-3-one (Ex. 9-3)
3-(4′-chloro-4-methylbiphenyl-3-yl)-4-hydroxy-1,6,6-trimethyl-5,6-dihydropyridin-2(1H)-one (Ex. 9-4) 9
3-(4′-chloro-4-methylbiphenyl-3-yl)-4-hydroxyquinolin-2(1H)-one (Ex. 9-5)
7-chloro-4-hydroxy-3-[4-methyl-4′-(trifluoromethyl)biphenyl-3-yl]quinolin-2(1H)-one (Ex. 9-6)
7-chloro-3-(3′,4′-dichloro-4-methylbiphenyl-3-yl)-4-hydroxyquinolin-2(1H)-one (Ex. 9-7)
3-(4′-chloro-4-methylbiphenyl-3-yl)-7-fluoro-4-hydroxyquinolin-2(1H)-one (Ex. 9-8)
7-fluoro-3-(4′-fluoro-4-methylbiphenyl-3-yl)-4-hydroxyquinolin-2(1H)-one (Ex. 9-9)
7-fluoro-4-hydroxy-3-[4-methyl-4′-(trifluoromethyl)biphenyl-3-yl]quinolin-2(1H)-one (Ex. 9-10)
7-chloro-3-(4′-chloro-4-methylbiphenyl-3-yl)-4-hydroxyquinolin-2(1H)-one (Ex. 9-11)
7-chloro-3-(4′-chloro-4,6-dimethylbiphenyl-3-yl)-4-hydroxyquinolin-2(1H)-one (Ex. 9-12)
7-chloro-3-(4,4′-dichlorobiphenyl-3-yl)-4-hydroxyquinolin-2(1H)-one (Ex. 9-13)
3-(4′-chloro-4-methylbiphenyl-3-yl)-4-hydroxy-5,5,6,6-tetramethyl-5,6-dihydropyridin-2(1H)-one (Ex. 9-14)
3-(4,4′-dichlorobiphenyl-3-yl)-4-hydroxy-5,5,6,6-tetramethyl-5,6-dihydro-2H-pyran-2-one (Ex. 10-1)
3-(4′-chloro-4-methylbiphenyl-3-yl)-4-hydroxy-5,5,6,6-tetramethyl-5,6-dihydro-2H-pyran-2-one (Ex. 10-2)
4-(4′-chloro-4-methylbiphenyl-3-yl)-2,6,6-trimethyl-1,2-oxazinane-3,5-dione (Ex. 11-1)

Saturated or unsaturated hydrocarbon radicals such as alkyl, alkanediyl or alkenyl may in each case be straight-chain or branched as far as this is possible, also in combination with heteroatoms, such as, for example, in alkoxy.

Unless indicated otherwise, optionally substituted radicals may be mono- or polysubstituted, where in the case of polysubstitution the substituents may be identical or different.

The present invention also comprises all compounds resulting from all possible combinations of the abovementioned possible, preferred and particularly preferred meanings of the substituents.

Particular embodiments of the invention additionally consist of compounds resulting from combinations of the substituent meanings disclosed directly in the examples.

The present invention likewise embraces the use of the physiologically acceptable salts of the compounds.

Physiologically acceptable salts of the compounds according to the invention include acid addition salts of mineral acids, carboxylic acids and sulphonic acids, e.g. salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of conventional bases, such as, by way of example and preferably, alkali metal salts (e.g. sodium and potassium salts), alkaline earth metal salts (e.g. calcium and magnesium salts) and ammonium salts derived from ammonia or organic amines having 1 to 16 C atoms, such as, by way of example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

The present invention furthermore provides medicaments comprising at least one compound according to the invention and at least one or more further active compounds, in particular for the prophylaxis and/or therapy of tumour disorders.

The compounds according to the invention can act systemically and/or locally. For this purpose, they can be administered in a suitable manner, such as, for example, orally, parenterally, pulmonarily, nasally, sublingually, lingually, buccally, rectally, dermally, transdermally, conjunctivally, otically, as or as an implant or stent.

For these administration routes, the compounds according to the invention can be administered in suitable administration forms.

Suitable for oral administration are administration forms working according to the prior art, which release the compounds according to the invention rapidly and/or in modified form and comprise the compounds according to the invention in crystalline and/or amorphized and/or dissolved form, such as, for example, tablets (non-coated or coated tablets, for example coated with enteric, slowly dissolving or insoluble coats which control the release of the compound according to the invention), tablets which decompose rapidly in the oral cavity or films/wafers, films/lyophylizates, capsules (for example hard gelatin capsules or soft gelatin capsules), sugar-coated tablets, granules, pellets, powders, emulsions, suspensions, aerosols or solutions.

Parenteral administration can take place with circumvention of an absorption step (for example intravenous, intraarterial, intracardiac, intraspinal or intralumbar) or with involvement of an absorption (for example intramuscular, subcutaneous, intracutaneous, percutaneous or intraperitoneal). For parenteral administration, suitable administration forms are, inter alia, injection and infusion preparations in the form of solutions, suspensions, emulsions, lyophilizates or sterile powders.

Suitable for the other administration routes are, for example, pharmaceutical forms for inhalation (inter alia powder inhalers, nebulizers), nasal drops, nasal solutions, nasal sprays; tablets, films/wafers or capsules to be applied lingually, sublingually or buccally, suppositories, ear or eye preparations, vaginal capsules, aqueous suspensions (lotions, shake lotions), lipophilic suspensions, ointments, creams, transdermal therapeutic systems (such as, for example, patches), milk, pastes, foams, dusting powders, implants or stents.

The compounds according to the invention can be converted into the administration forms mentioned. This may take place in a manner known per se by mixing with inert non-toxic, pharmaceutically acceptable auxiliaries. These auxiliaries include, inter alia, carriers (for example microcrystalline cellulose, lactose, mannitol), solvents (for example liquid polyethylene glycols), emulsifiers and dispersants or wetting agents (for example sodium dodecylsulphate, polyoxysorbitan oleate), binders (for example polyvinylpyrrolidone), synthetic and natural polymers (for example albumin), stabilizers (e.g. antioxidants such as, for example, ascorbic acid), colorants (e.g. inorganic pigments such as, for example, iron oxides) and taste and/or odour corrigents.

The present invention furthermore provides medicaments comprising at least one compound according to the invention, usually together with one or more inert non-toxic, pharmaceutically suitable auxiliaries, and their use for the purposes mentioned above.

Formulation of the compounds according to the invention to give pharmaceutical products takes place in a manner known per se by converting the active ingredient(s) with the excipients customary in pharmaceutical technology into the desired administration form.

Excipients which can be employed in this connection are, for example, carrier substances, fillers, disintegrants, binders, humectants, lubricants, absorbents and adsorbents, diluents, solvents, cosolvents, emulsifiers, solubilizers, masking flavours, colorants, preservatives, stabilizers, wetting agents, salts to alter the osmotic pressure or buffers.

Reference should be made in this connection to Remington's Pharmaceutical Science, 15th ed. Mack Publishing Company, East Pennsylvania (1980).

The pharmaceutical formulations may be

in solid form, for example as tablets, coated tablets, pills, suppositories, capsules, transdermal systems or
in semisolid form, for example as ointments, creams, gels, suppositories, emulsions or
in liquid form, for example as solutions, tinctures, suspensions or emulsions.

Excipients in the context of the invention may be, for example, salts, saccharides (mono-, di-, tri-, oligo-, and/or polysaccharides), proteins, amino acids, peptides, fats, waxes, oils, hydrocarbons and derivatives thereof, where the excipients may be of natural origin or may be obtained by synthesis or partial synthesis.

Suitable for oral or peroral administration are in particular tablets, coated tablets, capsules, pills, powders, granules, pastilles, suspensions, emulsions or solutions. Suitable for parenteral administration are in particular suspensions, emulsions and especially solutions.

The present invention relates to the use of the compounds of the formulae (I), (I-1) and (I-2) for the prophylaxis and therapy of human disorders, in particular of tumour disorders.

The compounds of the formulae (I), (I-1) and (I-2) can be used in particular for inhibiting or reducing cell proliferation and/or cell division and/or to induce apoptosis.

The compounds according to the invention are suitable in particular for the prophylaxis and/or therapy of hyper-proliferative disorders such as, for example,
psoriasis,
keloids and other skin hyperplasias,
benign prostate hyperplasias (BPH),
solid tumours and
haematological tumours.

Solid tumours which can be treated in accordance with the invention are, for example, tumours of the breast, the respiratory tract, the brain, the reproductive organs, the gastrointestinal tract, the urogenital tract, the eye, the liver, the skin, the head and the neck, the thyroid gland, the parathyroid gland, the bones and the connective tissue and metastases of these tumours.

Haematological tumours which can be treated in accordance with the invention are, for example, multiple myelomas, lymphomas or leukaemias.

Breast tumours which can be treated are, for example:
breast carcinomas with positive hormone receptor status
breast carcinomas with negative hormone receptor status
Her-2 positive breast carcinomas
hormone receptor and Her-2 negative breast carcinomas
BRCA-associated breast carcinomas
inflammatory breast carcinomas.

Tumours of the respiratory tract which can be treated are, for example,
non-small-cell bronchial carcinomas and
small-cell bronchial carcinomas.

Tumours of the brain which can be treated are, for example,
gliomas,
glioblastomas,
astrocytomas,
meningiomas and
medulloblastomas.

Tumours of the male reproductive organs which can be treated are, for example:
prostate carcinomas,
malignant testicular tumours and
penis carcinomas.

Tumours of the female reproductive organs which can be treated are, for example:
endometrial carcinomas
cervix carcinomas
ovarial carcinomas
vaginal carcinomas
vulvar carcinomas

Tumours of the gastrointestinal tract which can be treated are, for example:
colorectal carcinomas
anal carcinomas
stomach carcinomas
pancreas carcinomas
oesophagus carcinomas
gall bladder carcinomas
carcinomas of the small intestine
salivary gland carcinomas
neuroendocrine tumours
gastrointestinal stroma tumours

Tumours of the urogenital tract which can be treated are, for example:
urinary bladder carcinoma
kidney cell carcinoma
carcinomas of the renal pelvis and lower urinary tract

Tumours of the eye which can be treated are, for example:
retinoblastomas
intraocular melanomas

Tumours of the liver which can be treated are, for example:
hepatocellular carcinomas
cholangiocellular carcinomas

Tumours of the skin which can be treated are, for example:
malignant melanomas
basaliomas
spinaliomas
Kaposi sarcomas
Merkel cell carcinomas

Tumours of the head and neck which can be treated are, for example:
larynx carcinomas
carcinomas of the pharynx and the oral cavity

Sarcomas which can be treated are, for example:
soft tissue sarcomas
osteosarcomas

Lymphomas which can be treated are, for example:
non-Hodgkin lymphomas
Hodgkin lymphomas
cutaneous lymphomas
lymphomas of the central nervous system
AIDS-associated lymphomas

Leukaemias which can be treated are, for example:
acute myeloid leukaemias
chronic myeloid leukaemias
acute lymphatic leukaemias
chronic lymphatic leukaemias
hairy cell leukaemias

Particularly advantageously, the compounds of the formulae (I), (I-1) and (I-2) can be used for the prophylaxis and/or therapy of:

breast carcinomas, in particular of hormone receptor negative, hormone receptor positive or BRCA-associated breast carcinomas, and also
pancreas carcinomas, kidney cell carcinomas, hepatocellular carcinomas, malignant melanomas and other skin tumours, non-small-cell bronchial carcinomas, endometrial carcinomas, colorectal carcinomas and prostate carcinomas.

These disorders are well-characterized in man, but also exist in other mammals.

The present application furthermore provides the compounds of the formulae (I), (I-1) and (I-2) for use as medicaments, in particular for the prophylaxis and/or therapy of tumour disorders.

The present application furthermore provides the compounds of the formulae (I), (I-1) and (I-2) for the prophylaxis and/or therapy of breast carcinomas, pancreas carcinomas, kidney cell carcinomas, hepatocellular carcinomas, malignant melanomas and other skin tumours, non-small-cell bronchial carcinomas, endometrial carcinomas, colorectal carcinomas or prostate carcinomas.

The invention furthermore provides the use of the compounds of the general formula (I) according to the invention, in particular also of the formulae (I-1) and (1-2), for preparing a medicament.

The present application furthermore provides the use of the compounds of the formulae (I), (I-1) and (I-2) for preparing a medicament for the prophylaxis and/or therapy of tumour disorders.

The present application furthermore provides the use of the compounds of the formulae (I), (I-1) and (I-2) for preparing a medicament for the prophylaxis and/or therapy of breast carcinomas, pancreas carcinomas, kidney cell carcinomas, hepatocellular carcinomas, malignant melanomas and other skin tumours, non-small-cell bronchial carcinomas, endometrial carcinomas, colorectal carcinomas or prostate carcinomas.

The present application furthermore provides the use of the compounds of the formulae (I), (I-1) and (I-2) for the prophylaxis and/or therapy of tumour disorders.

The present application furthermore provides the use of the compounds of the formulae (I), (I-1) and (I-2) for the prophylaxis and/or therapy of breast carcinomas, pancreas carcinomas, kidney cell carcinomas, hepatocellular carcinomas, malignant melanomas and other skin tumours, non-small-cell bronchial carcinomas, endometrial carcinomas, colorectal carcinomas or prostate carcinomas.

The present application furthermore provides pharmaceutical formulations in the form of tablets comprising a compound of the formula (I), (I-1) or (1-2) for the prophylaxis and/or therapy of breast carcinomas, pancreas carcinomas, kidney cell carcinomas, hepatocellular carcinomas, malignant melanomas and other skin tumours, non-small-cell bronchial carcinomas, endometrial carcinomas, colorectal carcinomas or prostate carcinomas.

The invention furthermore provides the use of the compounds according to the invention for treating disorders associated with proliferative processes.

The compounds according to the invention can be employed by themselves or, if required, in combination with one or more other pharmacologically active substances, as long as this combination does not lead to unwanted and unacceptable side effects. Accordingly, the present invention furthermore provides medicaments comprising at least one of the compounds according to the invention and one or more further active compounds, in particular for prophylaxis and/or therapy of the abovementioned diseases.

For example, the compounds of the present invention can be combined with known antihyperproliferative, cytostatic or cytotoxic substances for treatment of cancer disorders. The combination of the compounds according to the invention with other substances customary for cancer therapy or else with radiotherapy is indicated in particular.

Suitable active compounds for combinations which may be mentioned by way of example are:

afinitor, aldesleukin, alendronic acid, alfaferone, alitretinoin, allopurinol, aloprim, aloxi, altretamine, aminoglutethimide, amifostine, amrubicin, amsacrine, anastrozole, anzmet, aranesp, arglabin, arsenic trioxide, aromasin, 5-azacytidine, azathioprine, BCG or tice-BCG, bestatin, beta-methasone acetate, betamethasone sodium phosphate, bexarotene, bleomycin sulphate, broxuridine, bortezomib, busulphan, calcitonin, campath, capecitabine, carboplatin, casodex, cefesone, celmoleukin, cerubidin, chlorambucil, cisplatin, cladribin, clodronic acid, cyclophosphamide, cytarabine, dacarbazine, dactinomycin, daunoxome, decadron, decadron phosphate, delestrogen, denileukin diftitox, depomedrol, deslorelin, dexrazoxane, diethylstilbestrol, diflucan, docetaxel, doxifluridine, doxorubicin, dronabinol, DW-166HC, eligard, elitek, ellence, emend, epirubicin, epoetin-alfa, epogen, eptaplatin, ergamisol, estrace, estradiol, estramustine sodium phosphate, ethynylestradiol, ethyol, etidronic acid, etopophos, etoposide, fadrozole, farstone, filgrastim, finasteride, fligrastim, floxuridine, fluconazole, fludarabin, 5-fluorodeoxyuridine monophosphate, 5-fluoruracil (5-FU), fluoxymesterone, flutamide, formestane, fosteabine, fotemustine, fulvestrant, gammagard, gemcitabine, gemtuzumab, gleevec, gliadel, goserelin, granisetron hydrochloride, histrelin, hycamtin, hydrocortone, erythro-hydroxynonyladenine, hydroxyurea, ibritumomab tiuxetan, idarubicin, ifosfamide, interferon-alpha, interferon-alpha-2, interferon-alpha-2α, interferon-alpha-213, interferon-alpha-n1, interferon-alpha-n3, interferon-beta, interferon-gamma-1α, interleukin-2, intron A, iressa, irinotecan, kytril, lapatinib, lentinan sulphate, letrozole, leucovorin, leuprolide, leuprolide acetate, levamisole, levofolic acid calcium salt, levothroid, levoxyl, lomustine, lonidamine, marinol, mechlorethamine, mecobalamin, medroxyprogesterone acetate, megestrol acetate, melphalan, menest, 6-mercaptopurine, mesna, methotrexate, metvix, miltefosine, minocycline, mitomycin C, mitotane, mitoxantrone, modrenal, myocet, nedaplatin, neulasta, neumega, neupogen, nilutamide, nolvadex, NSC-631570, OCT-43, octreotide, ondansetron hydrochloride, orapred, oxaliplatin, paclitaxel, pediapred, pegaspargase, pegasys, pentostatin, picibanil, pilocarpine hydrochloride, pirarubicin, plicamycin, porfimer sodium, prednimustine, prednisolone, prednisone, premarin, procarbazine, procrit, raltitrexed, RDEA119, rebif, rhenium-186 etidronate, rituximab, roferon-A, romurtide, salagen, sandostatin, sargramostim, semustine, sizofuran, sobuzoxane, solu-medrol, streptozocin, strontium-89 chloride, Synthroid, tamoxifen, tamsulosin, tasonermin, tastolactone, taxoter, tece-leukin, temozolomide, teniposide, testosterone propionate, testred, thioguanine, thiotepa, thyro-tropin, tiludronic acid, topotecan, toremifen, tositumomab, tastuzumab, treosulphan, tretinoin, trexall, trimethylmelamine, trimetrexate, triptorelin acetate, triptorelin pamoate, UFT, uridine, valrubicin, vesnarinone, vinblastine, vincristine, vindesine, vinorelbine, virulizin, zinecard, zinostatin-stimalamer, zofran; ABI-007, acolbifen, actimmune, affinitak, aminopterin, arzoxifen, asoprisnil, atamestane, atrasentan, BAY 43-9006 (sorafenib), avastin, CCI-779, CDC-501, celebrex, cetuximab, crisnatol, cyproterone acetate, decitabine, DN-101, doxorubicin-MTC, dSLIM, dutasteride, edotecarin, eflornithine, exatecan, fenretinide, histamine dihydrochloride, histrelin hydrogel implant, holmium-166 DOTMP, ibandronic acid, interferon-gamma, intron-PEG, ixabepilone, keyhole limpet hemocyanine, L-651582, lanreotide, lasofoxifen, libra, lonafarnib, miproxifen, minodronate, MS-209, liposomal MTP-PE, MX-6, nafarelin, nemorubicin, neovastat, nolatrexed, oblimersen, onko-TCS, osidem, paclitaxel polyglutamate, pamidronate disodium, PN-401, QS-21, quazepam, R-1549, raloxifen, ranpirnas, 13-cis-retinoic acid, satraplatin, seocalcitol, T-138067, tarceva, taxoprexin, thymosin-alpha-1, tiazofurin, tipifarnib, tirapazamine, TLK-286, toremifen, transMID-107R, valspodar, vapreotide, vatalanib, verteporfin, vinflunin, Z-100, zoledronic acid and combinations of these.

In a preferred embodiment, the compounds of the present invention can be combined with antihyperproliferative agents, which can be, by way of example—without this list being conclusive:

aminoglutethimide, L-asparaginase, azathioprine, 5-azacytidine, bleomycin, busulphan, carbo-platin, carmustine, chlorambucil, cisplatin, colaspase, cyclophosphamide, cytarabine, dacarbazine, dactinomycin, daunorubicin, diethylstilbestrol, 2′, T-difluorodeoxycytidine, docetaxel, doxorubicin (adriamycin), epirubicin, epothilone and its derivatives, erythro-hydroxynonyladenin, ethynyl-estradiol, etoposide, fludarabin phosphate, 5-fluorodeoxyuridine, 5-fluorodeoxyuridine mono-phosphate, 5-fluorouracil, fluoxymesterone, flutamide, hexamethylmelamine, hydroxyurea, hydroxyprogesterone caproate, idarubicin, ifosfamide, interferon, irinotecan, leucovorin, lomustine, mechlorethamine, medroxyprogesterone acetate, megestrol acetate, melphalan, 6-mer-captopurine, mesna, methotrexate, mitomycin C, mitotane, mitoxantrone, paclitaxel, pentostatin, N-phosphonoacetyl L-aspartate (PALA), plicamycin, prednisolone, prednisone, procarbazine, raloxifen, semustine, streptozocin, tamoxifen, teniposide, testosterone propionate, thioguanine, thiotepa, topotecan, trimethylmelamine, uridine, vinblastine, vincristine, vindesine and vinorelbine.

The compounds according to the invention can also be combined in a very promising manner with biological therapeutics, such as antibodies (e.g. avastin, rituxan, erbitux, herceptin) and recombinant proteins.

The compounds according to the invention may also achieve positive effects in combination with other therapies directed against angiogenesis, such as, for example, with avastin, axitinib, regorafenib, recentin, sorafenib or sunitinib. Combinations with inhibitors of the proteasome and of mTOR and antihormones and steroidal metabolic enzyme inhibitors are particularly suitable because of their favourable profile of side effects.

Generally, the following aims can be pursued with the combination of compounds of the present invention with other agents having a cytostatic or cytotoxic action:
an improved activity in slowing down the growth of a tumour, in reducing its size or even in its complete elimination compared with treatment with an individual active compound;
the possibility of employing the chemotherapeutics used in a lower dosage than in monotherapy;
the possibility of a more tolerable therapy with few side effects compared with individual administration;
the possibility of treatment of a broader spectrum of tumour diseases;
achievement of a higher rate of response to the therapy;
a longer survival time of the patient compared with present-day standard therapy.

The compounds according to the invention can moreover also be employed in combination with radiotherapy and/or surgical intervention.

### COMPARATIVE EXAMPLES

Table C.1 lists related structures of the prior art and indicates which patent discloses the preparation.

TABLE V.1

| | | disclosed |
|---|---|---|
| Ex. | Structure/Name | in |
| C-1 | 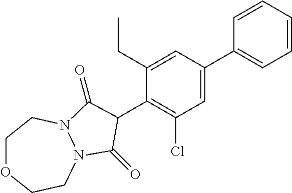 [SMILES: CCc1cc(-c2ccccc2)cc(Cl)c1C1C(=O)N2CCOCCN2C1=O] | WO 01/17973 |
| C-2 | 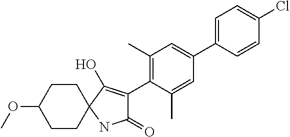 [SMILES: COC1CCC2(CC1)NC(=O)C(c1c(C)cc(-c3ccc(Cl)cc3)cc1C)=C2O] | WO 99/43649 I-1-a-2 |
| C-3 | 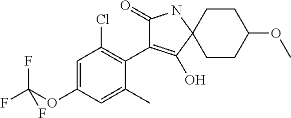 [SMILES: COC1CCC2(CC1)NC(=O)C(c1c(C)cc(OC(F)(F)F)cc1Cl)=C2O] | WO 08/067873 I-a-39 |
| C-4 | 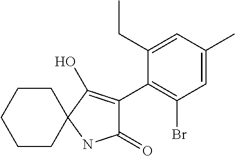 [SMILES: CCc1cc(C)cc(Br)c1C1=C(O)C2(CCCCC2)NC1=O] | WO 97/02243 I-a-27 |

### Compounds of the Formula (I-1)

The compounds of the formula (I-1) according to the invention are known and/or can be prepared via synthesis routes A and/or B.

### Synthesis Route A

An aryl bromide derivative of the formula (II)

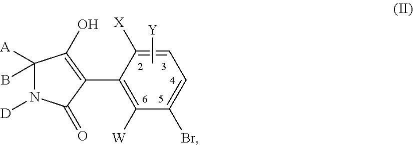

in which A, B, D, W, X and Y have the meanings given above is reacted in a Suzuki coupling with compounds of the formula (III)

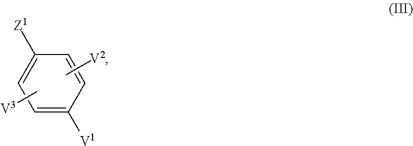

in which V¹, V² and V³ have the meanings given above and Z¹ represents B(OH)₂, a boronic acid ester, preferably boronic acid pinacol ester, or —BF₃⁻K⁺.

The Suzuki couplings are generally carried out in inert solvents in the presence of a catalyst, optionally in the presence of an additional reagent, preferably in a temperature range of from room temperature to 130° C. at atmospheric pressure. The reactions can also be carried out in a closed vessel with heating in a microwave oven.

Suitable catalysts are, for example, palladium catalysts customary for Suzuki reaction conditions; preference is given to catalysts such as, for example, dichlorobis(triphenylphosphine)palladium, tetrakistriphenylphosphinepalladium(0), palladium on carbon, palladium(II) acetate, palladium(II) acetate/triscyclohexylphosphine, palladium(II) acetoacetonate/tri-tert-butylphosphonium tetrafluoroborate, dichloro[1,1′-bis(diphenylphosphino)ferrocene]palladium dichloromethane complex or palladium(II) acetate having a ligand such as dicyclohexyl[2′,4′,6′-tri(propan-2-yl)biphenyl-2-yl]phosphane.

Suitable additional reagents are, for example, potassium acetate or caesium acetate, caesium carbonate, potassium carbonate or sodium carbonate, potassium tert-butoxide, caesium fluoride, potassium phosphate or sodium hydroxide or potassium hydroxide; preference is given to additional reagents such as, for example, caesium carbonate and/or aqueous sodium hydroxide solution.

Suitable inert solvents are, for example, ethers such as dioxane, tetrahydrofuran or 1,2-dimethoxyethane, hydrocarbons such as benzene, xylene or toluene, or carboxamides such as dimethylformamide, dimethylacetamide or N-methylpyrrolidone, or alkyl sulphoxides such as dimethyl sulphoxide, or mixtures of these solvents with alcohols such as methanol or ethanol and/or water; preference is given to 1,2-dimethoxyethane.

The compounds of the formula (II) are known and/or can be prepared by reacting compounds of the formula (IV)

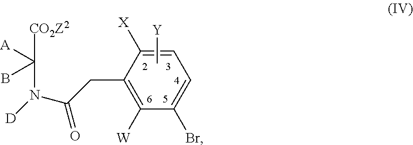

in which A, B, D, W, X and Y have the meanings given above and
Z² represents C₁-C₆-alkyl, preferably ethyl or methyl,
under Dieckmann condensation conditions.

Dieckmann condensations are generally carried out in inert solvents in the presence of a base, preferably in a temperature range of from room temperature to 130° C. at atmospheric pressure. Suitable bases are, for example, alkali- or alkaline earth alkoxides such as sodium tert-butoxide or potassium tert-butoxide, sodium methoxide or ethoxide; preference is given to potassium tert-butoxide.

Suitable inert solvents are, for example, ethers such as dioxane, tetrahydrofuran or 1,2-dimethoxyethane, hydrocarbons such as benzene, xylene or toluene, or carboxamides such as dimethylformamide, dimethylacetamide or N-methylpyrrolidone, or alkyl sulphoxides such as dimethyl sulphoxide, or alcohols such as methanol or ethanol; preference is given to dimethylformamide.

The compounds of the formula (IV) are known and/or can be prepared by reacting compounds of the formula (V) or a salt of compounds of the formula (V)

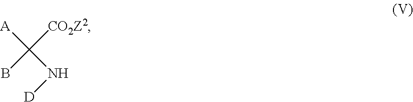

in which A, B, D and Z² have the meanings given above with compounds of the formula (VI)

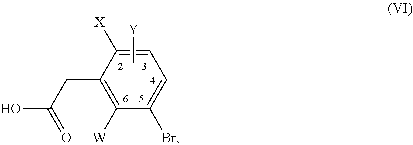

in which X, Y and W have the meanings given above under amide coupling conditions.

The reaction is generally carried out in inert solvents by reacting compounds of the formula (VI) initially with thionyl chloride or an equivalent reagent known to the person skilled in the art and in the second step with compounds of the formula (V) or a salt of compounds of the formula (V) in the presence of a base such as, for example, triethylamine or potassium carbonate.

In an alternative process, the reaction can be carried out in inert solvents in the presence of a dehydrating agent, if appropriate in the presence of a base, preferably in a temperature range of from −30° C. to 50° C. at atmospheric pressure.

Suitable inert solvents are, for example, halogenated hydrocarbons such as dichloromethane or trichloromethane, hydrocarbons such as benzene or toluene, nitromethane, tetrahydrofuran, 1,4-dioxane, dimethylformamide or acetonitrile. It is also possible to use mixture of the solvents. Particular preference is given to acetonitrile, dichloromethane, dimethylformamide, tetrahydro-furan or toluene.

Suitable bases are, for example, alkali metal carbonates such as, for example, sodium carbonate or potassium carbonate or sodium bicarbonate or potassium bicarbonate, or organic bases such as trialkylamines, for example triethylamine, N-methylmorpholine, N-methylpiperidine, 4-dimethyl-aminopyridine or diisopropylethylamine.

Suitable dehydrating agents are here, for example, carbodiimides such as, for example, N,N′-diethyl-, N,N,′-dipropyl-, N,N′-diisopropyl-, N,N′-dicyclohexylcarbodiimide, N-(3-dimethylamino-isopropyl)-N′-ethylcarbodiimide hydrochloride (EDC), N-cyclohexylcarbodiimide-N′-propyloxymethyl-polystyrene (PS carbodiimide) or carbonyl compounds such as carbonyldiimidazole, or 1,2-oxazolium compounds such as 2-ethyl-5-phenyl-1,2-oxazolium 3-sulphate or 2-tert-butyl-5-methylisoxazolium perchlorate, or acylamino compounds such as 2-ethoxy-1-ethoxy-carbonyl-1,2-dihydroquinoline, or propanephosphonic anhydride, or isobutyl chloroformate, or bis-(2-oxo-3-oxazolidinyl)phosphoryl chloride, or O-(benzotriazol-1-yl)-N,N,N′,N′ tetramethyluronium hexafluorophosphate (HBTU), 2-(2-oxo-1-(2H)-pyridyl)-1,1,3,3-tetramethyluronium tetra-fluoroborate (TPTU) or O-(7-azabenzotriazol-1-yl) N,N,N′,N′-tetramethyluronium hexafluoro-phosphate (HATU), or 1-hydroxybenzotriazole (HOBt), or benzotriazol-1-yloxy-tris(dimethylamino)phosphonium hexafluorophosphate (BOP), or benzotriazol-1-yloxytris(pyrrolidino)phosphonium hexafluorophosphate (PyBOP), or N-hydroxysuccinimide, or mixtures of these, with bases.

The condensation is preferably carried out using PyBOP, TBTU or using EDC in the presence of HOBt.

The process described above is illustrated by the synthesis scheme below:

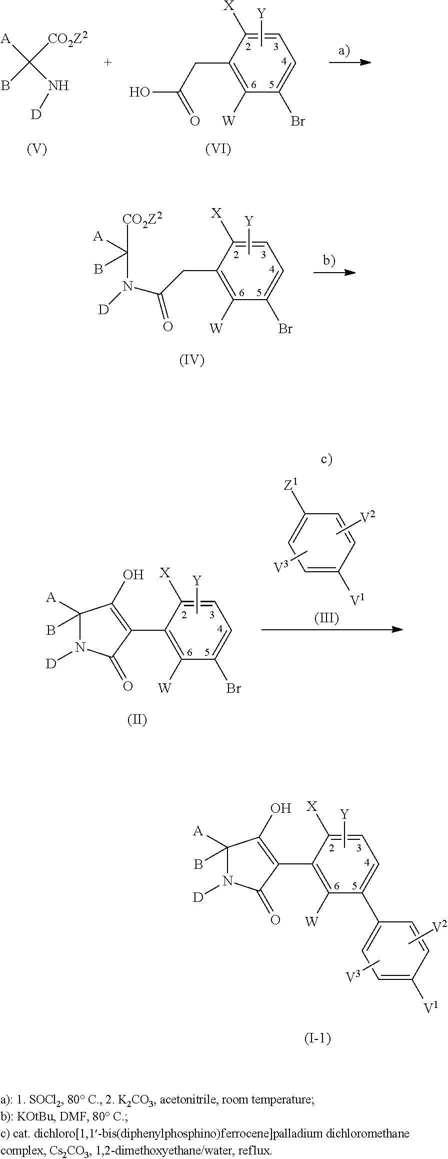

### Synthesis Route B

Alternatively, the compounds of the formula (I-1) according to the invention can be prepared by reacting a compound of the formula (VII)

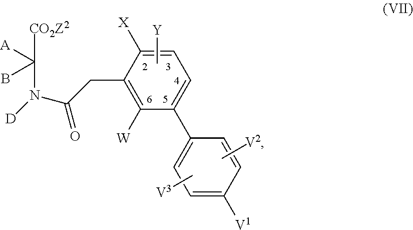

in which A, B, D, W, X, Y, V¹, V², V³ and Z² have the meanings given above under the conditions of a Dieckmann condensation given above.

The compounds of the formula (VII) are known and/or can be prepared by reacting compounds of the formula (V) or a salt of compounds of the formula (V) in which A, B, D and Z² have the meanings given above with compounds of the formula (VIII)

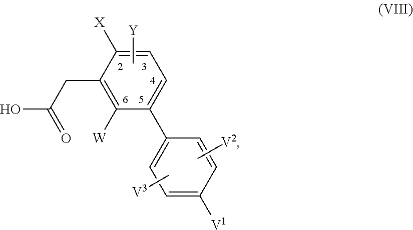

in which X, Y, W, V¹, V² and V³ have the meanings given above, under the amide coupling conditions given above.

The compounds of the formula (VIII) are known and/or can be prepared by reacting compounds of the formula (IX)

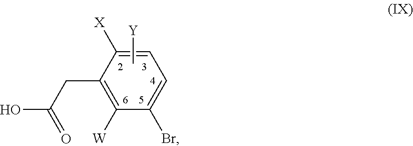

in which X, Y and W have the meanings given above in a Suzuki reaction under the conditions given above with compounds of the formula (III) in which V¹, V², V³ and Z¹ have the meanings given above.

The process described above is illustrated by the synthesis scheme below:

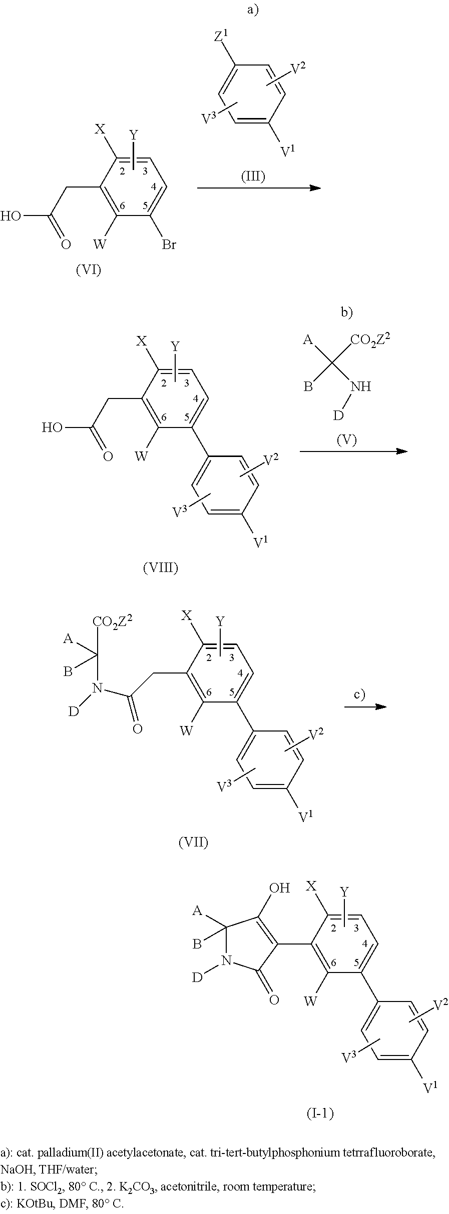

The compounds of the formulae (I-1) and (II) prepared by the above process optionally carry protective groups which can be removed under conditions known to the person skilled in the art, giving further compounds of the formulae (I-1) and (II).

Thus, for example, it is possible to prepare compounds of the formula (I-1a) according to the invention

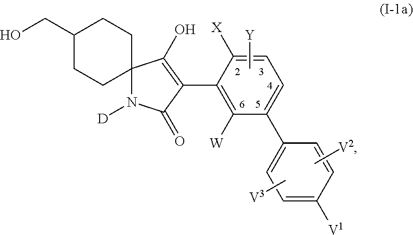

in which D, X, Y, W, V¹, V² and V³ have the meanings given above by cleaving the ether in compounds of the formula (I-1b)

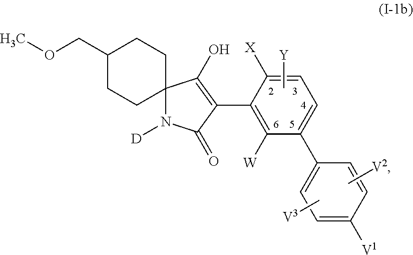

in which D, X, Y, W, V¹, V² and V³ have the meanings given above.

The reaction is generally carried out in inert solvents by reacting the compounds of the formula (I-1b) with sodium iodide/trimethylsilyl chloride or with trimethylsilyl iodide or with boron tribromide or with boron trichloride or with hydrogen bromide/acetic acid or with aluminium tribromide/ethanethiol or an equivalent reagent known to the person skilled in the art, if appropriate in the presence of a base, in a temperature range of from −78° C. to the reflux temperature of the respective solvent at atmospheric pressure.

Suitable inert solvents are, for example, halogenated hydrocarbons such as dichloromethane or trichloromethane, hydrocarbons such as benzene or toluene, tetrahydrofuran, 1,4-dioxane, dimethylformamide or acetonitrile. It is also possible to use mixtures of the solvents. Particular preference is given to acetonitrile and dichloromethane.

Suitable bases are, for example, alkali metal carbonates, such as, for example, sodium carbonate or potassium carbonate or sodium bicarbonate or potassium bicarbonate, or organic bases such as trialkylamines, for example triethylamine, N-methylmorpholine, N-methylpiperidine, 4-dimethyl-aminopyridine or diisopropylethylamine.

Preferably, the cleavage is carried out using sodium iodide/trimethylsilyl chloride in acetonitrile.

Thus, it is also possible to prepare, for example, compounds of the formula (IIa) according to the invention

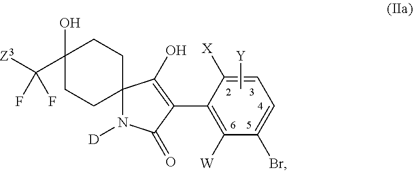

in which D, X, Y and W have the meanings given above and
Z³ represents fluorine or trifluoromethyl by reacting ketones of the formula (IIb)

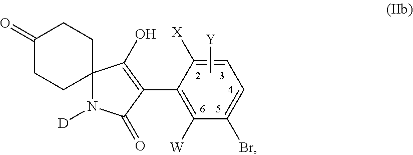

in which D, X, Y and W have the meanings given above with (trifluoromethyl)trimethylsilane or (pentafluoroethyl)trimethylsilane.

The reactions of the ketones of the formula (IIb) with (trifluoromethyl)trimethylsilane or (pentafluoroethyl)trimethylsilane to give the compounds of the formula (IIa) are generally carried out in inert solvents, in the presence of a catalyst, preferably in a temperature range of from −20° C. to 100° C. at atmospheric pressure. Suitable catalysts are, for example, alkali metal or alkaline earth metal carbonates such as sodium carbonate, potassium carbonate or caesium carbonate. It is furthermore possible to use alkali metal or alkaline earth metal fluorides such as lithium fluoride and caesium fluoride and also fluoride salts of organic bases such as, for example, tetraethylammonium fluoride or tetrabutylammonium fluoride to catalyse the desired reaction. Suitable inert solvents are, for example, ethers such as dioxane, tetrahydrofuran or 1,2-dimethoxyethane, or carboxamides such as dimethylformamide, dimethylacetamide or N-methylpyrrolidone, or alkyl sulphoxides such as dimethyl sulphoxide; preference is given to dimethylformamide. The silyl derivatives of the formula (IIa) obtained in the first instance are then cleaved using methods known to the person skilled in the art (see Protective Groups in Organic Synthesis; Theodora W. Greene).

The compounds of the formula (IIb) are known and/or can be prepared by removing the ketal protective group in compounds of the formula (IIc)

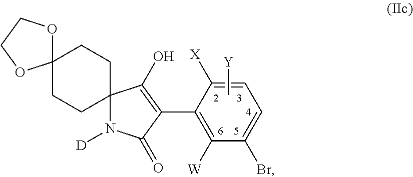

in which D, W, X and Y have the meanings given above by methods known to the person skilled in the art (see Protective Groups in Organic Synthesis; Theodora W. Greene).

### Abbreviations and Acronyms:

Ac acetyl
Bn benzyl
Bu butyl
cat. catalytic
CI chemical ionization (in MS)
DMF dimethylformamide
DMSO dimethyl sulphoxide
EI electron impact ionization (in MS)
eq. equivalent(s)
ESI electrospray ionization (in MS)
Et ethyl
EtOAc ethyl acetate
h hour(s)
HPLC high-pressure, high-performance liquid chromatography
conc. concentrated
LC-MS liquid chromatography-coupled mass spectrometry
Me methyl
min minute(s)
MS mass spectrometry
NMR nuclear magnetic resonance spectrometry
Ph phenyl
RT room temperature
Rₜ retention time (in HPLC)
THF tetrahydrofuran
UV ultraviolet spectrometry
aq. aqueous, aqueous solution

### LC-MS and HPLC Methods:

### Method 1 (UPLC-MS)

Instrument: Waters Acquity HPLC-MS SQD 3001; column: Acquity HPLC BEH C18 1.7 50×2.1 mm; mobile phase A: water+0.1% formic acid, mobile phase B: acetonitrile; gradient: 0-1.6 min 1-99% B, 1.6-2.0 min 99% B; flow rate 0.8 ml/min; temperature: 60° C.; injection: 2 μl; DAD scan: 210-400 nM.

### Method 2 (UPLC-MS):

Instrument: Waters Acquity HPLC-MS ZQ4000; column: Acquity UPLC BEH C18 1.7 50×2.1 mm; mobile phase A: water+0.05% formic acid, mobile phase B: acetonitrile+0.05% formic acid; gradient: 0-1.6 min 1-99% B, 1.6-2.0 min 99% B; flow rate 0.8 ml/min; temperature: 60° C.; injection: 2 μl; DAD scan: 210-400 nM.

### Method 3 (UPLC-MS):

Instrument: Waters Acquity UPLC-MS SQD 3001; column: Acquity UPLC BEH C18 1.7 50×2.1 mm; mobile phase A: water+0.1% formic acid, mobile phase B: acetonitrile; gradient: 0-1.6 min 1-99% B, 1.6-2.0 min 99% B; flow rate 0.8 ml/min; temperature: 60° C.; injection: 2 μl; DAD scan: 210-400 nm.

### Method 4 (HPLC-MS):

Instrument MS: Waters ZQ; Instrument HPLC: Waters UPLC Acquity; column: Acquity BEH C18 (Waters), 50 mm×2.1 mm, 1.7 μm; mobile phase A: water+0.1% formic acid, mobile phase B: acetonitrile (Lichrosolv Merck); gradient: 0.0 min 99% A—1.6 min 1% A—1.8 min 1% A—1.81 min 99% A—2.0 min 99% A; oven: 60° C.; flow rate: 0.800 ml/min; UV detection PDA 210-400 nm.

Table 1 lists some of the structures of the formula (I-1) of the prior art and indicates which patent discloses the preparation.

TABLE 1

| | | | Analysis |
|---|---|---|---|
| | | | ¹H-NMR: δ [ppm] |
| Ex. | Structure/Name | disclosed in | retention time, [M + H]⁺, Method |
| 1-1 | 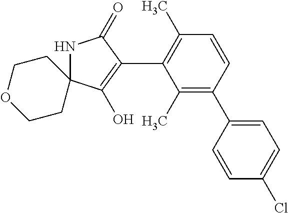 [SMILES: Cc1ccc(-c2ccc(Cl)cc2)c(C)c1C1=C(O)C2(CCOCC2)NC1=O] | WO 99/48869 I-1-a-15 | (300 MHz, DMSO-d₆): 1.23-1.36 (m, 2H), 1.98 (s, 3H), 2.03-2.19 (m, 5H), 3.62-3.76 (m, 2H), 3.80- 3.91 (m, 2H), 7.05 (d, 1H), 7.13 (d, 1H), 7.26-7.35 (m, 2H), 7.45- 7.53 (m, 2H), 8.40 (s, 1H), 10.89 (br. s., 1H). 1.14 min, 384, Method 1 |
| | 3-(4′-chloro-2,4-dimethylbiphenyl-3-yl)- | | |
| | 4-hydroxy-8-oxa-1-azaspiro[4.5]dec-3- | | |
| | en-2-one | | |
| 1-2 | 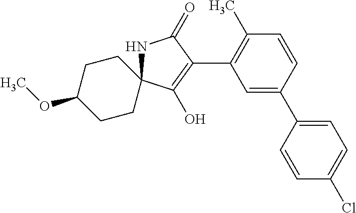 [SMILES: CO[C@H]1CC[C@]2(CC1)NC(=O)C(c1cc(-c3ccc(Cl)cc3)ccc1C)=C2O] | WO 99/48869 I-1-a-16 | (300 MHz, DMSO-d₆): 1.39-1.62 (m, 4H), 1.84-2.05 (m, 4H), 2.18 (s, 3H), 3.07-3.20 (m, 1H), 3.26 (s, 3H), 7.30 (d, 1H), 7.34 (d, 1H), 7.45-7.53 (m, 3H), 7.62-7.68 (m, 2H), 8.18 (br. s, 1H), 10.82 (br. s, 1H). 1.20 min, 398, Method 1 |
| | (5s,8s)-3-(4′-chloro-4-methylbiphenyl-3- | | |
| | yl)-4-hydroxy-8-methoxy-1- | | |
| | azaspiro[4.5]dec-3-en-2-one | | |
| 1-3 | 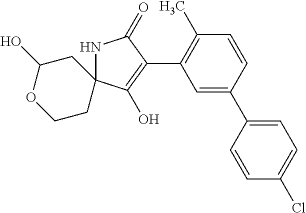 [SMILES: Cc1ccc(-c2ccc(Cl)cc2)cc1C1=C(O)C2(CCOC(O)C2)NC1=O] | WO 99/48869 I-1-a-17 | |
| | 3-(4′-chloro-4-methylbiphenyl-3-yl)-4- | | |
| | hydroxy-7-methyl-8-oxa-1- | | |
| | azaspiro[4.5]dec-3-en-2-one | | |
| 1-4 | 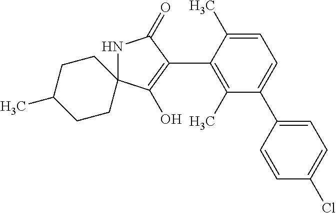 [SMILES: Cc1ccc(-c2ccc(Cl)cc2)c(C)c1C1=C(O)C2(CCC(C)CC2)NC1=O] | WO 99/48869 I-1-a-2 | |
| | 3-(4′-chloro-2,4-dimethylbiphenyl-3-yl)- | | |
| | 4-hydroxy-8-methyl-1-azaspiro[4.5]dec- | | |
| | 3-en-2-one | | |
| 1-5 | 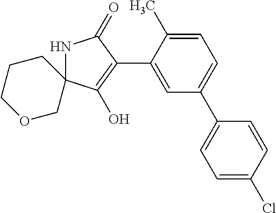 [SMILES: Cc1ccc(-c2ccc(Cl)cc2)cc1C1=C(O)C2(CCCOC2)NC1=O] | WO 99/48869 I-1-a-21 | (300 MHz, DMSO-d₆): 1.47-1.63 (m, 2H), 1.85-2.03 (m, 1H), 2.05- 2.21 (m, 4H), 3.27-3.40 (m, 1H), 3.44-3.52 (m, 1H), 3.67-3.74 (m, 1H), 3.80-3.89 (m, 1H), 7.28 (d, 1H), 7.36 (d, 1H), 7.42-7.52 (m, 3H), 7.60-7.68 (m, 2H), 7.95 (br. s., 1H), 11.08 (s, 1H). 1.14 min, 370, Method 1 |
| | 3-(4′-chloro-4-methylbiphenyl-3-yl)-4- | | |
| | hydroxy-7-oxa-1-azaspiro[4.5]dec-3-en- | | |
| | 2-one | | |
| 1-6 | 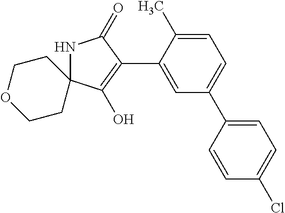 [SMILES: Cc1ccc(-c2ccc(Cl)cc2)cc1C1=C(O)C2(CCOCC2)NC1=O] | WO 99/48869 I-1-a-22 | (300 MHz, DMSO-d₆): 1.25-1.35 (m, 2H), 2.06-2.23 (m, 5H), 3.63- 3.76 (m, 2H), 3.81-3.91 (m, 2H), 7.31 (d, 1H), 7.36 (d, 1H), 7.46- 7.53 (m, 3H), 7.62-7.69 (m, 2H), 8.46 (s, 1H), 10.98 (br. s., 1H). 1.11 min, 370, Method 1 |
| | 3-(4′-chloro-4-methylbiphenyl-3-yl)-4- | | |
| | hydroxy-8-oxa-1-azaspiro[4.5]dec-3-en- | | |
| | 2-one | | |
| 1-7 | 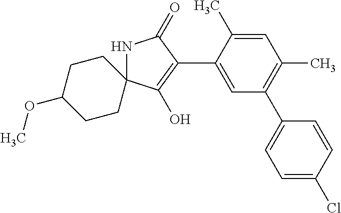 [SMILES: COC1CCC2(CC1)NC(=O)C(c1cc(-c3ccc(Cl)cc3)c(C)cc1C)=C2O] | WO 99/48869 I-1-a-25 | (300 MHz, DMSO-d₆): 1.36-1.59 (m, 4H), 1.82-2.02 (m, 4H), 2.15 (s, 3H), 2.21 (s, 3H), 3.05-3.18 (m, 1H), 3.25 (s, 3H), 6.89 (s, 1H), 7.14 (s, 1H), 7.31-7.38 (m, 2H), 7.44-7.52 (m, 2H), 8.14 (s, 1H), 10.73 (br. s., 1H). 1.26 min, 412, Method 1 |
| | 3-(4′-chloro-4,6-dimethylbiphenyl-3-yl)- | | |
| | 4-hydroxy-8-methoxy-1- | | |
| | azaspiro[4.5]dec-3-en-2-one | | |
| 1-8 | 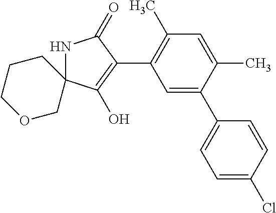 [SMILES: Cc1cc(C)c(-c2ccc(Cl)cc2)cc1C1=C(O)C2(CCCOC2)NC1=O] | WO 99/48869 I-1-a-27 | (400 MHz, DMSO-d₆): 1.47-1.62 (m, 2H), 1.88-2.01 (m, 1H), 2.07- 2.13 (m, 1H), 2.14 (s, 3H), 2.21 (s, 3H), 3.29-3.38 (m, 1H), 3.47 (dd, 1H), 3.71 (d, 1H), 3.81-3.88 (m, 1H), 6.90 (s, 1H), 7.15 (s, 1H), 7.33-7.37 (m, 2H), 7.46-7.51 (m, 2H), 8.14 (s, 1H), 10.86 (br. s., 1H). 1.20 min, 384, Method 1 |
| | 3-(4′-chloro-4,6-dimethylbiphenyl-3-yl)- | | |
| | 4-hydroxy-7-oxa-1-azaspiro[4.5]dec-3- | | |
| | en-2-one | | |
| 1-9 | 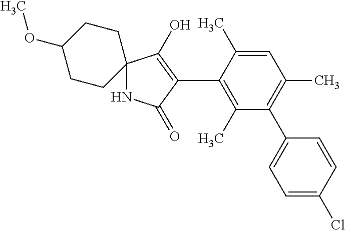 [SMILES: COC1CCC2(CC1)NC(=O)C(c1c(C)cc(C)c(-c3ccc(Cl)cc3)c1C)=C2O] | WO 99/48869 I-1-a-3 | |
| | 3-(4′-chloro-2,4,6-trimethylbiphenyl-3-yl)- | | |
| | 4-hydroxy-8-methoxy-1- | | |
| | azaspiro[4.5]dec-3-en-2-one | | |
| 1-10 | 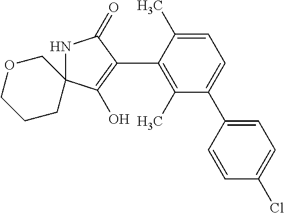 [SMILES: Cc1ccc(-c2ccc(Cl)cc2)c(C)c1C1=C(O)C2(CCCOC2)NC1=O] | WO 99/48869 I-1-a-7 | (400 MHz, DMSO-d₆): 1.49-1.65 (m, 2H), 1.91-2.03 (m, 4H), 2.07- 2.19 (m, 4H), 3.29-3.40 (m, 1H), 3.45-3.53 (m, 1H), 3.68-3.75 (m, 1H), 3.82-3.90 (m, 1H), 7.05 (d, 1H), 7.13 (d, 1H), 7.27-7.33 (m, 2H), 7.46-7.51 (m, 2H), 8.16 (br. s., 1H), 10.89 (br. s., 1H). 1.16, 1.17 min, 384, Method 2 |
| | 3-(4′-chloro-2,4-dimethylbiphenyl-3-yl)- | | |
| | 4-hydroxy-7-oxa-1-azaspiro[4.5]dec-3- | | |
| | en-2-one | | |
| 1-11 | 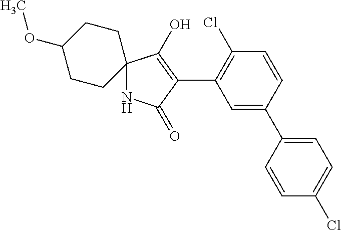 [SMILES: COC1CCC2(CC1)NC(=O)C(c1cc(-c3ccc(Cl)cc3)ccc1Cl)=C2O] | WO 99/488690 I-1-a-9 | (300 MHz, DMSO-d₆): 1.39-1.61 (m, 4H), 1.82-2.03 (m, 4H), 3.07- 3.20 (m, 1H), 3.26 (s, 3H), 7.46- 7.57 (m, 4H), 7.58-7.64 (m, 1H), 7.65-7.74 (m, 2H), 8.22 (br. s., 1H), 11.10 (br. s., 1H). 1.21 min, 418, Method 1 |
| | 3-(4,4′-dichlorobiphenyl-3-yl)-4-hydroxy- | | |
| | 8-methoxy-1-azaspiro[4.5]dec-3-en-2-one | | |
| 1-12 | 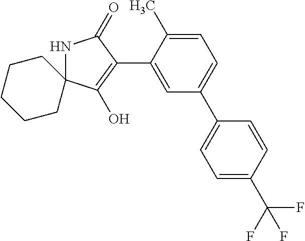 [SMILES: Cc1ccc(-c2ccc(C(F)(F)F)cc2)cc1C1=C(O)C2(CCCCC2)NC1=O] | WO 99/48869 T13 | |
| | 4-hydroxy-3-[4 methyl-4′- | | |
| | (trifluoromethyl)biphenyl-3-yl]-1- | | |
| | azaspiro[4.5]dec-3-en-2-one | | |
| 1-13 | 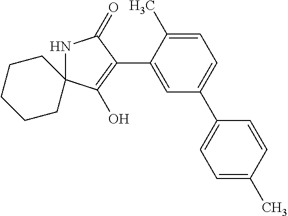 [SMILES: Cc1ccc(-c2ccc(C)c(C3=C(O)C4(CCCCC4)NC3=O)c2)cc1] | WO 99/48869 T16 | |
| | 3-(4,4′-dimethylbiphenyl-3-yl)-4- | | |
| | hydroxy-1-azaspiro[4.5]dec-3-en-2-one | | |
| 1-14 | 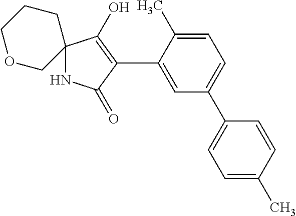 [SMILES: Cc1ccc(-c2ccc(C)c(C3=C(O)C4(CCCOC4)NC3=O)c2)cc1] | WO 99/48869 T16 | |
| | 3-(4,4′-dimethylbiphenyl-3-yl)-4- | | |
| | hydroxy-7-oxa-1-azaspiro[4.5]dec-3-en- | | |
| | 2-one | | |
| 1-15 | 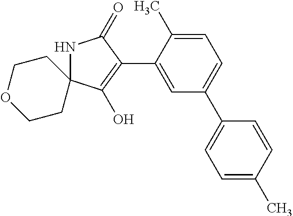 [SMILES: Cc1ccc(-c2ccc(C)c(C3=C(O)C4(CCOCC4)NC3=O)c2)cc1] | WO 99/48869 T16 | |
| | 3-(4,4′-dimethylbiphenyl-3-yl)-4- | | |
| | hydroxy-8-oxa-1-azaspiro[4.5]dec-3-en- | | |
| | 2-one | | |
| 1-16 | 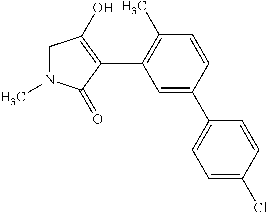 [SMILES: Cc1ccc(-c2ccc(Cl)cc2)cc1C1=C(O)CN(C)C1=O] | WO 99/48869 T3 | |
| | 3-(4′-chloro-4-methylbiphenyl-3-yl)-4- | | |
| | hydroxy-1-methyl-1,5-dihydro-2H- | | |
| | pyrrol-2-one | | |
| 1-17 | 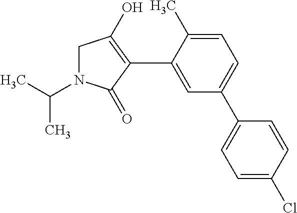 [SMILES: Cc1ccc(-c2ccc(Cl)cc2)cc1C1=C(O)CN(C(C)C)C1=O] | WO 99/48869 T3 | |
| | 3-(4′-chloro-4-methylbiphenyl-3-yl)-4- | | |
| | hydroxy-1-(propan-2-yl)-1,5-dihydro-2H- | | |
| | pyrrol-2-one | | |
| 1-18 | 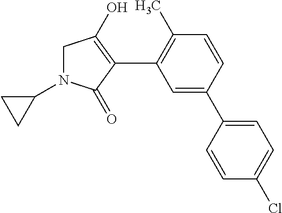 [SMILES: Cc1ccc(-c2ccc(Cl)cc2)cc1C1=C(O)CN(C2CC2)C1=O] | WO 99/48869 T3 | |
| | 3-(4′-chloro-4-methylbiphenyl-3-yl)-1- | | |
| | cyclopropyl-4-hydroxy-1,5-dihydro-2H- | | |
| | pyrrol-2-one | | |
| 1-19 | 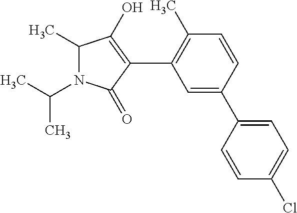 [SMILES: Cc1ccc(-c2ccc(Cl)cc2)cc1C1=C(O)C(C)N(C(C)C)C1=O] | WO 99/48869 T3 | |
| | 3-(4′-chloro-4-methylbiphenyl-3-yl)-4- | | |
| | hydroxy-5-methyl-1-(propan-2-yl)-1,5- | | |
| | dihydro-2H-pyrrol-2-one | | |
| 1-20 | 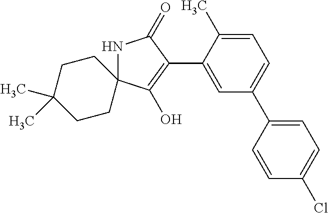 [SMILES: Cc1ccc(-c2ccc(Cl)cc2)cc1C1=C(O)C2(CCC(C)(C)CC2)NC1=O] | WO 99/48869 T4 | (400 MHz, DMSO-d₆): 0.94 (s, 3H), 0.96 (s, 3H), 1.20-1.35 (m, 4H), 1.56-1.67 (m, 2H), 1.98- 2.08 (m, 2H), 2.19 (s, 3H), 7.30 (d, 1H), 7.35 (d, 1H), 7.46-7.52 (m, 3H), 7.62-7.67 (m, 2H), 8.19 (s, 1H), 10.78 (s, 1H). 1.40, 396, Method 2 |
| | 3-(4′-chloro-4-methylbiphenyl-3-yl)-4- | | |
| | hydroxy-8,8-dimethyl-1- | | |
| | azaspiro[4.5]dec-3-en-2-one | | |
| 1-21 | 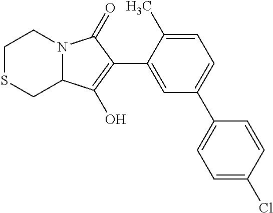 [SMILES: Cc1ccc(-c2ccc(Cl)cc2)cc1C1=C(O)C2CSCCN2C1=O] | WO 99/48869 T4 | (300 MHz, DMSO-d₆): 2.21 (s, 3H), 2.41-2.67 (m, 3H), 2.93- 3.05 (m, 1H), 3.08-3.17 (m, 1H), 4.09 (dd, 1H), 4.35-4.44 (m, 1H), 7.31 (d, 1H), 7.39 (d, 1H), 7.46- 7.53 (m, 3H), 7.61-7.68 (m, 2H), 11.25 (br. s., 1H). 1.23 min, 372, Method 1 |
| | 7-(4′-chloro-4-methylbiphenyl-3-yl)-8- | | |
| | hydroxy-1,3,4,8a-tetrahydro-6H- | | |
| | pyrrolo[2,1-c][1,4]thiazin-6-one | | |
| 1-22 | 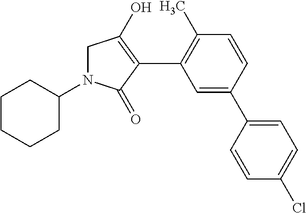 [SMILES: Cc1ccc(-c2ccc(Cl)cc2)cc1C1=C(O)CN(C2CCCCC2)C1=O] | WO 99/48869 T4 | |
| | 3-(4′-chloro-4-methylbiphenyl-3-yl)-1- | | |
| | cyclohexyl-4-hydroxy-1,5-dihydro-2H- | | |
| | pyrrol-2-one | | |
| 1-23 | 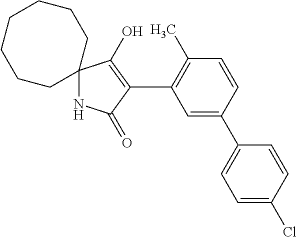 [SMILES: Cc1ccc(-c2ccc(Cl)cc2)cc1C1=C(O)C2(CCCCCCC2)NC1=O] | WO 99/48869 T4 | |
| | 3-(4′-chloro-4-methylbiphenyl-3-yl)-4- | | |
| | hydroxy-1-azaspiro[4.7]dodec-3-en-2-one | | |
| 1-24 | 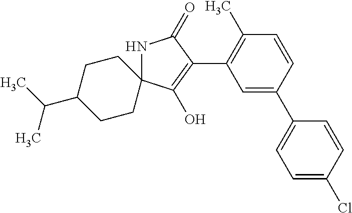 [SMILES: Cc1ccc(-c2ccc(Cl)cc2)cc1C1=C(O)C2(CCC(C(C)C)CC2)NC1=O] | WO 99/48869 T4 | (300 MHz, DMSO-d₆): 0.87 (s, 3H), 0.90 (s, 3H), 0.97-1.12 (m, 1H), 1.30-1.50 (m, 5H), 1.59- 1.72 (m, 2H), 1.81-1.96 (m, 2H), 2.19 (s, 3H), 7.30 (d, 1H), 7.34 (d, 1H), 7.46-7.52 (m, 3H), 7.62- 7.68 (m, 2H), 8.17 (s, 1H), 10.73 (s, 1H). 1.45 min, 410, Method 1 |
| | 3-(4′-chloro-4-methylbiphenyl-3-yl)-4- | | |
| | hydroxy-8-(propan-2-yl)-1- | | |
| | azaspiro[4.5]dec-3-en-2-one | | |
| 1-25 | 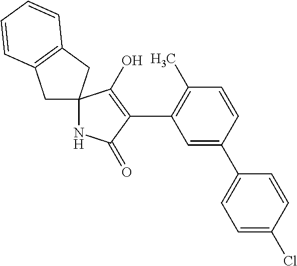 [SMILES: Cc1ccc(-c2ccc(Cl)cc2)cc1C1=C(O)C2(Cc3ccccc3C2)NC1=O] | WO 99/48869 T4 | |
| | 4′-(4′-chloro-4-methylbiphenyl-3-yl)-3′- | | |
| | hydroxy-1,3-dihydrospiro[inden-2,2′- | | |
| | pyrrol]-5′(1′H)-one | | |
| 1-26 | 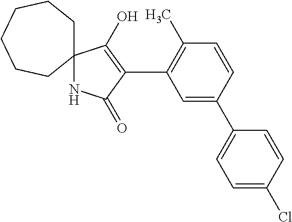 [SMILES: Cc1ccc(-c2ccc(Cl)cc2)cc1C1=C(O)C2(CCCCCC2)NC1=O] | WO 99/48869 T4 | |
| | 3-(4′-chloro-4-methylbiphenyl-3-yl)-4- | | |
| | hydroxy-1-azaspiro[4.6]undec-3-en-2-one | | |
| 1-27 | 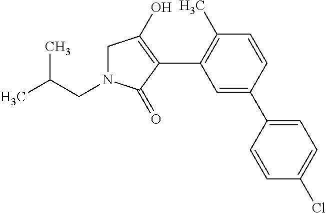 [SMILES: Cc1ccc(-c2ccc(Cl)cc2)cc1C1=C(O)CN(CC(C)C)C1=O] | WO 99/48869 T4 | |
| | 3-(4′-chloro-4-methylbiphenyl-3-yl)-4- | | |
| | hydroxy-1-(2-methylpropyl)-1,5-dihydro- | | |
| | 2H-pyrrol-2-one | | |
| 1-28 | 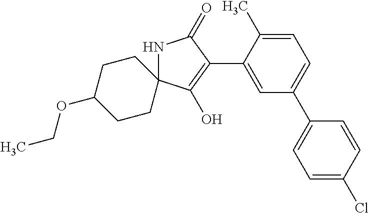 [SMILES: CCOC1CCC2(CC1)NC(=O)C(c1cc(-c3ccc(Cl)cc3)ccc1C)=C2O] | WO 99/48869 T4 | |
| | 3-(4′-chloro-4-methylbiphenyl-3-yl)-8- | | |
| | ethoxy-4-hydroxy-1-azaspiro[4.5]dec-3- | | |
| | en-2-one | | |
| 1-29 | 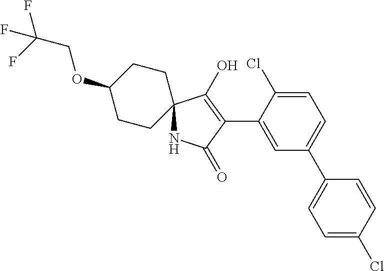 [SMILES: O=C1N[C@]2(CC[C@@H](OCC(F)(F)F)CC2)C(O)=C1c1cc(-c2ccc(Cl)cc2)ccc1Cl] | WO 09/039975 I-1-a-18 | (400 MHz, DMSO-d₆): 1.44-1.53 (m, 2H), 1.56-1.69 (m, 2H), 1.85- 2.05 (m, 4H), 3.42-3.52 (m, 1H), 4.10 (q, 2H), 7.49-7.56 (m, 4H), 7.61 (dd, 1H), 7.67-7.71 (m, 2H), 8.26 (br. s., 1H), 11.13 (s, 1H). 1.32 min, 486, Method 1 |
| | (5s,8s)-3-(4,4′-dichlorobiphenyl-3-yl)-4- | | |
| | hydroxy-8-(2,2,2-trifluoroethoxy)-1- | | |
| | azaspiro[4.5]dec-3-en-2-one | | |
| 1-30 | 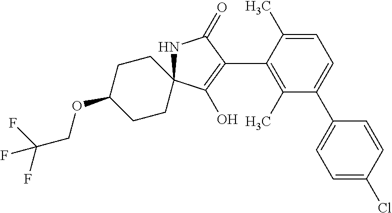 [SMILES: Cc1ccc(-c2ccc(Cl)cc2)c(C)c1C1=C(O)[C@]2(CC[C@H](OCC(F)(F)F)CC2)NC1=O] | WO 09/039975 I-1-a-25 | (300 MHz, DMSO-d₆): 1.39-1.52 (m, 2H), 1.54-1.72 (m, 2H), 1.84- 2.06 (m, 7H), 2.12 (s, 3H), 3.40- 3.54 (m, 1H), 4.10 (q, 2H), 7.04 (d, 1H), 7.12 (d, 1H), 7.27-7.33 (m, 2H), 7.45-7.52 (m, 2H), 8.16 (s, 1H), 10.76 (s, 1H). 1.35 min, 480, Method 2 |
| | (5s,8s)-3-(4′-chloro-2,4- | | |
| | dimethylbiphenyl-3-yl)-4-hydroxy-8- | | |
| | (2,2,2-trifluoroethoxy)-1- | | |
| | azaspiro[4.5]dec-3-en-2-one | | |
| 1-31 | 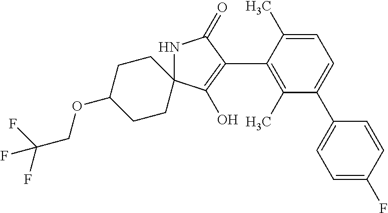 [SMILES: Cc1ccc(-c2ccc(F)cc2)c(C)c1C1=C(O)C2(CCC(OCC(F)(F)F)CC2)NC1=O] | WO 09/039975 I-1-a-3 | |
| | 3-(4′-fluoro-2,4-dimethylbiphenyl-3-yl)-4- | | |
| | hydroxy-8-(2,2,2-trifluoroethoxy)-1- | | |
| | azaspiro[4.5]dec-3-en-2-one | | |
| 1-32 | 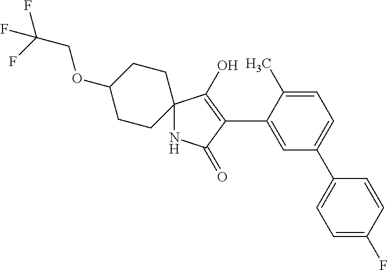 [SMILES: Cc1ccc(-c2ccc(F)cc2)cc1C1=C(O)C2(CCC(OCC(F)(F)F)CC2)NC1=O] | WO 09/039975 I-1-a-31 | (300 MHz, DMSO-d₆): 1.16-1.28 (m, 0.7H), 1.41-1.53 (m, 1.3H), 1.59-1.71 (m, 1.3H), 1.75-2.14 (m, 4.7H), 2.18 (s, 3H), 3.40-3.53 (m, 0.7H), 3.72-3.81 (m, 0.3H), 4.00-4.17 (m, 2H), 7.22-7.33 (m, 4H), 7.43-7.49 (m, 1H), 7.61- 7.69 (m, 2H), 8.20 (br. s., 1H), 10.83 (br. s., 1H). 1.32 min, 466, Method 1 |
| | 3-(4′-fluoro-4-methylbiphenyl-3-yl)-4- | | |
| | hydroxy-8-(2,2,2-trifluoroethoxy)-1- | | |
| | azaspiro[4.5]dec-3-en-2-one | | |
| 1-33 | 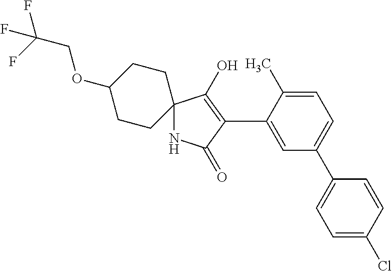 [SMILES: Cc1ccc(-c2ccc(Cl)cc2)cc1C1=C(O)C2(CCC(OCC(F)(F)F)CC2)NC1=O] | WO 09/039975 T1 | (300 MHz, DMSO-d₆): 1.14-1.24 (m, 0.7H), 1.40-1.53 (m, 1.3H), 1.55-1.73 (m, 1.3H), 1.79-2.14 (m, 4.7H), 2.18 (s, 3H), 3.41-3.54 (m, 0.7H), 3.73-3.80 (m, 0.3H), 4.00-4.17 (m, 2H), 7.27-7.36 (m, 2H), 7.45-7.58 (m, 3H), 7.61- 7.68 (m, 2H), 8.19 (br. s., 1H), 10.87 (br. s., 1H). 1.37 min, 480, Method 1 |
| | 3-(4′-chloro-4-methylbiphenyl-3-yl)-4- | | |
| | hydroxy-8-(2,2,2-trifluoroethoxy)-1- | | |
| | azaspiro[4.5]dec-3-en-2-one | | |
| 1-34 | 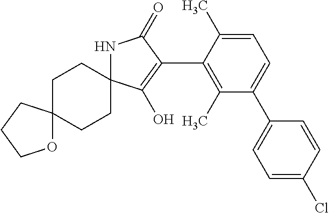 [SMILES: Cc1ccc(-c2ccc(Cl)cc2)c(C)c1C1=C(O)C2(CCC3(CCCO3)CC2)NC1=O] | WO 09/015801 I-1-a-1 | (300 MHz, DMSO-d₆): 1.19-1.31 (m, 2H), 1.56-1.67 (m, 4H), 1.70- 1.80 (m, 2H), 1.80-1.92 (m, 2H), 1.98 (s, 3H), 2.09-2.24 (m, 5H), 3.72 (t, 2H), 7.04 (d, 1H), 7.12 (d, 1H), 7.27-7.34 (m, 2H), 7.46- 7.52 (m, 2H), 8.13 (s, 1H), 10.70 (s, 1H). 1.29 min, 438, Method 1 |
| | 11-(4′-chloro-2,4-dimethylbiphenyl-3-yl)- | | |
| | 12-hydroxy-1-oxa-9- | | |
| | azadispiro[4.2.4.2]tetradec-11-en-10-one | | |
| 1-35 | 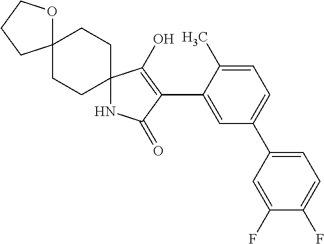 [SMILES: Cc1ccc(-c2ccc(F)c(F)c2)cc1C1=C(O)C2(CCC3(CCCO3)CC2)NC1=O] | WO 09/015801 I-1-a-45 | (300 MHz, DMSO-d₆): 1.19-1.31 (m, 1.6H), 1.39-1.50 (m, 0.4H), 1.55-1.68 (m, 4H), 1.70-1.92 (m, 4H), 2.10-2.24 (m, 5H), 3.67- 3.77 (m, 2H), 7.29 (d, 1H), 7.35 (d, 1H), 7.44-7.53 (m, 3H), 7.64- 7.74 (m, 1H), 8.10 (br. s., 1H), 10.80 (br. s., 1H). 1.23 min, 426, Method 1 |
| | 11-(3′,4′-difluoro-4-methylbiphenyl-3-yl)- | | |
| | 12-hydroxy-1-oxa-9-aza- | | |
| | dispiro[4.2.4.2]tetradec-11-en-10-one | | |
| 1-36 | 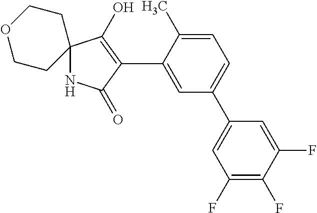 [SMILES: Cc1ccc(-c2cc(F)c(F)c(F)c2)cc1C1=C(O)C2(CCOCC2)NC1=O] | WO 08/067911 I-1-a-28 | |
| | 4-hydroxy-3-(3′,4′,5′-trifluoro-4- | | |
| | methylbiphenyl-3-yl)-8-oxa-1- | | |
| | azaspiro[4.5]dec-3-en-2-one | | |
| 1-37 | 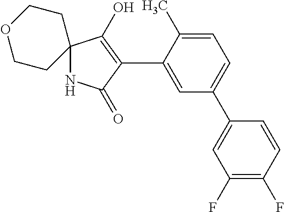 [SMILES: Cc1ccc(-c2ccc(F)c(F)c2)cc1C1=C(O)C2(CCOCC2)NC1=O] | WO 08/067911 I-1-a-3 | |
| | 3-(3′,4′-difluoro-4-methylbiphenyl-3-yl)- | | |
| | 4-hydroxy-8-oxa-1-azaspiro[4.5]dec-3- | | |
| | en-2-one | | |
| 1-38 | 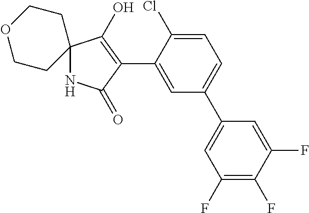 [SMILES: O=C1NC2(CCOCC2)C(O)=C1c1cc(-c2cc(F)c(F)c(F)c2)ccc1Cl] | WO 08/067911 I-1-a-30 | |
| | 3-(4-chloro-3′,4′,5′-trifluorobiphenyl-3- | | |
| | yl)-4-hydroxy-8-oxa-1-azaspiro[4.5]dec- | | |
| | 3-en-2-one | | |
| 1-39 | 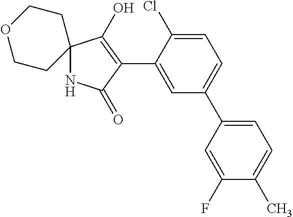 [SMILES: Cc1ccc(-c2ccc(Cl)c(C3=C(O)C4(CCOCC4)NC3=O)c2)cc1F] | WO 08/067911 I-1-a-45 | |
| | 3-(4-chloro-3′-fluoro-4′-methylbiphenyl- | | |
| | 3-yl)-4-hydroxy-8-oxa-1- | | |
| | azaspiro[4.5]dec-3-en-2-one | | |
| 1-40 | 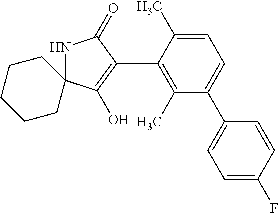 [SMILES: Cc1ccc(-c2ccc(F)cc2)c(C)c1C1=C(O)C2(CCCCC2)NC1=O] | WO 08/067911 I-1-a-53 | (300 MHz, DMSO-d₆): 1.12-1.28 (m, 1H), 1.31-1.44 (m, 2H), 1.55- 1.72 (m, 5H), 1.75-1.92 (m, 2H), 1.97 (s, 3H), 2.12 (s, 3H), 7.03 (d, 1H), 7.10 (d, 1H), 7.20-7.34 (m, 4H), 8.06 (br. s., 1H), 10.65 (br. s., 1H). 1.25 min, 366, Method 1 |
| | 3-(4′-fluoro-2,4-dimethylbiphenyl-3-yl)- | | |
| | 4-hydroxy-1-azaspiro[4.5]dec-3-en-2-one | | |
| 1-41 | 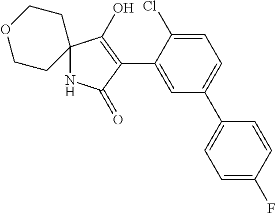 [SMILES: O=C1NC2(CCOCC2)C(O)=C1c1cc(-c2ccc(F)cc2)ccc1Cl] | WO 08/067911 T1 and T2 | |
| | 3-(4-chloro-4′-fluorobiphenyl-3-yl)-4- | | |
| | hydroxy-8-oxa-1-azaspiro[4.5]dec-3-en- | | |
| | 2-one | | |
| 1-42 | 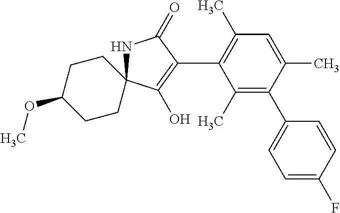 [SMILES: CO[C@H]1CC[C@]2(CC1)NC(=O)C(c1c(C)cc(C)c(-c3ccc(F)cc3)c1C)=C2O] | WO 08/067910 I-1-a-5 | |
| | (5s,8s)-3-(4′-fluoro-2,4,6- | | |
| | trimethylbiphenyl-3-yl)-4-hydroxy-8- | | |
| | methoxy-1-azaspiro[4.5]dec-3-en-2-one | | |
| 1-43 | 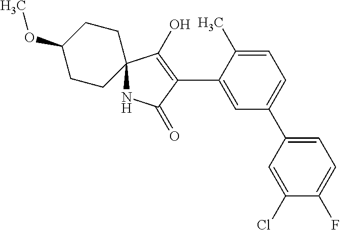 [SMILES: CO[C@H]1CC[C@]2(CC1)NC(=O)C(c1cc(-c3ccc(F)c(Cl)c3)ccc1C)=C2O] | WO 08/067910 I-a-14 | |
| | (5s,8s)-3-(3′-chloro-4′-fluoro-4- | | |
| | methylbiphenyl-3-yl)-4-hydroxy-8- | | |
| | methoxy-1-azaspiro[4.5]dec-3-en-2-one | | |
| 1-44 | 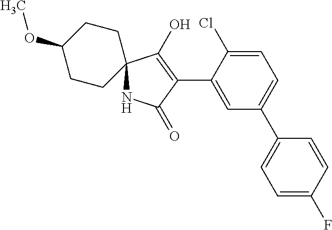 [SMILES: CO[C@H]1CC[C@]2(CC1)NC(=O)C(c1cc(-c3ccc(F)cc3)ccc1Cl)=C2O] | WO 08/067910 I-a-9 | |
| | (5s,8s)-3-(4-chloro-4′-fluorobiphenyl-3- | | |
| | yl)-4-hydroxy-8-methoxy-1- | | |
| | azaspiro[4.5]dec-3-en-2-one | | |
| 1-45 | 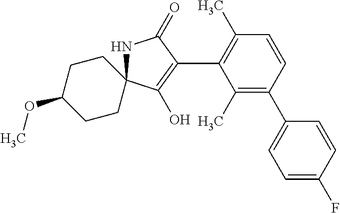 [SMILES: CO[C@H]1CC[C@]2(CC1)NC(=O)C(c1c(C)ccc(-c3ccc(F)cc3)c1C)=C2O] | WO 08/067910 I-a-2 | |
| | (5s,8s)-3-(4′-fluoro-2,4- | | |
| | dimethylbiphenyl-3-yl)-4-hydroxy-8- | | |
| | methoxy-1-azaspiro[4.5]dec-3-en-2-one | | |
| 1-46 | 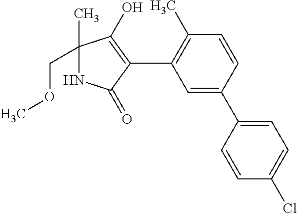 [SMILES: COCC1(C)NC(=O)C(c2cc(-c3ccc(Cl)cc3)ccc2C)=C1O] | WO 07/140881 I-a-1 | |
| | 3-(4′-chloro-4-methylbiphenyl-3-yl)-4- | | |
| | hydroxy-5-(methoxymethyl)-5-methyl- | | |
| | 1,5-dihydro-2H-pyrrol-2-one | | |
| 1-47 | 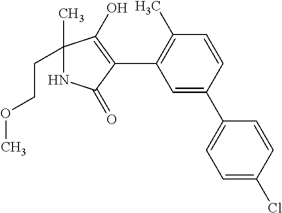 [SMILES: COCCC1(C)NC(=O)C(c2cc(-c3ccc(Cl)cc3)ccc2C)=C1O] | WO 07/140881 I-a-21 | |
| | 3-(4′-chloro-4-methylbiphenyl-3-yl)-4- | | |
| | hydroxy-5-(2-methoxyethyl)-5-methyl- | | |
| | 1,5-dihydro-2H-pyrrol-2-one | | |
| 1-48 | 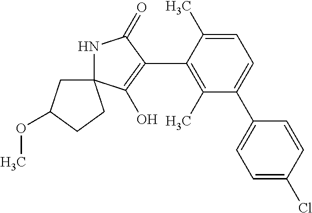 [SMILES: COC1CCC2(C1)NC(=O)C(c1c(C)ccc(-c3ccc(Cl)cc3)c1C)=C2O] | WO 07/073856 T1 | |
| | 3-(4′-chloro-2,4-dimethylbiphenyl-3-yl)- | | |
| | 4-hydroxy-7-methoxy-1- | | |
| | azaspiro[4.4]non-3-en-2-one | | |
| 1-49 | 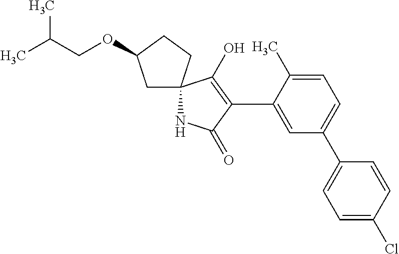 [SMILES: Cc1ccc(-c2ccc(Cl)cc2)cc1C1=C(O)[C@@]2(CC[C@H](OCC(C)C)C2)NC1=O] | WO 07/073856 T5 | |
| | rel-(5R,7R)-3-(4′-chloro-4- | | |
| | methylbiphenyl-3-yl)-4-hydroxy-7-(2- | | |
| | methylpropoxy)-1-azaspiro[4.4]non-3-en- | | |
| | 2-one | | |
| 1-50 | 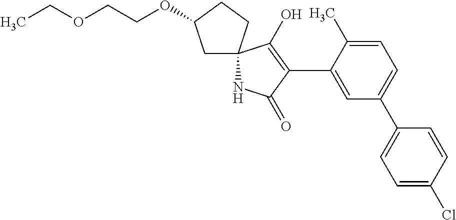 [SMILES: CCOCCO[C@@H]1CC[C@@]2(C1)NC(=O)C(c1cc(-c3ccc(Cl)cc3)ccc1C)=C2O] | WO 07/073856 T9 | |
| | rel-(5R,7S)-3-(4′-chloro-4- | | |
| | methylbiphenyl-3-yl)-7-(2- | | |
| | ethoxyethoxy)-4-hydroxy-1- | | |
| | azaspiro[4.4]non-3-en-2-one | | |
| 1-51 | 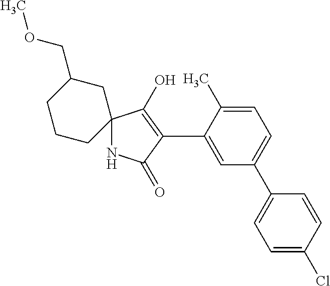 [SMILES: COCC1CCCC2(C1)NC(=O)C(c1cc(-c3ccc(Cl)cc3)ccc1C)=C2O] | WO 07/048545 I-1-a-20 | |
| | 3-(4′-chloro-4-methylbiphenyl-3-yl)-4- | | |
| | hydroxy-7-(methoxymethyl)-1- | | |
| | azaspiro[4.5]dec-3-en-2-one | | |
| 1-52 | 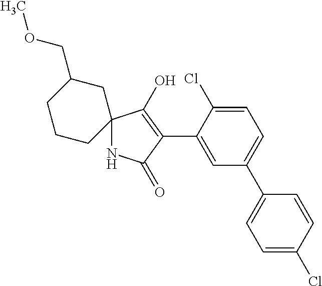 [SMILES: COCC1CCCC2(C1)NC(=O)C(c1cc(-c3ccc(Cl)cc3)ccc1Cl)=C2O] | WO 07/048545 I-1-a-24 | |
| | 3-(4,4′-dichlorobiphenyl-3-yl)-4-hydroxy- | | |
| | 7-(methoxymethyl)-1-azaspiro[4.5]dec-3- | | |
| | en-2-one | | |
| 1-53 | 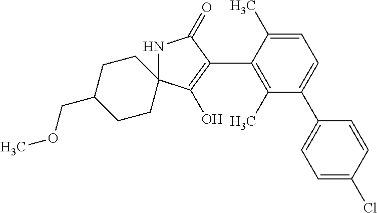 [SMILES: COCC1CCC2(CC1)NC(=O)C(c1c(C)ccc(-c3ccc(Cl)cc3)c1C)=C2O] | WO 07/048545 I-1-a-28 | (300 MHz, DMSO-d₆): 1.21-1.46 (m, 3.7H), 1.48-1.77 (m, 1.3H), 1.64-1.77 (m, 2H), 1.78-1.96 (m, 2H), 1.98 (s, 3H), 2.13 (s, 3H), 3.16 (d, 1.4H), 3.24 (s, 2.1H), 3.26 (s, 0.9H), 3.36 (d, 0.6H), 7.04 (d, 1H), 7.12 (d, 1H), 7.26-7.34 (m, 2H), 7.45-7.52 (m, 2H), 7.98 (s, 0.3H), 8.08 (s, 0.7H), 10.68 (br. s., 1H). 1.27 min, 426, Method 1 |
| | 3-(4′-chloro-2,4-dimethylbiphenyl-3-yl)- | | |
| | 4-hydroxy-8-(methoxymethyl)-1- | | |
| | azaspiro[4.5]dec-3-en-2-one | | |
| 1-54 | 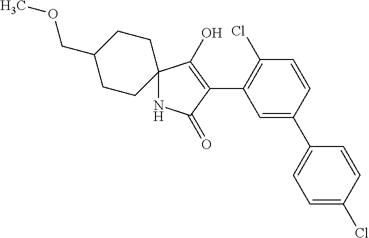 [SMILES: COCC1CCC2(CC1)NC(=O)C(c1cc(-c3ccc(Cl)cc3)ccc1Cl)=C2O] | WO 07/048545 I-1-a-34 | (400 MHz, DMSO-d₆): 1.22-1.46 (m, 3.8H), 1.50-1.59 (m, 1.2H), 1.59-1.75 (m, 2H), 1.75-1.93 (m, 2H), 3.16 (d, 1.6H), 3.24 (s, 2.4H), 3.26 (s, 0.6H), 3.33-3.38 (m, 0.4H), 7.49-7.56 (m, 4H), 7.61 (dd, 1H), 7.67-7.72 (m, 2H), 8.08 (s, 0.2H), 8.18 (s, 0.8H), 11.01 (s, 0.2H), 11.05 (s, 0.8H). 1.25 min, 432, Method 1 |
| | 3-(4,4′-dichlorobiphenyl-3-yl)-4-hydroxy- | | |
| | 8-(methoxymethyl)-1-azaspiro[4.5]dec-3- | | |
| | en-2-one | | |
| 1-55 | 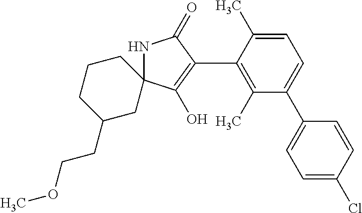 [SMILES: COCCC1CCCC2(C1)NC(=O)C(c1c(C)ccc(-c3ccc(Cl)cc3)c1C)=C2O] | WO 07/048545 I-1-a-42 | |
| | 3-(4′-chloro-2,4-dimethylbiphenyl-3-yl)- | | |
| | 4-hydroxy-7-(2-methoxyethyl)-1- | | |
| | azaspiro[4.5]dec-3-en-2-one | | |
| 1-56 | 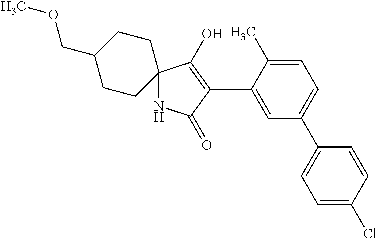 [SMILES: COCC1CCC2(CC1)NC(=O)C(c1cc(-c3ccc(Cl)cc3)ccc1C)=C2O] | WO 07/048545 I-1-a-5 | (300 MHz, DMSO-d₆): 1.22-1.46 (m, 3.8H), 1.48-1.63 (m, 1.2H), 1.65-1.76 (m, 2H), 1.81-1.96 (m, 2H), 2.19 (s, 3H), 3.16 (d, 1.6H), 3.24 (s, 2.4H), 3.27 (s, 0.6H), 3.38 (d, 0.4H), 7.30 (d, 1H), 7.34 (d, 1H), 7.45-7.53 (m, 3H), 7.61- 7.68 (m, 2H), 8.05 (s, 0.2H), 8.13 (s, 0.8H), 10.77 (br. s., 1H). 1.25 min, 412, Method 1 |
| | 3-(4′-chloro-4-methylbiphenyl-3-yl)-4- | | |
| | hydroxy-8-(methoxymethyl)-1- | | |
| | azaspiro[4.5]dec-3-en-2-one | | |
| 1-57 | 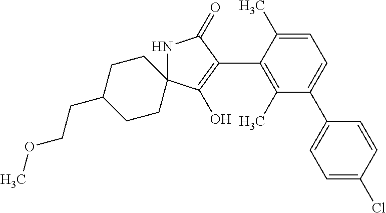 [SMILES: COCCC1CCC2(CC1)NC(=O)C(c1c(C)ccc(-c3ccc(Cl)cc3)c1C)=C2O] | WO 07/048545 I-1-a-53 | (400 MHz, DMSO-d₆): 1.21-1.49 (m, 6H), 1.50-1.72 (m, 3H), 1.74- 1.93 (m, 2H), 1.98 (s, 2.4H), 2.05 (s, 0.6H), 2.12 (s, 2.4H), 2.16 (s, 0.6H), 3.22 (s, 2.4H), 3.23 (s, 0.6H), 3.32-3.39 (m, 2H), 7.04 (d, 1H), 7.12 (d, 1H), 7.27-7.34 (m, 2H), 7.45-7.52 (m, 2H), 7.97 (br. s., 0.2H), 8.09 (br. s., 0.8H), 10.64 (br. s., 0.8H), 10.75 (br. s., 0.2H). 1.31 min, 440, Method 1 |
| | 3-(4′-chloro-2,4-dimethylbiphenyl-3-yl)- | | |
| | 4-hydroxy-8-(2-methoxyethyl)-1- | | |
| | azaspiro[4.5]dec-3-en-2-one | | |
| 1-58 | 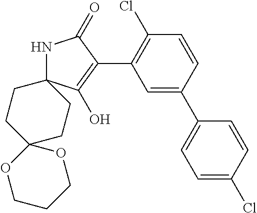 [SMILES: O=C1NC2(CCC3(CC2)OCCCO3)C(O)=C1c1cc(-c2ccc(Cl)cc2)ccc1Cl] | WO 06/089633 I-1-a-15 | |
| | 3-(4,4′-dichlorobiphenyl-3-yl)-4-hydroxy- | | |
| | 9,13-dioxa-1-azadispiro[4.2.5.2]penta- | | |
| | dec-3-en-2-one | | |
| 1-59 | 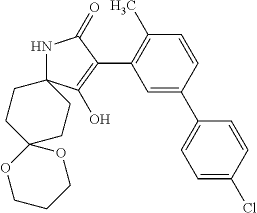 [SMILES: Cc1ccc(-c2ccc(Cl)cc2)cc1C1=C(O)C2(CCC3(CC2)OCCCO3)NC1=O] | WO 06/089633 I-1-a-16 | |
| | 3-(4′-chloro-4-methylbiphenyl-3-yl)-4- | | |
| | hydroxy-9,13-dioxa-1- | | |
| | azadispiro[4.2.5.2]pentadec-3-en-2-one | | |
| 1-60 | 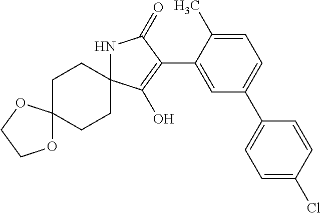 [SMILES: Cc1ccc(-c2ccc(Cl)cc2)cc1C1=C(O)C2(CCC3(CC2)OCCO3)NC1=O] | WO 06/089633 I-1-a-27 | (300 MHz, DMSO-d₆): 1.38-1.49 (m, 2H), 1.65-1.76 (m, 2H), 1.82- 2.03 (m, 3H), 2.06-2.22 (m, 4H), 3.89 (s, 4H), 7.27-7.36 (m, 2H), 7.46-7.54 (m, 3H), 7.61-7.69 (m, 2H), 8.25 (br. s., 1H), 10.90 (br. s., 1H). 1.20, 426, Method 2 |
| | 11-(4′-chloro-4-methylbiphenyl-3-yl)-12- | | |
| | hydroxy-1,4-dioxa-9- | | |
| | azadispiro[4.2.4.2]tetradec-11-en-10-one | | |
| 1-61 | 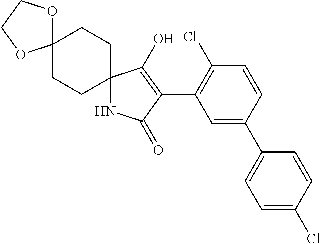 [SMILES: O=C1NC2(CCC3(CC2)OCCO3)C(O)=C1c1cc(-c2ccc(Cl)cc2)ccc1Cl] | WO 06/089633 I-1-a-49 | |
| | 11-(4,4′-dichlorobiphenyl-3-yl)-12- | | |
| | hydroxy-1,4-dioxa-9-aza- | | |
| | dispiro[4.2.4.2]tetradec-11-en-10-one | | |
| 1-62 | 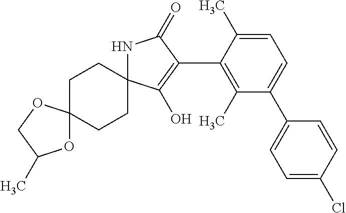 [SMILES: Cc1ccc(-c2ccc(Cl)cc2)c(C)c1C1=C(O)C2(CCC3(CC2)OCC(C)O3)NC1=O] | WO 06/089633 I-1-a-51 | |
| | 11-(4′-chloro-2,4-dimethylbiphenyl-3-yl)- | | |
| | 12-hydroxy-2-methyl-1,4-dioxa-9- | | |
| | azadispiro[4.2.4.2]tetradec-11-en-10-one | | |
| 1-63 | 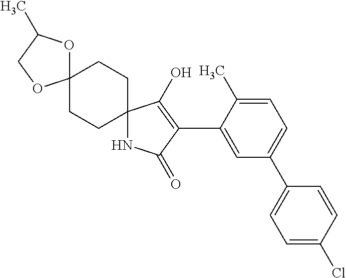 [SMILES: Cc1ccc(-c2ccc(Cl)cc2)cc1C1=C(O)C2(CCC3(CC2)OCC(C)O3)NC1=O] | WO 06/089633 I-1-a-52 | |
| | 11-(4′-chloro-4-methylbiphenyl-3-yl)-12- | | |
| | hydroxy-2-methyl-1,4-dioxa-9- | | |
| | azadispiro[4.2.4.2]tetradec-11-en-10-one | | |
| 1-64 | 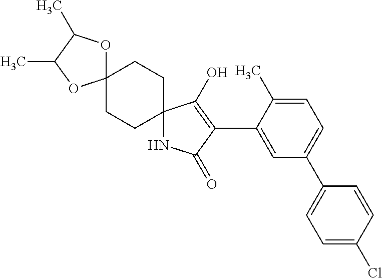 [SMILES: Cc1ccc(-c2ccc(Cl)cc2)cc1C1=C(O)C2(CCC3(CC2)OC(C)C(C)O3)NC1=O] | WO 06/089633 I-1-a-54 | |
| | 11-(4′-chloro-4-methylbiphenyl-3-yl)-12- | | |
| | hydroxy-2,3-dimethyl-1,4-dioxa-9- | | |
| | azadispiro[4.2.4.2]tetradec-11-en-10-one | | |
| 1-65 | 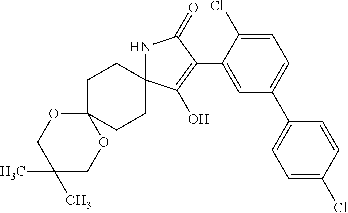 [SMILES: CC1(C)COC2(CCC3(CC2)NC(=O)C(c2cc(-c4ccc(Cl)cc4)ccc2Cl)=C3O)OC1] | WO 06/089633 I-1-a-64 | |
| | 3-(4,4′-dichlorobiphenyl-3-yl)-4-hydroxy- | | |
| | 11,11-dimethyl-9,13-dioxa-1- | | |
| | azadispiro[4.2.5.2]pentadec-3-en-2-one | | |
| 1-66 | 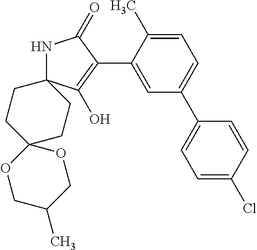 [SMILES: Cc1ccc(-c2ccc(Cl)cc2)cc1C1=C(O)C2(CCC3(CC2)OCC(C)CO3)NC1=O] | WO 06/089633 I-1-a-65 | |
| | 3-(4′-chloro-4-methylbiphenyl-3-yl)-4- | | |
| | hydroxy-11-methyl-9,13-dioxa-1- | | |
| | azadispiro[4.2.5.2]pentadec-3-en-2-one | | |
| 1-67 | 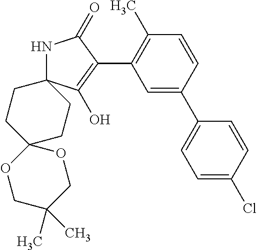 [SMILES: Cc1ccc(-c2ccc(Cl)cc2)cc1C1=C(O)C2(CCC3(CC2)OCC(C)(C)CO3)NC1=O] | WO 06/089633 I-1-a-67 | |
| | 3-(4′-chloro-4-methylbiphenyl-3-yl)-4- | | |
| | hydroxy-11,11-dimethyl-9,13-dioxa-1- | | |
| | azadispiro[4.2.5.2]pentadec-3-en-2-one | | |
| 1-68 | 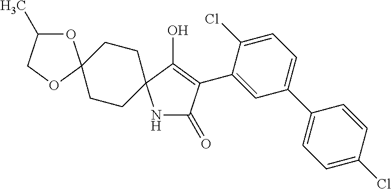 [SMILES: CC1COC2(CCC3(CC2)NC(=O)C(c2cc(-c4ccc(Cl)cc4)ccc2Cl)=C3O)O1] | WO 06/089633 I-1-a-76 | |
| | 11-(4,4′-dichlorobiphenyl-3-yl)-12- | | |
| | hydroxy-2-methyl-1,4-dioxa-9- | | |
| | azadispiro[4.2.4.2]tetradec-11-en-10-one | | |
| 1-69 | 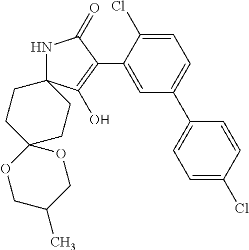 [SMILES: CC1COC2(CCC3(CC2)NC(=O)C(c2cc(-c4ccc(Cl)cc4)ccc2Cl)=C3O)OC1] | WO 06/089633 I-1-a-79 | |
| | 3-(4,4′-dichlorobiphenyl-3-yl)-4-hydroxy- | | |
| | 11-methyl-9,13-dioxa-1- | | |
| | azadispiro[4.2.5.2]pentadec-3-en-2-one | | |
| 1-70 | 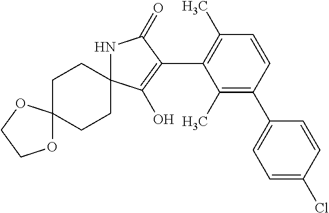 [SMILES: Cc1ccc(-c2ccc(Cl)cc2)c(C)c1C1=C(O)C2(CCC3(CC2)OCCO3)NC1=O] | WO 06/089633 T1 | |
| | 11-(4′-chloro-2,4-dimethylbiphenyl-3-yl)- | | |
| | 12-hydroxy-1,4-dioxa-9- | | |
| | azadispiro[4.2.4.2]tetradec-11-en-10-one | | |
| 1-71 | 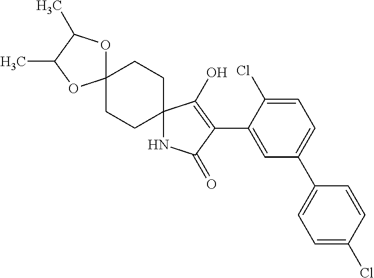 [SMILES: CC1OC2(CCC3(CC2)NC(=O)C(c2cc(-c4ccc(Cl)cc4)ccc2Cl)=C3O)OC1C] | WO 06/089633 T3 | |
| | 11-(4,4′-dichlorobiphenyl-3-yl)-12- | | |
| | hydroxy-2,3-dimethyl-1,4-dioxa-9- | | |
| | azadispiro[4.2.4.2]tetradec-11-en-10-one | | |
| 1-72 | 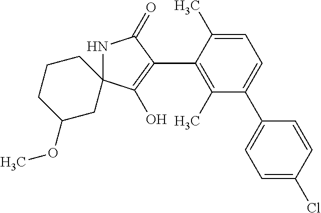 [SMILES: COC1CCCC2(C1)NC(=O)C(c1c(C)ccc(-c3ccc(Cl)cc3)c1C)=C2O] | WO 06/000355 T1 | |
| | 3-(4′-chloro-2,4-dimethylbiphenyl-3-yl)- | | |
| | 4-hydroxy-7-methoxy-1- | | |
| | azaspiro[4.5]dec-3-en-2-one | | |
| 1-73 | 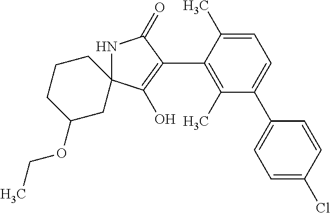 [SMILES: CCOC1CCCC2(C1)NC(=O)C(c1c(C)ccc(-c3ccc(Cl)cc3)c1C)=C2O] | WO 06/000355 T2 | |
| | 3-(4′-chloro-2,4-dimethylbiphenyl-3-yl)- | | |
| | 7-ethoxy-4-hydroxy-1-azaspiro[4.5]dec- | | |
| | 3-en-2-one | | |
| 1-74 | 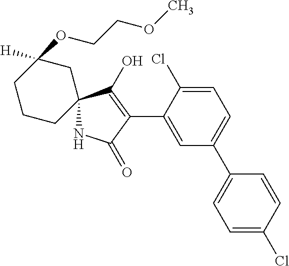 [SMILES: COCCO[C@@H]1CCC[C@]2(C1)NC(=O)C(c1cc(-c3ccc(Cl)cc3)ccc1Cl)=C2O] | WO 06/000355 T8 | |
| | rel-(5R,7R)-3-(4,4′-dichlorobiphenyl-3- | | |
| | yl)-4-hydroxy-7-(2-methoxyethoxy)-1- | | |
| | azaspiro[4.5]dec-3-en-2-one | | |
| 1-75 | 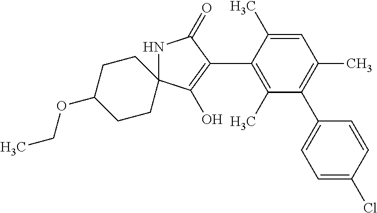 [SMILES: CCOC1CCC2(CC1)NC(=O)C(c1c(C)cc(C)c(-c3ccc(Cl)cc3)c1C)=C2O] | WO 03/059065 I-1-a-16 | |
| | 3-(4′-chloro-2,4,6-trimethylbiphenyl-3- | | |
| | yl)-8-ethoxy-4-hydroxy-1- | | |
| | azaspiro[4.5]dec-3-en-2-one | | |
| 1-76 | 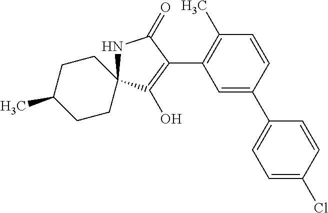 [SMILES: Cc1ccc(-c2ccc(Cl)cc2)cc1C1=C(O)[C@]2(CC[C@H](C)CC2)NC1=O] | WO 03/059065 I-1-a-17 | (300 MHz, DMSO-d₆): 0.91 (d, 3H), 1.29-1.44 (m, 5H), 1.55- 1.68 (m, 2H), 1.82-1.98 (m, 2H), 2.19 (s, 3H), 7.30 (d, 1H), 7.34 (d, 1H), 7.45-7.53 (m, 3H), 7.61- 7.69 (m, 2H), 8.13 (s, 1H), 10.75 (br. s., 1H). 1.34 min, 382, Method 1 |
| | 3-(4′-chloro-4-methylbiphenyl-3-yl)-4- | | |
| | hydroxy-8-methyl-1-azaspiro[4.5]dec-3- | | |
| | en-2-one | | |
| 1-77 | 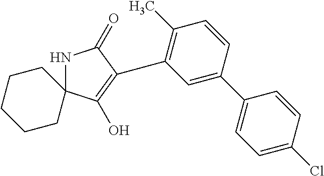 [SMILES: Cc1ccc(-c2ccc(Cl)cc2)cc1C1=C(O)C2(CCCCC2)NC1=O] | WO 03/059065 I-1-a18 | (300 MHz, DMSO-d₆): 1.10-1.29 (m, 1H), 1.33-1.43 (m, 2H), 1.52- 1.73 (m, 5H), 1.78-1.92 (m, 2H), 2.19 (s, 3H), 7.29 (d, 1H), 7.35 (d, 1H), 7.45-7.52 (m, 3H), 7.62- 7.68 (m, 2H), 8.10 (br. s., 1H), 10.82 (br. s, 1H). 1.32 min, 368, Method 1 |
| | 3-(4′-chloro-4-methylbiphenyl-3-yl)-4- | | |
| | hydroxy-1-azaspiro[4.5]dec-3-en-2-one | | |
| 1-78 | 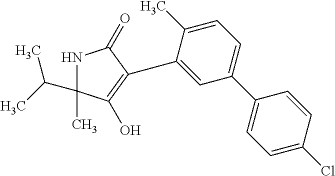 [SMILES: Cc1ccc(-c2ccc(Cl)cc2)cc1C1=C(O)C(C)(C(C)C)NC1=O] | WO 03/059065 I-1-a-19 | |
| | 3-(4′-chloro-4-methylbiphenyl-3-yl)-4- | | |
| | hydroxy-5-methyl-5-(propan-2-yl)-1,5- | | |
| | dihydro-2H-pyrrol-2-one | | |
| 1-79 | 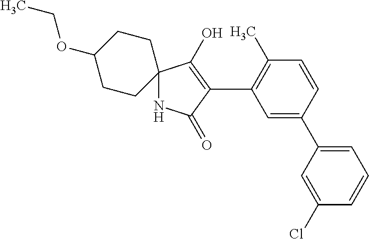 [SMILES: CCOC1CCC2(CC1)NC(=O)C(c1cc(-c3cccc(Cl)c3)ccc1C)=C2O] | WO 03/059065 I-1-a-22 | |
| | 3-(3′-chloro-4-methylbiphenyl-3-yl)-8- | | |
| | ethoxy-4-hydroxy-1-azaspiro[4.5]dec-3- | | |
| | en-2-one | | |
| 1-80 | 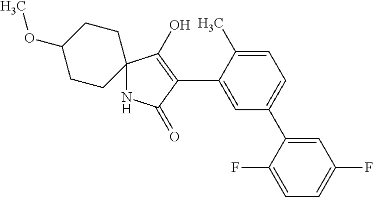 [SMILES: COC1CCC2(CC1)NC(=O)C(c1cc(-c3cc(F)ccc3F)ccc1C)=C2O] | WO 03/059065 I-1-a-29 | |
| | 3-(2′,5′-difluoro-4-methylbiphenyl-3-yl)- | | |
| | 4-hydroxy-8-methoxy-1- | | |
| | azaspiro[4.5]dec-3-en-2-one | | |
| 1-81 | 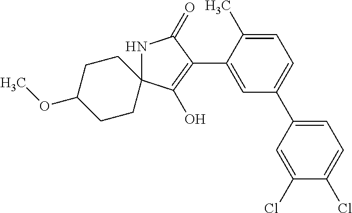 [SMILES: COC1CCC2(CC1)NC(=O)C(c1cc(-c3ccc(Cl)c(Cl)c3)ccc1C)=C2O] | WO 03/059065 I-1-a-31 | |
| | 3-(3′,4′-dichloro-4-methylbiphenyl-3-yl)- | | |
| | 4-hydroxy-8-methoxy-1- | | |
| | azaspiro[4.5]dec-3-en-2-one | | |
| 1-82 | 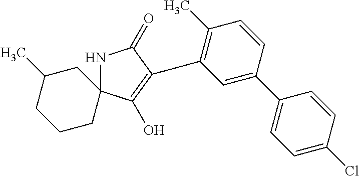 [SMILES: Cc1ccc(-c2ccc(Cl)cc2)cc1C1=C(O)C2(CCCC(C)C2)NC1=O] | WO 03/059065 I-1-a-33 | |
| | 3-(4′-chloro-4-methylbiphenyl-3-yl)-4- | | |
| | hydroxy-7-methyl-1-azaspiro[4.5]dec-3- | | |
| | en-2-one | | |
| 1-83 | 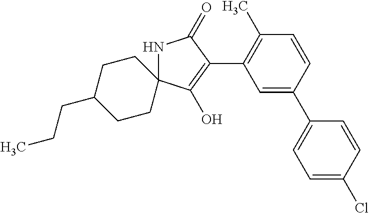 [SMILES: CCCC1CCC2(CC1)NC(=O)C(c1cc(-c3ccc(Cl)cc3)ccc1C)=C2O] | WO 03/059065 I-1-a-35 | |
| | 3-(4′-chloro-4-methylbiphenyl-3-yl)-4- | | |
| | hydroxy-8-propyl-1-azaspiro[4.5]dec-3- | | |
| | en-2-one | | |
| 1-84 | 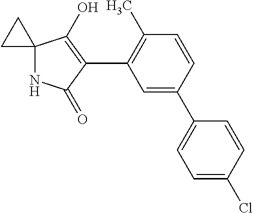 [SMILES: Cc1ccc(-c2ccc(Cl)cc2)cc1C1=C(O)C2(CC2)NC1=O] | WO 03/059065 I-1-a-36 | |
| | 6-(4′-chloro-4-methylbiphenyl-3-yl)-7- | | |
| | hydroxy-4-azaspiro[2.4]hept-6-en-5-one | | |
| 1-85 | 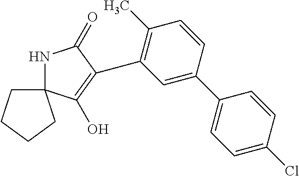 [SMILES: Cc1ccc(-c2ccc(Cl)cc2)cc1C1=C(O)C2(CCCC2)NC1=O] | WO 03/059065 I-1-a-37 | (300 MHz, DMSO-d₆): 1.54-1.90 (m, 6H), 1.99-2.13 (m, 2H), 2.19 (s, 3H), 7.31 (d, 1H), 7.35 (d, 1H), 7.45-7.52 (m, 3H), 7.62-7.69 (m, 2H), 7.92 (s, 1H), 10.82 (br. s., 1H). 1.25 min, 354, Method 1 |
| | 3-(4′-chloro-4-methylbiphenyl-3-yl)-4- | | |
| | hydroxy-1-azaspiro[4.4]non-3-en-2-one | | |
| 1-86 | 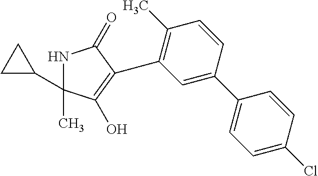 [SMILES: Cc1ccc(-c2ccc(Cl)cc2)cc1C1=C(O)C(C)(C2CC2)NC1=O] | WO 03/059065 I-1-a-39 | |
| | 3-(4′-chloro-4-methylbiphenyl-3-yl)-5- | | |
| | cyclopropyl-4-hydroxy-5-methyl-1,5- | | |
| | dihydro-2H-pyrrol-2-one | | |
| 1-87 | 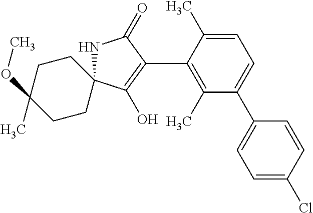 [SMILES: CO[C@]1(C)CC[C@]2(CC1)NC(=O)C(c1c(C)ccc(-c3ccc(Cl)cc3)c1C)=C2O] | PCT/EP/2009 008260 I-a-10 | (300 MHz, DMSO-d₆): 1.08 (s, 3H), 1.10-1.21 (m, 2H), 1.57- 1.81 (m, 4H), 1.98 (s, 3H), 2.01- 2.11 (m, 2H), 2.12 (s, 3H), 3.08 (s, 3H), 7.04 (d, 1H), 7.12 (d, 1H), 7.27-7.33 (m, 2H), 7.46-7.52 (m, 2H), 8.14 (s, 1H), 10.72 (s, 1H). 1.27 min, 426, Method 1 |
| | (5r,8r)-3-(4′-chloro-2,4- | | |
| | dimethylbiphenyl-3-yl)-4-hydroxy-8- | | |
| | methoxy-8-methyl-1-azaspiro[4.5]dec-3- | | |
| | en-2-one | | |
| 1-88 | 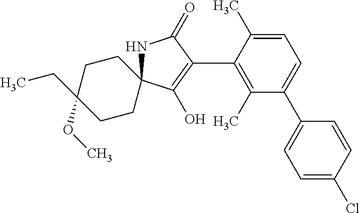 [SMILES: CC[C@]1(OC)CC[C@@]2(CC1)NC(=O)C(c1c(C)ccc(-c3ccc(Cl)cc3)c1C)=C2O] | PCT/EP/2009 008260 I-a-34 | |
| | (5r,8r)-3-(4′-chloro-2,4- | | |
| | dimethylbiphenyl-3-yl)-8-ethyl-4- | | |
| | hydroxy-8-methoxy-1-azaspiro[4.5]dec- | | |
| | 3-en-2-one | | |
| 1-89 | 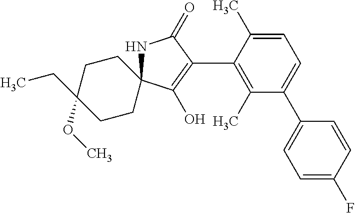 [SMILES: CC[C@]1(OC)CC[C@@]2(CC1)NC(=O)C(c1c(C)ccc(-c3ccc(F)cc3)c1C)=C2O] | PCT/EP/2009 008260 T3 | |
| | (5r,8r)-8-ethyl-3-(4′-fluoro-2,4- | | |
| | dimethylbiphenyl-3-yl)-4-hydroxy-8- | | |
| | methoxy-1-azaspiro[4.5]dec-3-en-2-one | | |

### Starting Materials and Intermediates for Compounds of the Formula (I-1)

### Example 1A

### (4′-Chloro-4-methylbiphenyl-3-yl)acetyl chloride

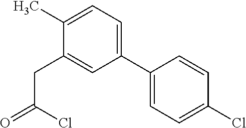 [SMILES: Cc1ccc(-c2ccc(Cl)cc2)cc1CC(=O)Cl]

5.00 g (19.18 mmol) of (4′-chloro-4-methylbiphenyl-3-yl)acetic acid (EP 2029531 A1 and US 2009/298828 A1) were dissolved in 36.51 g (306.84 mmol) of thionyl chloride. The reaction mixture was stirred at 80° C. for four hours and then concentrated under reduced pressure. Drying under fine vacuum gave 5.4 g (100% of theory) of the title compound as a brownish oil.

¹H-NMR (300 MHz, CDCl₃): δ [ppm]=2.36 (s, 3H), 4.22 (s, 2H), 7.29 (d, 1H), 7.35-7.55 (m, 6H).

### Example 2A

### Methyl cis-1-{[(4′-chloro-4-methylbiphenyl-3-yl)acetyl]amino}-4-(trifluoromethyl)cyclohexane-carboxylate

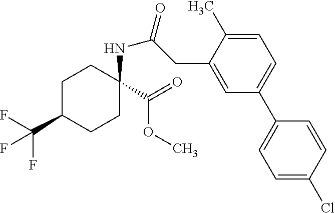 [SMILES: COC(=O)[C@]1(NC(=O)Cc2cc(-c3ccc(Cl)cc3)ccc2C)CC[C@@H](C(F)(F)F)CC1]

At room temperature, 5.00 g (19.09 mmol) of methyl cis-1-amino-4-(trifluoromethyl)cyclohexane-carboxylate hydrochloride (EP 1220841 A2 and WO 2001/23354 A3), 4.83 g (47.73 mmol) of triethylamine and 117 mg (0.955 mmol) of N,N-dimethylaminopyridine were dissolved in 40 ml of dichloromethane. A solution of 5.33 g (19.09 mmol) of (4′-chloro-4-methylbiphenyl-3-yl)acetyl chloride (Example 1A) in 40 ml of dichloromethane was then added dropwise to the mixture. The resulting reaction mixture was stirred at room temperature overnight. For work-up, the mixture was diluted with dichloromethane and the organic phase was washed with aqueous 5% strength citric acid. The mixture was dried over sodium sulphate and the solvent was evaporated, and the residue was purified by chromatography on silica gel (mobile phase: hexane/ethyl acetate gradient). Evaporation and drying gave 6.36 g (71% of theory) of the title compound.

¹H-NMR (300 MHz, DMSO-d₆): δ [ppm]=1.35-1.80 (m, 6H), 2.05-2.18 (m, 2H), 2.24 (s, 3H), 2.25-2.40 (m, 1H), 3.49 (s, 3H), 3.56 (s, 2H), 7.19 (d, 1H), 7.40 (dd, 1H), 7.42-7.52 (m, 3H), 7.56-7.65 (m, 2H), 8.34 (s, 1H).

LC-MS (Method 3): Rₜ=1.50 min; MS (ESIpos): m/z=468 [M+H]⁺.

### Example 3A

### (4,4′-Dichlorobiphenyl-3-yl)acetyl chloride

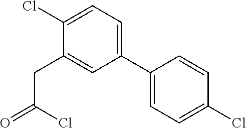 [SMILES: O=C(Cl)Cc1cc(-c2ccc(Cl)cc2)ccc1Cl]

40.40 g (143.70 mmol) of (4,4′-dichlorobiphenyl-3-yl)acetic acid (EP 1943218 A2 and US 2009/215624 A1) were dissolved in 350 g (2946 mmol) of thionyl chloride. The reaction mixture was stirred at 80° C. for six hours and then concentrated under reduced pressure. Drying under fine vacuum gave 43.10 g (100% of theory) of the title compound as a brownish oil.

¹H-NMR (300 MHz, CDCl₃): δ [ppm]=4.35 (s, 2H), 7.37-7.60 (m, 7H).

### Example 4A

### Methyl cis-1-{[(4,4′-dichlorobiphenyl-3-yl)acetyl]amino}-4-methoxycyclohexanecarboxylate

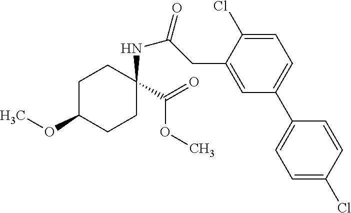 [SMILES: COC(=O)[C@]1(NC(=O)Cc2cc(-c3ccc(Cl)cc3)ccc2Cl)CC[C@@H](OC)CC1]

At room temperature, 35.24 g (157.52 mmol) of methyl cis-1-amino-4-methoxycyclohexane-carboxylate hydrochloride (EP 1791816 A1 and WO 2006/29799 A1) and 31.88 g (315.00 mmol) of triethylamine were dissolved in 350 ml of dichloromethane. With ice-cooling, a solution of 42.90 g (143.2 mmol) of (4,4′-dichlorobiphenyl-3-yl)acetyl chloride (Example 3A) in 350 ml of dichloromethane was then added dropwise to the mixture. The resulting reaction mixture was stirred at room temperature for three days. For work-up, the mixture was diluted with dichloromethane and the organic phase was washed with aqueous saturated sodium bicarbonate solution, aqueous 5% strength citric acid and water. After drying over sodium sulphate, the solvent was evaporated and the residue was triturated with diethyl ether. The product was filtered off and dried, giving 54.56 g (85% of theory) of the title compound.

¹H-NMR (400 MHz, DMSO-d₆): δ [ppm]=1.31-1.47 (m, 2H), 1.56-1.70 (m, 2H), 1.71-1.84 (m, 2H), 1.97-2.10 (m, 2H), 3.07-3.17 (m, 1H), 3.19 (s, 3H), 3.48 (s, 3H), 3.68 (s, 2H), 7.44-7.56 (m, 4H), 7.62-7.69 (m, 3H), 8.35 (s, 1H).

LC-MS (Method 2): Rₜ=1.34 min; MS (ESIpos): m/z=450 [M+H]⁺.

### Example 5A

### (3-Bromo-2,6-dimethylphenyl)acetyl chloride

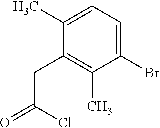

6.00 g (24.68 mmol) of (3-bromo-2,6-dimethylphenyl)acetic acid (WO 97/36868) were dissolved in 29.36 g (246.81 mmol) of thionyl chloride. The reaction mixture was stirred at 80° C. for four hours and then concentrated under reduced pressure. Drying under fine vacuum gave 6.36 g (99% of theory) of the title compound as a brownish oil.

¹H-NMR (400 MHz, CDCl₃): δ [ppm]=2.29 (s, 3H), 2.41 (s, 3H), 4.28 (s, 2H), 6.94 (d, 1H), 7.45 (d, 1H).

### Example 6A

### Methyl cis-1-{[(3-bromo-2,6-dimethylphenyl)acetyl]amino}-4-methoxycyclohexanecarboxylate

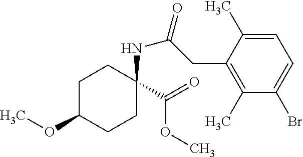 [SMILES: COC(=O)[C@]1(NC(=O)Cc2c(C)ccc(Br)c2C)CC[C@@H](OC)CC1]

At room temperature, 1.87 g (8.34 mmol) of methyl cis-1-amino-4-methoxycyclohexane-carboxylate hydrochloride (EP 1791816 A1 and WO 2006/29799 A1), 2.11 g (20.84 mmol) of triethylamine and 0.051 g (0.417 mmol) of N,N-dimethylaminopyridine were dissolved in 22 ml of dichloromethane. With ice-cooling, a solution of 2.18 g (8.34 mmol) of (3-bromo-2,6-dimethylphenyl)acetyl chloride (Example 5A) in 22 ml of dichloromethane was then added dropwise to the mixture. The resulting reaction mixture was stirred at room temperature overnight. For work-up, the mixture was diluted with dichloromethane and the organic phase was washed with aqueous saturated sodium bicarbonate solution, aqueous 5% strength citric acid and water. After drying over sodium sulphate, the solvent was evaporated and the residue was purified by chromatography on silica gel (mobile phase: ethyl acetate). Evaporation and drying gave 3.02 g (88% of theory) of the title compound.

¹H-NMR (400 MHz, DMSO-d₆): δ [ppm]=1.28-1.46 (m, 2H), 1.54-1.70 (m, 2H), 1.71-1.86 (m, 2H), 1.94-2.07 (m, 2H), 2.17 (s, 3H), 2.29 (s, 3H), 3.06-3.18 (m, 1H), 3.20 (s, 3H), 3.48 (s, 3H), 3.63 (s, 2H), 6.90 (d, 1H), 7.31 (d, 1H), 8.26 (s, 1H).

LC-MS (Method 2): Rₜ=1.23 min; MS (ESIpos): m/z=414 [M+H]⁺.

### Example 7A

### (5s,8s)-3-(3 Bromo-2,6-dimethylphenyl)-4-hydroxy-8-methoxy-1-azaspiro[4.5]dec-3-en-2-one

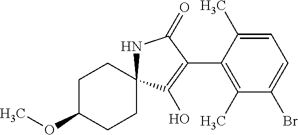 [SMILES: CO[C@H]1CC[C@]2(CC1)NC(=O)C(c1c(C)ccc(Br)c1C)=C2O]

1.63 g (14.55 mmol) of potassium tert-butoxide were added to 3.00 g (7.28 mmol) of methyl cis-1-{[(3-bromo-2,6-dimethylphenyl)acetyl]amino}-4-methoxycyclohexanecarboxylate (Example 6A) in 30 ml of N,N-dimethylformamide. The reaction mixture was stirred at 80° C. for 30 minutes. For work-up, the cold reaction mixture was poured onto 150 ml of ice-water and acidified with aqueous hydrochloric acid. The crude product was filtered off. Drying gave 2.61 g (94% of theory) of the title compound.

¹H-NMR (300 MHz, DMSO-d₆): δ [ppm]=1.33-1.58 (m, 4H), 1.79-1.99 (m, 4H), 2.00 (s, 3H), 2.12 (s, 3H), 3.03-3.17 (m, 1H), 3.23 (s, 3H), 6.96 (d, 1H), 7.39 (d, 1H), 8.15 (s, 1H), 10.81 (s, 1H).

LC-MS (Method 2): Rₜ=1.01 min; MS (ESIpos): m/z=382 [M+H]⁺.

### Example 8A

### (5-Bromo-2-methylphenyl)acetyl chloride

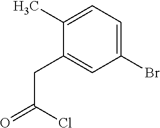

6.00 g (26.19 mmol) of (5-bromo-2-methylphenyl)acetic acid (EP 1791816 A1 and WO 2006/29799 A1) were dissolved in 31.20 g (261.92 mmol) of thionyl chloride. The reaction mixture was stirred at 80° C. for two hours and then concentrated under reduced pressure. Drying under fine vacuum gave 6.29 g (97% of theory) of the title compound as a brownish oil.

¹H-NMR (300 MHz, CDCl₃): δ [ppm]=2.26 (s, 3H), 4.12 (s, 2H), 7.09 (d, 1H), 7.34 (d, 1H), 7.37 (dd, 1H).

### Example 9A

### Methyl 8-{[(5-bromo-2-methylphenyl)acetyl]amino}-1,4-dioxaspiro[4.5]decane-8-carboxylate

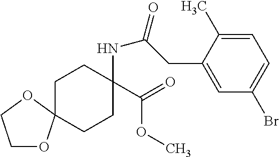 [SMILES: COC(=O)C1(NC(=O)Cc2cc(Br)ccc2C)CCC2(CC1)OCCO2]

At room temperature, 5.47 g (25.41 mmol) of methyl 8-amino-1,4-dioxaspiro[4.5]decane-8-carboxylate [T. Satoh et al., Tetrahedron 63 (2007), 4806-4813], 3.86 g (38.12 mmol) of triethylamine and 155 mg (1.27 mmol) of N,N-dimethylaminopyridine were dissolved in 45 ml of dichloromethane. A solution of 6.29 g (25.41 mmol) of (5-bromo-2-methylphenyl)acetyl chloride (Example 8A) in 45 ml of dichloromethane was then added dropwise to the mixture. The resulting reaction mixture was stirred at room temperature overnight. For work-up, the mixture was diluted with dichloromethane and the organic phase was washed with aqueous saturated sodium bicarbonate solution. After drying over sodium sulphate, the solvent was evaporated and the residue was purified by chromatography on silica gel (mobile phase: hexane/ethyl acetate gradient/1% triethylamine). Evaporation and drying gave 3.64 g (34% of theory) of the title compound which was used without further characterisation for the next step.

### Example 10A

### 11-(5-Bromo-2-methylphenyl)-12-hydroxy-1,4-dioxa-9-azadispiro[4.2.4.2]tetradec-11-en-10-one

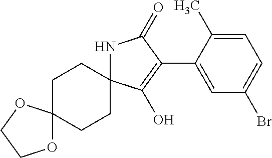 [SMILES: Cc1ccc(Br)cc1C1=C(O)C2(CCC3(CC2)OCCO3)NC1=O]

1.92 g (17.08 mmol) of potassium tert-butoxide were added to 3.64 g (8.54 mmol) of methyl 8-{[(5-bromo-2-methylphenyl)acetyl]amino}-1,4-dioxaspiro[4.5]decane-8-carboxylate (Example 9A) in 43 ml of N,N-dimethylformamide. The reaction mixture was stirred at 80° C. for 30 minutes. For work-up, the cold reaction mixture was poured onto 500 ml of ice-water and acidified with aqueous hydrochloric acid to pH=4. The crude product was filtered off. Drying gave 2.49 g (74% of theory) of the title compound which was used without further characterisation for the next step.

### Example 11A

### 3-(5-Bromo-2-methylphenyl)-4-hydroxy-1-azaspiro[4.5]dec-3-ene-2,8-dione

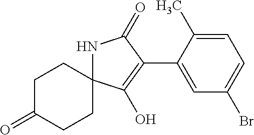 [SMILES: Cc1ccc(Br)cc1C1=C(O)C2(CCC(=O)CC2)NC1=O]

192 mg (1.01 mmol) of 4-toluenesulphonic acid monohydrate were added to 2.49 g (6.32 mmol) of 11-(5-bromo-2-methylphenyl)-12-hydroxy-1,4-dioxa-9-azadispiro[4.2.4.2]tetradec-11-en-10-one (Example 10A) in 26 ml of acetone and 13 ml of water. The reaction mixture was stirred at 80° C. overnight. For work-up, the cold reaction mixture was diluted with water and the acetone was removed on a rotary evaporator. The precipitated crude product was extracted with ethyl acetate. The organic phase was washed with saturated aqueous sodium chloride solution, dried over sodium sulphate and concentrated under reduced pressure. Drying gave 1.97 g (89% of theory) of the title compound.

¹H-NMR (400 MHz, DMSO-d₆): δ [ppm]=1.68-1.78 (m, 2H), 2.09 (s, 3H), 2.21-2.34 (m, 4H), 2.64-2.78 (m, 2H), 7.15 (d, 1H), 7.23 (d, 1H), 7.35 (dd, 1H), 8.53 (s, 1H), 11.12 (s, 1H).

LC-MS (Method 2): Rₜ=0.87 min; MS (ESIpos): m/z=352 [M+H]⁺.

### Example 12A

### (5r,8r)-3-(5-Bromo-2-methylphenyl)-4,8-dihydroxy-8 (trifluoromethyl)-1-azaspiro[4.5]dec-3-en-2-one

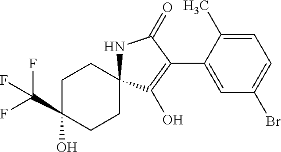 [SMILES: Cc1ccc(Br)cc1C1=C(O)[C@]2(CC[C@](O)(C(F)(F)F)CC2)NC1=O]

521 mg (1.60 mmol) of caesium carbonate and 975 mg (6.85 mmol) of (trifluoromethyl)trimethylsilane were added to 400 mg (1.14 mmol) of 3-(5-bromo-2-methylphenyl)-4-hydroxy-1-azaspiro[4.5]dec-3-ene-2,8-dione (Example 11A) in 8.3 ml of N,N-dimethylformamide. The reaction mixture was stirred at room temperature for three hours. The mixture was then diluted with water, acidified with aqueous citric acid to pH=4.5 and extracted with ethyl acetate. The organic phase was washed with water, dried over sodium sulphate and concentrated under reduced pressure. The residue was dissolved in 5 ml of tetrahydrofuran, 1 ml of 4N aqueous hydrochloric acid was added and the mixture was stirred at room temperature for one hour and then diluted with water. The crude product was extracted with ethyl acetate, and the organic phase was dried over sodium sulphate. After concentration under reduced pressure, the residue was purified by chromatography on silica gel (mobile phase: hexane/ethyl acetate gradient). Evaporation and drying gave 367 mg (76% of theory) of the title compound.

¹H-NMR (400 MHz, methanol-d₄): δ [ppm]=1.39-1.50 (m, 2H), 1.84-1.98 (m, 4H), 2.15 (s, 3H), 2.30-2.43 (m, 2H), 7.15 (d, 1H), 7.27 (d, 1H), 7.34 (dd, 1H).

LC-MS (Method 3): Rₜ=0.96 min; MS (ESIpos): m/z=420 [M+H]⁺.

### Example 13A

### Methyl 8-{[(3-bromo-2,6-dimethylphenyl)acetyl]amino}-1,4-dioxaspiro[4.5]decane-8-carboxylate

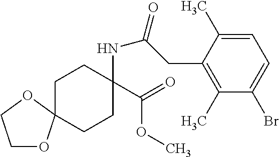 [SMILES: COC(=O)C1(NC(=O)Cc2c(C)ccc(Br)c2C)CCC2(CC1)OCCO2]

At room temperature, 5.23 g (24.32 mmol) of methyl 8-amino-1,4-dioxaspiro[4.5]decane-8-carboxylate [T. Satoh et al., Tetrahedron 63 (2007), 4806-4813], 3.69 g (36.47 mmol) of triethylamine and 150 mg (1.22 mmol) of N,N-dimethylaminopyridine were dissolved in 45 ml of dichloromethane. A solution of 6.36 g (25.32 mmol) of (3-bromo-2,6-dimethylphenyl)acetyl chloride (Example 5A) in 45 ml of dichloromethane was then added dropwise to the mixture. The resulting reaction mixture was stirred at room temperature overnight. For work-up, the mixture was diluted with dichloromethane and the organic phase was washed with aqueous saturated sodium bicarbonate solution. After drying over sodium sulphate, the solvent was evaporated and the residue was purified by chromatography on silica gel (mobile phase: hexane/ethyl acetate gradient/1% triethylamine). Evaporation and drying gave 7.33 g (69% of theory) of the title compound which was used without further characterisation for the next step.

### Example 14A

### 11-(3-Bromo-2,6-dimethylphenyl)-12-hydroxy-1,4-dioxa-9-azadispiro[4.2.4.2]tetradec-11-en-10-one

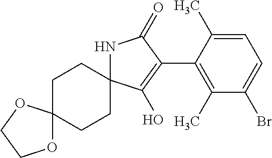 [SMILES: Cc1ccc(Br)c(C)c1C1=C(O)C2(CCC3(CC2)OCCO3)NC1=O]

3.74 g (33.29 mmol) of potassium tert-butoxide were added to 7.33 g (16.65 mmol) of methyl 8-{[(3-bromo-2,6-dimethylphenyl)acetyl]amino}-1,4-dioxaspiro[4.5]decane-8-carboxylate (Example 13A) in 40 ml of N,N-dimethylformamide. The reaction mixture was stirred at 80° C. for 30 minutes. For work-up, the cold reaction mixture was poured onto 500 ml of ice-water and acidified with aqueous hydrochloric acid to pH=4. The crude product was filtered off. Drying gave 5.48 g (81% of theory) of the title compound which was used without further characterisation for the next step.

### Example 15A

### 3-(3-Bromo-2,6-dimethylphenyl)-4-hydroxy-1-azaspiro[4.5]dec-3-ene-2,8-dione

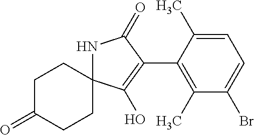 [SMILES: Cc1ccc(Br)c(C)c1C1=C(O)C2(CCC(=O)CC2)NC1=O]

87 mg (0.46 mmol) of 4-toluenesulphonic acid monohydrate were added to 1.16 g (2.84 mmol) of 11-(3-bromo-2,6-dimethylphenyl)-12-hydroxy-1,4-dioxa-9-azadispiro[4.2.4.2]tetradec-11-en-10-one (Example 14A) in 17 ml of acetone and 9 ml of water. The reaction mixture was stirred at 80° C. overnight. For work-up, the cold reaction mixture was diluted with water and the acetone was removed on a rotary evaporator. The precipitated product was filtered off. Drying gave 0.93 g (90% of theory) of the title compound.

¹H-NMR (300 MHz, DMSO-d₆): δ [ppm]=1.65-1.81 (m, 2H), 2.03 (s, 3H), 2.15 (s, 3H), 2.20-2.35 (m, 4H), 2.61-2.81 (m, 2H), 6.98 (d, 1H), 7.41 (d, 1H), 8.46 (s, 1H), 11.05 (s, 1H).

LC-MS (Method 3): Rₜ=0.90 min; MS (ESIpos): m/z=366 [M+H]⁺.

### Example 16A

### (5r,8r)-3-(3 Bromo-2,6-dimethylphenyl)-4,8-dihydroxy-8-(trifluoromethyl)-1-azaspiro[4.5]dec-3-en-2-one

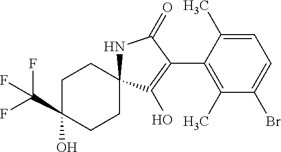 [SMILES: Cc1ccc(Br)c(C)c1C1=C(O)[C@]2(CC[C@](O)(C(F)(F)F)CC2)NC1=O]

500 mg (1.54 mmol) of caesium carbonate and 937 mg (6.59 mmol) of (trifluoromethyl)trimethyl-silane were added to 400 mg (1.10 mmol) of 3-(3-bromo-2,6-dimethylphenyl)-4-hydroxy-1-azaspiro[4.5]dec-3-ene-2,8-dione (Example 15A) in 8 ml of N,N-dimethylformamide. The reaction mixture was stirred at room temperature for three hours. The mixture was then diluted with water, acidified with aqueous citric acid to pH=4.5 and extracted with ethyl acetate. The organic phase was washed with water, dried over sodium sulphate and concentrated under reduced pressure. The residue was dissolved in 10 ml of tetrahydrofuran, 2 ml of 4N aqueous hydrochloric acid were added, and the mixture was stirred at room temperature for one hour and the diluted with water. The crude product was extracted with ethyl acetate, and the organic phase was dried over sodium sulphate. The residue was, after evaporation under reduced pressure, purified by chromatography on silica gel (mobile phase: hexane/ethyl acetate gradient). Evaporation and drying gave 298 mg (62% of theory) of the title compound.

¹H-NMR (300 MHz, methanol-d₄): δ [ppm]=1.40-1.55 (m, 2H), 1.83-2.00 (m, 4H), 2.12 (s, 3H), 2.26 (s, 3H), 2.32-2.47 (m, 2H), 6.99 (d, 1H), 7.42 (d, 1H).

LC-MS (Method 3): Rₜ=1.02 min; MS (ESIpos): m/z=436 [M+H]⁺.

### Example 17A

### (5-Bromo-4-fluoro-2-methylphenyl)acetyl chloride

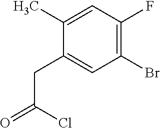

22.00 g (89.00 mmol) of (5-bromo-4-fluoro-2-methylphenyl)acetic acid (WO 2010/52161 A2) were dissolved in 170.00 g (1425.00 mmol) of thionyl chloride. The reaction mixture was stirred at 80° C. for six hours and then concentrated under reduced pressure. Drying under fine vacuum gave 23.47 g (99% of theory) of the title compound as a brownish oil.

¹H-NMR (300 MHz, CDCl₃): δ [ppm]=2.27 (s, 3H), 4.10 (s, 2H), 7.00 (d, 1H), 7.38 (d, 1H).

### Example 18A

### Methyl 8-{[(5-bromo-4-fluoro-2-methylphenyl)acetyl]amino}-1,4-dioxaspiro[4.5]decane-8-carboxylate

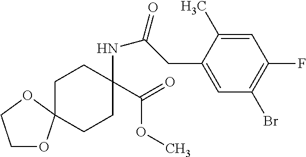 [SMILES: COC(=O)C1(NC(=O)Cc2cc(Br)c(F)cc2C)CCC2(CC1)OCCO2]

At room temperature, 19.03 g (88.39 mmol) of methyl 8-amino-1,4-dioxaspiro[4.5]decane-8-carboxylate, 13.42 g (132.59 mmol) of triethylamine and 540 mg (4.42 mmol) of N,N-dimethylaminopyridine were dissolved in 163 ml of dichloromethane. A solution of 23.47 g (88.39 mmol) of (5-bromo-4-fluoro-2-methylphenyl)acetyl chloride (Example 17A) in 163 ml of dichloromethane was then added dropwise to the mixture. The resulting reaction mixture was stirred at room temperature overnight. For work-up, the mixture was diluted with dichloromethane and the organic phase was washed with aqueous saturated sodium bicarbonate solution. After drying over sodium sulphate, the solvent was evaporated and the residue was purified by crystallization from ethyl acetate/hexane (1:1). Drying gave 25.71 g (65% of theory) of the title compound which was used without further characterisation for the next step.

### Example 19A

### 11-(5-Bromo-4-fluoro-2-methylphenyl)-12-hydroxy-1,4-dioxa-9-azadispiro[4.2.4.2]tetradec-11-en-10-one

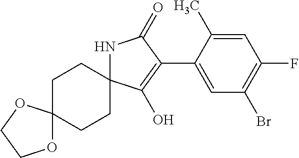 [SMILES: Cc1cc(F)c(Br)cc1C1=C(O)C2(CCC3(CC2)OCCO3)NC1=O]

14.60 g (130.09 mmol) of potassium tert-butoxide were added to 28.90 g (65.05 mmol) of methyl 8-{[(5-bromo-4-fluoro-2-methylphenyl)acetyl]amino}-1,4-dioxaspiro[4.5]decane-8-carboxylate (Example 18A) in 325 ml of N,N-dimethylformamide. The reaction mixture was stirred at 80° C. for 60 minutes. For work-up, the cold reaction mixture was poured onto 3000 ml of ice-water and acidified with aqueous hydrochloric acid to pH=4.5. The crude product was filtered off. Drying gave 24.40 g (91% of theory) of the title compound which was used without further characterisation for the next step.

### Example 20A

### 3-(5-Bromo-4-fluoro-2-methylphenyl)-4-hydroxy-1-azaspiro[4.5]dec-3-ene-2,8-dione

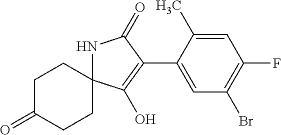 [SMILES: Cc1cc(F)c(Br)cc1C1=C(O)C2(CCC(=O)CC2)NC1=O]

1.80 g (9.47 mmol) of 4-toluenesulphonic acid monohydrate were added to 24.40 g (59.19 mmol) of 11-(5-bromo-4-fluoro-2-methylphenyl)-12-hydroxy-1,4-dioxa-9-azadispiro[4.2.4.2]tetradec-11-en-10-one (Example 19A) in 438 ml of acetone and 219 ml of water. The reaction mixture was stirred at 80° C. for twelve hours. For work-up, the cold reaction mixture was diluted with water and the acetone was removed on a rotary evaporator. The product was filtered off. Drying gave 21.50 g (99% of theory) of the title compound.

¹H-NMR (400 MHz, DMSO-d₆): δ [ppm]=1.65-1.80 (m, 2H), 2.11 (s, 3H), 2.17-2.34 (m, 4H), 2.60-2.80 (m, 2H), 7.24 (d, 1H), 7.33 (d, 1H), 8.53 (s, 1H), 11.13 (s, 1H).

LC-MS (Method 3): Rₜ=0.90 min; MS (ESIpos): m/z=368 [M+H]⁺.

### Example 21A

### (5r,8r)-3-(5-Bromo-4-fluoro-2-methylphenyl)-4,8-dihydroxy-8-(trifluoromethyl)-1-azaspiro[4.5]dec-3-en-2-one

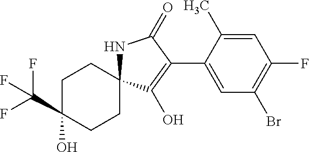 [SMILES: Cc1cc(F)c(Br)cc1C1=C(O)[C@]2(CC[C@](O)(C(F)(F)F)CC2)NC1=O]

496 mg (1.52 mmol) of caesium carbonate and 927 mg (6.52 mmol) of (trifluoromethyl)trimethyl-silane were added to 400 mg (1.09 mmol) of 3-(5-bromo-4-fluoro-2-methylphenyl)-4-hydroxy-1-azaspiro[4.5]dec-3-ene-2,8-dione (Example 20A) in 7.9 ml of N,N-dimethylformamide. The reaction mixture was stirred at room temperature for three hours. The mixture was then diluted with water, acidified with aqueous citric acid to pH=4.5 and extracted with ethyl acetate. The organic phase was washed with water, dried over sodium sulphate and concentrated under reduced pressure. The residue was dissolved in 10 ml of tetrahydrofuran, 2 ml of 4N aqueous hydrochloric acid were added, and the mixture was stirred at room temperature for one hour and then diluted with water. The crude product was extracted with ethyl acetate, and the organic phase was dried over sodium sulphate. The residue was, after evaporation under reduced pressure, purified by chromatography on silica gel (mobile phase: hexane/ethyl acetate gradient). Evaporation and drying gave 382 mg (80% of theory) of the title compound.

¹H-NMR (300 MHz, methanol-d₄): δ [ppm]=1.39-1.51 (m, 2H), 1.84-1.97 (m, 4H), 2.17 (s, 3H), 2.29-2.46 (m, 2H), 7.10 (d, 1H), 7.34 (d, 1H).

LC-MS (Method 3): Rₜ=0.98 min; MS (ESIpos): m/z=438 [M+H]⁺.

### Example 22A

### (3′,4′-Difluoro-4-methylbiphenyl-3-yl)acetic acid

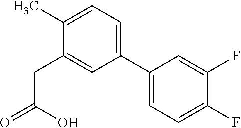 [SMILES: Cc1ccc(-c2ccc(F)c(F)c2)cc1CC(=O)O]

23.75 g (103.68 mmol) of (5-bromo-2-methylphenyl)acetic acid and 16.37 g (103.68 mmol) of (3,4-difluorophenyl)boronic acid were dissolved in 63 ml of aqueous 3.3 N sodium hydroxide solution, and 668 mg (2.07 mmol) of tetra-n-butylammonium bromide and 55 mg of palladium on carbon (10%) were added. The reaction mixture was stirred at 80° C. for five hours and then diluted with water to double the original volume. The mixture was acidified with concentrated aqueous hydrochloric acid and then extracted with ethyl acetate, and the organic phase was dried over sodium sulphate and concentrated under reduced pressure. The crude product was purified by trituration with hexane/methyl tert-butyl ether (9:1). Drying gave 19.77 g (73% of theory) of the title compound.

¹H-NMR (400 MHz, DMSO-d₆): δ [ppm]=2.22 (s, 3H), 3.63 (s, 2H), 7.22 (d, 1H), 7.40-7.53 (m, 4H), 7.64-7.74 (m, 1H).

### Example 23A

### (3′,4′-Difluoro-4-methylbiphenyl-3-yl)acetyl chloride

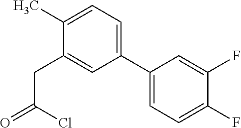 [SMILES: Cc1ccc(-c2ccc(F)c(F)c2)cc1CC(=O)Cl]

9.70 g (36.99 mmol) of (3′,4′-difluoro-4-methylbiphenyl-3-yl)acetic acid (Example 22A) were dissolved in 70.41 g (591.78 mmol) of thionyl chloride. The reaction mixture was stirred at 80° C. for four hours and then concentrated under reduced pressure. Drying under fine vacuum gave 10.28 g (99% of theory) of the title compound as a brownish oil.

¹H-NMR (400 MHz, CDCl₃): δ [ppm]=2.36 (s, 3H), 4.23 (s, 2H), 7.17-7.45 (m, 6H).

### Example 24A

### Methyl cis-1-{[(3′,4′-difluoro-4-methylbiphenyl-3-yl)acetyl]amino}-4-(trifluoromethyl)cyclo-hexanecarboxylate

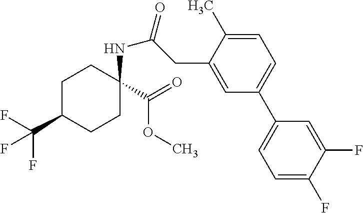 [SMILES: COC(=O)[C@]1(NC(=O)Cc2cc(-c3ccc(F)c(F)c3)ccc2C)CC[C@@H](C(F)(F)F)CC1]

At room temperature, 6.00 g (22.93 mmol) of methyl cis-1-amino-4-(trifluoromethyl)cyclo-hexanecarboxylate hydrochloride (EP 1220841 A2 and WO 2001/23354 A3), 5.80 g (57.32 mmol) of triethylamine and 140 mg (1.15 mmol) of N,N-dimethylaminopyridine were dissolved in 60 ml of dichloromethane. A solution of 6.44 g (22.93 mmol) of (3′,4′-difluoro-4-methylbiphenyl-3-yl)acetyl chloride (Example 23A) in 60 ml of dichloromethane was then added dropwise to the mixture. The resulting reaction mixture was stirred at room temperature overnight. For work-up, the mixture was diluted with dichloromethane and the organic phase was washed with aqueous saturated sodium bicarbonate solution and with aqueous 5% strength citric acid. After drying over sodium sulphate, the solvent was evaporated and the residue purified by chromatography on silica gel (mobile phase: hexane/ethyl acetate gradient). Evaporation and drying gave 6.57 g (61% of theory) of the title compound which was used without further characterisation for the next step.

### Example 25A

### (5r,8r)-3-(5-Bromo-2-methylphenyl)-4,8-dihydroxy-8-(pentafluoroethyl)-1-azaspiro[4.5]dec-3-en-2-one

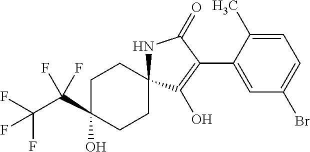 [SMILES: Cc1ccc(Br)cc1C1=C(O)[C@]2(CC[C@](O)(C(F)(F)C(F)(F)F)CC2)NC1=O]

912 mg (2.80 mmol) of caesium carbonate and 2.31 g (11.99 mmol) of (pentafluoroethyl)-trimethylsilane were added to 700 mg (2.00 mmol) of 3-(5-bromo-2-methylphenyl)-4-hydroxy-1-azaspiro[4.5]dec-3-ene-2,8-dione (Example 11A) in 14.6 ml of N,N-dimethylformamide. The reaction mixture was stirred at room temperature for five days. The mixture was then diluted with water, acidified with aqueous citric acid to pH=4.5 and extracted with ethyl acetate. The organic phase was washed with water, dried over sodium sulphate and concentrated under reduced pressure. The residue was dissolved in 50 ml of tetrahydrofuran, 10 ml of 2N aqueous hydrochloric acid were added and the mixture was stirred at room temperature for forty hours and then diluted with water. The crude product was extracted with ethyl acetate and washed with water, and the organic phase was dried over sodium sulphate. The residue was, after evaporation under reduced pressure, purified by chromatography on silica gel (mobile phase: hexane/ethyl acetate gradient). Evaporation and drying gave 490 mg (52% of theory) of the title compound.

¹H-NMR (300 MHz, methanol-d₄): δ [ppm]=1.39-1.50 (m, 2H), 1.91-2.03 (m, 4H), 2.15 (s, 3H), 2.32-2.48 (m, 2H), 7.15 (d, 1H), 7.26 (d, 1H), 7.34 (dd, 1H).

LC-MS (Method 3): Rₜ=1.13 min; MS (ESIpos): m/z=472 [M+H]⁺.

### Example 26A

### (5r,8r)-3-(5-Bromo-4-fluoro-2-methylphenyl)-4,8-dihydroxy-8-(pentafluoroethyl)-1-azaspiro[4.5]dec-3-en-2-one

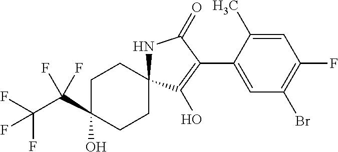 [SMILES: Cc1cc(F)c(Br)cc1C1=C(O)[C@]2(CC[C@](O)(C(F)(F)C(F)(F)F)CC2)NC1=O]

867 mg (2.66 mmol) of caesium carbonate and 2.19 g (11.41 mmol) of (pentafluoroethyl)-trimethylsilane were added to 700 mg (1.90 mmol) of 3-(5-bromo-4-fluoro-2-methylphenyl)-4-hydroxy-1-azaspiro[4.5]dec-3-ene-2,8-dione (Example 20A) in 14 ml of N,N-dimethylformamide. The reaction mixture was stirred at room temperature for three days. The mixture was then diluted with water, acidified with aqueous citric acid to pH=4.5 and extracted with ethyl acetate. The organic phase was washed with water, dried over sodium sulphate and concentrated under reduced pressure. The residue was dissolved in 50 ml of tetrahydrofuran, 10 ml of 2N aqueous hydrochloric acid were added, and the mixture was stirred at room temperature for twenty hours and then diluted with water. The crude product was extracted with ethyl acetate and washed with water, and the organic phase was dried over sodium sulphate. The residue was, after evaporation under reduced pressure, purified by chromatography on silica gel (mobile phase: hexane/ethyl acetate gradient). Evaporation and drying gave 595 mg (64% of theory) of the title compound.

¹H-NMR (300 MHz, methanol-d₄): δ [ppm]=1.38-1.50 (m, 2H), 1.92-2.00 (m, 4H), 2.16 (s, 3H), 2.31-2.47 (m, 2H), 7.09 (d, 1H), 7.33 (d, 1H).

LC-MS (Method 3): Rₜ=1.08 min; MS (ESIpos): m/z=488 [M+H]⁺.

### Example 27A

### Methyl cis-1-{[(5-bromo-2-methylphenyl)acetyl]amino}-4-(trifluoromethyl)cyclohexane-carboxylate

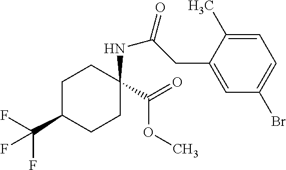 [SMILES: COC(=O)[C@]1(NC(=O)Cc2cc(Br)ccc2C)CC[C@@H](C(F)(F)F)CC1]

At room temperature, 10.00 g (38.22 mmol) of methyl cis-1-amino-4-(trifluoromethyl)cyclo-hexanecarboxylate hydrochloride (EP 1220841 A2 and WO 2001/23354 A3), 9.67 g (95.54 mmol) of triethylamine and 233 mg (1.91 mmol) of N,N-dimethylaminopyridine were dissolved in 95 ml of dichloromethane. A solution of 9.46 g (38.22 mmol) of (5-bromo-2-methylphenyl)acetyl chloride (Example 8A) in 95 ml of dichloromethane was then added dropwise to the mixture. The resulting reaction mixture was stirred at room temperature overnight. For work-up, the mixture was diluted with dichloromethane and the organic phase was washed with aqueous saturated sodium bicarbonate solution and with aqueous 5% strength citric acid. After drying over sodium sulphate, the solvent was evaporated and the residue was purified by chromatography on silica gel (mobile phase: dichloromethane/methanol gradient). Evaporation and drying gave 8.84 g (53% of theory) of the title compound which was used without further characterisation for the next step.

### Example 28A

### (5s,8s)-3-(5-Bromo-2-methylphenyl)-4-hydroxy-8 (trifluoromethyl)-1-azaspiro[4.5]dec-3-en-2-one

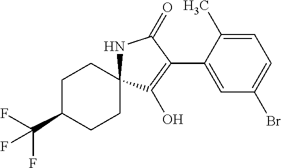 [SMILES: Cc1ccc(Br)cc1C1=C(O)[C@]2(CC[C@H](C(F)(F)F)CC2)NC1=O]

4.53 g (40.34 mmol) of potassium tert-butoxide were added to 8.80 g (20.17 mmol) of methyl cis-1-{[(5-bromo-2-methylphenyl)acetyl]amino}-4-(trifluoromethyl)cyclohexanecarboxylate (Example 27A) in 100 ml of N,N-dimethylformamide. The reaction mixture was stirred at 80° C. for 60 minutes. For work-up, the cold reaction mixture was poured onto 800 ml of ice-water and acidified with aqueous hydrochloric acid. The crude product was filtered off and purified by chromatography on silica gel (mobile phase: hexane/ethyl acetate gradient). Drying gave 5.23 g (64% of theory) of the title compound.

¹H-NMR (400 MHz, DMSO-d₆): δ [ppm]=1.40-1.50 (m, 2H), 1.58-1.72 (m, 2H), 1.77-1.86 (m, 2H), 1.86-1.96 (m, 2H), 2.07 (s, 3H), 2.12-2.28 (m, 1H), 7.14 (d, 1H), 7.19 (d, 1H), 7.33 (dd, 1H), 8.33 (s, 1H), 11.01 (s, 1H).

LC-MS (Method 2): Rₜ=1.18 min; MS (ESIpos): m/z=406 [M+H]⁺.

### Example 29A

### Methyl 1-{[(5-bromo-3-fluoro-2-methylphenyl)acetyl]amino}-4-(trifluoromethyl)cyclohexane-carboxylate

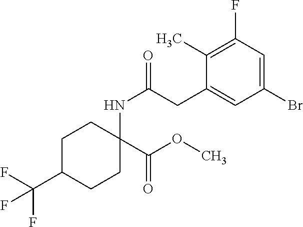 [SMILES: COC(=O)C1(NC(=O)Cc2cc(Br)cc(F)c2C)CCC(C(F)(F)F)CC1]

17.3 g (70.0 mmol) of (5-bromo-3-fluoro-2-methylphenyl)acetic acid (described in WO2009/049851, page 95) were dissolved in 29 ml (399 mmol) of thionyl chloride. The reaction mixture was stirred at 80° C. for 1 h and then concentrated. The residue was dissolved in 210 ml of acetonitrile, 30 ml of this solution were added dropwise with ice-cooling to a mixture of 2.00 g (7.64 mmol) of methyl cis-1-amino-4-(trifluoromethyl)cyclohexanecarboxylate hydrochloride (described in EP 1220841 A2 and WO 2001/23354 A3) and 3.70 g (26.8 mmol) of potassium carbonate in 15 ml of acetonitrile and the mixture was stirred at room temperature overnight. The mixture was then concentrated, taken up in water and extracted with dichloromethane, and the combined organic phases were washed with 1N aqueous hydrogen chloride solution and saturated aqueous sodium bicarbonate solution, dried over magnesium sulphate, filtered and concentrated. This gave 2.25 g (65% of theory) of the title compound as a mixture of diastereomers.

¹H-NMR (400 MHz, DMSO-d₆): δ [ppm]=1.42-1.58 (m, 2.3H), 1.63-1.83 (m, 3.7H), 2.06 (d, 0.45H), 2.10 (d, 2.55H), 2.09-2.19 (m, 1.7H), 2.25-2.45 (m, 1.3H), 3.51 (s, 0.3H), 3.56 (s, 3H), 3.60 (s, 1.7H), 7.25-7.27 (m, 0.15H), 7.29-7.31 (m, 0.85H), 7.36 (dd, 1H), 8.42 (s, 0.85H), 8.58 (s, 0.15H).

LC-MS (Method 1): Rₜ=1.37 min; MS (ESIpos): m/z=454 [M+H]⁺.

### Example 30A

### 3-(5-Bromo-3-fluoro-2-methylphenyl)-4-hydroxy-8-(trifluoromethyl)-1-azaspiro[4.5]dec-3-en-2-one

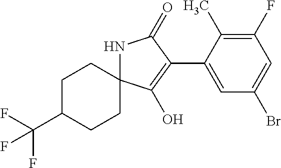 [SMILES: Cc1c(F)cc(Br)cc1C1=C(O)C2(CCC(C(F)(F)F)CC2)NC1=O]

1.11 g (9.91 mmol) of potassium tert-butoxide were added to 2.25 g (4.95 mmol) of the compound from Example 29A in 10 ml of N,N-dimethylformamide. The reaction mixture was heated at 80° C. for 15 minutes. After cooling, the mixture was added to ice-water, 1N aqueous hydrogen chloride solution was added dropwise and the mixture was stirred for 0.5 h. The precipitate was filtered off with suction, washed with water and dried. This gave 1.91 g (90% pure, 82% of theory) of the title compound as a mixture of diastereomers.

¹H-NMR (400 MHz, DMSO-d₆): δ [ppm]=1.46-1.54 (m, 1.7H), 1.59-1.76 (m, 2.3H), 1.81-2.00 (m, 4H), 2.00 (d, 3H), 2.17-2.44 (m, 1H), 7.11-7.14 (m, 1H), 7.41 (dd, 1H), 7.88 (s, 0.15H), 8.44 (s, 0.85H), 11.22 (s, 1H).

LC-MS (Method 1): Rₜ=1.18, 1.21 min; MS (ESIpos): m/z=422 [M+H]⁺.

### Example 31A

### (5s,8s)-Methyl 1-{[(5-bromo-2-chlorophenyl)acetyl]amino}-4-(trifluoromethyl)cyclohexane-carboxylate

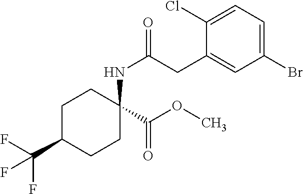 [SMILES: COC(=O)[C@]1(NC(=O)Cc2cc(Br)ccc2Cl)CC[C@@H](C(F)(F)F)CC1]

9.00 g (36.1 mmol) of (5-bromo-2-chlorophenyl)acetic acid (described in WO1998/05638, page 114) were dissolved in 15 ml (206 mmol) of thionyl chloride. The reaction mixture was stirred at 80° C. for 1 h and then concentrated. The residue was dissolved in 85 ml of acetonitrile. 2.62 g (10.0 mmol) of methyl cis-1-amino-4-(trifluoromethyl)cyclohexanecarboxylate hydrochloride (described in EP 1220841 A2 and WO 2001/23354 A3) were taken up in saturated aqueous sodium bicarbonate solution, extracted with ethyl acetate, dried over sodium sulphate, filtered and concentrated. The residue was dissolved in 20 ml of acetonitrile, and 4.84 g (35.0 mmol) of potassium carbonate were added. 30 ml of the solution of the acid chloride were added dropwise with ice-cooling, and the mixture was stirred at room temperature overnight. The mixture was then concentrated, taken up in water and extracted with dichloromethane, and the combined organic phases were washed with 1N aqueous hydrogen chloride solution and saturated aqueous sodium bicarbonate solution, dried over magnesium sulphate, filtered and concentrated. This gave 2.80 g of the title compound.

LC-MS (Method 1): Rₜ=1.35 min; MS (ESIpos): m/z=456 [M+H]⁺.

### Example 32A

### 3-(5-Bromo-2-chlorophenyl)-4-hydroxy-8-(trifluoromethyl)-1-azaspiro[4.5]dec-3-en-2-one

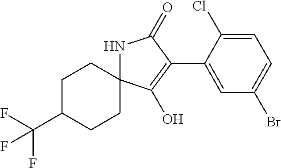 [SMILES: O=C1NC2(CCC(C(F)(F)F)CC2)C(O)=C1c1cc(Br)ccc1Cl]

1.38 g (12.3 mmol) of potassium tert-butoxide were added to 2.80 g (6.13 mmol) of the compound from Example 31A in 20 ml of N,N-dimethylformamide. The reaction mixture was heated at 80° C. for 15 minutes. After cooling, the mixture was added to ice-water, 1N aqueous hydrogen chloride solution was added dropwise and the mixture was stirred for 0.5 h. The precipitate was filtered off with suction, washed with water and dried. This gave 2.17 g (83% of theory) of the title compound as a mixture of diastereomers.

¹H-NMR (300 MHz, DMSO-d₆): δ [ppm]=1.43-1.54 (m, 1.7H), 1.57-1.77 (m, 2.3H), 1.79-2.02 (m, 4H), 2.17-2.37 (m, 1H), 7.39-7.45 (m, 2H), 7.51 (dd, 1H), 7.83 (s, 0.15H), 8.40 (s, 0.85H), 11.33 (s, 1H).

LC-MS (Method 1): Rₜ=1.16, 1.20 min; MS (ESIpos): m/z=424 [M+H]⁺.

### Example 33A

### (5s,8s)-3-(5-Bromo-2,6-dimethylphenyl)-4-hydroxy-8 (trifluoromethyl)-1-azaspiro[4.5]dec-3-en-2-one

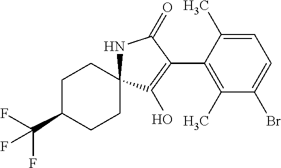 [SMILES: Cc1ccc(Br)c(C)c1C1=C(O)[C@]2(CC[C@H](C(F)(F)F)CC2)NC1=O]

Analogously to Example 32A, the title compound was obtained as a cis/trans isomer mixture of melting point 262° C.

¹H-NMR (300 MHz, DMSO-d₆): δ [ppm]=1.47-1.52 (m, 1H), 1.66-1.75 (m, 2H), 1.85-2.00 (m, 5H), 2.05, 2.17 (2 s, each 3H), 2.21-2.40 (m, 1H), 7.00 (d, 1H), 7.43 (d, 1H), 7.76, 8.30 (2 s, 1H), 10.91, 10.92 (2 s, 1H).

### Example 34A

### Methyl 1-{[(5-bromo-4-fluoro-2-methylphenyl)acetyl]amino}-4-(trifluoromethyl)cyclohexane-carboxylate

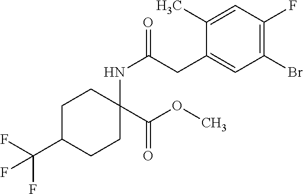 [SMILES: COC(=O)C1(NC(=O)Cc2cc(Br)c(F)cc2C)CCC(C(F)(F)F)CC1]

1.80 g (7.29 mmol) of (5-bromo-4-fluoro-2-methylphenyl)acetic acid (described in WO2009/049851) were dissolved in 3.0 ml (41.5 mmol) of thionyl chloride. The reaction mixture was stirred at 80° C. for 2 h and then concentrated. 0.35 g of the residue was dissolved in 1 ml of acetonitrile, with ice-cooling, this solution was added dropwise to a mixture of 0.52 g (2.00 mmol) of methyl cis-1-amino-4-(trifluoromethyl)cyclohexanecarboxylate hydrochloride (described in EP 1220841 A2 and WO 2001/23354 A3) and 0.65 g (4.67 mmol) of potassium carbonate in 8.0 ml of acetonitrile and the mixture was stirred at room temperature for two days. Water was then added, the mixture was extracted with dichloromethane, and the combined organic phases were washed with 1N aqueous hydrogen chloride solution and saturated aqueous sodium bicarbonate solution, dried, filtered and concentrated. This gave 271 mg (90% pure, 40% of theory) of the title compound as a mixture of diastereomers.

¹H-NMR (300 MHz, DMSO-d₆): δ [ppm]=1.40-1.58 (m, 2.3H), 1.62-1.84 (m, 3.7H), 2.08-2.20 (m, 1.7H), 2.18 (s, 0.45H), 2.21 (s, 2.55H), 2.25-2.45 (m, 1.3H), 3.43 (s, 0.3H), 3.52 (s, 1.7H), 3.56 (s, 3H), 7.20 (d, 1H), 7.46 (d, 0.15H), 7.51 (d, 0.85H), 8.38 (s, 0.85H), 8.54 (s, 0.15H). LC-MS (Method 2): Rₜ=1.35 min; MS (ESIpos): m/z=454 [M+H]⁺.

### Example 35A

### 3-(5-Bromo-4-fluoro-2-methylphenyl)-4-hydroxy-8-(trifluoromethyl)-1-azaspiro[4.5]dec-3-en-2-one

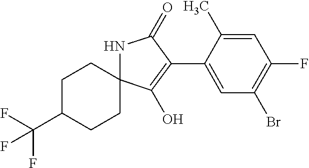 [SMILES: Cc1cc(F)c(Br)cc1C1=C(O)C2(CCC(C(F)(F)F)CC2)NC1=O]

Under nitrogen, 129 mg (1.15 mmol) of potassium tert-butoxide were added to 262 mg (0.58 mmol) of the compound from Example 34A in 1.5 ml of N,N-dimethylformamide. The reaction mixture was heated at 80° C. for 15 minutes. After cooling, the mixture was added to ice-water, 1N aqueous hydrogen chloride solution was added dropwise and the mixture was stirred for 0.5 h. The precipitate was filtered off with suction, washed with water and dried. This gave 217 mg (89% of theory) of the title compound as a mixture of diastereomers.

¹H-NMR (300 MHz, DMSO-d₆): δ [ppm]=1.44-1.54 (m, 1.7H), 1.58-1.77 (m, 2.3H), 1.80-2.02 (m, 4H), 2.13 (s, 3H), 2.17-2.37 (m, 1H), 7.27 (d, 1H), 7.34 (d, 1H), 7.83 (s, 0.15H), 8.40 (s, 0.85H), 11.08 (s, 1H).

LC-MS (Method 1): Rₜ=1.18 min; MS (ESIpos): m/z=422 [M+H]⁺.

### Example 36A

### Methyl 1-{[(4′-chloro-4,6-dimethylbiphenyl-3-yl)acetyl]amino}-4-(trifluoromethyl)cyclohexane-carboxylate

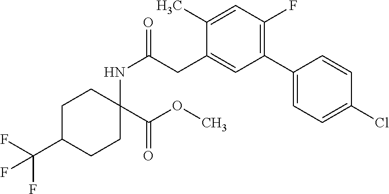 [SMILES: COC(=O)C1(NC(=O)Cc2cc(-c3ccc(Cl)cc3)c(F)cc2C)CCC(C(F)(F)F)CC1]

4.40 g (16.0 mmol) of (4′-chloro-4,6-dimethylbiphenyl-3-yl)acetic acid (described in WO99/48869, Example XXVII-1, page 186) were dissolved in 6.7 ml (91.3 mmol) of thionyl chloride. The reaction mixture was stirred at 80° C. for 1 h and then concentrated. The residue was dissolved in 28 ml of acetonitrile, 5 ml of this solution were added dropwise with ice-cooling to a mixture of 714 mg (2.73 mmol) of methyl cis-1-amino-4-(trifluoromethyl)cyclohexanecarboxylate hydrochloride (described in EP 1220841 A2 and WO 2001/23354 A3) and 880 mg (6.37 mmol) of potassium carbonate in 5 ml of acetonitrile and the mixture was stirred at room temperature for seven days. Water was then added, the mixture was extracted with dichloromethane, and the combined organic phases were washed with 1N aqueous hydrogen chloride solution and saturated aqueous sodium bicarbonate solution, dried, filtered and concentrated. This gave 571 mg (90% pure, 59% of theory) of the title compound as a mixture of diastereomers.

¹H-NMR (300 MHz, DMSO-d₆): δ [ppm]=1.38-1.57 (m, 2.3H), 1.60-1.82 (m, 3.7H), 2.07-2.41 (m, 9H), 3.42 (s, 0.3H), 3.47 (s, 0.45H), 3.48 (s, 2.55H), 3.51 (s, 1.7H), 6.99-7.10 (m, 2H), 7.27-7.35 (m, 2H), 7.44-7.51 (m, 2H), 8.32 (s, 0.85H), 8.46 (s, 0.15H).

LC-MS (Method 1): Rₜ=1.58 min; MS (ESIpos): m/z=482 [M+H]⁺.

### Example 37A

### Methyl cis-1-{[(4′-chloro-4-methylbiphenyl-3-yl)acetyl]amino}-4-(methoxymethyl)cyclohexane-carboxylate

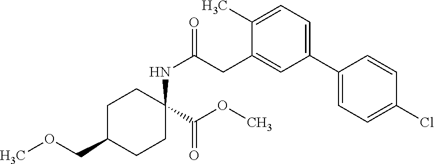 [SMILES: COC[C@H]1CC[C@](NC(=O)Cc2cc(-c3ccc(Cl)cc3)ccc2C)(C(=O)OC)CC1]

28.3 g (119 mmol) of methyl cis-1-amino-4-(methoxymethyl)cyclohexanecarboxylate hydrochloride (described in WO 2007/048545) were dissolved in 100 ml of water, 20.0 g (238 mmol) of sodium bicarbonate were added and the mixture was extracted repeatedly with ethyl acetate. The combined organic phases were dried over magnesium sulphate, filtered and concentrated. This gave 9.88 g of methyl cis-1-amino-4-(methoxymethyl)cyclohexanecarboxylate. 7.46 g (54.0 mmol) of potassium carbonate were added to 5.93 g (29.4 mmol) of methyl cis-1-amino-4-(methoxymethyl)cyclohexanecarboxylate in 50 ml of acetonitrile. With ice-cooling, a solution of 6.85 g (24.5 mmol) of the compound from Example 1A in 50 ml of acetonitrile was added, and the mixture was stirred at room temperature for one day. The mixture was then concentrated, water was added to the residue, the mixture was extracted with dichloromethane, and the combined organic phases were washed with 1N aqueous hydrogen chloride solution and saturated aqueous sodium bicarbonate solution, dried over magnesium sulphate, filtered and concentrated. This gave 10.5 g of the title compound which were reacted without any further purification.

¹H-NMR (300 MHz, DMSO-d₆): δ [ppm]=1.05-1.25 (m, 2H), 1.44-1.65 (m, 5H), 2.01-2.12 (m, 2H), 2.28 (s, 3H), 3.04 (d, 2H), 3.17 (s, 3H), 3.51 (s, 3H), 3.58 (s, 2H), 7.23 (d, 1H), 7.43 (dd, 1H), 7.47-7.57 (m, 3H), 7.62-7.68 (m, 2H), 8.21 (s, 1H).

LC-MS (Method 1): Rₜ=1.43 min; MS (ESIpos): m/z=444 [M+H]⁺.

### Example 38A

### (4′-Chloro-3′-fluoro-4-methylbiphenyl-3-yl)acetic acid

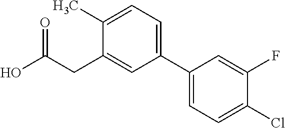 [SMILES: Cc1ccc(-c2ccc(Cl)c(F)c2)cc1CC(=O)O]

Under argon, 33.5 g (192 mmol) of (4-chloro-3-fluorophenyl)boronic acid were added to a solution of 40.0 g (175 mmol) of (5-bromo-2-methylphenyl)acetic acid (EP 1791816 and WO 2006/29799) in a mixture of 437 ml (437 mmol) of degassed 1N aqueous sodium hydroxide solution, 160 ml of degassed water and 160 ml of degassed tetrahydrofuran. The mixture was stirred for 10 minutes, 507 mg (1.75 mmol) of tri-tert-butylphosphonium tetrafluoroborate and 532 mg (1.75 mmol) of palladium(II) acetylacetonate were added and the mixture was stirred at room temperature for 20 h. Toluene and water were then added, the pH was adjusted to 1-2 using concentrated aqueous hydrogen chloride solution, the mixture was stirred for 10 minutes, the phases were separated, the aqueous phase was extracted twice with toluene, and the combined organic phases were dried over sodium sulphate, filtered and concentrated. The residue was triturated in 300 ml of a 6/1 mixture of n-hexane/tert-butyl methyl ether for 30 minutes, filtered off with suction, washed with n-hexane and dried under reduced pressure. This gave 38.0 g (78% of theory) of the title compound.

¹H-NMR (300 MHz, DMSO-d₆): δ [ppm]=2.27 (s, 3H), 3.67 (s, 2H), 7.27 (d, 1H), 7.49-7.59 (m, 3H), 7.61-7.75 (m, 2H), 12.4 (s, 1H).

LC-MS (Method 1): Rₜ=1.31 min; MS (ESIneg): m/z=277 [M−H]⁻.

### Example 39A

### Methyl cis-1-{[(4′-chloro-3′-fluoro-4-methylbiphenyl-3-yl)acetyl]amino}-4-methoxycyclohexane-carboxylate

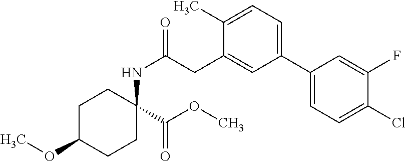 [SMILES: COC(=O)[C@]1(NC(=O)Cc2cc(-c3ccc(Cl)c(F)c3)ccc2C)CC[C@@H](OC)CC1]

10.0 g (35.9 mmol) of the compound from Example 38A were dissolved in 14.9 ml (205 mmol) of thionyl chloride. The reaction mixture was stirred at 90° C. for 1 h and then concentrated. This gave 10.8 g (100% of theory) (4′-chloro-3′-fluoro-4-methylbiphenyl-3-yl)acetyl chloride. 10.6 g (35.7 mmol) of (4′-chloro-3′-fluoro-4-methylbiphenyl-3-yl)acetyl chloride were dissolved in 120 ml of acetonitrile. 12.0 g (53.7 mmol) of methyl cis-1-amino-4-methoxycyclohexanecarboxylate hydrochloride (described in EP 1791816 and WO 2006/29799) were taken up in ethyl acetate, and saturated aqueous sodium bicarbonate solution was added. The phases were separated and the aqueous phase was extracted twice with ethyl acetate. The combined organic phases were dried over sodium sulphate, filtered and concentrated. This gave 8.50 g of methyl cis-1-amino-4-methoxycyclohexanecarboxylate. 17.3 g (125 mmol) of potassium carbonate were added to 8.02 g (42.8 mmol) of methyl cis-1-amino-4-methoxycyclohexanecarboxylate in 120 ml of acetonitrile. With ice-cooling, the solution of the acid chloride was added dropwise, and the mixture was stirred at room temperature overnight. The mixture was then concentrated, water was added to the residue, the mixture was extracted with dichloromethane, and the combined organic phases were washed with 1N aqueous hydrogen chloride solution and saturated aqueous sodium bicarbonate solution, dried over sodium sulphate, filtered and concentrated. This gave 15.7 g (98% of theory) of the title compound which were reacted without any further purification.

¹H-NMR (300 MHz, DMSO-d₆): δ [ppm]=1.30-1.47 (m, 2H), 1.60-1.74 (m, 2H), 1.75-1.85 (m, 2H), 1.99-2.11 (m, 2H), 2.28 (s, 3H), 3.09-3.20 (m, 1H), 3.21 (s, 3H), 3.52 (s, 3H), 3.58 (s, 2H), 7.24 (d, 1H), 7.46-7.55 (m, 2H), 7.57 (d, 1H), 7.61-7.72 (m, 2H), 8.30 (s, 1H).

LC-MS (Method 2): Rₜ=1.36 min; MS (ESIpos): m/z=448 [M+H]⁺.

### Example 40A

### (5s,8s)-3-(5-Bromo-2 chlorophenyl)-4-hydroxy-8-methoxy-1-azaspiro[4.5]dec-3-en-2-one

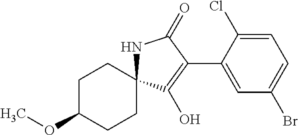 [SMILES: CO[C@H]1CC[C@]2(CC1)NC(=O)C(c1cc(Br)ccc1Cl)=C2O]

The title compound was prepared starting with methyl cis-1-amino-4-methoxycyclohexane-carboxylate hydrochloride (described in EP 1791816 and WO 2006/29799) and (5-bromo-2-chlorophenyl)acetic acid (described in WO1998/05638, page 114) analogously to the synthesis of the compounds from Example 31A and Example 32A. This gave 2.99 g of the title compound.

¹H-NMR (400 MHz, DMSO-d₆): δ [ppm]=1.40-1.56 (m, 4H), 1.82-2.01 (m, 4H), 3.07-3.18 (m, 1H), 3.26 (s, 3H), 7.38-7.44 (m, 2H), 7.50 (dd, 1H), 8.26 (s, 1H), 11.21 (s, 1H).

LC-MS (Method 1): Rₜ=0.98 min; MS (ESIpos): m/z=386 [M+H]⁺.

### Example 41A

### (5s,8s)-3-(5-Bromo 4 chloro 2 methylphenyl)-4-hydroxy-8-methoxy-1-azaspiro[4.5]dec-3-en-2-one

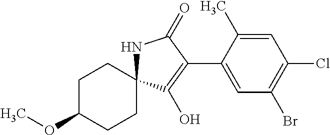 [SMILES: CO[C@H]1CC[C@]2(CC1)NC(=O)C(c1cc(Br)c(Cl)cc1C)=C2O]

The title compound was prepared starting with methyl cis-1-amino-4-methoxycyclohexane-carboxylate hydrochloride (described in EP 1791816 and WO 2006/29799) and (5-bromo-4-chloro-2-methylphenyl)acetic acid (preparation analogously to the synthesis sequence described in WO 1997/01535 for the preparation of Examples XXIV-1, XXV-1 and XXVI-1) analogously to the synthesis of the compound from Example 40A. This gave 0.86 g of the title compound.

¹H-NMR (300 MHz, DMSO-d₆): δ [ppm]=1.38-1.59 (m, 4H), 1.82-2.02 (m, 4H), 2.11 (s, 3H), 3.06-3.18 (m, 1H), 3.26 (s, 3H), 7.39 (s, 1H), 7.50 (s, 1H), 8.26 (s, 1H), 11.04 (s, 1H).

LC-MS (Method 1): Rₜ=1.07 min; MS (ESIpos): m/z=402 [M+H]⁺.

### Example 42A

### Methyl cis-1-{[(5-bromo-3-fluoro-2-methylphenyl)acetyl]amino}-4-methoxycyclohexane-carboxylate

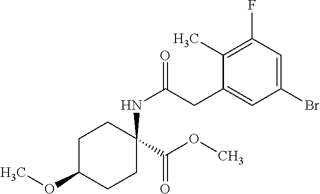 [SMILES: COC(=O)[C@]1(NC(=O)Cc2cc(Br)cc(F)c2C)CC[C@@H](OC)CC1]

2.21 g (8.94 mmol) of (5-bromo-3-fluoro-2-methylphenyl)acetic acid (described in WO2009/049851, page 95) were dissolved in 3.7 ml (51.0 mmol) of thionyl chloride. The reaction mixture was stirred at 80° C. for 1 h and then concentrated. The residue was dissolved in 15 ml of acetonitrile. 3.00 g (13.4 mmol) of methyl cis-1-amino-4-methoxycyclohexanecarboxylate hydrochloride (described in EP 1791816 and WO 2006/29799) were dissolved in 30 ml of acetonitrile, and 4.33 g (31.3 mmol) of potassium carbonate were added. The solution of the acid chloride was added dropwise with ice-cooling and stirred at room temperature overnight. The mixture was then concentrated to half its original volume, taken up in ice-water and extracted with dichloromethane, and the combined organic phases were washed with 1N aqueous hydrogen chloride solution and saturated aqueous sodium bicarbonate solution, dried over magnesium sulphate, filtered and concentrated. The crude product was purified by preparative HPLC [column: Xbridge C18, 5 μm, 150 mm×30 mm; mobile phase: water/acetonitrile gradient with addition of 0.1% formic acid], giving 1.59 g (43% of theory) of the title compound.

¹H-NMR (400 MHz, DMSO-d₆): δ [ppm]=1.32-1.44 (m, 2H), 1.62-1.72 (m, 2H), 1.77-1.86 (m, 2H), 1.99-2.06 (m, 2H), 2.09 (d, 3H), 3.11-3.21 (m, 1H), 3.23 (s, 3H), 3.54 (s, 3H), 3.59 (s, 2H), 7.27-7.30 (m, 1H), 7.36 (dd, 1H), 8.36 (s, 1H).

LC-MS (Method 1): Rₜ=1.20 min; MS (ESIpos): m/z=416 [M+H]⁺.

### Example 43A

### (5s,8s)-3-(5-Bromo-3-fluoro-2-methylphenyl)-4-hydroxy-8-methoxy-1-azaspiro[4.5]dec-3-en-2-one

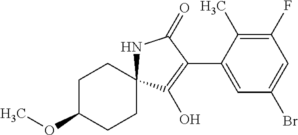 [SMILES: CO[C@H]1CC[C@]2(CC1)NC(=O)C(c1cc(Br)cc(F)c1C)=C2O]

The title compound was prepared analogously to the synthesis of the compounds from Example 32A starting with the compound from Example 42A. This gave 1.40 g (95% pure, 91% of theory) of the title compound.

¹H-NMR (400 MHz, DMSO-d₆): δ [ppm]=1.39-1.59 (m, 4H), 1.83-2.02 (m, 4H), 2.00 (d, 3H), 3.06-3.19 (m, 1H), 3.26 (s, 3H), 7.10-7.13 (m, 1H), 7.40 (dd, 1H), 8.29 (s, 1H), 11.12 (s, 1H).

LC-MS (Method 1): Rₜ=1.01 min; MS (ESIpos): m/z=384 [M+H]⁺.

### Example 44A

### Methyl 1-{[(5-bromo-2-methylphenyl)acetyl]amino}cyclohexanecarboxylate

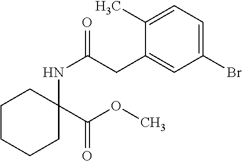 [SMILES: COC(=O)C1(NC(=O)Cc2cc(Br)ccc2C)CCCCC1]

2.06 g (9.00 mmol) of (5-bromo-2-methylphenyl)acetic acid (described in EP 1791816 and WO 2006/29799) were dissolved in 3.7 ml (51.3 mmol) of thionyl chloride. The reaction mixture was stirred at 80° C. for 2 h and then concentrated. The residue was dissolved in 20 ml of dichloromethane. 2.09 g (10.8 mmol) of methyl-1-amino-cyclohexanecarboxylate hydrochloride were dissolved in 25 ml of dichloromethane, 55 mg (0.45 mmol) of 4-dimethylaminopyridine and 3.1 ml (22.5 mmol) of triethylamine were added and the mixture was stirred at room temperature for 0.5 h. The solution of the acid chloride was added dropwise and the mixture was stirred at room temperature for 36 h. The mixture was then diluted with dichloromethane, washed with water, 1N aqueous hydrogen chloride solution and saturated aqueous sodium bicarbonate solution, dried, filtered and concentrated. This gave 2.80 g (84% of theory) of the title compound.

¹H-NMR (300 MHz, DMSO-d₆): δ [ppm]=1.15-1.31 (m, 1H), 1.37-1.58 (m, 5H), 1.59-1.73 (m, 2H), 1.86-1.99 (m, 2H), 2.19 (s, 3H), 3.51 (s, 2H), 3.54 (s, 3H), 7.10 (d, 1H), 7.30 (dd, 1H), 7.39 (d, 1H), 8.26 (s, 1H).

LC-MS (Method 1): Rₜ=1.30 min; MS (ESIpos): m/z=368 [M+H]⁺.

### Example 45A

### 3-(5-Bromo-2-methylphenyl)-4-hydroxy-1-azaspiro[4.5]dec-3-en-2-one

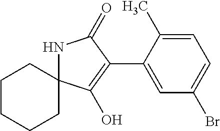 [SMILES: Cc1ccc(Br)cc1C1=C(O)C2(CCCCC2)NC1=O]

The title compound was prepared analogously to the synthesis of the compounds from Example 32A starting with the compound from Example 44A. This gave 2.33 g (90% of theory) of the title compound.

¹H-NMR (400 MHz, DMSO-d₆): δ [ppm]=1.09-1.27 (m, 1H), 1.30-1.41 (m, 2H), 1.51-1.72 (m, 5H), 1.76-1.90 (m, 2H), 2.11 (s, 3H), 7.17 (d, 1H), 7.22 (d, 1H), 7.36 (dd, 1H), 8.19 (s, 1H), 10.87 (s, 1H).

LC-MS (Method 1): Rₜ=1.09 min; MS (ESIpos): m/z=336 [M+H]⁺.

### Example 46A

### Methyl 1-{[(5-bromo-2-methylphenyl)acetyl]amino}-4-(2-methoxyethyl)cyclohexanecarboxylate

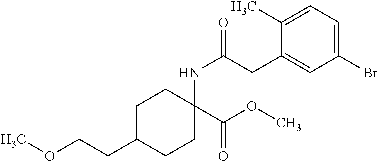 [SMILES: COCCC1CCC(NC(=O)Cc2cc(Br)ccc2C)(C(=O)OC)CC1]

2.18 g (9.53 mmol) of (5-bromo-2-methylphenyl)acetic acid (described in EP 1791816 and WO 2006/29799) were dissolved in 4.0 ml (54.3 mmol) of thionyl chloride. The reaction mixture was stirred at 80° C. for 1 h and then concentrated. The residue was dissolved in 20 ml of acetonitrile. 2.00 g (7.94 mmol) of methyl-1-amino-4-(2-methoxyethyl)cyclohexanecarboxylate hydrochloride (described in WO2007/048545, Example XIV-5, page 145) were dissolved in 15 ml of acetonitrile, and 3.84 g (27.8 mmol) of potassium carbonate were added. The solution of the acid chloride was added dropwise and the mixture was stirred at room temperature overnight. The mixture was then concentrated, and the residue was taken up in water, extracted with dichloromethane, washed with 1N aqueous hydrogen chloride solution and saturated aqueous sodium bicarbonate solution, dried over magnesium sulphate, filtered and concentrated. This gave 1.72 g (51% of theory) of the title compound as a mixture of diastereomers.

LC-MS (Method 1): Rₜ=1.28 min; MS (ESIpos): m/z=426 [M+H]⁺.

### Example 47A

### 3-(5-Bromo-2-methylphenyl)-4-hydroxy-8-(2-methoxyethyl)-1-azaspiro[4.5]dec-3-en-2-one

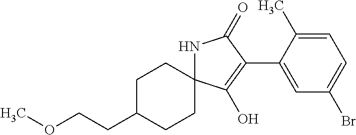 [SMILES: COCCC1CCC2(CC1)NC(=O)C(c1cc(Br)ccc1C)=C2O]

The title compound was prepared analogously to the synthesis of the compounds from Example 32A starting with the compound from Example 46A. This gave 1.17 g (90% pure, 67% of theory) of the title compound as a mixture of diastereomers.

¹H-NMR (400 MHz, DMSO-d₆): δ [ppm]=1.22-1.48 (m, 6H), 1.49-1.72 (m, 3H), 1.74-2.01 (m, 2H), 2.08 (s, 0.3H), 2.11 (s, 2.7H), 3.22 (s, 2.7H), 3.23 (0.3H), 3.33-3.39 (m, 2H), 7.17 (d, 1H), 7.22 (d, 1H), 7.36 (dd, 1H), 8.18 (s, 0.9H), 8.19 (s, 0.1H), 10.85 (s, 0.9H), 10.87 (s, 0.1H).

LC-MS (Method 1): Rₜ=1.10 min; MS (ESIpos): m/z=394 [M+H]⁺.

### Example 48A

### Methyl cis-1-{[(5-bromo-2-chlorophenyl)acetyl]amino}-4-(methoxymethyl)cyclohexane-carboxylate

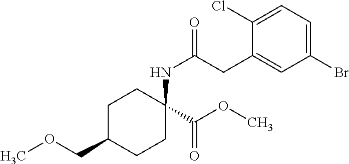 [SMILES: COC[C@H]1CC[C@](NC(=O)Cc2cc(Br)ccc2Cl)(C(=O)OC)CC1]

55.3 g (232 mmol) of methyl cis-1-amino-4-(methoxymethyl)cyclohexanecarboxylate hydrochloride (described in WO 2007/048545, page 144) were dissolved in 200 ml of water, 39.0 g (465 mmol) of sodium bicarbonate were added and the mixture was extracted repeatedly with ethyl acetate. The combined organic phases were dried over magnesium sulphate, filtered and concentrated. This gave 17.2 g of methyl cis-1-amino-4-(methoxymethyl)cyclohexanecarboxylate. 9.00 g (36.1 mmol) of (5-bromo-2-chlorophenyl)acetic acid (described in WO1998/05638, page 114) were dissolved in 15 ml (206 mmol) of thionyl chloride. The reaction mixture was stirred at 80° C. for 1 h and then concentrated. The residue was dissolved in 85 ml of acetonitrile. 4.84 g (35.0 mmol) of potassium carbonate were added to 2.01 g (10.0 mmol) of methyl cis-1-amino-4-(methoxymethyl)cyclohexanecarboxylate in 20 ml of acetonitrile. With ice-cooling, 30 ml (12.0 mmol) of the solution of the acid chloride were added dropwise, and the mixture was stirred at room temperature overnight. The mixture was then concentrated, water was added to the residue, the mixture was extracted dichloromethane, and the combined organic phases were washed with 1N aqueous hydrogen chloride solution and saturated aqueous sodium bicarbonate solution, dried over magnesium sulphate, filtered and concentrated. This gave 4.34 g (100% of theory) of the title compound which were reacted without any further purification.

¹H-NMR (300 MHz, DMSO-d₆): δ [ppm]=1.12-1.30 (m, 2H), 1.47-1.67 (m, 5H), 2.02-2.13 (m, 2H), 3.14 (d, 2H), 3.22 (s, 3H), 3.55 (s, 3H), 3.65 (s, 2H), 7.38 (d, 1H), 7.47 (dd, 1H), 7.60 (d, 1H), 8.30 (s, 1H).

LC-MS (Method 1): Rₜ=1.25 min; MS (ESIpos): m/z=432 [M+H]⁺.

### Example 49A

### (5s,8s)-3-(5-Bromo-2 chlorophenyl)-4-hydroxy-8-(methoxymethyl)-1-azaspiro[4.5]dec-3-en-2-one

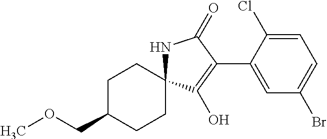 [SMILES: COC[C@H]1CC[C@]2(CC1)NC(=O)C(c1cc(Br)ccc1Cl)=C2O]

The title compound was prepared analogously to the synthesis of the compounds from Example 32A starting with the compound from Example 48A. This gave 3.43 g (84% of theory) of the title compound.

¹H-NMR (400 MHz, DMSO-d₆): δ [ppm]=1.22-1.44 (m, 4H), 1.49-1.61 (m, 1H), 1.65-1.74 (m, 2H), 1.79-1.89 (m, 2H), 3.15 (d, 2H), 3.23 (s, 3H), 7.40 (d, 1H), 7.41 (d, 1H), 7.50 (dd, 1H), 8.20 (s, 1H), 11.17 (s, 1H).

LC-MS (Method 1): Rₜ=1.05 min; MS (ESIpos): m/z=400 [M+H]⁺.

### Example 50A

### Methyl cis-1-{[(5-bromo-2-methylphenyl)acetyl]amino}-4-(methoxymethyl)cyclohexane-carboxylate

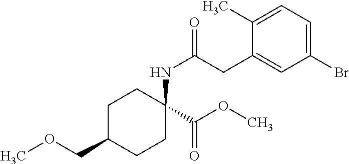 [SMILES: COC[C@H]1CC[C@](NC(=O)Cc2cc(Br)ccc2C)(C(=O)OC)CC1]

55.3 g (232 mmol) of methyl cis-1-amino-4-(methoxymethyl)cyclohexanecarboxylate hydrochloride (described in WO 2007/048545, page 144) were dissolved in 200 ml of water, 39.0 g (465 mmol) of sodium bicarbonate were added and the mixture was extracted repeatedly with ethyl acetate. The combined organic phases were dried over magnesium sulphate, filtered and concentrated. This gave 17.2 g of methyl cis-1-amino-4-(methoxymethyl)cyclohexanecarboxylate. 1.90 g (8.30 mmol) of (5-bromo-2-methylphenyl)acetic acid (described in EP 1791816 and WO 2006/29799) were dissolved in 3.5 ml (47.3 mmol) of thionyl chloride. The reaction mixture was stirred at 80° C. for 1 h and then concentrated. The residue was dissolved in 15 ml of acetonitrile. 2.52 g (18.2 mmol) of potassium carbonate were added to 2.00 g (9.94 mmol) of methyl cis-1-amino-4-(methoxymethyl)cyclohexanecarboxylate in 20 ml of acetonitrile. With ice-cooling, the solution of the acid chloride was added dropwise and stirred at room temperature for 24 h. The mixture was then concentrated, water was added to the residue, the mixture was extracted with dichloromethane, and the combined organic phases were washed with 1N aqueous hydrogen chloride solution and saturated aqueous sodium bicarbonate solution, dried over magnesium sulphate, filtered and concentrated. This gave 3.11 g (91% of theory) of the title compound which were reacted without any further purification.

¹H-NMR (300 MHz, DMSO-d₆): δ [ppm]=1.07-1.29 (m, 2H), 1.47-1.66 (m, 5H), 2.01-2.12 (m, 2H), 2.20 (s, 3H), 3.13 (d, 2H), 3.22 (s, 3H), 3.51 (s, 2H), 3.54 (s, 3H), 7.10 (d, 1H), 7.30 (dd, 1H), 7.41 (d, 1H), 8.22 (s, 1H).

LC-MS (Method 1): Rₜ=1.25 min; MS (ESIpos): m/z=412 [M+H]⁺.

### Example 51A

### (5s,8s)-3-(5-Bromo-2-methylphenyl)-4-hydroxy-8-(methoxymethyl)-1-azaspiro[4.5]dec-3-en-2-one

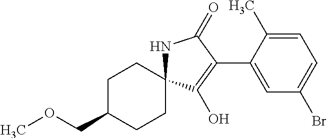 [SMILES: COC[C@H]1CC[C@]2(CC1)NC(=O)C(c1cc(Br)ccc1C)=C2O]

The title compound was prepared analogously to the synthesis of the compounds from Example 2032A starting with the compound from Example 50A. This gave 2.83 g (96% of theory) of the title compound.

¹H-NMR (300 MHz, DMSO-d₆): δ [ppm]=1.22-1.44 (m, 4H), 1.46-1.63 (m, 1H), 1.65-1.75 (m, 2H), 1.80-1.85 (m, 2H), 2.11 (s, 3H), 3.15 (d, 2H), 3.23 (s, 3H), 7.17 (d, 1H), 7.22 (d, 1H), 7.36 (dd, 1H), 8.16 (s, 1H), 10.89 (s, 1H).

LC-MS (Method 1): Rₜ=1.05 min; MS (ESIpos): m/z=380 [M+H]⁺.

### Example 52A

### Methyl cis-1-{[(5-bromo-2-methylphenyl)acetyl]amino}-4-methylcyclohexanecarboxylate

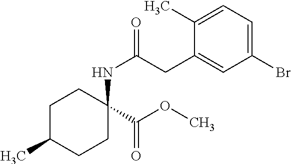 [SMILES: COC(=O)[C@]1(NC(=O)Cc2cc(Br)ccc2C)CC[C@@H](C)CC1]

6.39 g (27.9 mmol) of (5-bromo-2-methylphenyl)acetic acid (described in EP 1791816 and WO 2006/29799) were dissolved in 11.6 ml (159 mmol) of thionyl chloride. The reaction mixture was stirred at 80° C. for 1.5 h and then concentrated, taken up in toluene and concentrated. The residue was dissolved in 38 ml of acetonitrile. 8.69 g (41.8 mmol) of methyl-1-amino-4-methylcyclohexanecarboxylate hydrochloride (described in EP596298) were dissolved in 64 ml of acetonitrile, and 13.5 g (97.6 mmol) of potassium carbonate were added. The solution of the acid chloride was added dropwise with ice-cooling, and the mixture was stirred at room temperature for 5 days. The mixture was then added to ice-water and extracted with dichloromethane, and the extracts were washed with 0.5N aqueous hydrogen chloride solution and saturated aqueous sodium bicarbonate solution, dried, filtered and concentrated. This gave 5.98 g (56% of theory) of the title compound.

¹H-NMR (400 MHz, DMSO-d₆): δ [ppm]=0.86 (d, 3H), 1.06-1.20 (m, 2H), 1.27-1.40 (m, 1H), 1.55-1.52 (m, 2H), 1.54-1.64 (m, 2H), 2.00-2.08 (m, 2H), 2.20 (s, 3H), 3.51 (s, 2H), 3.54 (s, 3H), 7.10 (d, 1H), 7.30 (dd, 1H), 7.42 (d, 1H), 8.21 (s, 1H).

LC-MS (Method 1): Rₜ=1.38 min; MS (ESIpos): m/z=382 [M+H]⁺.

### Example 53A

### (5s,8s)-3-(5-Bromo-2-methylphenyl)-4-hydroxy-8-methyl-1-azaspiro[4.5]dec-3-en-2-one

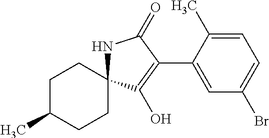 [SMILES: Cc1ccc(Br)cc1C1=C(O)[C@]2(CC[C@H](C)CC2)NC1=O]

The title compound was prepared analogously to the synthesis of the compounds from Example 32A starting with the compound from Example 52A. This gave 5.32 g (97% of theory) of the title compound.

¹H-NMR (400 MHz, DMSO-d₆): δ [ppm]=0.90 (d, 3H), 1.27-1.40 (m, 5H), 1.57-1.65 (m, 2H), 1.83-1.94 (m, 2H), 2.11 (s, 3H), 7.17 (d, 1H), 7.22 (d, 1H), 7.36 (dd, 1H), 8.16 (s, 1H), 10.86 (s, 1H).

LC-MS (Method 1): Rₜ=1.18 min; MS (ESIpos): m/z=350 [M+H]⁺.

### Example 54A

Methyl cis-1-{[(4′-chloro-4,6-dimethylbiphenyl-3-yl)acetyl]amino}-4-methoxycyclohexane-carboxylate

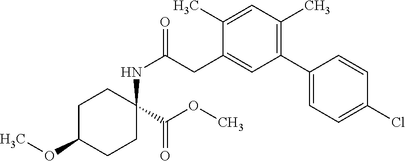 [SMILES: COC(=O)[C@]1(NC(=O)Cc2cc(-c3ccc(Cl)cc3)c(C)cc2C)CC[C@@H](OC)CC1]

10 g (40 mmol) of (4′-chloro-4,6-dimethylbiphenyl-3-yl)acetic acid (described in WO99/48869 Example XXVII-1) were dissolved in 15.2 ml (200 mmol) of thionyl chloride. The reaction mixture was stirred at 80° C. for 1 h and then concentrated. The residue was dissolved in 40 ml of acetonitrile. 17.88 g (80 mmol) of methyl cis-1-amino-4-methoxycyclohexanecarboxylate hydrochloride (described in EP 1791816 and WO 2006/29799) were dissolved in 80 ml of acetonitrile, and 22 g (160 mmol) of potassium carbonate were added. The solution of the acid chloride was added dropwise with ice-cooling, and the mixture was stirred at room temperature for one hour. The mixture was then triturated with 500 ml of ice-water and extracted with dichloromethane, and the combined organic phases were washed with 1N hydrochloric acid, dried over magnesium sulphate, filtered and concentrated. The crude product was purified by column chromatography on silica gel using methylene chloride/ethyl acetate 3:1 as mobile phase. This gave 11.13 g (62% of theory) of the title compound of melting point 76° C.

¹H-NMR (400 MHz, CDCl₃): δ [ppm]=1.14-1.24 (m, 1H), 1.30-1.38 (m, 1H), 1.72-1.91 (m, 4H), 1.87-2.05 (m, 1H), 2.06-2.11 (m, 1H), 2.16, 2.32 (2 s, each 3H), 3.13-3.21 (m, 1H), 3.31 (s, 3H), 3.56 (s, 2H), 3.67 (s, 3H), 7.04 (s, 1H), 7.13 (s, 1H), 7.22-7.26 (m, 3H), 7.36-7.40 (m, 2H).

### Example 55A

### Methyl (1S,3S)-1-{[(4′-chloro-4-methylbiphenyl-3-yl)acetyl]amino}-3-(trifluoromethyl)cyclo-hexanecarboxylate

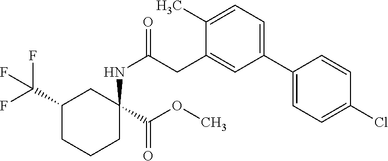 [SMILES: COC(=O)[C@]1(NC(=O)Cc2cc(-c3ccc(Cl)cc3)ccc2C)CCC[C@H](C(F)(F)F)C1]

2.62 g (11 mmol) of methyl-1-amino-3-trifluoromethylcyclohexanecarboxylate hydrochloride (preparation analogously to Example XIV-1 WO 2001/23354) were dissolved in 40 ml of tetrahydrofuran, 3.3 ml triethylamine were added and the mixture was stirred for 5 min. 2.61 g (10 mmol) of (4′-chloro-4-methylbiphenyl-3-yl)acetic acid (described in WO99/48869) were then added, and the mixture was stirred at RT for 15 min. A further 2.2 ml of triethylamine were added, and immediately 0.56 ml of phosphorus oxychloride was added such that the solution boiled gently. The mixture was boiled under reflux for a further 30 min. The mixture was then poured into 200 ml of ice-water and extracted with dichloromethane, and the combined organic phases were washed with 1N hydrochloric acid, dried over magnesium sulphate, filtered and concentrated. The crude product was purified by column chromatography on silica gel using n-hexane/ethyl acetate 2:1 as mobile phase. This gave 2.33 g (50% of theory) of the title compound of melting point 149° C.

¹H-NMR (400 MHz, CDCl₃): δ [ppm]=0.96-1.11 (m, 1H), 1.18-1.28 (m, 1H), 1.54-1.63 (m, 1H), 1.69-1.92 (m, 5H), 2.36 (s, 3H), 2.56-2.62 (m, 1H), 3.66 (s, 2H), 3.71 (s, 3H), 5.37 (s, 1H), 7.31-7.33 (m, 1H), 7.33-7.46 (m, 4H), 7.50-7.52 (m, 2H).

### Example 56A

### Methyl cis-1-{[(4,4′-dichlorobiphenyl-3-yl)acetyl]amino}-4-(trifluoromethyl)cyclohexane-carboxylate

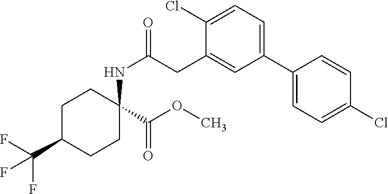 [SMILES: COC(=O)[C@]1(NC(=O)Cc2cc(-c3ccc(Cl)cc3)ccc2Cl)CC[C@@H](C(F)(F)F)CC1]

The title compound of melting point 187° C. was obtained analogously to Example 55A.

¹H-NMR (400 MHz, CDCl₃): δ [ppm]=1.27-1.35 (m, 2H), 1.71-1.85 (m, 4H), 1.98-2.08 (m, 1H), 2.23-2.29 (m, 2H), 3.67 (s, 3H), 3.77 (s, 2H), 5.66 (s, 1H), 7.39-7.51 (m, 6H), 7.64-7.69 (m, 1H).

### Example 57A

### Methyl cis-1-{[(4′-chloro-6-fluoro-4-methylbiphenyl-3-yl)acetyl]amino}-4-methoxycyclohexane-carboxylate

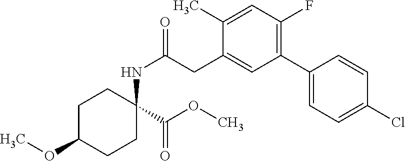 [SMILES: COC(=O)[C@]1(NC(=O)Cc2cc(-c3ccc(Cl)cc3)c(F)cc2C)CC[C@@H](OC)CC1]

The title compound of melting point 101° C. was obtained analogously to Example 55A.

¹H-NMR (400 MHz, CD₃CN): δ [ppm]=1.28-1.39 (m, 2H), 1.72-1.88 (m, 3H), 2.05-2.09 (m, 2H), 2.32 (s, 1H), 3.13-3.20 (m, 1H), 3.25 (s, 3H), 3.54 (s, 2H), 3.55 (s, 3H), 6.47 (s, 1H), 7.01-7.04 (d, 1H), 7.32-7.34 (d, 1H), 7.43-7.46 (m, 2H), 7.51-7.54 (m, 1H).

### Example 58A

### (5s,8s)-3-(5-Bromo-2-methylphenyl)-4-hydroxy-8-methoxy-1-azaspiro[4.5]dec-3-en-2-one

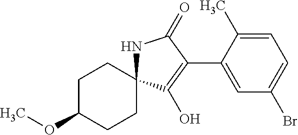 [SMILES: CO[C@H]1CC[C@]2(CC1)NC(=O)C(c1cc(Br)ccc1C)=C2O]

The title compound of melting point 218° C. was obtained analogously to Example 32A.

¹H-NMR (400 MHz, D₆-DMSO): δ [ppm]=1.42-1.57 (m, 4H), 1.87-1.99 (m, 4H), 2.10 (s, 3H), 3.09-3.16 (m, 1H), 3.25 (s, 3H), 7.15-7.17 (m, 1H), 7.21-7.22 (m, 1H), 7.34-7.36 (m, 1H), 8.13 (s, 1H), 10.83 (s, 1H).

### Example 59A

### Methyl (1S,3S)-1-{[(4,4′-dichlorobiphenyl-3-yl)acetyl]amino}-3-(trifluoromethyl)cyclohexane-carboxylate

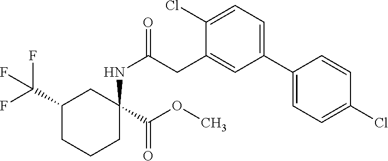 [SMILES: COC(=O)[C@]1(NC(=O)Cc2cc(-c3ccc(Cl)cc3)ccc2Cl)CCC[C@H](C(F)(F)F)C1]

The title compound of melting point 148° C. was obtained analogously to Example 55A.

¹H-NMR (400 MHz, CDCl₃): δ [ppm]=1.20-1.38 (m, 2H), 1.58-1.67 (m, 1H), 1.76-1.83 (m, 2H), 1.87-1.99 (m, 3H), 2.57-2.62 (m, 1H), 3.69 (s, 3H), 3.77 (s, 2H), 5.37 (s, 1H), 7.40-7.51 (m, 6H), 7.57-7.58 (m, 1H).

### Example 60A

### Methyl 1-{[(4,4′-dichlorobiphenyl-3-yl)acetyl]amino}-4,4-dimethylcyclohexanecarboxylate

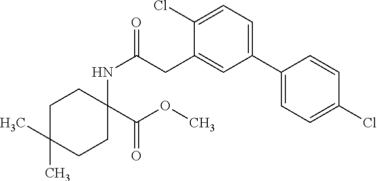 [SMILES: COC(=O)C1(NC(=O)Cc2cc(-c3ccc(Cl)cc3)ccc2Cl)CCC(C)(C)CC1]

The title compound of melting point 128° C. was obtained analogously to Examples 34 and V-58 of EP595130.

¹H-NMR (400 MHz, CDCl₃): δ [ppm]=0.85, 0.9 (2 s, each 3H), 1.09-1.17 (m, 2H), 1.19-1.29 (m, 2H), 1.90-1.94 (m, 4H), 3.67 (s, 3H), 3.75 (s, 2H), 5.70 (s, 1H), 7.40-7.45 (m, 3H), 7.48-7.51 (m, 3H), 7.58-7.59 (m, 1H).

### Example 61A

### Methyl 1-{[(4,4′-dichlorobiphenyl-3-yl)acetyl]amino}-4-methylcyclohexanecarboxylate

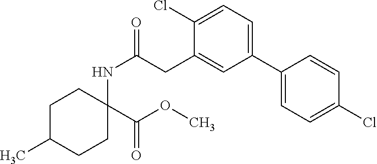 [SMILES: COC(=O)C1(NC(=O)Cc2cc(-c3ccc(Cl)cc3)ccc2Cl)CCC(C)CC1]

The title compound was obtained analogously to Example 55A as a viscous oil.

¹H-NMR (400 MHz, CDCl₃): δ [ppm]=0.83 (d, 3H), 0.85-0.93 (m, 1H), 1.26-1.39 (m, 1H), 1.55-1.62 (m, 3H), 1.69-1.77 (m, 2H), 2.09-2.12 (m, 2H), 3.66 (s, 3H), 3.76 (s, 2H), 5.69 (s, 1H), 7.40-7.51 (m, 6H), 7.58-7.59 (d, 1H).

### Example 62A

### Methyl 1-{[(4,4′-dichlorobiphenyl-3-yl)acetyl]amino}-cyclohexanecarboxylate

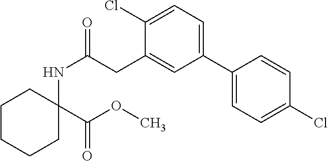 [SMILES: COC(=O)C1(NC(=O)Cc2cc(-c3ccc(Cl)cc3)ccc2Cl)CCCCC1]

The title compound of melting point 124° C. was obtained analogously to Example 55A.

¹H-NMR (400 MHz, CDCl₃): δ [ppm]=1.25-1.33 (m, 4H), 1.52-1.63 (m, 2H), 1.75-1.82 (m, 2H), 1.98-2.02 (m, 2H), 3.66 (s, 3H), 3.75 (s, 2H), 5.72 (s, 1H), 7.40-7.45 (m, 3H), 7.48-7.51 (m, 3H), 7.58-7.59 (m, 1H).

### Example 63A

### Methyl cis-1-{[(4,4′-dichlorobiphenyl-3-yl)acetyl]amino}-4-isopropylcyclohexanecarboxylate

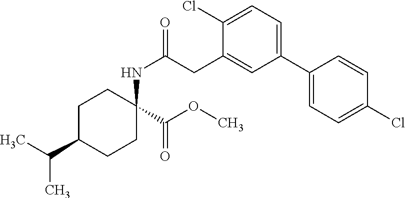 [SMILES: COC(=O)[C@]1(NC(=O)Cc2cc(-c3ccc(Cl)cc3)ccc2Cl)CC[C@@H](C(C)C)CC1]

The title compound of melting point 109° C. was obtained analogously to Example 55A.

¹H-NMR (400 MHz, CDCl₃): δ [ppm]=0.78 (d, 6H), 0.88-0.95 (m, 2H), 1.02-1.07 (m, 1H), 1.29-1.39 (m, 2H), 1.66-1.74 (m, 2H), 2.07-2.15 (m, 2H), 3.67 (s, 3H), 3.76 (s, 2H), 5.66 (s, 1H), 7.40-7.45 (m, 3H), 7.47-7.51 (m, 3H), 7.58-7.59 (m, 1H).

### Example 64A

### 3-(5-Bromo-2-methylphenyl)-8-ethoxy-4-hydroxy-1-azaspiro[4.5]dec-3-en-2-one

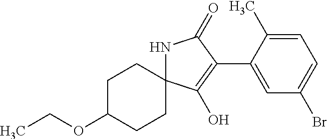 [SMILES: CCOC1CCC2(CC1)NC(=O)C(c1cc(Br)ccc1C)=C2O]

The title compound was prepared analogously to the synthesis of the compound from Example 40A as a cis/trans isomer mixture of melting point 138° C.

### Example 1-90

### =Compound According to WO08/067,911

### (5s,8s)-3-(4′-Chloro-3′-fluoro-4-methylbiphenyl-3-yl)-4-hydroxy-8-(trifluoromethyl)-1-azaspiro[4.5]dec-3-en-2-one

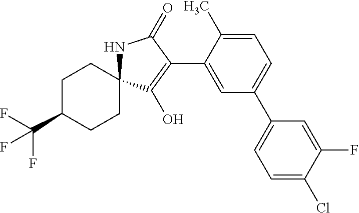 [SMILES: Cc1ccc(-c2ccc(Cl)c(F)c2)cc1C1=C(O)[C@]2(CC[C@H](C(F)(F)F)CC2)NC1=O]

Under argon, 1.01 g (1.24 mmol) of dichloro[1,1′-bis(diphenylphosphino)ferrocene]palladium dichloromethane complex were added to 5.00 g (12.4 mmol) of the compound from Example 28A in 500 ml of degassed 1,2-dimethoxyethane. The mixture was stirred at room temperature for 5 minutes, and 3.24 g (18.5 mmol) of (4-chloro-3-fluorophenyl)boronic acid and a solution of 14.1 g (43.3 mmol) of caesium carbonate in 30 ml of degassed water were then added. The reaction mixture was heated under reflux for 2 h. After cooling, 10 ml of concentrated aqueous hydrogen chloride solution were added, the aqueous phase was separated off, magnesium sulphate was added, the mixture was filtered off through silica gel, the filter cake was washed with ethyl acetate and the filtrate was concentrated. Purification of the crude product by chromatography on silica gel (mobile phase: hexane/ethyl acetate gradient) and by crystallization from ethyl acetate gave 2.48 g (44% of theory) of the title compound.

¹H-NMR (300 MHz, DMSO-d₆): δ [ppm]=1.45-1.57 (m, 2H), 1.62-1.79 (m, 2H), 1.81-2.05 (m, 4H), 2.19 (s, 3H), 2.20-2.33 (m, 1H), 7.32 (d, 1H), 7.41 (d, 1H), 7.49-7.58 (m, 2H), 7.64 (t, 1H), 7.70 (dd, 1H), 8.33 (s, 1H), 10.95 (s, 1H).

LC-MS (Method 1): Rₜ=1.34 min; MS (ESIpos): m/z=454 [M+H]⁺.

### Example 1-91

### (5s,8s)-3-(4′-Chloro 5 fluoro 4-methylbiphenyl-3-yl)-4-hydroxy-8-(trifluoromethyl)-1-azaspiro[4.5]dec-3-en-2-one

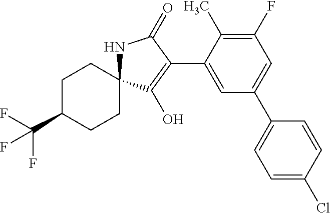 [SMILES: Cc1c(F)cc(-c2ccc(Cl)cc2)cc1C1=C(O)[C@]2(CC[C@H](C(F)(F)F)CC2)NC1=O]

Under argon, 250 mg (1.60 mmol) of (4-chlorophenyl)boronic acid and a solution of 1.22 g (3.73 mmol) of caesium carbonate in 2 ml of degassed water were added to 450 mg (1.07 mmol) of the compound from Example 30A and 43.5 mg (0.053 mmol) of dichloro[1,1′-bis(diphenylphosphino)ferrocene]palladium dichloromethane complex in 23 ml of degassed 1,2-dimethoxyethane. The reaction mixture was heated under reflux for 3 h. A further 83 mg (0.53 mmol) of (4-chlorophenyl)boronic acid and a spatula tip of dichloro[1,1′-bis(diphenylphosphino)ferrocene]palladium dichloromethane complex were then added, and the mixture was heated under reflux for 2 h. After cooling, 550 μl of concentrated aqueous hydrogen chloride solution and sodium sulphate were added, the mixture was filtered off through silica gel and sodium sulphate, the filter cake was washed with ethyl acetate and the filtrate was concentrated. The diastereomers were separated by two preparative HPLCs [1. column: Xbridge C18, 5 μm, 150 mm×30 mm; mobile phase: water/methanol gradient with addition of 0.1% formic acid; flow rate: 0.8 ml/min; temperature: RT; 2. column: Chiralpak IC, 5 μm, 150 mm×4.6 mm; mobile phase: hexane/ethanol=85/15 with addition of 0.1% trifluoroacetic acid; flow rate: 1 ml/min; temperature: RT]. The separated diastereomer was purified once more by preparative HPLC (XBridge C18, 5 μm, 100 mm×30 mm; mobile phase: water/acetonitrile gradient with addition of 0.1% formic acid), giving 55.2 mg (11% of theory) of the title compound.

¹H-NMR (300 MHz, DMSO-d₆): δ [ppm]=1.46-1.58 (m, 2H), 1.62-1.79 (m, 2H), 1.81-2.04 (m, 4H), 2.08 (d, 3H), 2.18-2.34 (m, 1H), 7.25 (d, 1H), 7.44 (dd, 1H), 7.48-7.54 (m, 2H), 7.67-7.74 (m, 2H), 8.41 (s, 1H), 11.11 (s, 1H).

LC-MS (Method 1): Rₜ=1.32 min; MS (ESIpos): m/z=454 [M+H]⁺.

### Example 1-92

### =Compound According to WO08/067,911

### (5s,8s)-3-(4,4′-Dichloro-3′-fluorobiphenyl-3-yl)-4-hydroxy-8-(trifluoromethyl)-1-azaspiro[4.5]dec-3-en-2-one

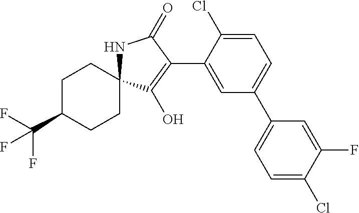 [SMILES: O=C1N[C@]2(CC[C@@H](C(F)(F)F)CC2)C(O)=C1c1cc(-c2ccc(Cl)c(F)c2)ccc1Cl]

Under argon 73.9 mg (0.42 mmol) of (4-chloro-3-fluorophenyl)boronic acid and a solution of 403 mg (1.24 mmol) of caesium carbonate in 0.9 ml of degassed water were added to 150 mg (0.35 mmol) of the compound from Example 32A and 14.4 mg (0.018 mmol) of dichloro[1,1′-bis(diphenylphosphino)ferrocene]palladium dichloromethane complex in 15 ml of degassed 1,2-dimethoxyethane. In a closed vessel, the reaction mixture was heated at 150° C. with microwave irradiation for 10 minutes. After cooling, 300 μl of concentrated aqueous hydrogen chloride solution and sodium sulphate were added, the mixture was filtered off through silica gel and sodium sulphate, the filter cake was washed with ethyl acetate and the filtrate was concentrated. The crude product was purified by preparative HPLC (XBridge C18, 5 μm, 100 mm×30 mm; mobile phase: water/acetonitrile gradient with addition of 0.1% formic acid), giving 45.3 mg (90% pure, 24% of theory) of the title compound.

¹H-NMR (300 MHz, DMSO-d₆): δ [ppm]=1.46-1.57 (m, 2H), 1.61-1.79 (m, 2H), 1.81-2.04 (m, 4H), 2.19-2.37 (m, 1H), 7.54-7.61 (m, 3H), 7.64-7.72 (m, 2H), 7.78 (dd, 1H), 8.40 (s, 1H), 11.22 (s, 1H).

LC-MS (Method 1): Rₜ=1.34 min; MS (ESIpos): m/z=474 [M+H]⁺.

### Example 1-93

### =Compound According to WO08/067,911

### (5s,8s)-3-(4′-Chloro-3′-fluoro-2,4-dimethylbiphenyl-3-yl)-4-hydroxy-8-(trifluoromethyl)-1-azaspiro[4.5]dec-3-en-2-one

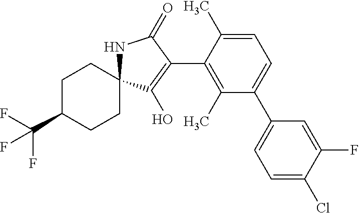 [SMILES: Cc1ccc(-c2ccc(Cl)c(F)c2)c(C)c1C1=C(O)[C@]2(CC[C@H](C(F)(F)F)CC2)NC1=O]

Under argon, 29.3 mg (0.036 mmol) of dichloro[1,1′-bis(diphenylphosphino)ferrocene]palladium dichloromethane complex were added to 150 mg (0.36 mmol) of the compound from Example 33A in 15 ml of degassed 1,2-dimethoxyethane. The mixture was stirred at room temperature for 5 minutes, and 93.8 mg (0.54 mmol) of (4-chloro-3-fluorophenyl)boronic acid and a solution of 409 mg (1.26 mmol) of caesium carbonate in 0.9 ml of degassed water were then added. In a closed vessel, the reaction mixture was heated at 150° C. with microwave irradiation for 10 minutes. After cooling, 300 μl of concentrated aqueous hydrogen chloride solution and magnesium sulphate were added, the mixture was filtered off through silica gel and magnesium sulphate, the filter cake was washed with ethyl acetate and the filtrate was concentrated. The crude product was purified by preparative HPLC (XBridge C18, 5 μm, 100 mm×30 mm; mobile phase: water/acetonitrile gradient with addition of 0.1% formic acid), giving 93.1 mg (56% of theory) of the title compound.

¹H-NMR (300 MHz, DMSO-d₆): δ [ppm]=1.43-1.56 (m, 1H), 1.60-1.79 (m, 2H), 1.80-2.08 (m, 8H), 2.13 (s, 3H), 2.20-2.40 (m, 1H), 7.06-7.18 (m, 3H), 7.28-7.34 (m, 1H), 7.64 (t, 1H), 7.76 (s, 0.5H), 8.29 (s, 0.5H), 10.85 (s, 1H).

LC-MS (Method 1): Rₜ=1.36 min; MS (ESIpos): m/z=468 [M+H]⁺.

### Example 1-94

### =Compound According to WO08/067,911

### 3-(4′-Chloro-3′,6-difluoro-4-methylbiphenyl-3-yl)-4-hydroxy-8-(trifluoromethyl)-1-azaspiro[4.5]dec-3-en-2-one

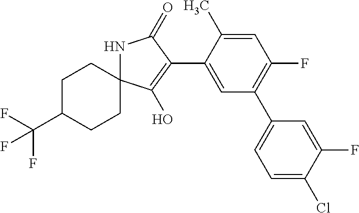 [SMILES: Cc1cc(F)c(-c2ccc(Cl)c(F)c2)cc1C1=C(O)C2(CCC(C(F)(F)F)CC2)NC1=O]

The title compound was prepared analogously to the synthesis of the compound from Example 1-93 starting with 103 mg (0.24 mmol) of the compound from Example 35A. This gave 55.1 mg (43% of theory) of the title compound as a mixture of diastereomers.

¹H-NMR (300 MHz, DMSO-d₆): δ [ppm]=1.46-1.54 (m, 2H), 1.62-1.76 (m, 2H), 1.81-2.02 (m, 4H), 2.20 (s, 3H), 2.20-2.31 (m, 1H), 7.20-7.28 (m, 2H), 7.41 (d, 1H), 7.58 (d, 1H), 7.69 (t, 1H), 8.36 (s, 1H), 11.00 (s, 1H).

LC-MS (Method 1): Rₜ=1.35 min; MS (ESIpos): m/z=472 [M+H]⁺.

### Example 1-95

### =Compound According to WO99/48869

### (5s,8s)-3-(4′-Chloro-2,4-dimethylbiphenyl-3-yl)-4-hydroxy-8-(trifluoromethyl)-1-azaspiro[4.5]dec-3-en-2-one

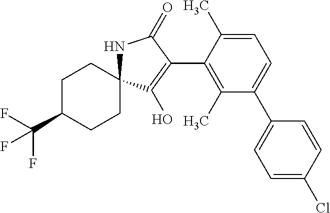 [SMILES: Cc1ccc(-c2ccc(Cl)cc2)c(C)c1C1=C(O)[C@]2(CC[C@H](C(F)(F)F)CC2)NC1=O]

The title compound was prepared analogously to the synthesis of the compound from Example 1-93 starting with 150 mg (0.36 mmol) of the compound from Example 33A. This gave 55.1 mg (43% of theory) of the title compound.

¹H-NMR (300 MHz, DMSO-d₆): δ [ppm]=1.44-1.54 (m, 1H), 1.60-1.77 (m, 2H), 1.81-2.06 (m, 8H), 2.13 (s, 3H), 2.20-2.44 (m, 1H), 7.05 (d, 1H), 7.13 (d, 1H), 7.28-7.33 (m, 2H), 7.46-7.52 (m, 2H), 7.76 (s, 0.5H), 8.29 (s, 0.5H), 10.85 (s, 1H).

LC-MS (Method 1): Rₜ=1.35 min; MS (ESIpos): m/z=450 [M+H]⁺.

### Example 1-96

### (5s,8s)-4-Hydroxy-8-(trifluoromethyl)-3-(3′,4′,5-trifluoro-4-methylbiphenyl-3-yl)-1-azaspiro[4.5]dec-3-en-2-one

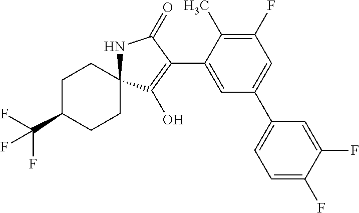 [SMILES: Cc1c(F)cc(-c2ccc(F)c(F)c2)cc1C1=C(O)[C@]2(CC[C@H](C(F)(F)F)CC2)NC1=O]

The title compound was prepared analogously to the synthesis of the compound from Example 1-91 starting with 450 mg (1.07 mmol) of the compound from Example 30A. This gave 194 mg (40% of theory) of the title compound.

¹H-NMR (300 MHz, DMSO-d₆): δ [ppm]=1.47-1.58 (m, 2H), 1.61-1.80 (m, 2H), 1.82-2.04 (m, 4H), 2.08 (d, 3H), 2.18-2.34 (m, 1H), 7.27 (s, 1H), 7.44-7.58 (m, 3H), 7.75-7.85 (m, 1H), 8.42 (s, 1H), 11.11 (s, 1H).

LC-MS (Method 3): Rₜ=1.28 min; MS (ESIpos): m/z=456 [M+H]⁺.

### Example 1-97

### =Compound According to WO99/48869

### 3-(4′-Chloro-4,6-dimethylbiphenyl-3-yl)-4-hydroxy-8 (trifluoromethyl)-1-azaspiro[4.5]dec-3-en-2-one

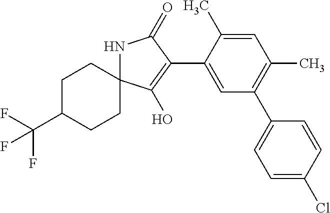 [SMILES: Cc1cc(C)c(-c2ccc(Cl)cc2)cc1C1=C(O)C2(CCC(C(F)(F)F)CC2)NC1=O]

233 mg (2.08 mmol) of potassium tert-butoxide were added to 557 mg (90%, 1.04 mmol) of the compound from Example 36A in 1.5 ml of N,N-dimethylformamide. The reaction mixture was stirred at 80° C. for 15 minutes. After cooling, the mixture was concentrated, the residue was dissolved in water and the solution was added dropwise to 2N aqueous hydrogen chloride solution. The solid was filtered off with suction, washed with water and dried. This gave 510 mg (90% pure, 98% of theory) of a 10/1 mixture of the (5s,8s)- and (5r,8r)-diastereomers of the title compound.

### Example 1-98

### =Compound According to WO99/48869

### (5s,8s)-3-(4′-Chloro-4,6-dimethylbiphenyl-3-yl)-4-hydroxy-8-(trifluoromethyl)-1-azaspiro[4.5]dec-3-en-2-one

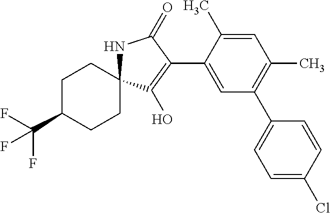 [SMILES: Cc1cc(C)c(-c2ccc(Cl)cc2)cc1C1=C(O)[C@]2(CC[C@H](C(F)(F)F)CC2)NC1=O]

By two preparative HPLCs [1. column: Chiralpak IA, 5 μm, 250 mm×20 mm; mobile phase: hexane/ethanol=85/15 with addition of 0.1% trifluoroacetic acid; flow rate: 20 ml/min; temperature: RT; 2. column: Xbridge C18, 5 μm, 150 mm×19 mm; mobile phase: water/acetonitrile gradient with addition of 0.2% ammonia; flow rate: 25 ml/min; temperature: RT], the diastereomers of the compound from Example 1-97 were separated. The ammonium salt of the main diastereomer were taken up in a mixture of 0.5N aqueous hydrogen chloride solution and ethyl acetate, the phases were separated, and the organic phase was washed with saturated sodium chloride solution, dried over magnesium sulphate, filtered and concentrated. This gave 220 mg of the title compound.

¹H-NMR (400 MHz, DMSO-d₆): δ [ppm]=1.42-1.51 (m, 2H), 1.62-1.75 (m, 2H), 1.80-1.89 (m, 2H), 1.89-2.00 (m, 2H), 2.15 (s, 3H), 2.21 (s, 3H), 2.23-2.31 (m, 1H), 6.90 (s, 1H), 7.15 (s, 1H), 7.33-7.37 (m, 2H), 7.46-7.51 (m, 2H), 8.29 (s, 1H), 10.83 (s, 1H).

LC-MS (Method 1): Rₜ=1.37 min; MS (ESIpos): m/z=450 [M+H]⁺.

### Example 1-99

### =Compound According to WO08/067,911

### (5s,8s)-3-(4 Chloro-3′,4′-difluorobiphenyl-3-yl)-4-hydroxy-8-(trifluoromethyl)-1-azaspiro[4.5]dec-3-en-2-one

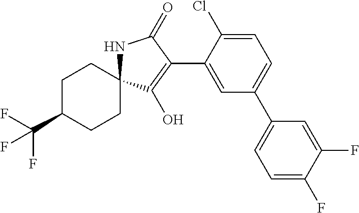 [SMILES: O=C1N[C@]2(CC[C@@H](C(F)(F)F)CC2)C(O)=C1c1cc(-c2ccc(F)c(F)c2)ccc1Cl]

The title compound was prepared analogously to the synthesis of the compound from Example 1-92 starting with 150 mg (0.35 mmol) of the compound from Example 32A. This gave 61.8 mg (90% pure, 34% of theory) of the title compound.

¹H-NMR (300 MHz, DMSO-d₆): δ [ppm]=1.45-1.57 (m, 2H), 1.60-1.79 (m, 2H), 1.80-2.04 (m, 4H), 2.17-2.37 (m, 1H), 7.47-7.58 (m, 4H), 7.64 (dd, 1H), 7.74-7.85 (m, 1H), 8.39 (s, 1H), 11.22 (s, 1H).

LC-MS (Method 1): Rₜ=1.29 min; MS (ESIpos): m/z=458 [M+H]⁺.

### Example 1-100

### =Compound According to WO99/48869

### 3-(4′-Chloro-6-fluoro-4-methylbiphenyl-3-yl)-4-hydroxy-8-(trifluoromethyl)-1-azaspiro[4.5]dec-3-en-2-one

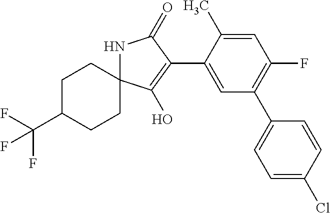 [SMILES: Cc1cc(F)c(-c2ccc(Cl)cc2)cc1C1=C(O)C2(CCC(C(F)(F)F)CC2)NC1=O]

The title compound was prepared analogously to the synthesis of the compound from Example 1-93 starting with 103 mg (0.24 mmol) of the compound from Example 35A. This gave 58 mg (43% of theory) of the title compound as a mixture of diastereomers.

¹H-NMR (400 MHz, DMSO-d₆): δ [ppm]=1.46-1.54 (m, 2H), 1.61-1.76 (m, 2H), 1.81-2.02 (m, 4H), 2.19 (s, 3H), 2.20-2.31 (m, 1H), 7.17-7.23 (m, 2H), 7.50-7.58 (m, 4H), 8.35 (s, 1H), 10.99 (s, 1H).

LC-MS (Method 1): Rₜ=1.33 min; MS (ESIpos): m/z=454 [M+H]⁺.

### Example 1-101

### =Compound According to WO08/067,911

### (5s,8s)-3-(3′,4′-Difluoro-2,4-dimethylbiphenyl-3-yl)-4-hydroxy-8-(trifluoromethyl)-1-azaspiro[4.5]dec-3-en-2-one

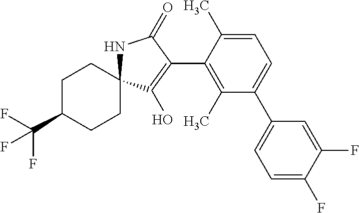 [SMILES: Cc1ccc(-c2ccc(F)c(F)c2)c(C)c1C1=C(O)[C@]2(CC[C@H](C(F)(F)F)CC2)NC1=O]

The title compound was prepared analogously to the synthesis of the compound from Example 1-92 starting with 150 mg (0.36 mmol) of the compound from Example 33A. This gave 95 mg (59% of theory) of the title compound.

¹H-NMR (300 MHz, DMSO-d₆): δ [ppm]=1.43-1.55 (m, 1H), 1.60-1.79 (m, 2H), 1.81-2.06 (m, 8H), 2.13 (s, 3H), 2.20-2.44 (m, 1H), 7.04-7.16 (m, 3H), 7.27-7.36 (m, 1H), 7.43-7.55 (m, 1H), 7.74 (s, 0.5H), 8.28 (s, 0.5H), 10.85 (s, 1H).

LC-MS (Method 1): Rₜ=1.31 min; MS (ESIpos): m/z=452 [M+H]⁺.

### Example 1-102

### (5s,8s)-3-(4′-Chloro-3′,5-difluoro-4-methylbiphenyl-3-yl)-4-hydroxy-8-(trifluoromethyl)-1-azaspiro[4.5]dec-3-en-2-one

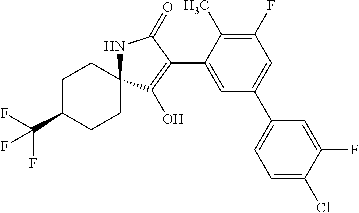 [SMILES: Cc1c(F)cc(-c2ccc(Cl)c(F)c2)cc1C1=C(O)[C@]2(CC[C@H](C(F)(F)F)CC2)NC1=O]

The title compound was prepared analogously to the synthesis of the compound from Example 1-91 starting with 450 mg (1.07 mmol) of the compound from Example 30A. After purification of the crude product by two chromatographies on silica gel (1. column: dichloromethane/methanol gradient; 2. column: hexane/ethyl acetate gradient), the product was subjected to fine purification by preparative HPLC [column: Xbridge C18, 5 μm, 150 mm×30 mm; mobile phase: water/acetonitrile gradient with addition of 0.1% formic acid; flow rate: 50 ml/min; temperature: RT]. This gave 130 mg (26% of theory) of the title compound.

¹H-NMR (300 MHz, DMSO-d₆): δ [ppm]=1.47-1.57 (m, 2H), 1.60-1.79 (m, 2H), 1.81-2.04 (m, 4H), 2.08 (d, 3H), 2.18-2.34 (m, 1H), 7.31 (d, 1H), 7.51 (dd, 1H), 7.57 (dd, 1H), 7.66 (t, 1H), 7.78 (dd, 1H), 8.38 (s, 1H), 11.12 (s, 1H).

LC-MS (Method 2): Rₜ=1.35 min; MS (ESIpos): m/z=472 [M+H]⁺.

### Example 1-103

### =Compound of Table 1, p. 25 of WO08/067,910

### (5s,8s)-3-(4,4′-Dichloro-3′-fluorobiphenyl-3-yl)-4-hydroxy-8-methoxy-1-azaspiro[4.5]dec-3-en-2-one

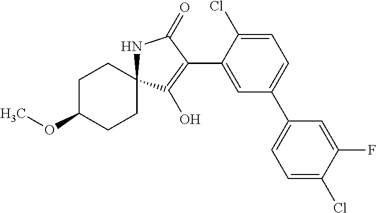 [SMILES: CO[C@H]1CC[C@]2(CC1)NC(=O)C(c1cc(-c3ccc(Cl)c(F)c3)ccc1Cl)=C2O]

The title compound was prepared analogously to the synthesis of the compound from Example 1-92 starting with 150 mg (0.39 mmol) of the compound from Example 40A. This gave 54.2 mg (32% of theory) of the title compound.

¹H-NMR (400 MHz, DMSO-d₆): δ [ppm]=1.42-1.59 (m, 4H), 1.84-2.03 (m, 4H), 3.09-3.18 (m, 1H), 3.26 (s, 3H), 7.53-7.59 (m, 3H), 7.65-7.70 (m, 2H), 7.78 (dd, 1H), 8.25 (s, 1H), 11.11 (s, 1H).

LC-MS (Method 1): Rₜ=1.22 min; MS (ESIpos): m/z=436 [M+H]⁺.

### Example 1-104

### =Compound of Table 1, p. 25 of WO08/067,910

### (5s,8s)-3-(4 Chloro-3′,4′-difluorobiphenyl-3-yl)-4-hydroxy-8-methoxy-1-azaspiro[4.5]dec-3-en-2-one

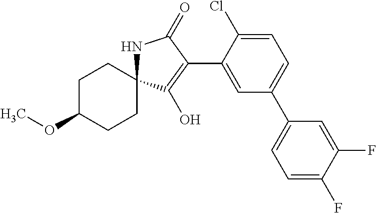 [SMILES: CO[C@H]1CC[C@]2(CC1)NC(=O)C(c1cc(-c3ccc(F)c(F)c3)ccc1Cl)=C2O]

The title compound was prepared analogously to the synthesis of the compound from Example 1-92 starting with 150 mg (0.39 mmol) of the compound from Example 40A. This gave 64.5 mg (40% of theory) of the title compound.

¹H-NMR (400 MHz, DMSO-d₆): δ [ppm]=1.42-1.59 (m, 4H), 1.84-2.03 (m, 4H), 3.09-3.18 (m, 1H), 3.26 (s, 3H), 7.48-7.56 (m, 4H), 7.63 (dd, 1H), 7.75-7.83 (m, 1H), 8.25 (s, 1H), 11.10 (s, 1H).

LC-MS (Method 1): Rₜ=1.16 min; MS (ESIpos): m/z=420 [M+H]⁺.

### Example 1-105

### (5s,8s)-3-(4′-Chloro-3′,5-difluoro-4-methylbiphenyl-3-yl)-4-hydroxy-8-methoxy-1-azaspiro[4.5]dec-3-en-2-one

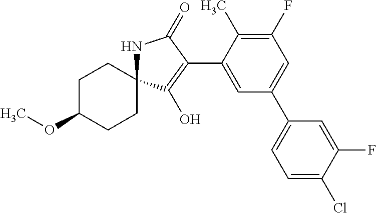 [SMILES: CO[C@H]1CC[C@]2(CC1)NC(=O)C(c1cc(-c3ccc(Cl)c(F)c3)cc(F)c1C)=C2O]

The title compound was prepared analogously to the synthesis of the compound from Example 1-92 starting with 150 mg (0.39 mmol) of the compound from Example 43A. This gave 80.4 mg (48% of theory) of the title compound.

¹H-NMR (300 MHz, DMSO-d₆): δ [ppm]=1.40-1.62 (m, 4H), 1.83-2.04 (m, 4H), 2.08 (d, 3H), 3.07-3.20 (m, 1H), 3.26 (s, 3H), 7.30 (d, 1H), 7.50 (d, 1H), 7.56 (dd, 1H), 7.66 (t, 1H), 7.78 (dd, 1H), 8.25 (s, 1H), 11.02 (s, 1H).

LC-MS (Method 1): Rₜ=1.24 min; MS (ESIpos): m/z=434 [M+H]⁺.

### Example 1-106

### (5s,8s)-3-(4′-Chloro 5 fluoro 4-methylbiphenyl-3-yl)-4-hydroxy-8-methoxy-1-azaspiro[4.5]dec-3-en-2-one

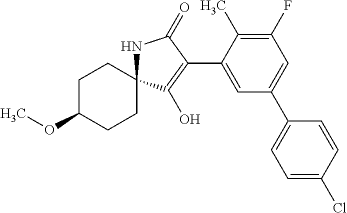 [SMILES: CO[C@H]1CC[C@]2(CC1)NC(=O)C(c1cc(-c3ccc(Cl)cc3)cc(F)c1C)=C2O]

The title compound was prepared analogously to the synthesis of the compound from Example 1-92 starting with 150 mg (0.39 mmol) of the compound from Example 43A. This gave 72.4 mg (48% of theory) of the title compound.

¹H-NMR (300 MHz, DMSO-d₆): δ [ppm]=1.41-1.61 (m, 4H), 1.83-2.04 (m, 4H), 2.08 (d, 3H), 3.07-3.20 (m, 1H), 3.26 (s, 3H), 7.23 (d, 1H), 7.43 (dd, 1H), 7.48-7.54 (m, 2H), 7.66-7.73 (m, 2H), 8.27 (s, 1H), 11.00 (s, 1H).

LC-MS (Method 1): Rₜ=1.23 min; MS (ESIpos): m/z=416 [M+H]⁺.

### Example 1-107

### (5s,8s)-4-Hydroxy-8-methoxy-3-(3′,4′,5-trifluoro-4-methylbiphenyl-3-yl)-1-azaspiro[4.5]dec-3-en 2-one

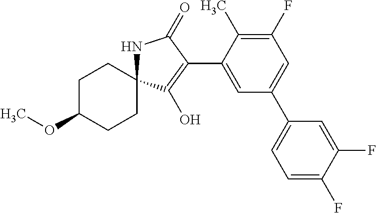 [SMILES: CO[C@H]1CC[C@]2(CC1)NC(=O)C(c1cc(-c3ccc(F)c(F)c3)cc(F)c1C)=C2O]

The title compound was prepared analogously to the synthesis of the compound from Example 1-92 starting with 150 mg (0.39 mmol) of the compound from Example 43A. This gave 20.3 mg (48% of theory) of the title compound.

¹H-NMR (300 MHz, DMSO-d₆): δ [ppm]=1.40-1.62 (m, 4H), 1.84-2.04 (m, 4H), 2.07 (d, 3H), 3.07-3.20 (m, 1H), 3.26 (s, 3H), 7.25 (d, 1H), 7.43-7.56 (m, 3H), 7.74-7.84 (m, 1H), 8.27 (s, 1H), 11.00 (s, 1H).

LC-MS (Method 1): Rₜ=1.18 min; MS (ESIpos): m/z=418 [M+H]⁺.

### Example 1-108

### =Compound According to WO08/067,910

### (5s,8s)-3-(4′,6-Dichloro-3′-fluoro-4-methylbiphenyl-3-yl)-4-hydroxy-8-methoxy-1-azaspiro[4.5]dec-3-en-2-one

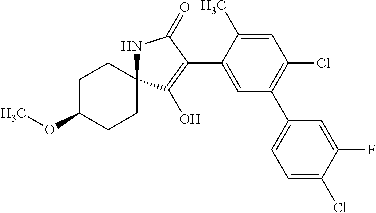 [SMILES: CO[C@H]1CC[C@]2(CC1)NC(=O)C(c1cc(-c3ccc(Cl)c(F)c3)c(Cl)cc1C)=C2O]

The title compound was prepared analogously to the synthesis of the compound from Example 1-92 starting with 150 mg (0.37 mmol) of the compound from Example 41A. This gave 73.8 mg (44% of theory) of the title compound.

¹H-NMR (300 MHz, DMSO-d₆): δ [ppm]=1.37-1.59 (m, 4H), 1.82-2.02 (m, 4H), 2.18 (s, 3H), 3.05-3.18 (m, 1H), 3.25 (s, 3H), 7.12 (s, 1H), 7.29 (dd, 1H), 7.42-7.51 (m, 2H), 7.67 (t, 1H), 8.23 (s, 1H), 10.96 (s, 1H).

LC-MS (Method 1): Rₜ=1.29 min; MS (ESIpos): m/z=450 [M+H]⁺.

### Example 1-109

### =Compound According to WO99/48869

### (5s,8s)-3-(4′,6-Dichloro-4-methylbiphenyl-3-yl)-4-hydroxy-8-methoxy-1-azaspiro[4.5]dec-3-en-2-one

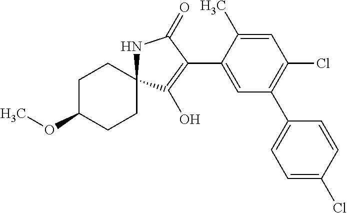 [SMILES: CO[C@H]1CC[C@]2(CC1)NC(=O)C(c1cc(-c3ccc(Cl)cc3)c(Cl)cc1C)=C2O]

The title compound was prepared analogously to the synthesis of the compound from Example 1-92 starting with 150 mg (0.37 mmol) of the compound from Example 41A. This gave 71.6 mg (44% of theory) of the title compound.

¹H-NMR (300 MHz, DMSO-d₆): δ [ppm]=1.37-1.59 (m, 4H), 1.82-2.02 (m, 4H), 2.18 (s, 3H), 3.05-3.18 (m, 1H), 3.25 (s, 3H), 7.09 (s, 1H), 7.40-7.48 (m, 3H), 7.49-7.55 (m, 2H), 8.21 (s, 1H), 10.96 (s, 1H).

LC-MS (Method 1): Rₜ=1.28 min; MS (ESIpos): m/z=432 [M+H]⁺.

### Example 1-110

### =Compound of Table 1, p. 41 in combination with Table 2, p. 45 of WO08/067,911

### (5s,8s)-3-(4′-Chloro-3′-fluoro-4-methylbiphenyl-3-yl)-4-hydroxy-8-methyl-1-azaspiro[4.5]dec-3-en-2-one

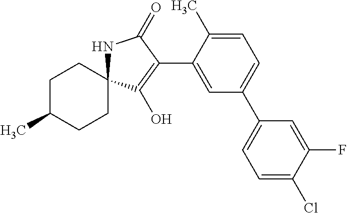 [SMILES: Cc1ccc(-c2ccc(Cl)c(F)c2)cc1C1=C(O)[C@]2(CC[C@H](C)CC2)NC1=O]

The title compound was prepared analogously to the synthesis of the compound from Example 1-93 starting with 150 mg (0.43 mmol) of the compound from Example 53A. This gave 33.5 mg (20% of theory) of the title compound.

¹H-NMR (400 MHz, DMSO-d₆): δ [ppm]=0.91 (d, 3H), 1.28-1.43 (m, 5H), 1.56-1.68 (m, 2H), 1.84-1.96 (m, 2H), 2.19 (s, 3H), 7.31 (d, 1H), 7.39 (d, 1H), 7.49-7.56 (m, 2H), 7.64 (t, 1H), 7.70 (dd, 1H), 8.12 (s, 1H), 10.76 (s, 1H).

LC-MS (Method 2): Rₜ=1.33 min; MS (ESIpos): m/z=400 [M+H]⁺.

### Example 1-111

### =Compound of Table 1, p. 41 in combination with Table 2, p. 47 of WO08/067,911

### (5s,8s)-3-(4′-Chloro-3′-fluoro-4-methylbiphenyl-3-yl)-4-hydroxy-8-(methoxymethyl)-1-azaspiro[4.5]dec-3-en-2-one

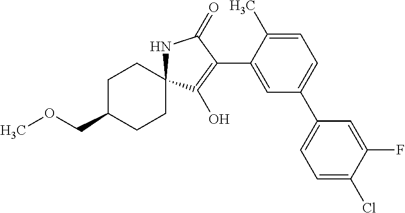 [SMILES: COC[C@H]1CC[C@]2(CC1)NC(=O)C(c1cc(-c3ccc(Cl)c(F)c3)ccc1C)=C2O]

Under argon, 1.10 g (6.31 mmol) of (4-chloro-3-fluorophenyl)boronic acid and a solution of 6.00 g (18.4 mmol) of caesium carbonate in 13 ml of degassed water were added to 2.00 g (5.26 mmol) of the compound from Example 51A and 215 mg (0.26 mmol) of dichloro[1,1′-bis(diphenylphosphino)ferrocene]palladium dichloromethane complex in 213 ml of degassed 1,2-dimethoxyethane. The reaction mixture was heated under reflux for 2 h. After cooling, 2.5 ml of concentrated aqueous hydrogen chloride solution and sodium sulphate were added, the mixture was filtered off through silica gel and sodium sulphate, the filter cake was washed with ethyl acetate and the filtrate was concentrated. The crude product was purified by chromatography on silica gel (mobile phase: hexane/ethyl acetate gradient). The product was then triturated with aqueous sodium bicarbonate solution, acidified with concentrated aqueous hydrogen chloride solution, filtered off with suction, washed with water and dried. This gave 1.50 g (65% of theory) of the title compound.

¹H-NMR (300 MHz, DMSO-d₆): δ [ppm]=1.21-1.47 (m, 4H), 1.48-1.64 (m, 1H), 1.65-1.78 (m, 2H), 1.81-1.97 (m, 2H), 2.19 (s, 3H), 3.16 (d, 2H), 3.24 (s, 3H), 7.31 (d, 1H), 7.40 (d, 1H), 7.48-7.57 (m, 2H), 7.60-7.74 (m, 2H), 8.13 (s, 1H), 10.79 (s, 1H).

LC-MS (Method 1): Rₜ=1.26 min; MS (ESIpos): m/z=430 [M+H]⁺.

### Example 1-112

### =Compound of Table 1, p. 41 in combination with Table 2, p. 47 of WO08/067,911

### (5s,8s)-3-(3′,4′-Difluoro-4-methylbiphenyl-3-yl)-4-hydroxy-8-(methoxymethyl)-1-azaspiro[4.5]dec-3-en-2-one

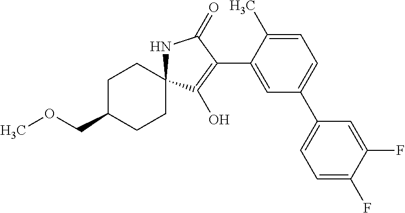 [SMILES: COC[C@H]1CC[C@]2(CC1)NC(=O)C(c1cc(-c3ccc(F)c(F)c3)ccc1C)=C2O]

The title compound was prepared analogously to the synthesis of the compound from Example 1-111 starting with 4.00 g (10.5 mmol) of the compound from Example 51A. After chromatography on silica gel, the product was stirred with water, sodium bicarbonate solution and 2N sodium hydroxide solution, acidified with concentrated aqueous hydrogen chloride solution, filtered off with suction and washed with water. This gave 2.86 g (65% of theory) of the title compound.

¹H-NMR (300 MHz, DMSO-d₆): δ [ppm]=1.21-1.47 (m, 4H), 1.48-1.64 (m, 1H), 1.65-1.78 (m, 2H), 1.81-1.97 (m, 2H), 2.18 (s, 3H), 3.16 (d, 2H), 3.24 (s, 3H), 7.30 (d, 1H), 7.36 (d, 1H), 7.43-7.54 (m, 3H), 7.64-7.76 (m, 1H), 8.14 (s, 1H), 10.78 (s, 1H).

LC-MS (Method 1): Rₜ=1.21 min; MS (ESIpos): m/z=414 [M+H]⁺.

### Example 1-113

### =Compound of Table 1, p. 40 in combination with Table 2, p. 47 der WO08/067,911

### (5s,8s)-3-(4,4′-Dichloro-3′-fluorobiphenyl-3-yl)-4-hydroxy-8-(methoxymethyl)-1-azaspiro[4.5]dec-3-en-2-one

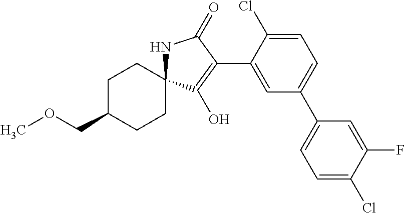 [SMILES: COC[C@H]1CC[C@]2(CC1)NC(=O)C(c1cc(-c3ccc(Cl)c(F)c3)ccc1Cl)=C2O]

The title compound was prepared analogously to the synthesis of the compound from Example 1-92 starting with 450 mg (1.12 mmol) of the compound from Example 49A. After chromatography, the product was dissolved in dichloromethane and concentrated. This gave 144 mg (28% of theory) of the title compound.

¹H-NMR (300 MHz, DMSO-d₆): δ [ppm]=1.21-1.47 (m, 4H), 1.48-1.64 (m, 1H), 1.65-1.78 (m, 2H), 1.80-1.94 (m, 2H), 3.16 (d, 2H), 3.24 (s, 3H), 7.51-7.60 (m, 3H), 7.62-7.71 (m, 2H), 7.77 (dd, 1H), 8.14 (s, 1H), 11.08 (s, 1H).

LC-MS (Method 1): Rₜ=1.27 min; MS (ESIpos): m/z=450 [M+H]⁺.

### Example 1-114

### =Compound of Table 1, p. 41 in combination with Table 2, p. 47 of WO08/067,911

### (5s,8s)-3-(4′-Chloro-3′-fluoro-4-methylbiphenyl-3-yl)-4-hydroxy-8-(2-methoxyethyl)-1-azaspiro[4.5]dec-3-en-2-one

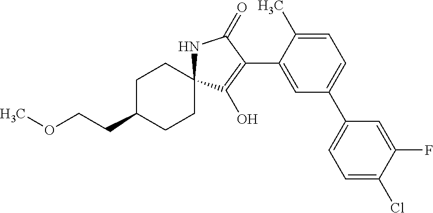 [SMILES: COCC[C@H]1CC[C@]2(CC1)NC(=O)C(c1cc(-c3ccc(Cl)c(F)c3)ccc1C)=C2O]

Under argon 332 mg (1.90 mmol) of (4-chloro-3-fluorophenyl)boronic acid and a solution of 1.45 g (4.44 mmol) of caesium carbonate in 2.2 ml of degassed water were added to 500 mg (1.27 mmol) of the compound from Example 47A and 51.8 mg (0.063 mmol) of dichloro[1,1′-bis(diphenylphosphino)ferrocene]palladium dichloromethane complex in 23 ml of degassed 1,2-dimethoxyethane. The reaction mixture was heated under reflux for 3 h. A further 111 mg (0.63 mmol) of (4-chloro-3-fluorophenyl)boronic acid and a spatula tip of dichloro[1,1′-bis(diphenylphosphino)ferrocene]palladium dichloromethane complex were then added, and the mixture was heated under reflux for 2 h. After cooling, 0.6 ml of concentrated aqueous hydrogen chloride solution and sodium sulphate were added, the mixture was filtered off through silica gel and sodium sulphate, the filter cake was washed with ethyl acetate and the filtrate was concentrated. The diastereomers were separated by two preparative HPLCs [1. column: Xbridge C18, 5 μm, 150 mm×30 mm; mobile phase: water/methanol gradient with addition of 0.1% formic acid; flow rate: 50 ml/min; temperature: RT; 2. column: Chiralpak IA, 5 μm, 250 mm×20 mm; mobile phase: hexane/ethanol=85/15 with addition of 0.1% trifluoroacetic acid; flow rate: 40 ml/min; temperature: RT]. This gave 191 mg (33% of theory) of the title compound.

¹H-NMR (300 MHz, DMSO-d₆): δ [ppm]=1.26-1.50 (m, 7H), 1.61-1.74 (m, 2H), 1.79-1.96 (m, 2H), 2.19 (s, 3H), 3.23 (s, 3H), 3.37 (t, 2H), 7.31 (d, 1H), 7.39 (d, 1H), 7.48-7.57 (m, 2H), 7.64 (t, 1H), 7.70 (dd, 1H), 8.16 (s, 1H), 10.74 (s, 1H).

LC-MS (Method 3): Rₜ=1.28 min; MS (ESIpos): m/z=444 [M+H]⁺.

### Example 1-115

### =Compound of Table 1, p. 41 in combination with Table 2, p. 44 of WO08/067,911

### 3-(3′,4′-Difluoro-4-methylbiphenyl-3-yl)-4-hydroxy-1-azaspiro[4.5]dec-3-en-2-one

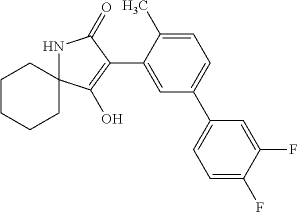 [SMILES: Cc1ccc(-c2ccc(F)c(F)c2)cc1C1=C(O)C2(CCCCC2)NC1=O]

The title compound was prepared analogously to the synthesis of the compound from Example 1-93 starting with 150 mg (0.45 mmol) of the compound from Example 45A. This gave 49.3 mg (30% of theory) of the title compound.

¹H-NMR (400 MHz, DMSO-d₆): δ [ppm]=1.13-1.25 (m, 1H), 1.34-1.43 (m, 2H), 1.54-1.71 (m, 5H), 1.78-1.90 (m, 2H), 2.18 (s, 3H), 7.29 (d, 1H), 7.36 (d, 1H), 7.46-7.52 (m, 3H), 7.67-7.74 (m, 1H), 8.14 (s, 1H), 10.76 (s, 1H).

LC-MS (Method 1): Rₜ=1.24 min; MS (ESIpos): m/z=370 [M+H]⁺.

### Example 1-116

### =Compound I-1-a-5 of WO07/048,545

### (5s,8s)-3-(4′-Chloro-4 methylbiphenyl-3-yl)-4-hydroxy-8-(methoxymethyl)-1-azaspiro[4.5]dec-3-en-2-one

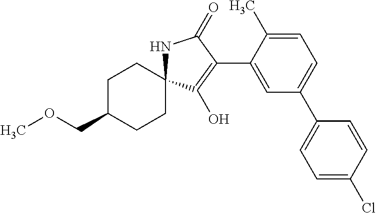 [SMILES: COC[C@H]1CC[C@]2(CC1)NC(=O)C(c1cc(-c3ccc(Cl)cc3)ccc1C)=C2O]

5.26 g (46.9 mmol) of potassium tert-butoxide were added to 10.4 g (23.4 mmol) of the compound from Example 37A in 35 ml of N,N-dimethylformamide. The reaction mixture was heated at 80° C. for 15 minutes. After cooling, the mixture was concentrated and the residue was taken up in water and added dropwise to 2N aqueous hydrogen chloride solution. The precipitate was filtered off with suction, washed with water and dried. This gave 9.3 g of the title compound.

¹H-NMR (300 MHz, DMSO-d₆): δ [ppm]=1.21-1.47 (m, 4H), 1.48-1.64 (m, 1H), 1.65-1.78 (m, 2H), 1.81-1.97 (m, 2H), 2.18 (s, 3H), 3.16 (d, 2H), 3.24 (s, 3H), 7.30 (d, 1H), 7.34 (d, 1H), 7.44-7.53 (m, 3H), 7.61-7.68 (m, 2H), 8.13 (s, 1H), 10.77 (s, 1H).

LC-MS (Method 2): Rₜ=1.25 min; MS (ESIpos): m/z=412 [M+H]⁺.

### Example 1-117

### =Compound According to WO07/048,545

### (5s,8s)-3-(4′-Chloro-4 methylbiphenyl-3-yl)-4-hydroxy-8-(hydroxymethyl)-1-azaspiro[4.5]dec-3-en-2-one

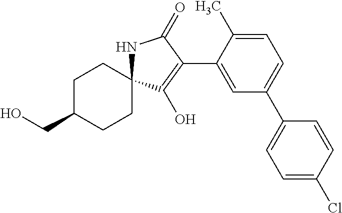 [SMILES: Cc1ccc(-c2ccc(Cl)cc2)cc1C1=C(O)[C@]2(CC[C@H](CO)CC2)NC1=O]

Under argon, a solution of 1.23 ml (9.71 mmol) of trimethylsilyl chloride in 1.5 ml of acetonitrile was slowly added dropwise to 2.00 g (4.86 mmol) of the compound from Example 1-116 and 1.46 g (9.71 mmol) of sodium iodide in 50 ml of acetonitrile. The reaction mixture was heated under reflux overnight. After cooling, the mixture was filtered and concentrated, the residue was dissolved in ethyl acetate and the solution was washed with water, dried over magnesium sulphate, filtered and concentrated. The crude product was purified by chromatography on silica gel (mobile phase: hexane/ethyl acetate gradient). Water and 2N aqueous sodium hydroxide solution were then added and the mixture was stirred, acidified with 2N aqueous hydrogen chloride solution, filtered off with suction, washed with water and dried. This gave 1.13 g (57% of theory) of the title compound.

¹H-NMR (300 MHz, DMSO-d₆): δ [ppm]=1.15-1.50 (m, 5H), 1.67-1.80 (m, 2H), 1.81-1.95 (m, 2H), 2.19 (s, 3H), 3.24 (s, 2H), 7.30 (d, 1H), 7.34 (d, 1H), 7.46-7.53 (m, 3H), 7.62-7.68 (m, 2H), 8.11 (s, 1H), 10.75 (s, 1H).

LC-MS (Method 1): Rₜ=1.13 min; MS (ESIpos): m/z=398 [M+H]⁺.

### Example 1-118

### =Compound of Table 1, p. 26 of WO08/067,910

### (5s,8s)-3-(4′-Chloro-3′-fluoro-4-methylbiphenyl-3-yl)-4-hydroxy-8-methoxy-1-azaspiro[4.5]dec-3-en-2-one

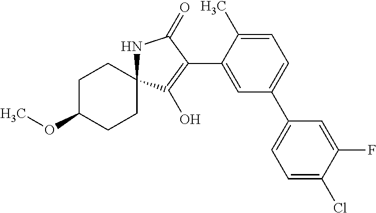 [SMILES: CO[C@H]1CC[C@]2(CC1)NC(=O)C(c1cc(-c3ccc(Cl)c(F)c3)ccc1C)=C2O]

Under nitrogen, 4.32 g (38.5 mmol) of potassium tert-butoxide were added to 15.7 g (35.0 mmol) of the compound from Example 39A in 60 ml of N,N-dimethylformamide. The reaction mixture was stirred at room temperature for 20 minutes. The reaction mixture was then added to ice-water, 160 ml 1N aqueous hydrogen chloride solution were added dropwise, the mixture was stirred for 30 minutes, filtered off with suction and the precipitate was washed with water and dried. This gave 14.2 g (97% of theory) of the title compound.

¹H-NMR (300 MHz, DMSO-d₆): δ [ppm]=1.40-1.62 (m, 4H), 1.85-2.04 (m, 4H), 2.19 (s, 3H), 3.07-3.20 (m, 1H), 3.27 (s, 3H), 7.31 (d, 1H), 7.39 (d, 1H), 7.48-7.57 (m, 2H), 7.60-7.73 (m, 2H), 8.20 (s, 1H), 10.82 (s, 1H).

LC-MS (Method 1): Rₜ=1.22 min; MS (ESIpos): m/z=416 [M+H]⁺.

### Example 1-119

### =Compound According to WO99/48869

### (5s,8s)-3-(4′-Chloro-4 methylbiphenyl-3-yl)-4-hydroxy-8-(trifluoromethyl)-1-azaspiro[4.5]dec-3-en-2-one

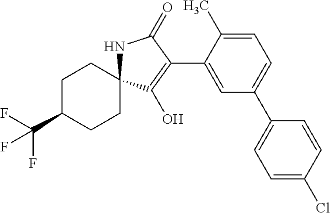 [SMILES: Cc1ccc(-c2ccc(Cl)cc2)cc1C1=C(O)[C@]2(CC[C@H](C(F)(F)F)CC2)NC1=O]

3.05 g (27.18 mmol) of potassium tert-butoxide were added to 6.36 g (13.59 mmol) of methyl cis-1-{[(4′-chloro-4-methylbiphenyl-3-yl)acetyl]amino}-4-(trifluoromethyl)cyclohexanecarboxylate (Example 2A) in 68 ml of N,N-dimethylformamide. The reaction mixture was stirred at 80° C. for 60 minutes. For work-up, the cold reaction mixture was poured onto 800 ml of ice-water and acidified with aqueous hydrochloric acid. The crude product was filtered off, dried and purified by chromatography on silica gel (hexane/ethyl acetate gradient). Evaporation gave 4.1 g (69% of theory) of the title compound.

¹H-NMR (400 MHz, DMSO-d₆): δ [ppm]=1.40-1.55 (m, 2H), 1.58-1.77 (m, 2H), 1.78-2.02 (m, 4H), 2.15 (s, 3H), 2.17-2.30 (m, 1H), 7.27 (d, 1H), 7.32 (d, 1H), 7.42-7.51 (m, 3H), 7.58-7.66 (m, 2H), 8.29 (s, 1H), 10.90 (s, 1H).

LC-MS (Method 3): Rₜ=1.32 min; MS (ESIpos): m/z=436 [M+H]⁺.

### Example 1-120

### =Compound According to WO99/48869

### (5s,8s)-3-(4,4′-Dichlorobiphenyl-3-yl)-4-hydroxy-8-methoxy-1-azaspiro[4.5]dec-3-en-2-one

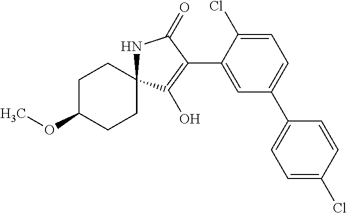 [SMILES: CO[C@H]1CC[C@]2(CC1)NC(=O)C(c1cc(-c3ccc(Cl)cc3)ccc1Cl)=C2O]

13.90 g (123.46 mmol) of potassium tert-butoxide were added to 27.80 g (61.73 mmol) of methyl cis-1-{[(4,4′-dichlorobiphenyl-3-yl)acetyl]amino}-4-methoxycyclohexanecarboxylate (Example 4A) in 310 ml of N,N-dimethylformamide. The reaction mixture was stirred at 80° C. for 60 minutes. For work-up, the cold reaction mixture was poured onto 4 l of ice-water and acidified with aqueous hydrochloric acid. The crude product was filtered off, dried and purified by trituration with diethyl ether. This gave 24.09 g (93% of theory) of the title compound.

¹H-NMR (400 MHz, DMSO-d₆): δ [ppm]=1.34-1.61 (m, 4H), 1.76-2.04 (m, 4H), 3.02-3.18 (m, 1H), 3.23 (s, 3H), 7.44-7.53 (m, 4H), 7.57 (dd, 1H), 7.62-7.70 (m, 2H), 8.18 (s, 1H), 11.05 (s, 1H).

LC-MS (Method 2): Rₜ=1.17 min; MS (ESIpos): m/z=418 [M+H]⁺.

### Example 1-121

### =Compound According to WO99/48869

### (5s,8s)-3-(4′-Chloro-2,4-dimethylbiphenyl-3-yl)-4-hydroxy-8-methoxy-1-azaspiro[4.5]dec-3-en-2-one

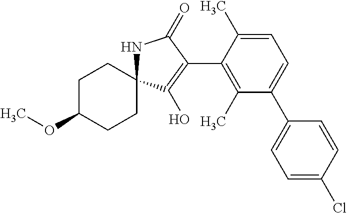 [SMILES: CO[C@H]1CC[C@]2(CC1)NC(=O)C(c1c(C)ccc(-c3ccc(Cl)cc3)c1C)=C2O]

Under argon, 43 mg (0.053 mmol) of dichloro[1,1′-bis(diphenylphosphino)ferrocene]palladium dichloromethane complex were added to 200 mg (0.53 mmol) of (5s,8s)-3-(3 bromo 2,6-dimethylphenyl)-4-hydroxy-8-methoxy-1-azaspiro[4.5]dec-3-en-2-one (Example 7A) in 23 ml of degassed 1,2-dimethoxyethane. The mixture was stirred at room temperature for 5 minutes, and 123 mg (0.79 mmol) of (4-chlorophenyl)boronic acid and a solution of 600 mg (1.84 mmol) of caesium carbonate in 1.4 ml of degassed water were then added. In a closed vessel, the reaction mixture was heated at 150° C. under microwave irradiation for 10 minutes. After cooling, 500 μl of concentrated aqueous hydrogen chloride solution and magnesium sulphate were added, the mixture was filtered off through silica gel and magnesium sulphate, the filter cake was washed with ethyl acetate and the filtrate was concentrated. The crude product was purified by preparative HPLC (C18 phase, mobile phase: water/acetonitrile gradient/0.1% formic acid), giving 65 mg (30% of theory) of the title compound.

¹H-NMR (300 MHz, DMSO-d₆): δ [ppm]=1.33-1.60 (m, 4H), 1.79-1.99 (m, 4H), 1.93 (s, 3H), 2.08 (s, 3H), 3.03-3.17 (m, 1H), 3.22 (s, 3H), 7.00 (d, 1H), 7.08 (d, 1H), 7.26 (“d”, 2H), 7.45 (“d”, 2H), 8.10 (s, 1H), 10.70 (s, 1H).

LC-MS (Method 2): Rₜ=1.23 min; MS (ESIpos): m/z=412 [M+H]⁺.

### Example 1-122

### (5r,8r)-3-(4′-Chloro-4-methylbiphenyl-3-yl)-4,8-dihydroxy-8-(trifluoromethyl)-1-azaspiro[4.5]dec-3-en-2-one

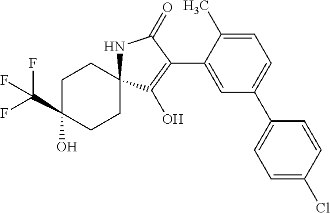 [SMILES: Cc1ccc(-c2ccc(Cl)cc2)cc1C1=C(O)[C@]2(CC[C@](O)(C(F)(F)F)CC2)NC1=O]

Under argon, 24 mg (0.030 mmol) of dichloro[1,1′-bis(diphenylphosphino)ferrocene]palladium dichloromethane complex were added to 125 mg (0.30 mmol) of (5r,8r)-3-(5 bromo-2-methylphenyl)-4,8-dihydroxy-8-(trifluoromethyl)-1-azaspiro[4.5]dec-3-en-2-one (Example 12A) in 13 ml of degassed 1,2-dimethoxyethane. The mixture was stirred at room temperature for 5 minutes, and 70 mg (0.45 mmol) of (4-chlorophenyl)boronic acid and a solution of 339 mg (1.04 mmol) of caesium carbonate in 815 μl degassed water were then added. In a closed vessel, the reaction mixture was heated at 150° C. under microwave irradiation for 10 minutes. After cooling, 100 μl of concentrated aqueous hydrogen chloride solution were added and the mixture was concentrated under reduced pressure. The residue was taken up in dichloromethane and washed with aqueous 5% strength citric acid (pH=4.0-4.5) and water. The organic phase was dried over sodium sulphate and evaporated. The crude product was purified by chromatography on silica gel (mobile phase: hexane/ethyl acetate gradient) and by HPLC chromatography (C18 phase, mobile phase: water/acetonitrile gradient/0.1% formic acid), giving 17.4 mg (13% of theory) of the title compound.

¹H-NMR (400 MHz, methanol-d₄): δ [ppm]=1.41-1.53 (m, 2H), 1.85-2.01 (m, 4H), 2.24 (s, 3H), 2.33-2.46 (m, 2H), 7.32 (d, 1H), 7.35-7.42 (m, 3H), 7.46 (dd, 1H), 7.58 (“d”, 2H).

LC-MS (Method 1): Rₜ=1.19 min; MS (ESIpos): m/z=452 [M+H]⁺.

### Example 1-123

### (5r,8r)-3-(4′-Chloro-3′-fluoro-4-methylbiphenyl-3-yl)-4,8-dihydroxy-8-(trifluoromethyl)-1-azaspiro[4.5]dec-3-en-2-one

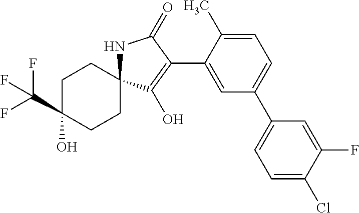 [SMILES: Cc1ccc(-c2ccc(Cl)c(F)c2)cc1C1=C(O)[C@]2(CC[C@](O)(C(F)(F)F)CC2)NC1=O]

Under argon, 24 mg (0.030 mmol) of dichloro[1,1′-bis(diphenylphosphino)ferrocene]palladium dichloromethane complex were added to 125 mg (0.30 mmol) of (5r,8r)-3-(5 bromo-2-methylphenyl)-4,8-dihydroxy-8-(trifluoromethyl)-1-azaspiro[4.5]dec-3-en-2-one (Example 12A) in 13 ml of degassed 1,2-dimethoxyethane. The mixture was stirred at room temperature for 5 minutes, and 78 mg (0.45 mmol) of (4-chloro-3-fluorophenyl)boronic acid and a solution of 340 mg (1.04 mmol) of caesium carbonate in 815 μl degassed water were then added. In a closed vessel, the reaction mixture was heated at 150° C. under microwave irradiation for 10 minutes. After cooling, 100 μl of concentrated aqueous hydrogen chloride solution were added and the mixture was concentrated under reduced pressure. The residue was taken up in dichloromethane and washed with aqueous 5% strength citric acid (pH=4.0-4.5) and water. The organic phase was dried over sodium sulphate and evaporated. The crude product was purified by chromatography on silica gel (mobile phase: hexane/ethyl acetate gradient), giving 68 mg (49% of theory) of the title compound.

¹H-NMR (300 MHz, methanol-d₄): δ [ppm]=1.41-1.56 (m, 2H), 1.85-1.99 (m, 4H), 2.25 (s, 3H), 2.32-2.48 (m, 2H), 7.10-7.25 (m, 1H), 7.34 (d, 1H), 7.40 (dd, 1H), 7.43-7.56 (m, 3H).

LC-MS (Method 3): Rₜ=1.19 min; MS (ESIpos): m/z=470 [M+H]⁺.

### Example 1-124

### (5r,8r)-3-(3′,4′-Difluoro-4-methylbiphenyl-3-yl)-4,8-dihydroxy-8-(trifluoromethyl)-1-azaspiro[4.5]dec-3-en-2-one

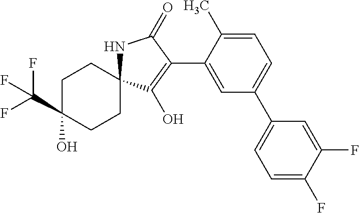 [SMILES: Cc1ccc(-c2ccc(F)c(F)c2)cc1C1=C(O)[C@]2(CC[C@](O)(C(F)(F)F)CC2)NC1=O]

Under argon, 23 mg (0.030 mmol) of dichloro[1,1′-bis(diphenylphosphino)ferrocene]palladium dichloromethane complex were added to 120 mg (0.29 mmol) of (5r,8r)-3-(5-Bromo-2-methylphenyl)-4,8-dihydroxy-8-(trifluoromethyl)-1-azaspiro[4.5]dec-3-en-2-one (Example 12A) in 13 ml of degassed 1,2-dimethoxyethane. The mixture was stirred at room temperature for 5 minutes, and 68 mg (0.43 mmol) of (3,4-difluorophenyl)boronic acid and a solution of 326 mg (1.00 mmol) of caesium carbonate in 780 μl degassed water were then added. In a closed vessel, the reaction mixture was heated at 150° C. under microwave irradiation for 10 minutes. After cooling, 100 μl of concentrated aqueous hydrogen chloride solution were added and the mixture was concentrated under reduced pressure. The residue was taken up in dichloromethane and washed with aqueous 5% strength citric acid (pH=4.0-4.5) and water. The organic phase was dried over sodium sulphate and evaporated. The crude product was purified by chromatography on silica gel (mobile phase: hexane/ethyl acetate gradient), giving 58 mg (45% of theory) of the title compound.

¹H-NMR (300 MHz, methanol-d₄): δ [ppm]=1.42-1.56 (m, 2H), 1.83-2.00 (m, 4H), 2.25 (s, 3H), 2.32-2.49 (m, 2H), 7.22-7.56 (m, 6H).

LC-MS (Method 3): Rₜ=1.13 min; MS (ESIpos): m/z=454 [M+H]⁺.

### Example 1-125

### (5r,8r)-3-(4′-Chloro-2,4-dimethylbiphenyl-3-yl)-4,8-dihydroxy-8-(trifluoromethyl)-1-azaspiro[4.5]dec-3-en-2-one

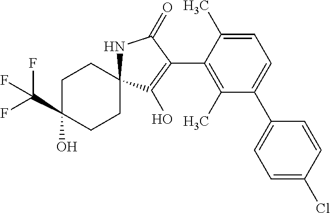 [SMILES: Cc1ccc(-c2ccc(Cl)cc2)c(C)c1C1=C(O)[C@]2(CC[C@](O)(C(F)(F)F)CC2)NC1=O]

Under argon, 8.3 mg (0.010 mmol) of dichloro[1,1′-bis(diphenylphosphino)ferrocene]palladium dichloromethane complex were added to 44 mg (0.10 mmol) of (5r,8r)-3-(3 bromo-2,6-dimethylphenyl)-4,8-dihydroxy-8-(trifluoromethyl)-1-azaspiro[4.5]dec-3-en-2-one (Example 16A) in 4.4 ml of degassed 1,2-dimethoxyethane. The mixture was stirred at room temperature for 5 minutes, and 24 mg (0.15 mmol) of (4-chlorophenyl)boronic acid and a solution of 116 mg (0.36 mmol) of caesium carbonate in 280 μl degassed water were then added. In a closed vessel, the reaction mixture was heated at 150° C. under microwave irradiation for 10 minutes. After cooling, 100 μl of concentrated aqueous hydrogen chloride solution were added and the mixture was concentrated under reduced pressure. The residue was taken up in dichloromethane and washed with aqueous 5% strength citric acid (pH=4.0-4.5) and water. The organic phase was dried over sodium sulphate and evaporated. The crude product was purified by chromatography on silica gel (mobile phase: hexane/ethyl acetate gradient), giving 8.6 mg (18% of theory) of the title compound.

¹H-NMR (300 MHz, methanol-d₄): δ [ppm]=1.39-1.53 (m, 2H), 1.84-1.98 (m, 4H), 2.03 (s, 3H), 2.19 (s, 3H), 2.31-2.48 (m, 2H), 7.05 (d, 1H), 7.13 (d, 1H), 7.25 (“d”, 2H), 7.38 (“d”, 2H).

LC-MS (Method 1): Rₜ=1.21 min; MS (ESIpos): m/z=466 [M+H]⁺.

### Example 1-126

### (5r,8r)-3-(4′-Chloro-3′-fluoro-2,4-dimethylbiphenyl-3-yl)-4,8-dihydroxy-8-(trifluoromethyl)-1-azaspiro[4.5]dec-3-en-2-one

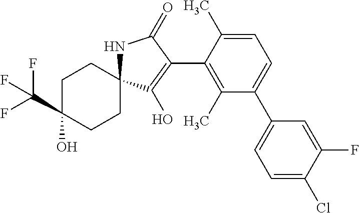 [SMILES: Cc1ccc(-c2ccc(Cl)c(F)c2)c(C)c1C1=C(O)[C@]2(CC[C@](O)(C(F)(F)F)CC2)NC1=O]

Under argon, 28 mg (0.034 mmol) of dichloro[1,1′-bis(diphenylphosphino)ferrocene]palladium dichloromethane complex were added to 149 mg (0.34 mmol) of (5r,8r)-3-(3 bromo-2,6-dimethylphenyl)-4,8-dihydroxy-8-(trifluoromethyl)-1-azaspiro[4.5]dec-3-en-2-one (Example 16A) in 15 ml of degassed 1,2-dimethoxyethane. The mixture was stirred at room temperature for 5 minutes, and 90 mg (0.52 mmol) of (4-chloro-3-fluorophenyl)boronic acid and a solution of 391 mg (1.20 mmol) of caesium carbonate in 940 μl degassed water were then added. In a closed vessel, the reaction mixture was heated at 150° C. under microwave irradiation for 10 minutes. After cooling, 100 μl of concentrated aqueous hydrogen chloride solution were added and the mixture was concentrated under reduced pressure. The residue was taken up in dichloromethane and washed with aqueous 5% strength citric acid (pH=4.0-4.5) and water. The organic phase was dried over sodium sulphate and evaporated. The crude product was purified by chromatography on silica gel (mobile phase: hexane/ethyl acetate gradient), giving 107 mg (64% of theory) of the title compound.

¹H-NMR (300 MHz, methanol-d₄): δ [ppm]=1.40-1.55 (m, 2H), 1.85-2.00 (m, 4H), 2.06 (s, 3H), 2.21 (s, 3H), 2.32-2.50 (m, 2H), 7.05-7.12 (m, 2H), 7.13-7.21 (m, 2H), 7.48 (t, 1H).

LC-MS (Method 3): Rₜ=1.22 min; MS (ESIpos): m/z=484 [M+H]⁺.

### Example 1-127

### (5r,8r)-3-(4′-Chloro-6-fluoro-4-methylbiphenyl-3-yl)-4,8-dihydroxy-8-(trifluoromethyl)-1-azaspiro[4.5]dec-3-en-2-one

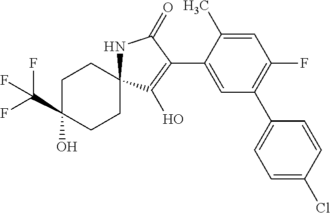 [SMILES: Cc1cc(F)c(-c2ccc(Cl)cc2)cc1C1=C(O)[C@]2(CC[C@](O)(C(F)(F)F)CC2)NC1=O]

Under argon, 36 mg (0.044 mmol) of dichloro[1,1′-bis(diphenylphosphino)ferrocene]palladium dichloromethane complex were added to 194 mg (0.44 mmol) of (5r,8r)-3-(5 bromo-4-fluoro-2-methylphenyl)-4,8-dihydroxy-8-(trifluoromethyl)-1-azaspiro[4.5]dec-3-en-2-one (Example 21A) in 19 ml of degassed 1,2-dimethoxyethane. The mixture was stirred at room temperature for 5 minutes, and 104 mg (0.66 mmol) of (4-chloro-phenyl)boronic acid and a solution of 505 mg (1.55 mmol) of caesium carbonate in 1200 μl degassed water were then added. In a closed vessel, the reaction mixture was heated at 150° C. under microwave irradiation for 10 minutes. After cooling, 150 μl of concentrated aqueous hydrogen chloride solution were added and the mixture was concentrated under reduced pressure. The residue was taken up in dichloromethane and washed with aqueous 5% strength citric acid (pH=4.0-4.5) and water. The organic phase was dried over sodium sulphate and evaporated. The crude product was purified by chromatography on silica gel (mobile phase: hexane/ethyl acetate gradient) and by HPLC chromatography (C18 phase, mobile phase: water/acetonitrile gradient/0.1% formic acid), giving 60 mg (29% of theory) of the title compound.

¹H-NMR (300 MHz, methanol-d₄): δ [ppm]=1.40-1.51 (m, 2H), 1.83-2.01 (m, 4H), 2.23 (s, 3H), 2.30-2.46 (m, 2H), 7.08 (d, 1H), 7.21 (d, 1H), 7.37-7.43 (m, 2H), 7.49-7.56 (m, 2H).

LC-MS (Method 1): Rₜ=1.21 min; MS (ESIpos): m/z=470 [M+H]⁺.

### Example 1-128

### =Compound According to WO08/067,911

### (5s,8s)-3-(3′,4′-Difluoro-4-methylbiphenyl-3-yl)-4-hydroxy-8-(trifluoromethyl)-1-azaspiro[4.5]dec-3-en-2-one

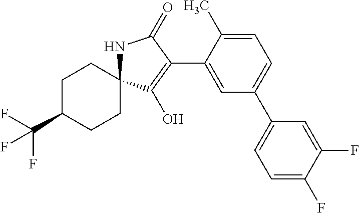 [SMILES: Cc1ccc(-c2ccc(F)c(F)c2)cc1C1=C(O)[C@]2(CC[C@H](C(F)(F)F)CC2)NC1=O]

3.14 g (28.00 mmol) of potassium tert-butoxide were added to 6.57 g (14.00 mmol) of methyl cis-1-{[(3′,4′-difluoro-4-methylbiphenyl-3-yl)acetyl]amino}-4-(trifluoromethyl)cyclohexane-carboxylate (Example 24A) in 70 ml of N,N-dimethylformamide. The reaction mixture was stirred at 80° C. for 60 minutes. For work-up, the cold reaction mixture was poured onto 1.21 of ice-water and acidified with aqueous hydrochloric acid. The crude product was filtered off and dried. The crude product was purified by HPLC chromatography (C18 phase, mobile phase: water/acetonitrile gradient/0.2% ammonia). To release the acid, the residue obtained was dissolved in 500 ml 26 mmolar aqueous sodium hydroxide solution, acidified with aqueous 1 N hydrochloric acid, washed with water, filtered off and dried. For further purification, the product was chromatographed on silica gel (mobile phase: hexane/ethyl acetate gradient). The product obtained in this manner was once more dissolved in 500 ml 26 mmolar aqueous sodium hydroxide solution, acidified with aqueous 1 N hydrochloric acid, washed with water, filtered off and dried. This gave 2.18 g (36% of theory) of the title compound.

¹H-NMR (300 MHz, DMSO-d₆): δ [ppm]=1.42-1.54 (m, 2H), 1.57-1.75 (m, 2H), 1.76-2.02 (m, 4H), 2.15 (s, 3H), 2.19-2.29 (m, 1H), 7.26 (d, 1H), 7.33 (d, 1H), 7.41-7.52 (m, 3H), 7.61-7.72 (m, 1H), 8.27 (s, 1H), 10.91 (s, 1H).

LC-MS (Method 3): Rₜ=1.26 min; MS (ESIpos): m/z=438 [M+H]⁺.

### Example 1-129

### =Compound According to WO08/067,911

### (5r,8r)-3-(3′,4′-Difluoro-4-methylbiphenyl-3-yl)-4-hydroxy-8-(trifluoromethyl)-1-azaspiro[4.5]dec-3-en-2-one

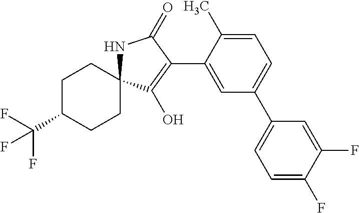 [SMILES: Cc1ccc(-c2ccc(F)c(F)c2)cc1C1=C(O)[C@]2(CC[C@@H](C(F)(F)F)CC2)NC1=O]

The title compound was obtained as minor component in the HPLC chromatography (C18 phase, mobile phase: water/acetonitrile gradient/0.2% ammonia) of (5s,8s)-3-(3′,4′-difluoro-4-methylbiphenyl-3-yl)-4-hydroxy-8 (trifluoromethyl)-1-azaspiro[4.5]dec-3-en-2-one (Example 1-128) as ammonium salt. To release the acid, 906 mg of the ammonium salt were dissolved in 170 ml 26 mmolar aqueous sodium hydroxide solution, acidified with aqueous 1 N hydrochloric acid, washed with water, filtered off and dried. This gave 855 mg of the title compound.

¹H-NMR (300 MHz, DMSO-d₆): δ [ppm]=1.54-1.71 (m, 2H), 1.76-2.07 (m, 6H), 2.15 (s, 3H), 2.26-2.42 (m, 1H), 7.27 (d, 1H), 7.32 (d, 1H), 7.41-7.53 (m, 3H), 7.61-7.80 (m, 2H), 10.90 (s, 1H).

LC-MS (Method 3): Rₜ=1.30 min; MS (ESIpos): m/z=438 [M+H]⁺.

### Example 1-130

### (5r,8r)-3-(4′-Chloro-3′-fluoro-4-methylbiphenyl-3-yl)-4,8-dihydroxy-8-(pentafluoroethyl)-1-azaspiro[4.5]dec-3-en-2-one

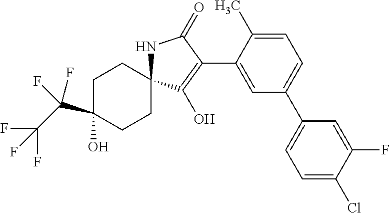 [SMILES: Cc1ccc(-c2ccc(Cl)c(F)c2)cc1C1=C(O)[C@]2(CC[C@](O)(C(F)(F)C(F)(F)F)CC2)NC1=O]

Under argon, 43 mg (0.052 mmol) of dichloro[1,1′-bis(diphenylphosphino)ferrocene]palladium dichloromethane complex were added to 245 mg (0.52 mmol) of (5r,8r)-3-(5 bromo-2-methylphenyl)-4,8-dihydroxy-8-(pentafluoroethyl)-1-azaspiro[4.5]dec-3-en-2-one (Example 25A) in 23 ml of degassed 1,2-dimethoxyethane. The mixture was stirred at room temperature for 5 minutes, and 136 mg (0.78 mmol) of (4-chloro-3-fluorophenyl)boronic acid and a solution of 594 mg (1.82 mmol) of caesium carbonate in 800 μl degassed water were then added. In a closed vessel, the reaction mixture was heated at 150° C. under microwave irradiation for 10 minutes. After cooling, 100 μl of concentrated aqueous hydrogen chloride solution were added and the mixture was concentrated under reduced pressure. The residue was taken up in dichloromethane and washed with aqueous 5% strength citric acid (pH=4.0-4.5) and water. The organic phase was dried over sodium sulphate and evaporated. The crude product was purified by chromatography on silica gel (mobile phase: hexane/ethyl acetate gradient) and by HPLC chromatography (C18 phase, mobile phase: water/acetonitrile gradient/0.1% formic acid), giving 57 mg (21% of theory) of the title compound.

¹H-NMR (300 MHz, DMSO-d₆): δ [ppm]=1.23-1.37 (m, 2H), 1.72-2.00 (m, 4H), 2.15 (s, 3H), 2.17-2.33 (m, 2H), 5.87 (s, 1H), 7.29 (d, 1H), 7.36 (d, 1H), 7.46-7.55 (m, 2H), 7.61 (t, 1H), 7.67 (dd, 1H), 8.36 (s, 1H), 10.95 (s, 1H).

LC-MS (Method 3): Rₜ=1.26 min; MS (ESIpos): m/z=520 [M+H]⁺.

### Example 1-131

### (5r,8r)-3-(4′-Chloro-6-fluoro-4-methylbiphenyl-3-yl)-4,8-dihydroxy-8-(pentafluoroethyl)-1-azaspiro[4.5]dec-3-en-2-one

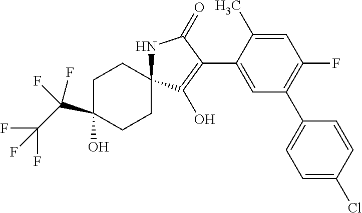 [SMILES: Cc1cc(F)c(-c2ccc(Cl)cc2)cc1C1=C(O)[C@]2(CC[C@](O)(C(F)(F)C(F)(F)F)CC2)NC1=O]

Under argon, 23 mg (0.028 mmol) of dichloro[1,1′-bis(diphenylphosphino)ferrocene]palladium dichloromethane complex were added to 139 mg (0.29 mmol) of (5r,8r)-3-(5 bromo-4-fluoro-2-methylphenyl)-4,8-dihydroxy-8-(pentafluoroethyl)-1-azaspiro[4.5]dec-3-en-2-one (Example 26A) in 13 ml of degassed 1,2-dimethoxyethane. The mixture was stirred at room temperature for 5 minutes, and 67 mg (0.43 mmol) of (4-chlorophenyl)boronic acid and a solution of 325 mg (1.00 mmol) of caesium carbonate in 800 μl degassed water were then added. In a closed vessel, the reaction mixture was heated at 150° C. under microwave irradiation for 13 minutes. After cooling, 100 μl of concentrated aqueous hydrogen chloride solution were added and the mixture was concentrated under reduced pressure. The residue was taken up in dichloromethane and washed with aqueous 5% strength citric acid (pH=4.0-4.5) and water. The organic phase was dried over sodium sulphate and evaporated. The crude product was purified by chromatography on silica gel (mobile phase: hexane/ethyl acetate gradient) and by HPLC chromatography (C18 phase, mobile phase: water/acetonitrile gradient/0.1% formic acid), giving 4 mg (3% of theory) of the title compound.

¹H-NMR (300 MHz, methanol-d₄): δ [ppm]=1.40-1.53 (m, 2H), 1.93-2.04 (m, 4H), 2.24 (s, 3H), 2.31-2.50 (m, 2H), 7.10 (d, 1H), 7.21 (d, 1H), 7.38-7.45 (m, 2H), 7.49-7.57 (m, 2H).

LC-MS (Method 3): Rₜ=1.28 min; MS (ESIpos): m/z=520 [M+H]⁺.

### Example 1-132

### =Compound of Table 1, p. 69 in combination with Table 5, p. 71 of WO99/48869

### (5s,8s)-3-(4′-Chloro-4,6-dimethylbiphenyl-3-yl)-4-hydroxy-8-methoxy-1-azaspiro[4.5]dec-3-en-2-one

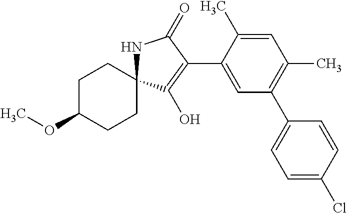 [SMILES: CO[C@H]1CC[C@]2(CC1)NC(=O)C(c1cc(-c3ccc(Cl)cc3)c(C)cc1C)=C2O]

7.00 g (55 mmol) of potassium tert-butoxide were initially charged in 18 ml of N,N-dimethylformamide (DMF). At 60° C., 11.13 g (26.8 mmol) of the compound from Example 54A in 23 ml of DMF were added dropwise, and the mixture was stirred at 80° C. for 1 h. The reaction mixture was poured onto ice-water, acidified with dilute hydrochloric acid and filtered off with suction, and the filter residue was rinsed and dried. The residue was chromatographed on silica gel using methylene chloride/ethyl acetate (3:2). This gave 5.67 g (51% of theory) of the title compound.

¹H-NMR (400 MHz, DMSO-d₆): δ [ppm]=1.40-1.55 (m, 4H), 1.87-1.99 (m, 4H), 2.15 (s, 3H), 2.21 (s, 3H), 3.08-3.16 (m, 1H), 3.26 (s, 3H), 6.90 (s, 1H), 7.24 (s, 1H), 7.34-7.36 (m, 2H), 7.47-7.49 (m, 2H), 8.14 (s, 1H).

LC-MS (Method 1): Rₜ=1.26 min; MS (ESIpos): m/z=412 [M+H]⁺.

### Example 1-133

### =Compound According to WO99/48869

### (5S,7S)-3 (4′-Chloro-4 methylbiphenyl-3-yl)-4-hydroxy-7-(trifluoromethyl)-1-azaspiro[4.5]dec-3-en-2-one

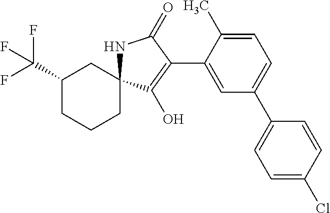 [SMILES: Cc1ccc(-c2ccc(Cl)cc2)cc1C1=C(O)[C@@]2(CCC[C@H](C(F)(F)F)C2)NC1=O]

1.30 g (27 mmol) of potassium tert-butoxide were initially charged in 4 ml of N,N-dimethylformamide (DMF). At 20-40° C., 2.33 g (5 mmol) of the compound from Example 55A in 5 ml DMF were added dropwise, and the mixture was stirred at 40° C. for 1 h. The reaction mixture was poured onto ice-water, acidified with dilute hydrochloric acid, filtered off with suction, rinsed and dried. The residue was chromatographed on silica gel using methylene chloride/acetone (5:1). This gave 0.9 g (41% of theory) of the title compound.

¹H-NMR (400 MHz, DMSO-d₆): δ [ppm]=1.24-1.26 (m, 1H), 1.42-1.45 (m, 1H), 1.56-1.59 (m, 1H), 1.67-1.70 (m, 1H), 1.83-1.94 (m, 4H), 2.19 (s, 3H), 2.67-2.70 (m, 1H), 7.30-7.32 (m, 1H), 7.38-7.39 (m, 1H), 7.49-7.51 (m, 3H), 7.65-7.68 (m, 2H), 8.23 (s, 1H).

LC-MS (Method 1): Rₜ=1.35 min; MS (ESIpos): m/z=436 [M+H]⁺.

### Example 1-134

### =Compound According to WO99/48869

### (5s,8s)-3-(4,4′-Dichlorobiphenyl-3-yl)-4-hydroxy-8 (trifluoromethyl)-1-azaspiro[4.5]dec-3-en-2-one

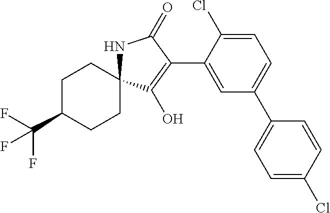 [SMILES: O=C1N[C@]2(CC[C@@H](C(F)(F)F)CC2)C(O)=C1c1cc(-c2ccc(Cl)cc2)ccc1Cl]

1.67 g (36 mmol) of potassium tert-butoxide were initially charged in 5 ml of N,N-dimethylformamide

(DMF). At 20-40° C., 3.13 g (6.4 mmol) of the compound from Example 56A in 9 ml DMF were added dropwise, and the mixture was stirred at 40° C. for 1 h. The reaction mixture was poured onto ice-water, acidified with dilute hydrochloric acid, filtered off with suction, rinsed and dried. The residue was chromatographed on silica gel using methylene chloride/acetone (5:1). This gave 1.77 g (60% of theory) of the title compound.

¹H-NMR (400 MHz, DMSO-d₆): δ [ppm]=1.50-1.53 (m, 2H), 1.68-1.74 (m, 2H), 1.85-1.98 (m, 4H), 2.23-2.34 (m, 1H), 7.50-7.56 (m, 4H), 7.61-7.63 (m, 1H), 7.69-7.71 (m, 1H), 7.73-7.78 (m, 1H), 8.37 (s, 1H).

LC-MS (Method 1): Rₜ=1.32 min; MS (ESIpos): m/z=456 [M+H]⁺.

### Example 1-135

### =Compound According to WO99/48869

### (5s,8s)-3-(4′-Chloro 6 fluoro 4-methylbiphenyl-3-yl)-4-hydroxy-8-methoxy-1-azaspiro[4.5]dec-3-en-2-one

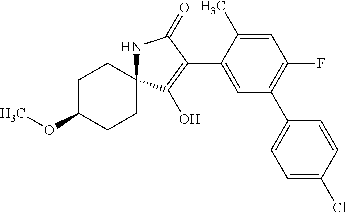 [SMILES: CO[C@H]1CC[C@]2(CC1)NC(=O)C(c1cc(-c3ccc(Cl)cc3)c(F)cc1C)=C2O]

72.5 g (21.2 mmol) of potassium tert-butoxide were initially charged in 10 ml of N,N-dimethylacetamide

(DMA). At 30° C., 4.3 g (9.6 mmol) of the compound from Example 57A in 10 ml of DMA were added dropwise, and the mixture was stirred at 30° C. for 4 h. The reaction mixture was poured onto ice-water, acidified with dilute hydrochloric acid and extracted with methylene chloride, and the extract was dried and evaporated. The residue was chromatographed on silica gel using methylene chloride/ethyl acetate (3:1). This gave 2.8 g (72% of theory) of the title compound.

¹H-NMR (400 MHz, DMSO-d₆): δ [ppm]=1.45-1.60 (m, 4H), 1.89-2.00 (m, 4H), 2.19 (s, 3H), 3.11-3.18 (m, 1H), 3.27 (s, 3H), 7.13-7.19 (m, 2H), 7.48-7.50 (m, 4H), 7.94 (s, 1H).

LC-MS (Method 2): Rₜ=1.21 min; MS (ESIpos): m/z=416 [M+H]⁺.

### Example 1-136

### Compound from Table 1, p. 26 of WO08/067,910

### (5s,8s)-3-(3′,4′-Difluoro-4-methylbiphenyl-3-yl)-4-hydroxy-8-methoxy-1-azaspiro[4.5]dec-3-en-2-one

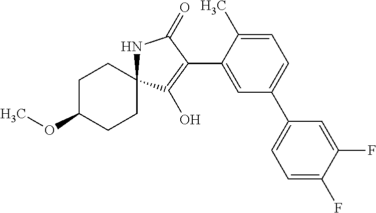 [SMILES: CO[C@H]1CC[C@]2(CC1)NC(=O)C(c1cc(-c3ccc(F)c(F)c3)ccc1C)=C2O]

0.59 g (1.5 mmol) of the compound from Example 58A, 0.36 g (2.3 mmol) of 3,4-trifluorophenylboronic acid and 0.8 g (7.5 mmol) of sodium carbonate were initially charged in 15 ml of water. 0.037 g (0.15 mmol) of palladium(II) nitrate dihydrate was added, and the mixture was heated at 130° C. under reflux overnight. The reaction mixture was then acidified with dilute hydrochloric acid and the precipitate was filtered off with suction. The aqueous phase was extracted with methylene chloride and the extract was dried and evaporated under reduced pressure. Purification was initially by MPLC on silica gel using a cyclohexane/acetone 0-50% gradient and then by RP chromatography using a water/acetonitrile 0-100% gradient. The residue was triturated with methyl tert-butyl ether and filtered off with suction. This gave 0.2 g (33% of theory) of the title compound.

¹H-NMR (400 MHz, DMSO-d₆): δ [ppm]=1.46-1.58 (m, 4H), 1.94-2.01 (m, 4H), 2.19 (s, 3H), 3.27 (s, 3H), 7.27-7.29 (m, 1H), 7.33-7.34 (m, 1H), 7.42-7.48 (m, 3H), 7.59-7.63 (m, 1H), 7.90 (s, 1H).

LC-MS (Method 1): Rₜ=1.19 min; MS (ESIpos): m/z=400 [M+H]⁺.

### Example 1-137

### =Compound According to WO99/48869

### (5S,7S)-3-(4,4′-Dichlorobiphenyl-3-yl)-4-hydroxy-7-(trifluoromethyl)-1-azaspiro[4.5]dec-3-en-2-one

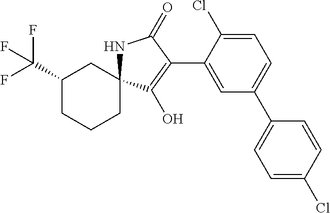 [SMILES: O=C1N[C@]2(CCC[C@H](C(F)(F)F)C2)C(O)=C1c1cc(-c2ccc(Cl)cc2)ccc1Cl]

The title compound was prepared analogously to the synthesis of the compound from Example 1-133 starting with the compound from Example 59A.

¹H-NMR (400 MHz, DMSO-d₆): δ [ppm]=1.24-1.26 (m, 1H), 1.42-1.46 (m, 1H), 1.57-1.60 (m, 1H), 1.66-1.73 (m, 1H), 1.81-1.96 (m, 4H), 2.67-2.78 (m, 1H), 7.52-7.56 (m, 4H), 7.61-7.63 (m, 1H), 7.70-7.72 (m, 2H), 8.27 (s, 1H).

LC-MS (Method 1): Rₜ=1.34 min; MS (ESIpos): m/z=456 [M+H]⁺.

### Example 1-138

### =Compound According to WO99/48869

### 3-(4,4′-Dichlorobiphenyl-3-yl)-4-hydroxy-8,8-dimethyl-1-azaspiro[4.5]dec-3-en-2-one

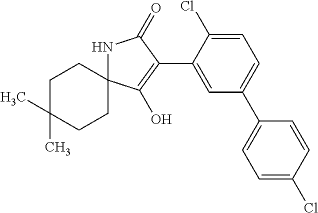 [SMILES: CC1(C)CCC2(CC1)NC(=O)C(c1cc(-c3ccc(Cl)cc3)ccc1Cl)=C2O]

The title compound was prepared analogously to the synthesis of the compound from Example 1-140 starting with the compound from Example 60A.

¹H-NMR (400 MHz, DMSO-d₆): δ [ppm]=0.94 (s, 3H), 0.95 (s, 3H), 1.24-1.33 (m, 4H), 1.57-1.63 (m, 2H), 1.91-2.09 (m, 2H), 7.48-7.55 (m, 4H), 7.59-7.65 (m, 1H), 7.68-7.71 (m, 2H), 8.20 (s, 1H).

LC-MS (Method 1): Rₜ=1.40 min; MS (ESIpos): m/z=416 [M+H]⁺.

### Example 1-139

### =Compound According to WO99/48869

### 3-(4,4′-Dichlorobiphenyl-3-yl)-4-hydroxy-8-methyl-1-azaspiro[4.5]dec-3-en-2-one

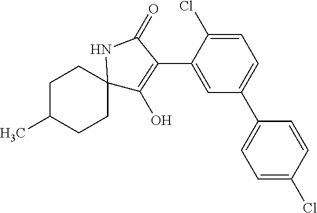 [SMILES: CC1CCC2(CC1)NC(=O)C(c1cc(-c3ccc(Cl)cc3)ccc1Cl)=C2O]

The title compound was prepared analogously to the synthesis of the compound from Example 1-140 starting with the compound from Example 61A.

¹H-NMR (400 MHz, DMSO-d₆): δ [ppm]=0.91 (d, 3H), 1.35-1.40 (m, 5H), 1.62 (m, 2H), 1.84-1.91 (m, 2H), 7.50-7.55 (m, 4H), 7.59-7.61 (m, 1H), 7.69-7.71 (m, 2H), 8.16 (s, 1H).

LC-MS (Method 2): Rₜ=1.36 min; MS (ESIpos): m/z=402 [M+H]⁺.

### Example 1-140

### =Compound According to WO99/48869

### 3-(4,4′-Dichlorobiphenyl-3-yl)-4-hydroxy-1-azaspiro[4.5]dec-3-en-2-one

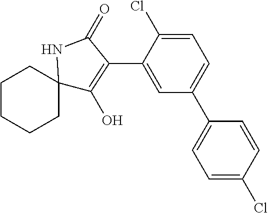 [SMILES: O=C1NC2(CCCCC2)C(O)=C1c1cc(-c2ccc(Cl)cc2)ccc1Cl]

2.31 g (18.9 mmol) of potassium tert-butoxide were initially charged in 7 ml of N,N-dimethylformamide (DMF). At 20-40° C., 3.54 g (8.4 mmol) of the compound from Example 62A in 8 ml DMF were added dropwise, and the mixture was stirred at 40° C. for 1 h. The reaction mixture was poured onto ice-water, acidified with dilute hydrochloric acid, filtered off with suction, rinsed and dried. The residue was chromatographed on silica gel using methylene chloride/acetone (3:1). This gave 2.79 g (85% of theory) of the title compound.

¹H-NMR (400 MHz, DMSO-d₆): δ [ppm]=1.16-1.26 (m, 1H), 1.31-1.41 (m, 2H), 1.55-1.66 (m, 5H), 1.79-1.91 (m, 2H), 7.50-7.54 (m, 4H), 7.60-7.62 (m, 1H), 7.68-7.71 (m, 2H), 8.30 (s, 1H).

LC-MS (Method 1): Rₜ=1.29 min; MS (ESIpos): m/z=388 [M+H]⁺.

### Example 1-141

### =Compound According to WO99/48869

### (5s,8s)-3-(4,4′-Dichlorobiphenyl-3-yl)-4-hydroxy-8-isopropyl-1-azaspiro[4.5]dec-3-en-2-one

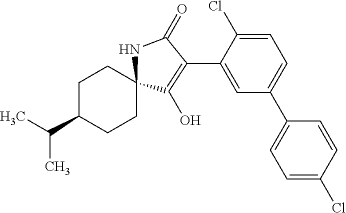 [SMILES: CC(C)[C@H]1CC[C@]2(CC1)NC(=O)C(c1cc(-c3ccc(Cl)cc3)ccc1Cl)=C2O]

The title compound was prepared analogously to the synthesis of the compound from Example 1-140 starting with the compound from Example 63A.

¹H-NMR (400 MHz, DMSO-d₆): δ [ppm]=0.88-0.89 (d, 6H), 0.98-1.08 (m, 1H), 1.32-1.47 (m, 5H), 1.64 (m, 2H), 1.81-1.88 (m, 2H), 7.50-7.55 (m, 4H), 7.59-7.61 (m, 1H), 7.69-7.71 (m, 2H), 8.18 (s, 1H).

LC-MS (Method 1): Rₜ=1.46 min; MS (ESIpos): m/z=430 [M+H]⁺.

### Example 1-142

### Compound of Table 1, p. 25 of WO08/067,9101

### (5s,8s)-3-(4′-Chloro-2′-fluoro-4-methylbiphenyl-3-yl)-4-hydroxy-8-methoxy-1-azaspiro[4.5]dec-3-en-2-one

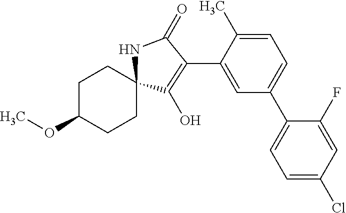 [SMILES: CO[C@H]1CC[C@]2(CC1)NC(=O)C(c1cc(-c3ccc(Cl)cc3F)ccc1C)=C2O]

0.1 mg of 1,1′-bis(diphenylphosphino)ferrocenedichloropalladium(II), 39.2 mg (0.225 mmol) of (4-chloro-2-fluorophenyl)boronic acid in 0.37 ml of 1,2-dimethoxyethane and 171 mg (0.525 mmol) of caesium carbonate in 0.29 ml of water were added to 54.9 mg (0.15 mmol) of (5s,8s)-3-(5-bromo-2-methylphenyl)-4-hydroxy-8-methoxy-1-azaspiro[4.5]dec-3-en-2-one (Example 58A) in 2.9 ml of N,N-dimethylformamide. The reaction mixture was stirred at 100° C. for 60 minutes. For work-up, 1 ml saturated aqueous sodium chloride solution was added to the cold reaction mixture, and the mixture was extracted with 3 ml of ethyl acetate. The organic phase was separated off and the solvent was evaporated. Work-up was by HPLC. This gave 13.2 mg (21% of theory) of the title compound.

LC-MS (Method 4): Rₜ=1.23 min; MS (ESIpos): m/z=416 [M+H]⁺.

### Example 1-143

### =Compound of Table 1, p. 69 in Combination with Table 10, p. 72 of WO99/48869)

### (5s,8s)-3-(2′,4′-Dichloro-4-methylbiphenyl-3-yl)-4-hydroxy-8-methoxy-1-azaspiro[4.5]dec-3-en-2-one

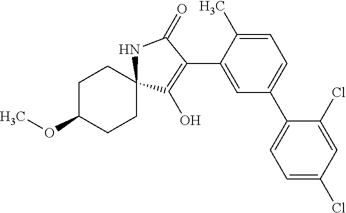 [SMILES: CO[C@H]1CC[C@]2(CC1)NC(=O)C(c1cc(-c3ccc(Cl)cc3Cl)ccc1C)=C2O]

The compound was prepared analogously to Example 1-142. This gave 10.1 mg (15% of theory) of the title compound.

LC-MS (Method 4): Rₜ=1.28 min; MS (ESIpos): m/z=432 [M+H]⁺.

### Example 1-144

### =Compound According to WO08/067,910

### (5s,8s)-3-(2′-Chloro-4′-fluoro-4-methylbiphenyl-3-yl)-4-hydroxy-8-methoxy-1-azaspiro[4.5]dec-3-en-2-one

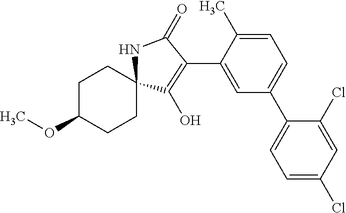 [SMILES: CO[C@H]1CC[C@]2(CC1)NC(=O)C(c1cc(-c3ccc(Cl)cc3Cl)ccc1C)=C2O]

The compound was prepared analogously to Example 1-142. This gave 10.0 mg (15% of theory) of the title compound.

LC-MS (Method 4): Rₜ=1.20 min; MS (ESIpos): m/z=416 [M+H]⁺.

### Example 1-145

### =Compound of Table 1, p. 41 in Combination with Table 2, p. 45 of WO08/067,911

### (5s,8s)-3-(3′,4′-Difluoro-4-methylbiphenyl-3-yl)-4-hydroxy-8-methyl-1-azaspiro[4.5]dec-3-en-2-one

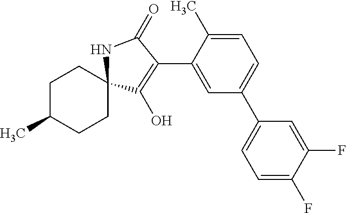 [SMILES: Cc1ccc(-c2ccc(F)c(F)c2)cc1C1=C(O)[C@]2(CC[C@H](C)CC2)NC1=O]

The title compound was prepared analogously to the synthesis of the compound from Example 1-90 starting with 1.35 g (3.85 mmol) of the compound from Example 53A. This gave 824 mg (54% of theory) of the title compound.

¹H-NMR (400 MHz, DMSO-d₆): δ [ppm]=0.92 (d, 3H), 1.28-1.42 (m, 5H), 1.56-1.68 (m, 2H), 1.84-1.96 (m, 2H), 2.18 (s, 3H), 7.30 (d, 1H), 7.36 (d, 1H), 7.45-7.54 (m, 3H), 7.66-7.74 (m, 1H), 8.13 (s, 1H), 10.75 (s, 1H).

LC-MS (Method 1): Rₜ=1.30 min; MS (ESIpos): m/z=384 [M+H]⁺.

### Example 1-146

### =Compound of Table 1 of WO99/48869)

### (5s,8s)-3-(4′-Chloro-4 methylbiphenyl-3-yl)-8-ethoxy-4-hydroxy-1-azaspiro[4.5]dec-3-en-2-one

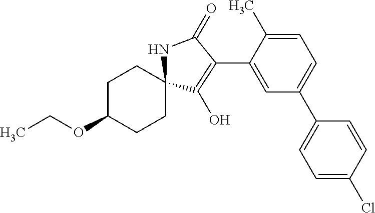 [SMILES: CCO[C@H]1CC[C@]2(CC1)NC(=O)C(c1cc(-c3ccc(Cl)cc3)ccc1C)=C2O]

Analogously to Example 1-118, the title compound was obtained after reversed phase chromatography with acetonitrile/water (gradient).

¹H-NMR (400 MHz, d₆-DMSO): δ [ppm]=1.12 (t, 3H), 1.44-1.47 (m, 2H), 1.51-1.61 (m, 2H), 1.91-1.98 (m, 4H), 2.19 (s, 3H), 3.22-3.27 (m, 1H), 3.47-3.52 (q, 2H), 7.31 (d, 1H), 7.34 (d, 1H), 7.48-7.52 (m, 3H), 7.64-7.67 (m, 2H), 8.18 (s, 1H), 10.81 (s, 1H).

LC-MS (Method 1): Rₜ=1.28 min; MS (ESIpos): m/z=412 [M+H]⁺.

### Example 1-147

### =Compound of Table 1 of WO99/48869

### (5r,8r)-3-(4′-Chloro-4 methylbiphenyl-3-yl)-8-ethoxy-4-hydroxy-1-azaspiro[4.5]dec-3-en-2-one

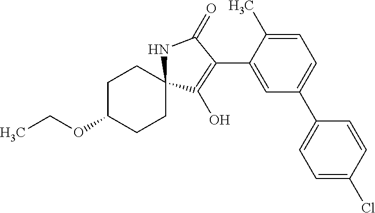 [SMILES: CCO[C@H]1CC[C@@]2(CC1)NC(=O)C(c1cc(-c3ccc(Cl)cc3)ccc1C)=C2O]

Analogously to Example 1-118, the title compound was obtained after reversed phase chromatography with acetonitrile/water (gradient).

¹H-NMR (400 MHz, d₆-DMSO): δ [ppm]=1.14 (t, 3H), 1.18-1.24 (m, 2H), 1.73-1.87 (m, 4H), 2.07-2.17 (m, 2H), 2.18 (s, 3H), 3.41-3.46 (m, 2H), 3.54 (m, 1H), 7.29-7.31 (m, 1H), 7.34 (d, 1H), 7.48-7.50 (m, 3H), 7.63-7.66 (m, 2H), 8.12 (s, 1H), 10.77 (s, 1H).

LC-MS (Method 1): Rₜ=1.28 min; MS (ESIpos): m/z=412 [M+H]⁺.

### Compounds of the Formula (I-2)

Table 2 lists the structures of some of the formula (I-2) of the prior art and indicates which patent discloses the preparation.

TABLE 2

| | | | Analysis |
|---|---|---|---|
| | | | ¹H—NMR: δ [ppm] |
| Ex. | Structure/Name | disclosed in | retention time, [M + H]⁺, Method |
| 2-1 | 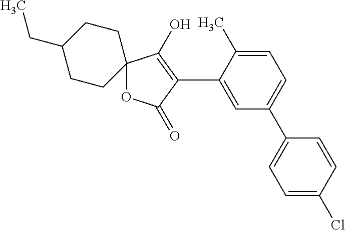 [SMILES: CCC1CCC2(CC1)OC(=O)C(c1cc(-c3ccc(Cl)cc3)ccc1C)=C2O] | WO 03/059065 I-2-a-12 | |
| | 3-(4′-chloro-4-methylbiphenyl-3-yl)- | | |
| | 8-ethyl-4-hydroxy-1- | | |
| | oxaspiro[4.5]dec-3-en-2-one | | |
| 2-2 | 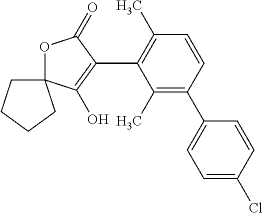 [SMILES: Cc1ccc(-c2ccc(Cl)cc2)c(C)c1C1=C(O)C2(CCCC2)OC1=O] | WO 03/059065 I-2-a-19 | |
| | 3-(4′-chloro-2,4-dimethylbiphenyl-3- | | |
| | yl)-4-hydroxy-1-oxaspiro[4.4]non- | | |
| | 32--en-2-one | | |
| 2-3 | 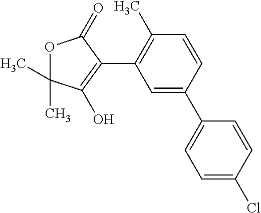 [SMILES: Cc1ccc(-c2ccc(Cl)cc2)cc1C1=C(O)C(C)(C)OC1=O] | WO 03/059065 I-2-a-2 | |
| | 3-(4′-chloro-4-methylbiphenyl-3-yl)- | | |
| | 4-hydroxy-5,5-dimethylfuran-2(5H)- | | |
| | one | | |
| 2-4 | 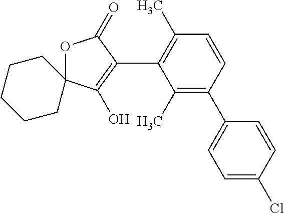 [SMILES: Cc1ccc(-c2ccc(Cl)cc2)c(C)c1C1=C(O)C2(CCCCC2)OC1=O] | WO 03/059065 I-2-a-20 | |
| | 3-(4′-chloro-2,4-dimethylbiphenyl-3- | | |
| | yl)-4-hydroxy-1-oxaspiro[4.5]dec-3- | | |
| | en-2-one | | |
| 2-5 | 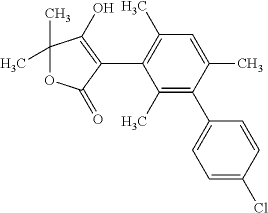 [SMILES: Cc1cc(C)c(-c2ccc(Cl)cc2)c(C)c1C1=C(O)C(C)(C)OC1=O] | WO 03/059065 I-2-a-21 | |
| | 3-(4′-chloro-2,4,6-trimethylbiphenyl- | | |
| | 3-yl)-4-hydroxy-5,5-dimethylfuran- | | |
| | 2(5H)-one | | |
| 2-6 | 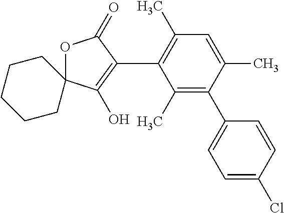 [SMILES: Cc1cc(C)c(-c2ccc(Cl)cc2)c(C)c1C1=C(O)C2(CCCCC2)OC1=O] | WO 03/059065 I-2-a-22 | |
| | 3-(4′-chloro-2,4,6-trimethylbiphenyl- | | |
| | 3-yl)-4-hydroxy-1-oxaspiro[4.5]dec- | | |
| | 3-en-2-one | | |
| 2-7 | 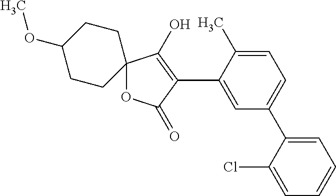 [SMILES: COC1CCC2(CC1)OC(=O)C(c1cc(-c3ccccc3Cl)ccc1C)=C2O] | WO 03/059065 I-2-a-32 | |
| | 3-(2′-chloro-4-methylbiphenyl-3-yl)- | | |
| | 4-hydroxy-8-methoxy-1- | | |
| | oxaspiro[4.5]dec-3-en-2-one | | |
| 2-8 | 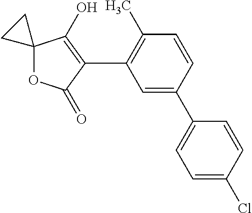 [SMILES: Cc1ccc(-c2ccc(Cl)cc2)cc1C1=C(O)C2(CC2)OC1=O] | WO 03/059065 I-2-a-6 | |
| | 6-(4′-chloro-4-methylbiphenyl-3-yl)- | | |
| | 7-hydroxy-4-oxaspiro[2.4]hept-6-en- | | |
| | 5-one | | |
| 2-9 | 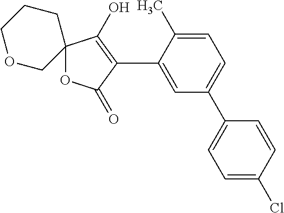 [SMILES: Cc1ccc(-c2ccc(Cl)cc2)cc1C1=C(O)C2(CCCOC2)OC1=O] | WO 03/059065 I-2-a-7 | |
| | 3-(4′-chloro-4-methylbiphenyl-3-yl)- | | |
| | 4-hydroxy-1,7-dioxaspiro[4.5]dec-3- | | |
| | en-2-one | | |
| 2-10 | 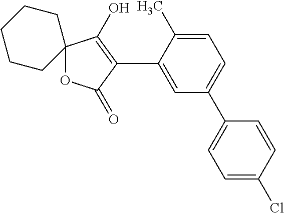 [SMILES: Cc1ccc(-c2ccc(Cl)cc2)cc1C1=C(O)C2(CCCCC2)OC1=O] | WO 03/059065 I-2-a-8 | |
| | 3-(4′-chloro-4-methylbiphenyl-3-yl)- | | |
| | 4-hydroxy-1-oxaspiro[4.5]dec-3-en- | | |
| | 2-one | | |
| 2-11 | 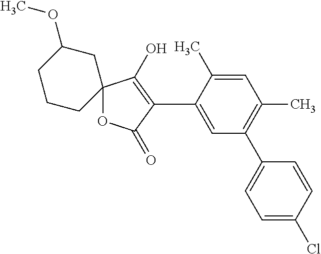 [SMILES: COC1CCCC2(C1)OC(=O)C(c1cc(-c3ccc(Cl)cc3)c(C)cc1C)=C2O] | WO 06/000355 I-2-a-2 | |
| | 3-(4′-chloro-4,6-dimethylbiphenyl-3- | | |
| | yl)-4-hydroxy-7-methoxy-1- | | |
| | oxaspiro[4.5]dec-3-en-2-one | | |
| 2-12 | 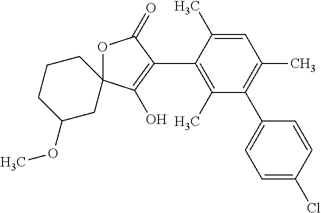 [SMILES: COC1CCCC2(C1)OC(=O)C(c1c(C)cc(C)c(-c3ccc(Cl)cc3)c1C)=C2O] | WO 06/000355 I-2-a-30 | |
| | 3-(4′-chloro-2,4,6-trimethylbiphenyl- | | |
| | 3-yl)-4-hydroxy-7-methoxy-1- | | |
| | oxaspiro[4.5]dec-3-en-2-one | | |
| 2-13 | 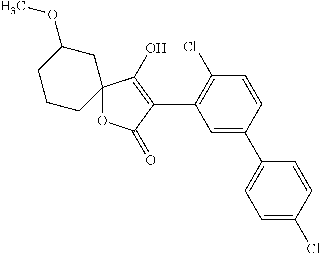 [SMILES: COC1CCCC2(C1)OC(=O)C(c1cc(-c3ccc(Cl)cc3)ccc1Cl)=C2O] | WO 06/000355 I-2-a-31 | |
| | 3-(4,4′-dichlorobiphenyl-3-yl)-4- | | |
| | hydroxy-7-methoxy-1- | | |
| | oxaspiro[4.5]dec-3-en-2-one | | |
| 2-14 | 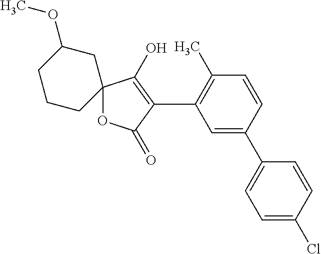 [SMILES: COC1CCCC2(C1)OC(=O)C(c1cc(-c3ccc(Cl)cc3)ccc1C)=C2O] | WO 06/000355 I-2-a-32 | |
| | 3-(4′-chloro-4-methylbiphenyl-3-yl)- | | |
| | 4-hydroxy-7-methoxy-1- | | |
| | oxaspiro[4.5]dec-3-en-2-one | | |
| 2-15 | 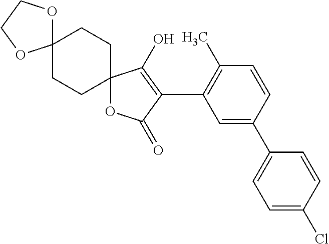 [SMILES: Cc1ccc(-c2ccc(Cl)cc2)cc1C1=C(O)C2(CCC3(CC2)OCCO3)OC1=O] | WO 06/089633 I-2-a-8 | |
| | 11-(4′-chloro-4-methylbiphenyl-3-yl)- | | |
| | 12-hydroxy-1,4,9-trioxadi- | | |
| | spiro[4.2.4.2]tetradec-11-en-10-one | | |
| 2-16 | 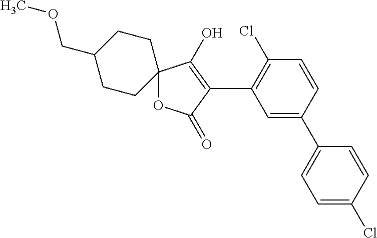 [SMILES: COCC1CCC2(CC1)OC(=O)C(c1cc(-c3ccc(Cl)cc3)ccc1Cl)=C2O] | WO 07/048545 I-2-a-16 | |
| | 3-(4,4′-dichlorobiphenyl-3-yl)-4- | | |
| | hydroxy-8-(methoxymethyl)-1- | | |
| | oxaspiro[4.5]dec-3-en-2-one | | |
| 2-17 | 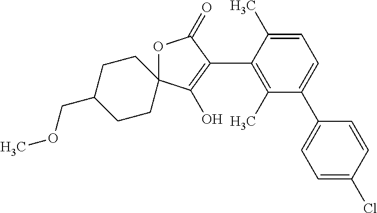 [SMILES: COCC1CCC2(CC1)OC(=O)C(c1c(C)ccc(-c3ccc(Cl)cc3)c1C)=C2O] | WO 07/048545 I-2-a-17 | |
| | 3-(4′-chloro-2,4-dimethylbiphenyl-3- | | |
| | yl)-4-hydroxy-8-(methoxymethyl)-1- | | |
| | oxaspiro[4.5]dec-3-en-2-one | | |
| 2-18 | 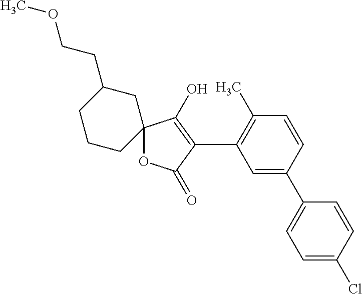 [SMILES: COCCC1CCCC2(C1)OC(=O)C(c1cc(-c3ccc(Cl)cc3)ccc1C)=C2O] | WO 07/048545 I-2-a-18 | |
| | 3-(4′-chloro-4-methylbiphenyl-3-yl)- | | |
| | 4-hydroxy-7-(2 methoxyethyl)-1- | | |
| | oxaspiro[4.5]dec-3-en-2-one | | |
| 2-19 | 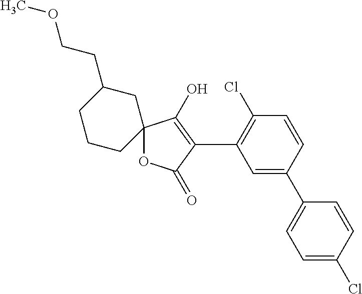 [SMILES: COCCC1CCCC2(C1)OC(=O)C(c1cc(-c3ccc(Cl)cc3)ccc1Cl)=C2O] | WO 07/048545 I-2-a-26 | |
| | 3-(4,4′-dichlorobiphenyl-3-yl)-4- | | |
| | hydroxy-7-(2 methoxyethyl)-1- | | |
| | oxaspiro[4.5]dec-3-en-2-one | | |
| 2-20 | 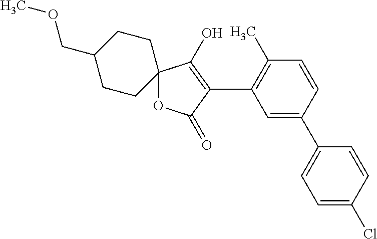 [SMILES: COCC1CCC2(CC1)OC(=O)C(c1cc(-c3ccc(Cl)cc3)ccc1C)=C2O] | WO 07/048545 I-2-a-8 | |
| | 3-(4′-chloro-4-methylbiphenyl-3-yl)- | | |
| | 4-hydroxy-8-(methoxymethyl)-1- | | |
| | oxaspiro[4.5]dec-3-en-2-one | | |
| 2-21 | 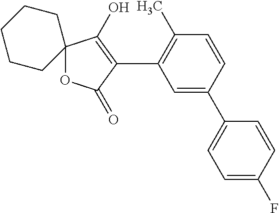 [SMILES: Cc1ccc(-c2ccc(F)cc2)cc1C1=C(O)C2(CCCCC2)OC1=O] | WO 08/067911 I-2-a-15 | |
| | 3-(4′-fluoro-4-methylbiphenyl-3-yl)- | | |
| | 4-hydroxy-1-oxaspiro[4.5]dec-3-en- | | |
| | 2-one | | |
| 2-22 | 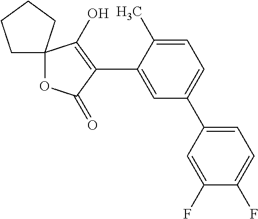 [SMILES: Cc1ccc(-c2ccc(F)c(F)c2)cc1C1=C(O)C2(CCCC2)OC1=O] | WO 08/067911 I-2-a-3 | |
| | 3-(3′,4′-difluoro-4-methylbiphenyl-3- | | |
| | yl)-4-hydroxy-1-oxaspiro[4.4]non-3- | | |
| | en-2-one | | |
| 2-23 | 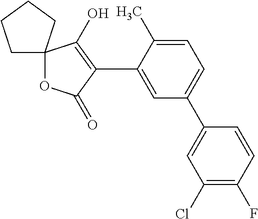 [SMILES: Cc1ccc(-c2ccc(F)c(Cl)c2)cc1C1=C(O)C2(CCCC2)OC1=O] | WO 08/067911 I-2-a-4 | |
| | 3-(3′-chloro-4′-fluoro-4- | | |
| | methylbiphenyl-3-yl)-4-hydroxy-1- | | |
| | oxaspiro[4.4]non-3-en-2-one | | |
| 2-24 | 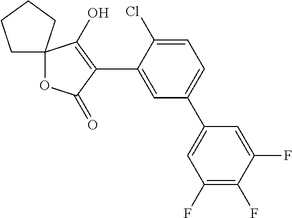 [SMILES: O=C1OC2(CCCC2)C(O)=C1c1cc(-c2cc(F)c(F)c(F)c2)ccc1Cl] | WO 08/067911 I-2-a-6 | |
| | 3-(4-chloro-3′,4′,5′-trifluorobiphenyl- | | |
| | 3-yl)-4-hydroxy-1-oxaspiro[4.4]non- | | |
| | 3-en-2-one | | |
| 2-25 | 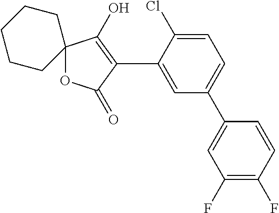 [SMILES: O=C1OC2(CCCCC2)C(O)=C1c1cc(-c2ccc(F)c(F)c2)ccc1Cl] | WO 08/067911 I-2-a-8 | |
| | 3-(4-chloro-3′,4′-difluorobiphenyl-3- | | |
| | yl)-4-hydroxy-1-oxaspiro[4.5]dec-3- | | |
| | en-2-one | | |
| 2-26 | 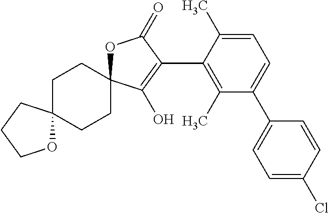 [SMILES: Cc1ccc(-c2ccc(Cl)cc2)c(C)c1C1=C(O)[C@]2(CC[C@]3(CCCO3)CC2)OC1=O] | WO 09/015801 I-2-a-19 | |
| | (5s,8r)-3-(4′-chloro-2,4- | | |
| | dimethylbiphenyl-3-yl)-4-hydroxy- | | |
| | 1,9-dioxadispiro[4.2.4.2]tetradec-3- | | |
| | en-2-one | | |
| 2-27 | 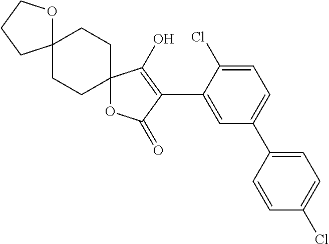 [SMILES: O=C1OC2(CCC3(CCCO3)CC2)C(O)=C1c1cc(-c2ccc(Cl)cc2)ccc1Cl] | WO 09/015801 I-2-a-21 | |
| | 3-(4,4′-dichlorobiphenyl-3-yl)-4- | | |
| | hydroxy-1,9-dioxadi- | | |
| | spiro[4.2.4.2]tetradec-3-en-2-one | | |
| 2-28 | 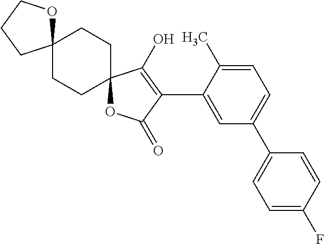 [SMILES: Cc1ccc(-c2ccc(F)cc2)cc1C1=C(O)[C@]2(CC[C@@]3(CCCO3)CC2)OC1=O] | WO 09/015801 I-2-a-22 | |
| | (5r,8s)-3-(4′-fluoro-4- | | |
| | methylbiphenyl-3-yl)-4-hydroxy-1,9- | | |
| | dioxadispiro[4.2.4.2]tetradec-3-en-2- | | |
| | one | | |
| 2-29 | 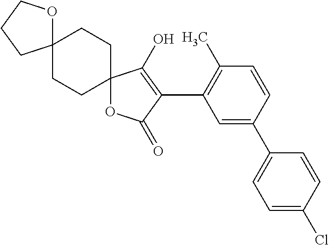 [SMILES: Cc1ccc(-c2ccc(Cl)cc2)cc1C1=C(O)C2(CCC3(CCCO3)CC2)OC1=O] | WO 09/015801 I-2-a-25 | |
| | 3-(4′-chloro-4-methylbiphenyl-3-yl)- | | |
| | 4-hydroxy-1,9-dioxadi- | | |
| | spiro[4.2.4.2]tetradec-3-en-2-one | | |
| 2-30 | 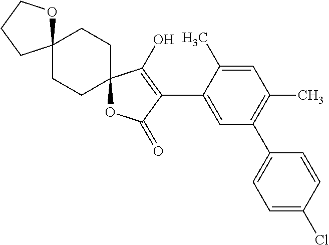 [SMILES: Cc1cc(C)c(-c2ccc(Cl)cc2)cc1C1=C(O)[C@]2(CC[C@@]3(CCCO3)CC2)OC1=O] | WO 09/015801 I-2-a 27 | (300 MHz, DMSO-d₆): 1.58- 1.81 (m, 8H), 1.83-2.07 (m, 4H), 2.16 (s, 3H), 2.22 (s, 3H), 3.73 (t, 2H), 6.95 (s, 1H), 7.18 (s, 1H), 7.32-7.38 (m, 2H), 7.45- 7.52 (m, 2H), 12.19 (br. s., 1H). 1.36 min, 439, Method 1 |
| | (5r,8s)-3-(4′-chloro-4,6-dimethyl- | | |
| | biphenyl-3-yl)-4-hydroxy-1,9-dioxa- | | |
| | dispiro[4.2.4.2]tetradec-3-en-2-one | | |
| 2-31 | 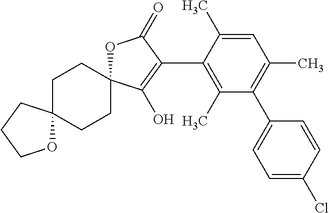 [SMILES: Cc1cc(C)c(-c2ccc(Cl)cc2)c(C)c1C1=C(O)[C@]2(CC[C@@]3(CCCO3)CC2)OC1=O] | WO 09/015801 I-2-a-42 | |
| | (5r,8s)-3-(4′-chloro-2,4,6- | | |
| | trimethylbiphenyl-3-yl)-4-hydroxy- | | |
| | 1,9-dioxadispiro[4.2.4.2]tetradec-3- | | |
| | en-2-one | | |
| 2-32 | 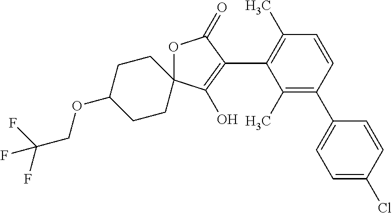 [SMILES: Cc1ccc(-c2ccc(Cl)cc2)c(C)c1C1=C(O)C2(CCC(OCC(F)(F)F)CC2)OC1=O] | WO 09/039975 I-2-a-6 | 1.43, 1.46 min, 481, Method 1 |
| | 3-(4′-chloro-2,4-dimethylbiphenyl-3- | | |
| | yl)-4-hydroxy-8-(2,2,2- | | |
| | trifluoroethoxy)-1-oxaspiro[4.5]dec- | | |
| | 3-en-2-one | | |
| 2-33 | 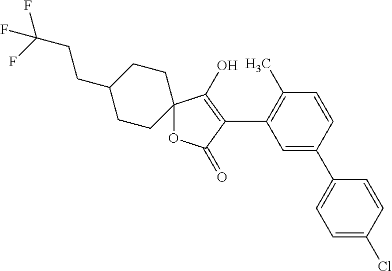 [SMILES: Cc1ccc(-c2ccc(Cl)cc2)cc1C1=C(O)C2(CCC(CCC(F)(F)F)CC2)OC1=O] | WO 09/039975 I-2-a-7 | (300 MHz, DMSO-d₆): 1.43- 1.62 (m, 2H), 1.63-1.85 (m, 2H), 1.92-2.03 (m, 2H), 2.04- 2.24 (m, 5H), 3.82-3.90 (m, 0.5H), 3.98-4.19 (m, 2.5H), 7.32- 7.37 (m, 1H), 7.42 (d, 1H), 7.47- 7.58 (m, 3H), 7.64-7.70 (m, 2H), 12.35 (br. s., 1H). 1.40, 1.42 min, 467 Method 1 |
| | 3-(4′-chloro-4-methylbiphenyl-3-yl)- | | |
| | 4-hydroxy-8-(2,2,2-trifluoroethoxy)- | | |
| | 1-oxaspiro[4.5]dec-3-en-2-one | | |
| 2-34 | 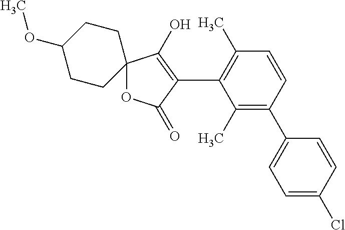 [SMILES: COC1CCC2(CC1)OC(=O)C(c1c(C)ccc(-c3ccc(Cl)cc3)c1C)=C2O] | WO 99/48869 I-2-a-2 | |
| | 3-(4′-chloro-2,4-dimethylbiphenyl-3- | | |
| | yl)-4-hydroxy-8-methoxy-1- | | |
| | oxaspiro[4.5]dec-3-en-2-one | | |
| 2-35 | 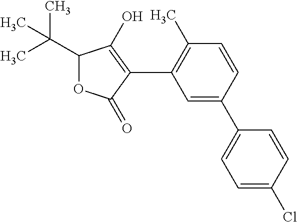 [SMILES: Cc1ccc(-c2ccc(Cl)cc2)cc1C1=C(O)C(C(C)(C)C)OC1=O] | WO 99/48869 T 25 | |
| | 5-tert-butyl-3-(4′-chloro-4- | | |
| | methylbiphenyl-3-yl)-4- | | |
| | hydroxyfuran-2(5H)-one | | |
| 2-36 | 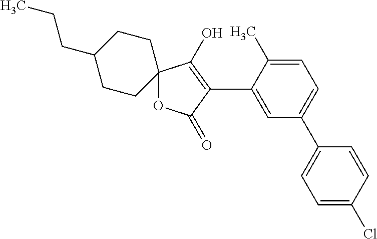 [SMILES: CCCC1CCC2(CC1)OC(=O)C(c1cc(-c3ccc(Cl)cc3)ccc1C)=C2O] | WO 99/48869 T 25 | |
| | 3-(4′-chloro-4-methylbiphenyl-3-yl)- | | |
| | 4-hydroxy-8-propyl-1- | | |
| | oxaspiro[4.5]dec-3-en-2-one | | |
| 2-37 | 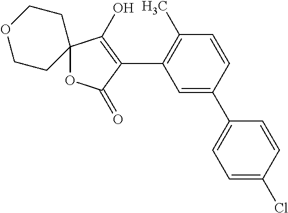 [SMILES: Cc1ccc(-c2ccc(Cl)cc2)cc1C1=C(O)C2(CCOCC2)OC1=O] | WO 99/48869 T 25 | |
| | 3-(4′-chloro-4-methylbiphenyl-3-yl)- | | |
| | 4-hydroxy-1,8-dioxaspiro[4.5]dec-3- | | |
| | en-2-one | | |
| 2-38 | 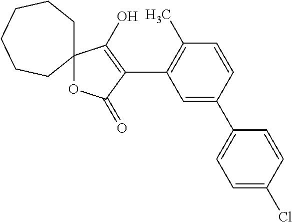 [SMILES: Cc1ccc(-c2ccc(Cl)cc2)cc1C1=C(O)C2(CCCCCC2)OC1=O] | WO 99/48869 T 25 | |
| | 3-(4′-chloro-4-methylbiphenyl-3-yl)- | | |
| | 4-hydroxy-1-oxaspiro[4.6]undec-3- | | |
| | en-2-one | | |
| 2-39 | 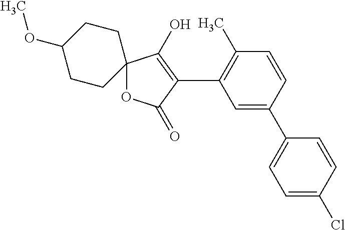 [SMILES: COC1CCC2(CC1)OC(=O)C(c1cc(-c3ccc(Cl)cc3)ccc1C)=C2O] | WO 99/48869 T 25 | |
| | 3-(4′-chloro-4-methylbiphenyl-3-yl)- | | |
| | 4-hydroxy-8-methoxy-1- | | |
| | oxaspiro[4.5]dec-3-en-2-one | | |
| 2-40 | 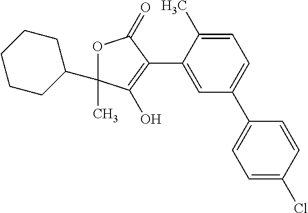 [SMILES: Cc1ccc(-c2ccc(Cl)cc2)cc1C1=C(O)C(C)(C2CCCCC2)OC1=O] | WO 99/48869 T 25 | |
| | 3-(4′-chloro-4-methylbiphenyl-3-yl)- | | |
| | 5-cyclohexyl-4-hydroxy-5- | | |
| | methylfuran-2(5H)-one | | |
| 2-41 | 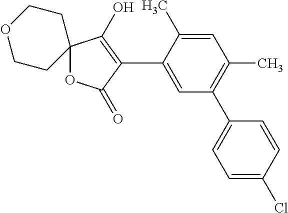 [SMILES: Cc1cc(C)c(-c2ccc(Cl)cc2)cc1C1=C(O)C2(CCOCC2)OC1=O] | WO 99/48869 T 26 | |
| | 3-(4′-chloro-4,6-dimethylbiphenyl-3- | | |
| | yl)-4-hydroxy-1,8- | | |
| | dioxaspiro[4.5]dec-3-en-2-one | | |
| 2-42 | 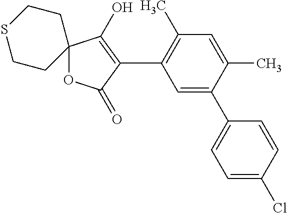 [SMILES: Cc1cc(C)c(-c2ccc(Cl)cc2)cc1C1=C(O)C2(CCSCC2)OC1=O] | WO 99/48869 T 26 | |
| | 3-(4′-chloro-4,6-dimethylbiphenyl-3- | | |
| | yl)-4-hydroxy-1-oxa-8- | | |
| | thiaspiro[4.5]dec-3-en-2-one | | |
| 2-43 | 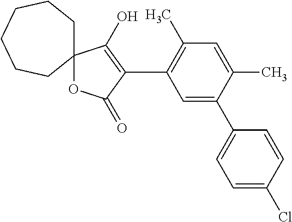 [SMILES: Cc1cc(C)c(-c2ccc(Cl)cc2)cc1C1=C(O)C2(CCCCCC2)OC1=O] | WO 99/48869 T 26 | |
| | 3-(4′-chloro-4,6-dimethylbiphenyl-3- | | |
| | yl)-4-hydroxy-1-oxaspiro[4.6]undec- | | |
| | 3-en-2-one | | |
| 2-44 | 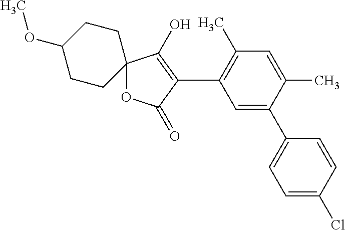 [SMILES: COC1CCC2(CC1)OC(=O)C(c1cc(-c3ccc(Cl)cc3)c(C)cc1C)=C2O] | WO 99/48869 T 26 | |
| | 3-(4′-chloro-4,6-dimethylbiphenyl-3- | | |
| | yl)-4-hydroxy-8-methoxy-1- | | |
| | oxaspiro[4.5]dec-3-en-2-one | | |
| 2-45 | 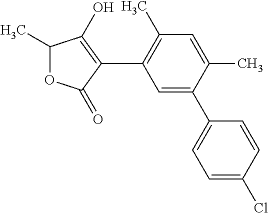 [SMILES: Cc1cc(C)c(-c2ccc(Cl)cc2)cc1C1=C(O)C(C)OC1=O] | WO 99/48869 T 26 | |
| | 3-(4′-chloro-4,6-dimethylbiphenyl-3- | | |
| | yl)-4-hydroxy-5-methylfuran-2(5H)- | | |
| | one | | |
| 2-46 | 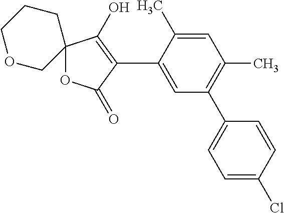 [SMILES: Cc1cc(C)c(-c2ccc(Cl)cc2)cc1C1=C(O)C2(CCCOC2)OC1=O] | WO 99/48869 T 26 | |
| | 3-(4′-chloro-4,6-dimethylbiphenyl-3- | | |
| | yl)-4-hydroxy-1,7- | | |
| | dioxaspiro[4.5]dec-3-en-2-one | | |
| 2-47 | 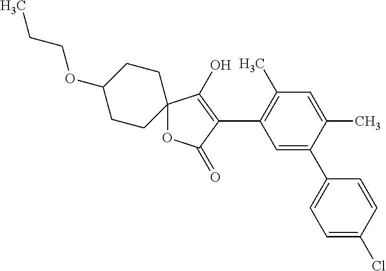 [SMILES: CCCOC1CCC2(CC1)OC(=O)C(c1cc(-c3ccc(Cl)cc3)c(C)cc1C)=C2O] | WO 99/48869 T 26 | |
| | 3-(4′-chloro-4,6-dimethylbiphenyl-3- | | |
| | yl)-4-hydroxy-8-propoxy-1- | | |
| | oxaspiro[4.5]dec-3-en-2-one | | |
| 2-48 | 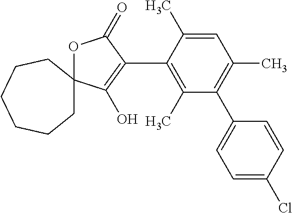 [SMILES: Cc1cc(C)c(-c2ccc(Cl)cc2)c(C)c1C1=C(O)C2(CCCCCC2)OC1=O] | WO 99/48869 T 27 | |
| | 3-(4′-chloro-2,4,6-trimethylbiphenyl- | | |
| | 3-yl)-4-hydroxy-1- | | |
| | oxaspiro[4.6]undec-3-en-2-one | | |
| 2-49 | 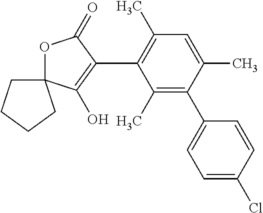 [SMILES: Cc1cc(C)c(-c2ccc(Cl)cc2)c(C)c1C1=C(O)C2(CCCC2)OC1=O] | WO 99/48869 T 27 | |
| | 3-(4′-chloro-2,4,6-trimethylbiphenyl- | | |
| | 3-yl)-4-hydroxy-1-oxaspiro[4.4]non- | | |
| | 3-en-2-one | | |
| 2-50 | 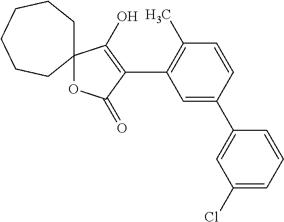 [SMILES: Cc1ccc(-c2cccc(Cl)c2)cc1C1=C(O)C2(CCCCCC2)OC1=O] | WO 99/48869 T 28 | |
| | 3-(3′-chloro-4-methylbiphenyl-3-yl)- | | |
| | 4-hydroxy-1-oxaspiro[4.6]undec-3- | | |
| | en-2-one | | |

### Compounds According to Formulae (I-3), (I-6), (I-7), (I-8), (I-9), (I-10) and (I-11)

Table 3 lists the structures of some of the formulae (I-3), (I-6), (I-7), (I-8), (I-9), (I-10) and (I-11) of the prior art and indicates which patent discloses the preparation.

TABLE 3

| | | | Analysis |
|---|---|---|---|
| | | | ¹H—NMR: δ [ppm] |
| Ex. | Structure/Name | disclosed in | retention time, [M + H]⁺, Method |
| 3-1 | 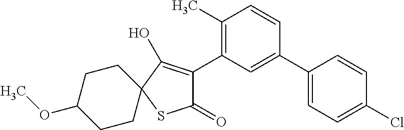 [SMILES: COC1CCC2(CC1)SC(=O)C(c1cc(-c3ccc(Cl)cc3)ccc1C)=C2O] | WO 03/059065 I-3-a-5 | |
| | 3-(4′-chloro-4-methylbiphenyl-3-yl)-4- | | |
| | hydroxy-8-methoxy-1-thiaspiro[4.5]dec- | | |
| | 3-en-2-one | | |
| 3-2 | 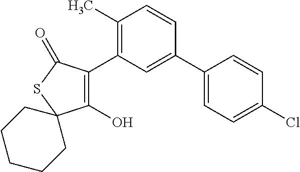 [SMILES: Cc1ccc(-c2ccc(Cl)cc2)cc1C1=C(O)C2(CCCCC2)SC1=O] | WO 99/48869 I-3-a-1 | |
| | 3-(4′-chloro-4-methylbiphenyl-3-yl)-4- | | |
| | hydroxy-1-thiaspiro[4.5]dec-3-en-2-one | | |
| 3-3 | 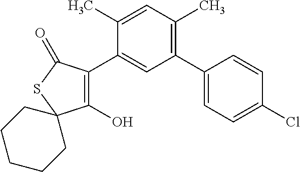 [SMILES: Cc1cc(C)c(-c2ccc(Cl)cc2)cc1C1=C(O)C2(CCCCC2)SC1=O] | WO 99/48869 I-3-a-2 | |
| | 3-(4′-chloro-4,6-dimethylbiphenyl-3-yl)- | | |
| | 4-hydroxy-1-thiaspiro[4.5]dec-3-en-2- | | |
| | one | | |
| 3-4 | 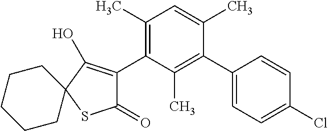 [SMILES: Cc1cc(C)c(-c2ccc(Cl)cc2)c(C)c1C1=C(O)C2(CCCCC2)SC1=O] | WO 99/48869 I-3-a-3 | |
| | 3-(4′-chloro-2,4,6-trimethylbiphenyl-3- | | |
| | yl)-4-hydroxy-1-thiaspiro[4.5]dec-3-en- | | |
| | one | | |
| 6-1 | 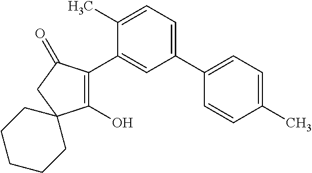 [SMILES: Cc1ccc(-c2ccc(C)c(C3=C(O)C4(CCCCC4)CC3=O)c2)cc1] | WO 03/059065 I-4-a-11 | |
| | 3-(4,4′-dimethylbiphenyl-3-yl)-4- | | |
| | hydroxyspiro[4.5]dec-3-en-2-one | | |
| 6-2 | 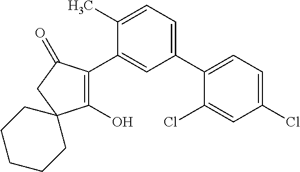 [SMILES: Cc1ccc(-c2ccc(Cl)cc2Cl)cc1C1=C(O)C2(CCCCC2)CC1=O] | WO 03/059065 I-4-a-13 | (400 MHz, DMSO-d₆): 1.13- 1.26 (m, 1H), 1.27-1.41 (m, 4H), 1.45-1.74 (m, 5H), 2.14 (s, 3H), 2.45-2.55 (m, 2H), 7.05 (d, 1H), 7.23-7.31 (m, 2H), 7.39 (d, 1H), 7.49 (dd, 1H), 7.71 (d, 1H), 12.03 (br. s., 1H). 1.46 min, 401, Method |
| | 3-(2′,4′-dichloro-4-methylbiphenyl-3- | | |
| | yl)-4-hydroxyspiro[4.5]dec-3-en-2-one | | |
| 6-3 | 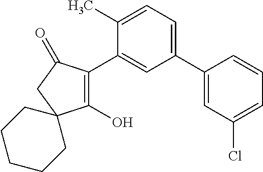 [SMILES: Cc1ccc(-c2cccc(Cl)c2)cc1C1=C(O)C2(CCCCC2)CC1=O] | WO 03/059065 I-4-a-5 | |
| | 3-(3′-chloro-4-methylbiphenyl-3-yl)-4- | | |
| | hydroxyspiro[4.5]dec-3-en-2-one | | |
| 6-4 | 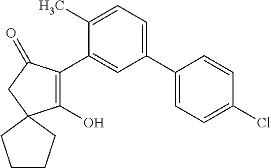 [SMILES: Cc1ccc(-c2ccc(Cl)cc2)cc1C1=C(O)C2(CCCC2)CC1=O] | WO 03/059065 I-4-a-6 | |
| | 3-(4′-chloro-4-methylbiphenyl-3-yl)-4- | | |
| | hydroxyspiro[4.4]non-3-en-2-one | | |
| 6-5 | 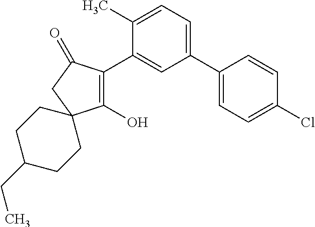 [SMILES: CCC1CCC2(CC1)CC(=O)C(c1cc(-c3ccc(Cl)cc3)ccc1C)=C2O] | WO 03/059065 I-4-a-7 | |
| | 3-(4′-chloro-4-methylbiphenyl-3-yl)-8- | | |
| | ethyl-4-hydroxyspiro[4.5]dec-3-en-2- | | |
| | one | | |
| 6-6 | 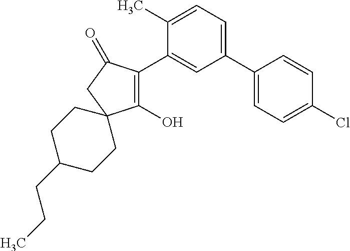 [SMILES: CCCC1CCC2(CC1)CC(=O)C(c1cc(-c3ccc(Cl)cc3)ccc1C)=C2O] | WO 03/059065 I-4-a-8 | |
| | 3-(4′-chloro-4-methylbiphenyl-3-yl)-4- | | |
| | hydroxy-8-propylspiro[4.5]dec-3-en-2- | | |
| | one | | |
| 6-7 | 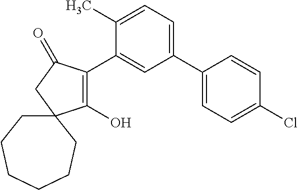 [SMILES: Cc1ccc(-c2ccc(Cl)cc2)cc1C1=C(O)C2(CCCCCC2)CC1=O] | WO 03/059065 I-4-a-9 | |
| | 3-(4′-chloro-4-methylbiphenyl-3-yl)-4- | | |
| | hydroxyspiro[4.6]undec-3-en-2-one | | |
| 6-8 | 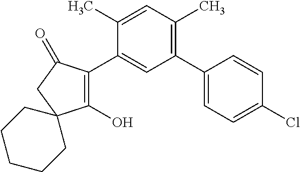 [SMILES: Cc1cc(C)c(-c2ccc(Cl)cc2)cc1C1=C(O)C2(CCCCC2)CC1=O] | WO 99/48869 I-7-a-1 | (300 MHz, DMSO-d₆): 1.13- 1.42 (m, 5H), 1.46-1.74 (m, 5H), 2.08 (s, 3H), 2.20 (s, 3H), 2.45-2.55 (m, 2H), 6.82 (s, 1H), 7.12 (s, 1H), 7.30-7.36 (m, 2H), 7.44-7.50 (m, 2H), 11.86 (br. s., 1H). 1.42 min, 381, Method 1 |
| | 3-(4′-chloro-4,6-dimethylbiphenyl-3-yl)- | | |
| | 4-hydroxyspiro[4.5]dec-3-en-2-one | | |
| 6-9 | 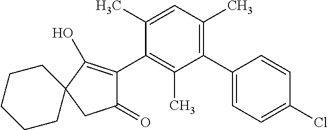 [SMILES: Cc1cc(C)c(-c2ccc(Cl)cc2)c(C)c1C1=C(O)C2(CCCCC2)CC1=O] | WO 99/48869 I-7-a-3 | |
| | 3-(4′-chloro-2,4,6-trimethylbiphenyl-3- | | |
| | yl)-4-hydroxyspiro[4.5]dec-3-en-2-one | | |
| 7-1 | 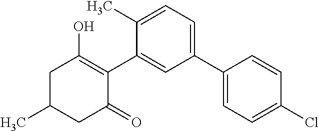 [SMILES: Cc1ccc(-c2ccc(Cl)cc2)cc1C1=C(O)CC(C)CC1=O] | WO 03/059065 I-5-a-12 | |
| | 2-(4′-chloro-4-methylbiphenyl-3-yl)-3- | | |
| | hydroxy-5-methylcyclohex-2-en-1-one | | |
| 7-2 | 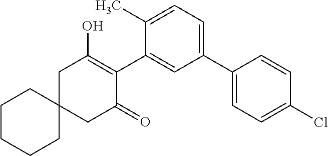 [SMILES: Cc1ccc(-c2ccc(Cl)cc2)cc1C1=C(O)CC2(CCCCC2)CC1=O] | WO 03/059065 I-5-a-8 | |
| | 3-(4′-chloro-4-methylbiphenyl-3-yl)-4- | | |
| | hydroxyspiro[5.5]undec-3-en-2-one | | |
| 7-3 | 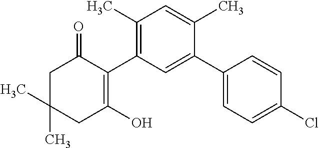 [SMILES: Cc1cc(C)c(-c2ccc(Cl)cc2)cc1C1=C(O)CC(C)(C)CC1=O] | WO 99/48869 I-8-a-2 | |
| | 2-(4′-chloro-4,6-dimethylbiphenyl-3-yl)- | | |
| | 3-hydroxy-5,5-dimethylcyclohex-2-en- | | |
| | 1-one | | |
| 7-4 | 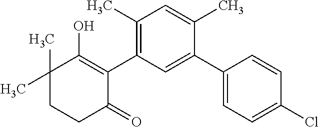 [SMILES: Cc1cc(C)c(-c2ccc(Cl)cc2)cc1C1=C(O)C(C)(C)CCC1=O] | WO 99/48869 I-8-a-3 | |
| | 2-(4′-chloro-4,6-dimethylbiphenyl-3-yl)- | | |
| | 3-hydroxy-4,4-dimethylcyclohex-2-en- | | |
| | 1-one | | |
| 7-5 | 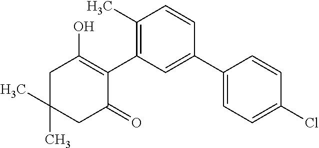 [SMILES: Cc1ccc(-c2ccc(Cl)cc2)cc1C1=C(O)CC(C)(C)CC1=O] | WO 99/48869 I-8-a-4 | |
| | 2-(4′-chloro-4-methylbiphenyl-3-yl)-3- | | |
| | hydroxy-5,5-dimethylcyclohex-2-en-1- | | |
| | one | | |
| 8-1 | 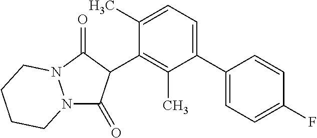 [SMILES: Cc1ccc(-c2ccc(F)cc2)c(C)c1C1C(=O)N2CCCCN2C1=O] | WO 05/016873 I-1-a-34 | |
| | 2-(4′-fluoro-2,4-dimethylbiphenyl-3- | | |
| | yl)tetrahydro-1H-pyrazolo[1,2- | | |
| | a]pyridazine-1,3(2H)-dione | | |
| 8-2 | 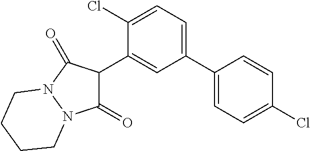 [SMILES: O=C1C(c2cc(-c3ccc(Cl)cc3)ccc2Cl)C(=O)N2CCCCN12] | WO 05/016873 I-1-a-38 | |
| | 2-(4,4′-dichlorobiphenyl-3- | | |
| | yl)tetrahydro-1H-pyrazolo[1,2- | | |
| | a]pyridazine-1,3(2H)-dione | | |
| 8-3 | 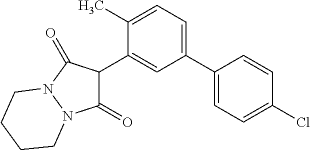 [SMILES: Cc1ccc(-c2ccc(Cl)cc2)cc1C1C(=O)N2CCCCN2C1=O] | WO 05/016873 I-1-a-4 | |
| | 2-(4′-chloro-4-methylbiphenyl-3- | | |
| | yl)tetrahydro-1H-pyrazolo[1,2- | | |
| | a]pyridazine-1,3(2H)-dione | | |
| 8-4 | 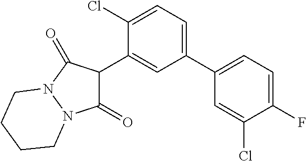 [SMILES: O=C1C(c2cc(-c3ccc(F)c(Cl)c3)ccc2Cl)C(=O)N2CCCCN12] | WO 05/016873 I-1-a-44 | |
| | 2-(3′,4-dichloro-4′-fluorobiphenyl-3- | | |
| | yl)tetrahydro-1H-pyrazolo[1,2- | | |
| | a]pyridazine-1,3(2H)-dione | | |
| 8-5 | 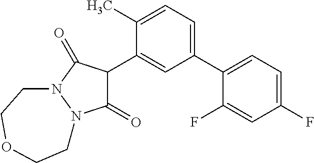 [SMILES: Cc1ccc(-c2ccc(F)cc2F)cc1C1C(=O)N2CCOCCN2C1=O] | WO 05/016873 I-2-a-15 | |
| | 8-(2′,4′-difluoro-4-methylbiphenyl-3- | | |
| | yl)tetrahydro-7H-pyrazolo[1,2- | | |
| | d][1,4,5]oxadiazepine-7,9(8H)-dione | | |
| 8-6 | 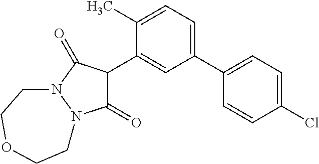 [SMILES: Cc1ccc(-c2ccc(Cl)cc2)cc1C1C(=O)N2CCOCCN2C1=O] | WO 05/016873 I-2-a-3 | |
| | 8-(4′-chloro-4-methylbiphenyl-3- | | |
| | yl)tetrahydro-7H-pyrazolo[1,2- | | |
| | d][1,4,5]oxadiazepine-7,9(8H)-dione | | |
| 8-7 | 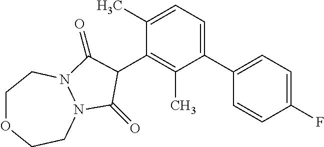 [SMILES: Cc1ccc(-c2ccc(F)cc2)c(C)c1C1C(=O)N2CCOCCN2C1=O] | WO 05/016873 I-2-a-34 | |
| | 8-(4′-fluoro-2,4-dimethylbiphenyl-3- | | |
| | yl)tetrahydro-7H-pyrazolo[1,2- | | |
| | d][1,4,5]oxadiazepine-7,9(8H)-dione | | |
| 8-8 | 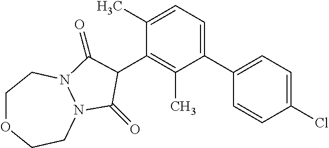 [SMILES: Cc1ccc(-c2ccc(Cl)cc2)c(C)c1C1C(=O)N2CCOCCN2C1=O] | WO 05/016873 I-2-a-35 | |
| | 8-(4′-chloro-2,4-dimethylbiphenyl-3- | | |
| | yl)tetrahydro-7H-pyrazolo[1,2- | | |
| | d][1,4,5]oxadiazepine-7,9(8H)-dione | | |
| 8-9 | 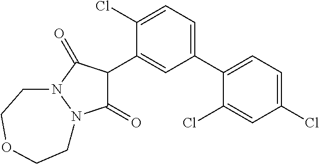 [SMILES: O=C1C(c2cc(-c3ccc(Cl)cc3Cl)ccc2Cl)C(=O)N2CCOCCN12] | WO 05/016873 I-2-a-40 | |
| | 8-(2′,4,4′-trichlorobiphenyl-3- | | |
| | yl)tetrahydro-7H-pyrazolo[1,2- | | |
| | d][1,4,5]oxadiazepine-7,9(8H)-dione | | |
| 8-10 | 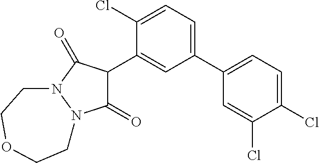 [SMILES: O=C1C(c2cc(-c3ccc(Cl)c(Cl)c3)ccc2Cl)C(=O)N2CCOCCN12] | WO 05/016873 I-2-a-41 | |
| | 8-(3′,4,4′-trichlorobiphenyl-3- | | |
| | yl)tetrahydro-7H-pyrazolo[1,2- | | |
| | d][1,4,5]oxadiazepine-7,9(8H)-dione | | |
| 8-11 | 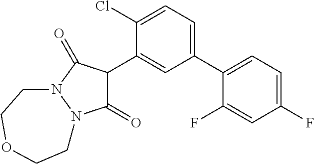 [SMILES: O=C1C(c2cc(-c3ccc(F)cc3F)ccc2Cl)C(=O)N2CCOCCN12] | WO 05/016873 I-2-a-47 | |
| | 8-(4-chloro-2′,4′-difluorobiphenyl-3- | | |
| | yl)tetrahydro-7H-pyrazolo[1,2- | | |
| | d][1,4,5]oxadiazepine-7,9(8H)-dione | | |
| 8-12 | 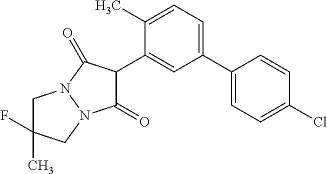 [SMILES: Cc1ccc(-c2ccc(Cl)cc2)cc1C1C(=O)N2CC(C)(F)CN2C1=O] | WO 05/016873 I-5-a-2 | |
| | 2-(4′-chloro-4-methylbiphenyl-3-yl)-6- | | |
| | fluoro-6-methyldihydro-1H,5H- | | |
| | pyrazolo[1,2-a]pyrazole-1,3(2H)-dione | | |
| 8-13 | 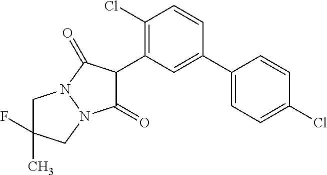 [SMILES: CC1(F)CN2C(=O)C(c3cc(-c4ccc(Cl)cc4)ccc3Cl)C(=O)N2C1] | WO 05/016873 I-5-a-4 | |
| | 2-(4,4′-dichlorobiphenyl-3-yl)-6-fluoro- | | |
| | 6-methyldihydro-1H,5H-pyrazolo[1,2- | | |
| | a]pyrazole-1,3(2H)-dione | | |
| 8-14 | 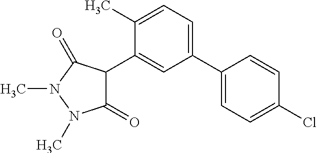 [SMILES: Cc1ccc(-c2ccc(Cl)cc2)cc1C1C(=O)N(C)N(C)C1=O] | WO 05/016873 I-6-a-2 | |
| | 4-(4′-chloro-4-methylbiphenyl-3-yl)-1,2- | | |
| | dimethyl-1H-pyrazole-3,5(2H,4H)- | | |
| | dione | | |
| 8-15 | 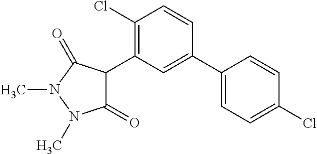 [SMILES: CN1C(=O)C(c2cc(-c3ccc(Cl)cc3)ccc2Cl)C(=O)N1C] | WO 05/016873 I-6-a-3 | |
| | 4-(4,4′-dichlorobiphenyl-3-yl)-1,2- | | |
| | dimethyl-1H-pyrazole-3,5(2H,4H)- | | |
| | dione | | |
| 8-16 | 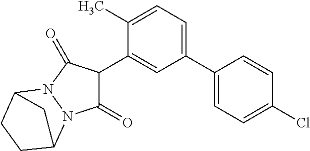 [SMILES: Cc1ccc(-c2ccc(Cl)cc2)cc1C1C(=O)N2C3CCC(C3)N2C1=O] | WO 05/016873 I-8-a-1 | |
| | 2-(4′-chloro-4-methylbiphenyl-3- | | |
| | yl)tetrahydro-1H-5,8- | | |
| | methanopyrazolo[1,2-a]pyridazine- | | |
| | 1,3(2H)-dione | | |
| 8-17 | 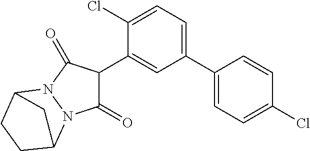 [SMILES: O=C1C(c2cc(-c3ccc(Cl)cc3)ccc2Cl)C(=O)N2C3CCC(C3)N12] | WO 05/016873 I-8-a-2 | |
| | 2-(4,4′-dichlorobiphenyl-3- | | |
| | yl)tetrahydro-1H-5,8- | | |
| | methanopyrazolo[1,2-a]pyridazine- | | |
| | 1,3(2H)-dione | | |
| 9-1 | 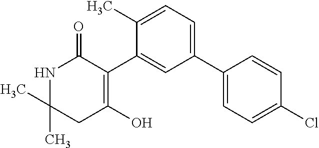 [SMILES: Cc1ccc(-c2ccc(Cl)cc2)cc1C1=C(O)CC(C)(C)NC1=O] | WO 01/79204 I-a-05 | |
| | 3-(4′-chloro-4-methylbiphenyl-3-yl)-4- | | |
| | hydroxy-6,6-dimethyl-5,6- | | |
| | dihydropyridin-2(1H)-one | | |
| 9-2 | 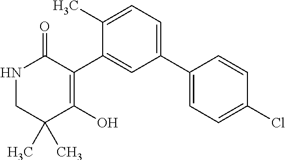 [SMILES: Cc1ccc(-c2ccc(Cl)cc2)cc1C1=C(O)C(C)(C)CNC1=O] | WO 01/79204 I-a-14 | |
| | 3-(4′-chloro-4-methylbiphenyl-3-yl)-4- | | |
| | hydroxy-5,5-dimethyl-5,6- | | |
| | dihydropyridin-2(1H)-one | | |
| 9-3 | 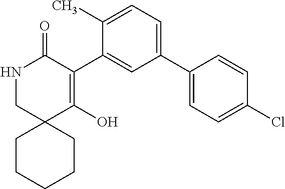 [SMILES: Cc1ccc(-c2ccc(Cl)cc2)cc1C1=C(O)C2(CCCCC2)CNC1=O] | WO 01/79204 I-a-18 | |
| | 4-(4′-chloro-4-methylbiphenyl-3-yl)-5- | | |
| | hydroxy-2-azaspiro[5.5]undec-4-en-3- | | |
| | one | | |
| 9-4 | 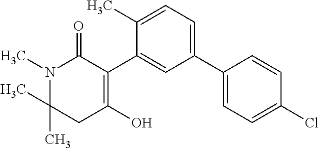 [SMILES: Cc1ccc(-c2ccc(Cl)cc2)cc1C1=C(O)CC(C)(C)N(C)C1=O] | WO 01/79204 I-a-25 | |
| | 3-(4′-chloro-4-methylbiphenyl-3-yl)-4- | | |
| | hydroxy-1,6,6-trimethyl-5,6- | | |
| 9-5 | 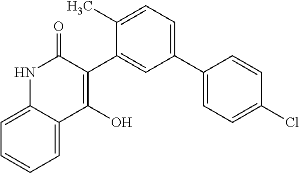 [SMILES: Cc1ccc(-c2ccc(Cl)cc2)cc1-c1c(O)c2ccccc2[nH]c1=O] | WO 03/010145 I-a-1 | |
| | 3-(4′-chloro-4-methylbiphenyl-3-yl)-4- | | |
| | hydroxyquinolin-2(1H)-one | | |
| 9-6 | 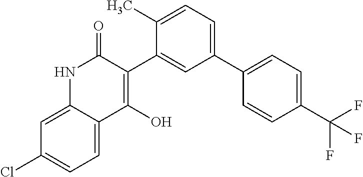 [SMILES: Cc1ccc(-c2ccc(C(F)(F)F)cc2)cc1-c1c(O)c2ccc(Cl)cc2[nH]c1=O] | WO 03/010145 I-a-10 | |
| | 7-chloro-4-hydroxy-3-[4-methyl-4′- | | |
| | (trifluoromethy)biphenyl-3-yl]quinolin- | | |
| | 2(1H)-one | | |
| 9-7 | 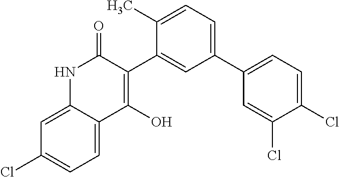 [SMILES: Cc1ccc(-c2ccc(Cl)c(Cl)c2)cc1-c1c(O)c2ccc(Cl)cc2[nH]c1=O] | WO 03/010145 I-a-12 | |
| | 7-chloro-3-(3′,4′-dichloro-4-methyl- | | |
| | biphenyl-3-yl)-4-hydroxyquinolin- | | |
| | 2(1H)-one | | |
| 9-8 | 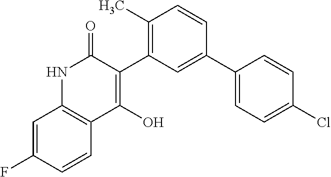 [SMILES: Cc1ccc(-c2ccc(Cl)cc2)cc1-c1c(O)c2ccc(F)cc2[nH]c1=O] | WO 03/010145 I-a-13 | |
| | 3-(4′-chloro-4-methylbiphenyl-3-yl)-7- | | |
| | fluoro-4-hydroxyquinolin-2(1H)-one | | |
| 9-9 | 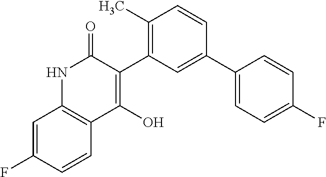 [SMILES: Cc1ccc(-c2ccc(F)cc2)cc1-c1c(O)c2ccc(F)cc2[nH]c1=O] | WO 03/010145 I-a-18 | |
| | 7 fluoro-3-(4′-fluoro-4-methylbiphenyl- | | |
| | 3-yl)-4-hydroxyquinolin-2(1H)-one | | |
| 9-10 | 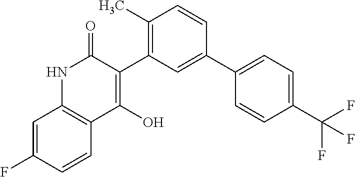 [SMILES: Cc1ccc(-c2ccc(C(F)(F)F)cc2)cc1-c1c(O)c2ccc(F)cc2[nH]c1=O] | WO 03/010145 I-a-19 | |
| | 7-fluoro-4-hydroxy-3-[4-methyl-4′- | | |
| | (trifluoromethyebiphenyl-3-yl]quinolin- | | |
| | 2(1H)-one | | |
| 9-11 | 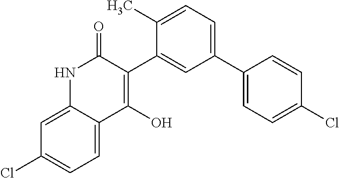 [SMILES: Cc1ccc(-c2ccc(Cl)cc2)cc1-c1c(O)c2ccc(Cl)cc2[nH]c1=O] | WO 03/010145 I-a-3 | (300 MHz, DMSO-d6): 2.13 (s, 3H), 7.21 (dd, 1H), 7.32-7.43 (m, 3H), 7.45-7.52 (m, 2H), 7.57 (dd, 1H), 7.64-7.72 (m, 2H), 7.94 (d, 1H), 10.33 (br. s., 1H), 11.53 (s, 1H). 1.34 min, 396, Method 1 |
| | 7-chloro-3-(4′-chloro-4-methylbiphenyl- | | |
| | 3-yl)-4-hydroxyquinolin-2(1H)-one | | |
| 9-12 | 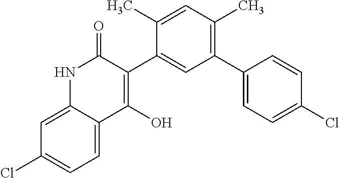 [SMILES: Cc1cc(C)c(-c2c(O)c3ccc(Cl)cc3[nH]c2=O)cc1-c1ccc(Cl)cc1] | WO 03/010145 I-a-7 | |
| | 7-chloro-3-(4′-chloro-4,6-dimethyl- | | |
| | biphenyl-3-yl)-4-hydroxyquinolin- | | |
| | 2(1H)-one | | |
| 9-13 | 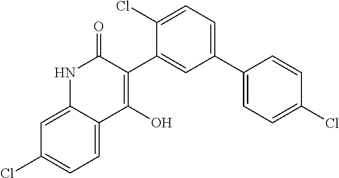 [SMILES: O=c1[nH]c2cc(Cl)ccc2c(O)c1-c1cc(-c2ccc(Cl)cc2)ccc1Cl] | WO 03/010145 I-a-8 | |
| | 7-chloro-3-(4,4′-dichlorobiphenyl-3-yl)- | | |
| | 4-hydroxyquinolin-2(1H)-one | | |
| 9-14 | 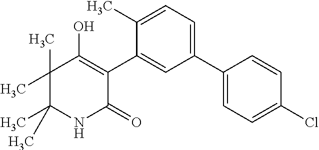 [SMILES: Cc1ccc(-c2ccc(Cl)cc2)cc1C1=C(O)C(C)(C)C(C)(C)NC1=O] | WO 07/068428 I-9-a-1 | |
| | 3-(4′-chloro-4-methylbiphenyl-3-yl)-4- | | |
| | hydroxy-5,5,6,6-tetramethyl-5,6- | | |
| | dihydropyridin-2(1H)-one | | |
| 10-1 | 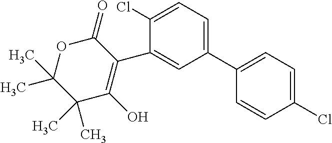 [SMILES: CC1(C)OC(=O)C(c2cc(-c3ccc(Cl)cc3)ccc2Cl)=C(O)C1(C)C] | WO 01/98288 I-a-31 | |
| | 3-(4,4′-dichlorobiphenyl-3-yl)-4- | | |
| | hydroxy-5,5,6,6-tetramethyl-5,6- | | |
| | dihydro-2H-pyran-2-one | | |
| 10-2 | 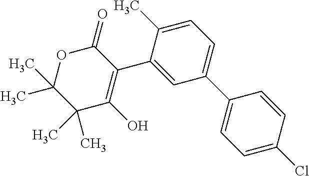 [SMILES: Cc1ccc(-c2ccc(Cl)cc2)cc1C1=C(O)C(C)(C)C(C)(C)OC1=O] | WO 01/98288 I-a-6 | |
| | 3-(4′-chloro-4-methylbiphenyl-3-yl)-4- | | |
| | hydroxy-5,5,6,6-tetramethyl-5,6- | | |
| | dihydro-2H-pyran-2-one | | |
| 11-1 | 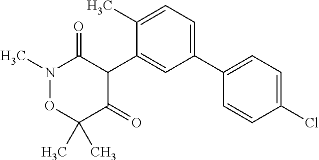 [SMILES: Cc1ccc(-c2ccc(Cl)cc2)cc1C1C(=O)N(C)OC(C)(C)C1=O] | WO 03/048138 I-a-7 | |
| | 4-(4′-chloro-4-methylbiphenyl-3-yl)- | | |
| | 2,6,6-trimethyl-1,2-oxazinane-3,5-dione | | |

### Assays

### Human ACC1 Enzyme Assay

The ACC1 inhibition data were obtained by two different assays (A1 and B1)

### Assay A1 (=(A1))

The inhibitory activity of the substances of this invention with regard to acetyl-CoA carboxylase 1 (ACC1) was measured using the ACC1 assay described in the paragraphs below. The basic principle of the assay is the measurement of adenosine diphosphate (ADP), which is formed as a by-product, by means of an HTRF®-based competitive immunoassay (HTRF=Homogeneous Time Resolved Fluorescence).

The enzyme used was C-terminally FLAG-tagged recombinant human ACC1 (GenBank Accession no. NM_{—}198834, amino acids 39-end), expressed in baculovirus-transfected insect cells (Hi5) and purified by affinity chromatography on Anti-FLAG®M2 affinity gel (Sigma-Aldrich). Alternatively, it is possible to use commercial C-terminally His-tagged ACC1 from BPS Bioscience (San Diego, Calif., catalogue no. 50200, amino acids 39-end). For the assay, 50 n1 of a 100-fold concentrated solution of the test substance in DMSO were pipetted into a black low-volume 384-well microtitre plate (Greiner Bio-One, Frickenhausen, Germany), 2 μl of a solution of ACC1 in assay buffer [50 mM HEPES/NaOH pH 7.5, 12 mM sodium bicarbonate, 2 mM MgCl₂, 2 mM potassium citrate, 0.005% (w/v) bovine serum albumin (BSA)] were added and the mixture was incubated for 15 min to allow pre-binding of the substances to the enzyme prior to the enzyme reaction. The enzyme reaction was then started by addition of 3 μl of a solution of adenosine triphosphate (ATP, 83.5 μM=>the final concentration in 5 μl assay volume is 50 μM, Amersham Pharmacia Biotech #27-2056-01) and acetyl-CoA (33.4 μM=>the final concentration in 5 μl assay volume is 20 μM, Roche Bioscience #10101893001) in assay buffer, and the resulting mixture was incubated at 22° C. for a reaction time of 20 min. The concentration of the ACC1 was adjusted to the respective activity of the enzyme and set such that the assay was carried out in the linear range. Typical concentrations were in the range of 2.5 ng/μl.

The reaction was stopped by successive addition of 2.5 μl of a solution of d2-labelled ADP (HTRF® Transscreener™ ADP kit, C is biointernational, Marcoule, France) in EDTA-containing HTRF® Transscreener™ ADP detection buffer (contained in the HTRF® Transscreener™ ADP kit, 50 mM HEPES pH 7.0, 60 mM EDTA, 0.1% (w/v) BSA, 0.02% sodium azide, 400 mM potassium fluoride) and 2.5 μl of a solution of europium cryptate-labelled anti-ADP antibody (HTRF® Transscreener™ ADP kit) in HTRF® Transscreener™ ADP detection buffer.

The resulting mixture was incubated at 22° C. for 1 h to enable the europium cryptate-labelled anti-ADP antibody to bind to the ADP formed by the enzyme reaction and the d2-labelled ADP. The amount of complex between d2-labelled ADP and europium cryptate-labelled anti-ADP antibody was then determined by measuring the resonance energy transfer from the europium cryptate to d2. To this end, the fluorescence emissions at 620 nm and 665 nm after excitation at 350 nm were measured in an HTRF measuring instrument, for example a Rubystar or Pherastar (both BMG Labtechnologies, Offenburg, Germany). The ratio of the emissions at 665 nm and at 622 nm was taken as a measure for the amount of the complex of d2-labelled ADP and europium cryptate-labelled anti-ADP antibody and thus indirectly as a measure for the amount of the unlabelled ADP formed in the enzyme reaction (higher ratio of the emissions at 665 nm and at 622 nm [symbol] more complex of d2-labelled ADP and europium cryptate-labelled anti-ADP antibody [symbol] less ADP). The data were normalized (enzyme reaction without inhibitor=0% inhibition, all other assay components but no enzyme=100% inhibition). Usually, the test substances were tested on the same microtitre plates at 10 different concentrations in the range from 20 μM to 1 nM (20 μM, 6.7 μM, 2.2 μM, 0.74 μM, 0.25 μM, 82 nM, 27 nM, 9.2 nM, 3.1 nM and 1 nM, the dilution series were prepared before the assay based on the 100 times concentrated solution by serial 1:3 dilutions) in two replications for each concentration, and the IC50 values were calculated with a 4-parameter fit using an inhouse software.

### Assay B1 (=(B1))

The hACC1-inhibitory action of the substances of the present invention was measured in the hACC1 assay described in the paragraphs below.

Essentially, the enzyme activity is measured by quantifying the adenosine diphosphate (ADP) formed as a byproduct of the enzyme reactions using the ADP-Glo™ detection system from Promega. In this test, initially the adenosine triphosphate (ATP) not consumed in the enzyme reaction is converted quantitatively with an adenylate cyclase (“ADP-GLO reagent”) into cAMP, the adenylate cyclase is then stopped and (“kinase detection reagent”) the ADP formed is subsequently converted into ATP, which is converted in a luciferase-based reaction into a glow luminescence signal.

The enzyme used was recombinant C-terminal FLAG-tagged human ACC1 (acetyl-coenzyme A carboxylase alpha transcript variant 1) (GenBank Accession No. NM_{—}198834) (amino acids 39-end) expressed in baculovirus-infected insect cells (Hi5) and purified by anti-FLAG affinity chromatography.

For the assay, 50 nl of a 100 times concentrated solution of the test substance in DMSO were pipetted into a white low-volume 384 well microtitre plate (Greiner Bio-One, Frickenhausen, Germany), 2.5 μl of a solution of hACC1 in assay buffer [50 mM HEPES/NaOH pH 7.5, 2 mM MgCl₂, 2 mM potassium citrate, 12 mM NaHCO₃, 2 mM dithiothreitol (DTT), 0.005% (w/v) bovine serum albumin (BSA)] were added and the mixture was incubated for 15 min to allow prebinding of the substances to the enzyme prior to the enzyme reaction. The enzyme reaction was then started by addition of 2.5 μl of a solution of adenosine triphosphate (ATP, 100 μM=>final concentration in 5 μl of assay volume: 50 μM) and acetyl-CoA (20 μM=>final concentration in 5 μl assay volume: 10 μM) in assay buffer, and the resulting mixture was incubated at 22° C. for the reaction time of 45 min. The concentration of the hACC1 was adapted to the respective activity of the enzyme and adjusted such that the assay operated in the linear range. Typical concentrations were in the range of 1.75 ng/μl. The reaction was stopped by addition of 2.5 μl of the “ADP-GLOreagent” (1:1.5 times diluted), and the resulting mixture was incubated at 22° C. for 1 h to convert the unreacted ATP completely into cAMP. 2.5 μl of the “kinase detection reagent” were then added (1.2 times more concentrated than recommended by the manufacturer), the resulting mixture was incubated at 22° C. for 1 h and the luminescence was then measured using a suitable measuring instrument (Viewlux or Topcount from Perkin-Elmer or Pherastar from BMG Labtechnologies). The amount of light emitted was taken as a measure for the amount of ADP formed and thus for the enzyme activity of the hACC1. The data were normalized (enzyme reaction without inhibitor=0% inhibition, all other assay components but no enzyme=100% inhibition). Usually, the test substances were tested on the same microtitre plates at 10 different concentrations in the range from 20 μM to 1 nM (20 μM, 6.7 μM, 2.2 μM, 0.74 μM, 0.25 μM, 82 nM, 27 nM, 9.2 nM, 3.1 nM and 1 nM, the dilution series were prepared before the assay based on the 100 times concentrated solution by serial 1:3 dilutions) in two replications for each concentration, and the 1050 values were calculated with a 4-parameter fit using an inhouse software.

### Human ACC2 Enzyme Assay

The ACC2 inhibition data were obtained by two different assays (A2 and B2)

### Assay A2 (=(A2))

The inhibitory activity of the substances of this invention with regard to acetyl-CoA carboxylase 2 (ACC2) was measured using the ACC2 assay described in the paragraphs below. The basic principle of the assay is the measurement of adenosine diphosphate (ADP), which is formed as a by-product, by means of an HTRF®-based competitive immunoassay (HTRF=Homogeneous Time Resolved Fluorescence).

The enzyme used was commercially available C-terminally His-tagged ACC2 from BPS Bioscience (San Diego, Calif., catalogue no. 50201, amino acids 39-end, expressed in baculovirus-infected Sf9 insect cells and purified by Ni-NTA affinity chromatography).

For the assay, 50 n1 of a 100-fold concentrated solution of the test substance in DMSO were pipetted into a black low-volume 384-well microtitre plate (Greiner Bio-One, Frickenhausen, Germany), 2 μl of a solution of ACC2 in assay buffer [50 mM HEPES/NaOH pH 7.5, 12 mM sodium bicarbonate, 2 mM MgCl₂, 2 mM potassium citrate, 0.005% (w/v) bovine serum albumin (BSA)] were added and the mixture was incubated for 15 min to allow pre-binding of the substances to the enzyme prior to the enzyme reaction. The enzyme reaction was then started by addition of 3 μl of a solution of adenosine triphosphate (ATP, 83.5 μM=>the final concentration in 5 μl assay volume is 50 μM, Amersham Pharmacia Biotech #27-2056-01) and acetyl-CoA (33.4 μM=>the final concentration in 5 μl assay volume is 20 μM, Roche Bioscience #10101893001) in assay buffer, and the resulting mixture was incubated at 22° C. for a reaction time of 20 min. The concentration of the ACC2 was adjusted to the respective activity of the enzyme and set such that the assay was carried out in the linear range. Typical concentrations were in the range of 0.6 ng/μl.

The reaction was stopped by successive addition of 2.5 μl of a solution of d2-labelled ADP (HTRF® Transscreener™ ADP kit, C is biointernational, Marcoule, France) in EDTA-containing HTRF® Transscreener™ ADP detection buffer (contained in the HTRF® Transscreener™ ADP kit, 50 mM HEPES pH 7.0, 60 mM EDTA, 0.1% (w/v) BSA, 0.02% sodium azide, 400 mM potassium fluoride) and 2.5 μl of a solution of europium cryptate-labelled anti-ADP antibody (HTRF® Transscreener™ ADP kit) in HTRF® Transscreener™ ADP detection buffer.

The resulting mixture was incubated at 22° C. for 1 h to allow binding of the europium cryptate-labelled anti-ADP antibody to the ADP formed by the enzyme reaction and the d2-labelled ADP. The amount of complex of d2-labelled ADP and europium cryptate-labelled anti-ADP antibody was then determined by measuring the resonance energy transfer of europium cryptate to d2. To this end, the fluorescence emissions at 620 nm and 665 nm after excitation at 350 nm were measured in an HTRF measuring instrument, for example a Rubystar or Pherastar (both BMG Labtechnologies, Offenburg, Germany). The ratio of the emissions at 665 nm and at 622 nm was taken as a measure of the amount of the complex of d2-labelled ADP and europium cryptate-labelled anti-ADP antibody and thus indirectly as a measure for the amount of unlabelled ADP formed in the enzyme reaction (higher ratio of the emissions at 665 nm and at 622 nm [symbol] more complex of d2-labelled ADP and europium cryptate-labelled anti-ADP antibody [symbol] less ADP). The data were normalized (enzyme reaction without inhibitor=0% inhibition, all other assay components but no enzyme=100% inhibition). The test substances were usually tested on the same microtitre plates at 10 different concentrations in the range from 20 μM to 1 nM (20 μM, 6.7 μM, 2.2 μM, 0.74 μM, 0.25 μM, 82 nM, 27 nM, 9.2 nM, 3.1 nM and 1 nM, the dilution series were prepared prior to the assay based on the 100-times concentrated solution by serial 1:3 dilutions) in two replications for each concentration, and IC50 values were calculated with a 4-parameter fit using an inhouse software.

### Assay B2 (=(B2))

The hACC2-inhibitory action of the substances of the present invention was measured in the hACC2 assay described in the paragraphs below.

Essentially, the enzyme activity is measured by quantifying the adenosine diphosphate (ADP) formed as a byproduct of the enzyme reactions using the ADP-Glo™ detection system from Promega. In this test, initially the adenosine triphosphate (ATP) not consumed in the enzyme reaction is converted quantitatively with an adenylate cyclase (“ADP-GLO reagent”) into cAMP, the adenylate cyclase is then stopped and (“kinase detection reagent”) the ADP formed is subsequently converted into ATP, which is converted in a luciferase-based reaction into a glow luminescence signal.

The enzyme used was recombinant C-terminal FLAG-tagged human ACC2 (acetyl-coenzyme A carboxylase 2) (GenBank Accession No. NP_{—}001084) (amino acids 27-end) expressed in baculovirus-infected insect cells (Hi5) and purified by anti-FLAG affinity chromatography.

For the assay, 50 n1 of a 100 times concentrated solution of the test substance in DMSO were pipetted into a white low-volume 384 well microtitre plate (Greiner Bio-One, Frickenhausen, Germany), 2.5 μl of a solution of hACC1 in assay buffer [50 mM HEPES/NaOH pH 7.5, 2 mM MgCl₂, 2 mM potassium citrate, 12 mM NaHCO₃, 2 mM dithiothreitol (DTT), 0.005% (w/v) bovine serum albumin (BSA)] were added and the mixture was incubated for 15 min to allow prebinding of the substances to the enzyme prior to the enzyme reaction. The enzyme reaction was then started by addition of 2.5 μl of a solution of adenosine triphosphate (ATP, 100 μM=>final concentration in 5 μl of assay volume: 50 μM) and acetyl-CoA (20 μM=>final concentration in 5 μl assay volume: 10 μM) in assay buffer, and the resulting mixture was incubated at 22° C. for the reaction time of 45 min. The concentration of the hACC2 was adapted to the respective activity of the enzyme and adjusted such that the assay operated in the linear range. Typical concentrations were in the range of 2 ng/μl. The reaction was stopped by addition of 2.5 μl of the “ADP-GLOreagent” (1:1.5 times diluted), and the resulting mixture was incubated at 22° C. for 1 h to convert the unreacted ATP completely into cAMP. 2.5 μl of the “kinase detection reagent” were then added (1.2 times more concentrated than recommended by the manufacturer), the resulting mixture was incubated at 22° C. for 1 h and the luminescence was then measured using a suitable measuring instrument (Viewlux or Topcount from Perkin-Elmer or Pherastar from BMG Labtechnologies). The amount of light emitted was taken as a measure for the amount of ADP formed and thus for the enzyme activity of the hACC2. The data were normalized (enzyme reaction without inhibitor=0% inhibition, all other assay components but no enzyme=100% inhibition). Usually, the test substances were tested on the same microtitre plates at 10 different concentrations in the range from 20 μM to 1 nM (20 μM, 6.7 μM, 2.2 μM, 0.74 μM, 0.25 μM, 82 nM, 27 nM, 9.2 nM, 3.1 nM and 1 nM, the dilution series were prepared before the assay based on the 100 times concentrated solution by serial 1:3 dilutions) in two replications for each concentration, and the IC₅₀ values were calculated with a 4-parameter fit using an inhouse software.

### Non-Human ACCase Assay

The assay was carried out at room temperature in a transparent 384-well microtitre plate. It determined the inorganic phosphate released from the ATP in the ACCase reaction.

The test mixture contained 50 mM Tris-HCl pH 8.3, 50 mM KCl, 2.5 mM MgCl₂, 0.5 mM ATP, 0.8 mM dithiothreitol (DTT), 30 mM NaHCO₃, 0.1 mM acetyl-CoA, 0.04% bovine serum albumin and 0.4 μg partially purified ACCase enzyme in a final volume of 40 μl. After 45 minutes of incubation, the reaction was stopped with 150 μl of malachite green solution, and the absorption at 620 was read after 30 minutes.

The malachite green (MG) solution was prepared by mixing 3 parts of 0.6 mM MG-HCl solution in distilled water with 1 part of 8.5 mM ammonium molybdate in 4 M HCl. The solution was allowed to stand for 30 minutes. After filtration through a 0.45 μm polytetrafluoroethylene (PTFE) filter, 0.1 part of Triton X-100 (1.5%) in distilled water was added.

ACCase enzyme was extracted from oat seedlings 9 days after sowing and partially purified by precipitation with 0-40% ammonium sulphate followed by ion exchange chromatography on Q-Sepharose.

### Mode-of-Action Experiment

Prior to the determination of the activity in the MCF-7 model, some of the test substances were examined in a “mode of action” experiment. The principle of this experiment is that short-term application of a test substance capable of inhibiting ACC1 and/or ACC2 in a living organism after oral administration reduces malonyl-CoA in a tumour. To this end, in the experiment 2 million human MCF-7 breast cancer cells were injected subcutaneously into female nude mice (NMRI-nude (nu/nu) mice, Taconic M&B A/S, 1 day beforehand administration of a pellet for the release of oestrogen over a period of at least 60 days). Once the tumour extended to an area of about 60-70 mm², the test substance was administered orally over a period of 1-3 days, and at defined points in time the intratumour content of malonyl-CoA was then determined and compared to the vehicle control. The method is described in Anal Chem 2008 Aug. 1; 80(15):5736-42. Epub 2008 Jul. 9.).

### Cell Assays

In accordance with the invention, the substances were tested in cell-based assays for the ability of the substances of inhibiting tumour cell proliferation after a 96-hour incubation with the substance. Cell viability was tested using the CellTiter-Glo® luminescent cell viability assay (Promega). The cells were sown at a density of 2000-5000 cells/well (depending on the cell line) in 100 μl growth medium on 96-well microtitre plates. For each cell line examined, cells were sown on a separate plate to determine the luminescence at t=0 hours and t=96 hours. After overnight incubation at 37° C., the luminescence values for the t=0 samples were determined. The dose plates for the t=96 hours points in time were treated with substances diluted with growth medium. The cells were then incubated at 37° C. for 96 hours, and the luminescence values for the t=96 hours sample were then determined. For data analysis, the t=0 values were subtracted from the t=96 hour values for treated and untreated samples. The differences in luminescence in percent between substance-treated samples and control values were used to determine the growth inhibition in percent.

The substances were tested in the following cell lines which represent the stated indications in an exemplary manner:

| Cell line | Source | Indication |
|---|---|---|
| MDA-MB-436 | ATCC | hormone receptor-negative breast carcinoma |
| MDA-MB-468 | ATCC | hormone receptor-negative breast carcinoma |
| HCC-1937 | ATCC | BRCA-associated breast carcinoma |
| MCF7 | ATCC | hormone receptor-positive breast carcinoma |
| Miapaca | ATCC | pancreas carcinoma |
| 786 O | ATCC | kidney cell carcinoma |
| PLC/PRF5 | ATCC | hepatocellular carcinoma |
| A431 | ATCC | skin carcinoma |
| MDA-MB-435 | ATCC | malignant melanoma |
| NCI-H2135 | ATCC | non-small-cell bronchial carcinoma |
| DLD1 | ATCC | colorectal carcinoma |
| PC3 | ATCC | prostate carcinoma |
| Du145 | NCI | prostate carcinoma |
| ECC1 | ATCC | endometrial carcinomas |
| KM12 | NCI | colorectal carcinoma |
| HEC1A | ATCC | endometrial carcinomas |

### Xenograft Model

Xenograft models in immunosuppressed mice were used to determine the antitumour activity in living organisms.

To this end, initially the maximum tolerable dose (MTD) was determined using the following protocol:

Over a period of 1, 2 or 3 weeks, a defined dose of the test substance was administered orally to female nude mice (NMRI-nude (nu/nu) mice, Taconic M&B A/S), and the mice were observed daily for mortality and body weight. The MTD was defined as the highest dose which could be administered without any animal dying during the treatment phase and the 7-day additional observation phase, and without any body weight loss of more than 10% compared to the initial weight.

Various xenograft models in which the test substances were administered in their MTD and in lower doses were then used to determine the antitumour activity. In addition to various other models, use was made primarily of the breast cancer model with human MCF-7 cells in female nude mice (NMRI-nude (nu/nu) mice, Taconic M&B A/S). To this end, on the day prior to the implantation of the tumour cells, a pellet for releasing oestrogen over a period of at least 60 days was administered subcutaneously to the mice. The next day, 2 million tumour cells were then injected subcutaneously into the side of each animal. Therapy with the test substance was initiated once the tumour extended to an area of about 25 mm². The therapy was then continued until an average tumour size of 120 mm² had been reached in the control group, which had only been given the vehicle of the test substance, or in one of the treatment groups, with tumour area and body weight being measured 2-3 times per week. At this point in time, the experiment was terminated in all groups and the excised tumours were weighed. The T/C value (average tumour weight in the therapy group divided by the average tumour weight in the vehicle control group) was calculated as primary success parameter.

### Analysis of the ACC1 Expression in Tumour Tissue and Normal Tissue

The ACC1 expression was determined using a microarray. To this end, the RNA of various tumour tissues and the corresponding normal tissues was isolated. The method made use of Trizol RNA extraction reagent (Invitrogen) and subsequent purification using the RNeasy mini kit (Qiagen). Moreover, a DNase I (Qiagen) digestion was carried out to eliminate genomic DNA. For quality control, the total RNA was analyzed with the aid of an RNA LabChip on an Agilent Bioanalyzer 2100 Platform (Agilent Technologies), and the RNA concentration was determined using the Peqlab NanoDrop system. For hybridization, the one-cycle eukaryotic target labelling assay from Affymetrix was used, and the array was then read on an AffymetrixGeneChip 3000 scanner (Affymetrix). Evaluation and quality control were carried out using the Expressionist Pro 4.0 Refiner (GeneData) software.

### Tablets Comprising Exemplary Compound 1-118

### a) Preparation of the Pharmaceutical Formulation by Direct Tabletting

Tablets according to the composition from the table mentioned below comprising exemplary compound 1-118 were prepared by direct tabletting.

| Starting materials | Mass/Tablet [mg] |
|---|---|
| exemplary compound 1-118 | 80.0 |
| mannitol, spray-dried | 67.0 |
| cellulose, microcrystalline | 40.0 |
| Na-croscarmellose | 10.0 |
| magnesium stearate | 3.0 |
| total | 200.0 |

The pharmaceutical formulation can be prepared by suitable processes, in particular by powder mixing and direct tabletting, in any scale.

To prepare 50 tablets,

3.351 g of mannitol, spray-dried
2.004 g of cellulose, microcrystalline

### 0.499 g of Na-croscarmellose and

3.992 g of exemplary compound 1-118
were premixed in a mortar by careful grinding. The mixture was transferred into a 100-ml screw-cap tube and homogenized in a Turbula mixer for 10 minutes. After addition of 0.149 g of magnesium stearate, the mixture was mixed in the Turbula mixer for another 1 min.

The moulding material obtained in this manner was tabletted in an eccentric tablet press (Korsch EK 2) to give biconvex tablets of a diameter of 8 mm and a curvature of 12 mm.

### b) Break Force

Break force (using a Schleuniger break force tester), mass and disintegration time in water at 37° C. (using the apparatus described in the monograph 2.9.1 European Pharmacopoeia) of the tablets obtained was tested at the beginning, in the middle and at the end of the tabletting process.

| Break force | Mass | Disintegration time | |
|---|---|---|---|
| beginning | 81N | 198.7 mg | 1:28 min |
| middle | 95N | 196.8 mg | 1:28 min. |
| end | 97N | 199.0 mg | 1:32 min. |
| mean | 91N | 198.2 mg | 1:29 min. |

c) In-Vitro Dissolution of the Pharmaceutical Formulation

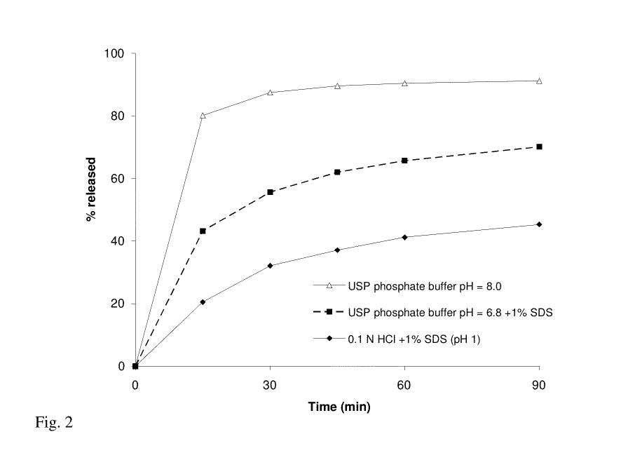

The in vitro release of the exemplary compound 1-118 from the tablets prepared was determined using apparatus 2 (paddle method) in accordance with USP. The release test was in each case carried out in 900 ml of various media at 37° C. and with a stirrer speed of 75 rotations per minute (FIG. 2). Each determination was carried out in three replications. The content was determined by HPLC.

The following results were obtained:

| | % released after | | | | |
|---|---|---|---|---|---|
| Medium | 15 min | 30 min | 45 min | 60 min | 90 min |
| 0.1N HCl + 1% SDS* | 20.5% | 32.1% | 37.1% | 41.2% | 45.3% |
| (pH 1) | | | | | |
| USP phosphate buffer | 43.2% | 55.6% | 62.0% | 65.7% | 70.1% |
| pH = 6.8 + 1% SDS* | | | | | |
| USP phosphate buffer | 80.1% | 87.5% | 89.6% | 90.4% | 91.2% |
| pH = 8.0 | | | | | |

*SDS = sodium laurylsulphate (added because of insufficient solubility at pH 1 and pH 6.8)

*SDS=sodium laurylsulphate (added because of insufficient solubility at pH 1 and pH 6.8)

### d) Short-Term Stability of the Pharmaceutical Formulation

The finished tablets were subjected to a 1-month short-term stability test at 25° C./60% relative humidity and at 40° C./75% relative humidity. Under either conditions, the tablets were stable with respect to content and degradation products, examined by HPLC.

### Results:

Table 4 summarizes the results of the enzyme assays and the mode-of-action test for the compounds of the formula (I-1).

TABLE 4

| | | | | | % Inhibition of |
|---|---|---|---|---|---|
| | ACC 1 | ACC 2 | ACC 1 | ACC 2 | Malonyl-CoA |
| | (=A1) | (=A2) | (=B1) | (=B2) | after 7 h, |
| Ex. | IC50 | IC50 | IC50 | IC50 | 10 mg/kg |
| No. | [μmol/l] | [μmol/l] | [μmol/l] | [μmol/l] | (vs Vehicle) |
| 1-1 | 0.46 | 8.8 | 0.245 | >20.0 | 39 |
| 1-2 | 0.28 | 0.37 | 0.084 | 0.822 | 96 |
| 1-3 | 0.45 | 5.4 | 0.643 | >20.0 | |
| 1-4 | 0.46 | 0.33 | 0.357 | 0.558 | |
| 1-5 | 0.287 | 8.242 | 0.246 | 11.0 | 82 |
| 1-6 | 0.30 | 5.2 | 0.133 | 9.34 | 68 |
| 1-7 | 1.7 | 0.087 | 1.46 | 0.328 | 89 |
| 1-8 | 0.79 | 0.81 | 5.81 | >20.0 | |
| 1-9 | 16.605 | 0.626 | 11.4 | 0.276 | |
| 1-10 | 0.550 | 6.402 | 0.500 | 9.46 | |
| 1-11 | 0.24 | 1.0 | 0.157 | 4.47 | 87 |
| 1-12 | 0.772 | 18.219 | 0.871 | >20.0 | |
| 1-13 | 0.353 | 18.936 | 0.216 | >20.0 | |
| 1-14 | 0.990 | >20 | 3.07 | >20.0 | |
| 1-15 | 0.742 | >20 | 0.522 | >20.0 | |
| 1-16 | 0.329 | >20 | 0.241 | >20.0 | |
| 1-17 | 0.334 | 4.021 | 0.553 | 11.4 | |
| 1-18 | 0.797 | 7.608 | 0.982 | 16.4 | |
| 1-19 | 0.874 | 6.965 | 0.532 | 20.0 | |
| 1-20 | 0.240 | 0.214 | 0.230 | 1.13 | 87 |
| 1-21 | 0.533 | 15.131 | 0.630 | 10.3 | |
| 1-22 | 0.969 | 0.695 | 2.68 | 16.3 | |
| 1-23 | 0.584 | 0.485 | 0.459 | 2.33 | |
| 1-24 | 0.34 | 0.21 | 0.230 | 0.517 | 91 |
| 1-25 | 0.495 | 3.356 | 0.288 | 3.78 | |
| 1-26 | 0.540 | 1.254 | 0.367 | 3.09 | |
| 1-27 | 0.472 | >20 | 1.94 | >20.0 | |
| 1-28 | 0.443 | 0.192 | 0.438 | 2.11 | |
| 1-29 | 0.27 | 1.9 | 0.153 | 2.62 | 93 |
| 1-30 | 0.542 | 0.300 | 0.481 | 0.976 | 82 |
| 1-31 | 0.963 | 5.956 | 0.811 | 7.89 | |
| 1-32 | 0.86 | 14 | 0.516 | 19.0 | |
| 1-33 | 0.359 | 0.535 | 0.234 | 2.01 | 88 |
| 1-34 | 0.47 | 9.4 | 0.297 | 11.4 | |
| 1-35 | 0.298 | 3.998 | 0.388 | 6.91 | 31 |
| 1-36 | 0.792 | >20 | 1.50 | >20.0 | |
| 1-37 | 0.810 | >20 | 0.850 | >20.0 | |
| 1-38 | 0.497 | >20 | 0.747 | >20.0 | |
| 1-39 | 0.663 | >20 | 1.98 | >20.0 | |
| 1-40 | 1.2 | 15 | 1.32 | >20.0 | |
| 1-41 | 0.751 | >20 | 1.14 | >20.0 | |
| 1-42 | >20 | 0.669 | — | — | |
| 1-43 | 0.304 | 10.240 | 4.93 | >20.0 | |
| 1-44 | 0.915 | >20 | 1.70 | >20.0 | |
| 1-45 | 0.776 | 3.608 | 0.701 | 8.83 | |
| 1-46 | 0.812 | 15.779 | 1.13 | >20.0 | |
| 1-47 | 0.923 | >20 | 3.19 | >20.0 | |
| 1-48 | 1.006 | 8.598 | 1.56 | 10.4 | |
| 1-49 | 0.24 | 9.9 | 10.6 | >20.0 | |
| 1-50 | 0.472 | 6.267 | 11.8 | >20.0 | |
| 1-51 | 0.473 | 4.913 | 0.415 | 5.45 | |
| 1-52 | 1.268 | >20 | 1.79 | 16.0 | |
| 1-53 | 0.506 | 0.214 | 0.243 | 0.632 | 43 |
| 1-54 | 0.22 | 1.1 | 0.157 | 1.35 | 61 |
| 1-55 | 0.918 | 0.465 | 0.924 | 3.29 | |
| 1-56 | 0.43 | 0.32 | 0.202 | 0.672 | 84 |
| 1-57 | 0.79 | 0.22 | 1.29 | 1.68 | |
| 1-58 | 0.362 | 11.486 | 0.434 | 19.9 | |
| 1-59 | 0.427 | 5.205 | 0.261 | 11.6 | |
| 1-60 | 0.237 | 2.334 | 0.189 | 5.99 | |
| 1-61 | 0.227 | 3.910 | 0.088 | 9.04 | |
| 1-62 | 0.690 | | 0.385 | 5.74 | |
| 1-63 | 0.391 | 4.216 | 0.277 | 7.27 | |
| 1-64 | 0.985 | 16.443 | 0.571 | >20.0 | |
| 1-65 | 0.934 | 9.465 | 1.16 | 14.7 | |
| 1-66 | 0.409 | 1.542 | 0.493 | 3.13 | |
| 1-67 | 0.668 | 1.443 | 0.918 | 5.20 | |
| 1-68 | 0.489 | 4.860 | 0.547 | 5.90 | |
| 1-69 | 0.647 | 10.997 | 0.838 | >20.0 | |
| 1-70 | 0.675 | 1.839 | 0.286 | 2.92 | |
| 1-71 | 0.661 | >20 | 0.912 | >20.0 | |
| 1-72 | 1.179 | 2.340 | 1.14 | 14.9 | |
| 1-73 | 0.912 | 4.971 | 0.563 | >20.0 | |
| 1-74 | 0.656 | 16.428 | 2.42 | >20.0 | |
| 1-75 | 7.975 | 0.376 | 5.68 | 0.803 | |
| 1-76 | 0.201 | 0.233 | 0.163 | 0.432 | 96 |
| 1-77 | 0.327 | 0.860 | 0.278 | 2.59 | 96 |
| 1-78 | 0.795 | 1.729 | 0.958 | 8.12 | |
| 1-79 | 0.848 | >20 | 2.32 | >20.0 | |
| 1-80 | 0.695 | >20 | 2.18 | >20.0 | |
| 1-81 | 0.327 | 1.414 | 0.428 | 2.61 | |
| 1-82 | 0.542 | 0.433 | 0.539 | 1.32 | |
| 1-83 | 0.459 | 0.349 | 0.582 | 1.88 | |
| 1-84 | 0.455 | | 0.650 | >20.0 | |
| 1-85 | 0.34 | 4.0 | 0.251 | 9.21 | |
| 1-86 | 0.541 | 3.302 | 0.575 | 6.12 | |
| 1-87 | 0.54 | 7.3 | 0.378 | 10.5 | 71 |
| 1-88 | 0.825 | 6.898 | 0.452 | 11.7 | |
| 1-89 | 0.858 | >20 | 2.07 | 13.6 | |

| Ex. | ACC 1 (=A1) | ACC 2 (=A2) | ACC 1 (=B1) | ACC 2 (=B2) |
|---|---|---|---|---|
| No. | IC50 [μmol/l] | IC50 [μmol/l] | IC50 [μmol/l] | IC50 [μmol/l] |
| 1-90 | 0.143 | 0.331 | 0.063 | 0.595 |
| 1-91 | 0.302 | 5.01 | 0.266 | 4.99 |
| 1-92 | 0.226 | 1.45 | 0.072 | 1.66 |
| 1-93 | 0.301 | 0.876 | 0.217 | 4.01 |
| 1-94 | 0.073 | 0.873 | 0.099 | 2.03 |
| 1-95 | 0.378 | 0.393 | 0.383 | 2.41 |
| 1-96 | 0.416 | >20.0 | 0.238 | >20.0 |
| 1-97 | 1.94 | 0.138 | 1.113 | 0.269 |
| 1-98 | 2.19 | 0.204 | 1.035 | 0.174 |
| 1-99 | 0.327 | 6.67 | 0.207 | 11.3 |
| 1-100 | 0.091 | 0.988 | 0.096 | 2.47 |
| 1-101 | 0.460 | 6.07 | 0.650 | 10.5 |
| 1-102 | 0.214 | 2.95 | 0.243 | 3.82 |
| 1-103 | 0.151 | 1.18 | 0.102 | 4.26 |
| 1-104 | 0.329 | 17.9 | 0.270 | >20.0 |
| 1-105 | 0.360 | 5.56 | 0.165 | 8.94 |
| 1-106 | 0.439 | 4.36 | 0.207 | 5.85 |
| 1-107 | 0.804 | >20.0 | 0.376 | >20.0 |
| 1-108 | 0.460 | 0.346 | 0.204 | 0.753 |
| 1-109 | 0.800 | 0.103 | 0.293 | 0.394 |
| 1-110 | 0.194 | 0.640 | 0.080 | 0.488 |
| 1-111 | 0.177 | 0.592 | 0.126 | 1.12 |
| 1-112 | 0.167 | 3.91 | 0.200 | 8.43 |
| 1-113 | 0.260 | 1.35 | 0.101 | 2.71 |
| 1-114 | 0.236 | 2.21 | 0.113 | 1.64 |
| 1-115 | 0.314 | 5.04 | 0.196 | 10.3 |
| 1-116 | 0.232 | 0.280 | 0.092 | 0.591 |
| 1-117 | 0.232 | 0.812 | 0.194 | 4.11 |
| 1-118 | 0.129 | 0.690 | 0.102 | 1.38 |
| 1-119 | 0.203 | 0.479 | 0.110 | 1.08 |
| 1-120 | 0.250 | 1.28 | 0.145 | 2.26 |
| 1-121 | 0.402 | — | 0.172 | 1.01 |
| 1-122 | 0.098 | 5.42 | 0.124 | 6.59 |
| 1-123 | 0.173 | 3.44 | 0.562 | 11.2 |
| 1-124 | 0.458 | >20.0 | 0.467 | >20.0 |
| 1-125 | 0.281 | 3.99 | 0.217 | 6.77 |
| 1-126 | 0.300 | 6.37 | 0.204 | 12.4 |
| 1-127 | 0.216 | 4.84 | 0.145 | 13.4 |
| 1-128 | 0.195 | 2.74 | 0.157 | 4.41 |
| 1-129 | 0.349 | 9.61 | 0.267 | 10.6 |
| 1-130 | 0.133 | 4.40 | 0.108 | 5.89 |
| 1-131 | 0.266 | 7.62 | 0.098 | 5.62 |
| 1-132 | 1.71 | 0.106 | 0.912 | 0.234 |
| 1-133 | 0.329 | 2.87 | 0.142 | 8.10 |
| 1-134 | 0.299 | 1.31 | 0.109 | 2.85 |
| 1-135 | 0.220 | 0.657 | 0.094 | 1.75 |
| 1-136 | 0.239 | 6.49 | 0.233 | 12.5 |
| 1-137 | 0.307 | 7.75 | 0.168 | 7.49 |
| 1-138 | 0.262 | 0.633 | 0.172 | 2.19 |
| 1-139 | 0.280 | 0.605 | 0.153 | 4.55 |
| 1-140 | 0.323 | 1.52 | — | — |
| 1-141 | 0.247 | 0.625 | 0.161 | 1.38 |
| 1-142 | 0.337 | 0.551 | 0.704 | 0.957 |
| 1-143 | 0.282 | 1.02 | 0.161 | 2.15 |
| 1-144 | 0.406 | 4.92 | 0.221 | 8.53 |
| 1-145 | 0.194 | 3.31 | — | — |
| 1-146 | 0.197 | 0.455 | 0.115 | 0.679 |
| 1-147 | 0.195 | 1.25 | 0.150 | 2.03 |

Table 5 summarizes the results of the enzyme assays and the mode-of-action test for the compounds of the formula (I-2).

TABLE 5

| | | | | | % Inhibition of |
|---|---|---|---|---|---|
| | ACC 1 | ACC 2 | ACC 1 | ACC 2 | Malonyl-CoA |
| | (=A1) | (=A2) | (=B1) | (=B2) | after 7 h, |
| Ex. | IC50 | IC50 | IC50 | IC50 | 10 mg/kg |
| No. | [μmol/l] | [μmol/l] | [μmol/l] | [μmol/l] | (vs Vehicle) |
| 2-1 | 0.243 | 4.150 | 1.14 | 15.2 | |
| 2-2 | 0.260 | 5.285 | 2.36 | >20.0 | |
| 2-3 | 0.581 | 11.696 | 3.73 | >20.0 | |
| 2-4 | 0.19 | 1.5 | 0.175 | 2.70 | |
| 2-5 | 1.478 | 5.715 | 1.26 | 7.34 | |
| 2-6 | 2.151 | 0.139 | 6.85 | 5.42 | |
| 2-7 | 0.500 | >20 | 2.71 | >20.0 | |
| 2-8 | 0.420 | >20 | 1.99 | >20.0 | |
| 2-9 | 0.334 | >20 | 2.22 | >20.0 | |
| 2-10 | 0.161 | 2.354 | 0.067 | 4.70 | |
| 2-11 | 0.666 | 1.547 | 1.26 | 12.4 | |
| 2-12 | 4.173 | 1.152 | 4.26 | 3.42 | |
| 2-13 | 0.816 | >20 | 1.02 | >20.0 | |
| 2-14 | 0.562 | 19.353 | 0.772 | >20.0 | |
| 2-15 | 0.204 | 3.676 | 0.111 | 8.02 | |
| 2-16 | 0.180 | 3.336 | 0.138 | 5.31 | |
| 2-17 | 0.158 | 0.652 | 0.058 | 1.18 | |
| 2-18 | 0.479 | 3.409 | 0.276 | 12.2 | |
| 2-19 | 0.530 | 10.007 | 0.812 | 13.6 | |
| 2-20 | 0.137 | 1.929 | 0.102 | 1.72 | |
| 2-21 | 0.40 | >20 | 0.874 | >20.0 | |
| 2-22 | 0.824 | >20 | 2.23 | >20.0 | |
| 2-23 | 1.369 | >20 | 2.09 | >20.0 | |
| 2-24 | 0.997 | >20 | 2.09 | >20.0 | |
| 2-25 | 0.442 | >20 | 1.20 | >20.0 | |
| 2-26 | 0.26 | 4.8 | 6.11 | >20.0 | |
| 2-27 | 0.822 | 4.146 | 0.801 | 7.01 | |
| 2-28 | 0.14 | 2.5 | 0.128 | 4.60 | |
| 2-29 | 0.321 | 0.789 | 0.206 | 3.00 | |
| 2-30 | 0.281 | 0.032 | 0.696 | 0.243 | 36 |
| 2-31 | 0.88 | 0.048 | 2.00 | 0.076 | |
| 2-32 | 0.45 | 1.6 | 0.339 | 2.82 | |
| 2-33 | 0.229 | 3.586 | 0.227 | 5.00 | 39 |
| 2-34 | 0.286 | 1.274 | — | — | |
| 2-35 | 1.024 | >20 | 1.19 | >20.0 | |
| 2-36 | n.d. | 1.936 | 0.447 | 2.41 | |
| 2-37 | 0.342 | >20 | 1.05 | >20.0 | |
| 2-38 | 0.416 | 3.785 | 0.393 | 7.38 | |
| 2-39 | 0.216 | 3.533 | 0.162 | 5.27 | |
| 2-40 | 1.216 | >20 | 1.51 | >20.0 | |
| 2-41 | 0.791 | 4.217 | 0.425 | 6.59 | |
| 2-42 | 0.357 | 1.021 | 0.246 | 1.56 | |
| 2-43 | 0.504 | 0.330 | 0.168 | 0.737 | |
| 2-44 | 0.297 | 0.160 | 0.220 | 0.244 | |
| 2-45 | 0.568 | 17 | 1.83 | 17.5 | |
| 2-46 | 0.580 | 7.610 | 0.420 | 5.26 | |
| 2-47 | 0.592 | 0.453 | 0.402 | 0.389 | |
| 2-48 | 1.800 | 0.204 | 1.54 | 0.417 | |
| 2-49 | 5.776 | 1.281 | 20.0 | >20.0 | |
| 2-50 | 0.903 | 8.593 | 1.30 | 15.4 | |

Table 6 summarizes the results of the enzyme assays for the compounds of the formulae (I-3), (I-6), (I-7), (I-8), (I-9), (I-10) and (I-11).

TABLE 6

| Ex. | ACC 1 (=A1) | ACC 2 (=A2) | ACC 1 (=B1) | ACC 2 (=B2) |
|---|---|---|---|---|
| No. | IC50 [μmol/l] | IC50 [μmol/l] | IC50 [μmol/l] | IC50 [μmol/l] |
| 3-1 | 0.853 | | 1.41 | 15.5 |
| 3-2 | 0.097 | 1.675 | — | — |
| 3-3 | 0.34 | 0.25 | 0.265 | 1.15 |
| 3-4 | 1.311 | 0.228 | — | — |
| 6-1 | 0.655 | 16.024 | 0.411 | 19.7 |
| 6-2 | 0.337 | 1.688 | 0.263 | 3.23 |
| 6-3 | 1.390 | 16.220 | 1.17 | >20.0 |
| 6-4 | 0.333 | 3.059 | 0.218 | 4.63 |
| 6-5 | 0.754 | 0.565 | 0.523 | 1.21 |
| 6-6 | 0.923 | 0.783 | 0.946 | 1.25 |
| 6-7 | 0.467 | 0.654 | 1.03 | 7.19 |
| 6-8 | 1.379 | 0.127 | 1.16 | 0.151 |
| 6-9 | >20 | 0.387 | 20.0 | 2.12 |
| 7-1 | 0.678 | >20 | 0.932 | >20.0 |
| 7-2 | 0.578 | >20 | 0.501 | >20.0 |
| 7-3 | 1.589 | 5.337 | 1.74 | 13.0 |
| 7-4 | 3.673 | 3.711 | 7.11 | >20.0 |
| 7-5 | 0.610 | >20 | 0.906 | >20.0 |
| 8-1 | 2.459 | >20 | 3.56 | >20.0 |
| 8-2 | 0.891 | >20 | 0.401 | >20.0 |
| 8-3 | 1.142 | >20 | 1.05 | >20.0 |
| 8-4 | 3.180 | >20 | 3.92 | >20.0 |
| 8-5 | 2.076 | >20 | 6.94 | >20.0 |
| 8-6 | 1.382 | >20 | 1.28 | >20.0 |
| 8-7 | 2.381 | >20 | 6.21 | >20.0 |
| 8-8 | 0.936 | 16.858 | 1.31 | >20.0 |
| 8-9 | 0.457 | >20 | 0.682 | >20.0 |
| 8-10 | 1.126 | >20 | 2.35 | >20.0 |
| 8-11 | 0.713 | >20 | 1.36 | >20.0 |
| 8-12 | 0.587 | >20 | 0.783 | >20.0 |
| 8-13 | 0.640 | >20 | 1.36 | >20.0 |
| 8-14 | 2.399 | >20 | 7.46 | >20.0 |
| 8-15 | 1.966 | >20 | 5.60 | >20.0 |
| 8-16 | 0.662 | >20 | 0.593 | >20.0 |
| 8-17 | 0.913 | >20 | 1.45 | >20.0 |
| 9-1 | 0.319 | >20 | 3.76 | >20.0 |
| 9-2 | 0.806 | 9.080 | 11.2 | >20.0 |
| 9-3 | 1.063 | 4.281 | 2.34 | >20.0 |
| 9-4 | 5.379 | >20 | 2.54 | >20.0 |
| 9-5 | 0.625 | >20 | 0.661 | >20.0 |
| 9-6 | 1.092 | 18.943 | 1.28 | >20.0 |
| 9-7 | 0.000 | 8.289 | 0.308 | 11.2 |
| 9-8 | 0.418 | >20 | 0.174 | >20.0 |
| 9-9 | 0.863 | >20 | 2.94 | >20.0 |
| 9-10 | 1.693 | >20 | 1.41 | >20.0 |
| 9-11 | 0.420 | 4.117 | 0.397 | 4.07 |
| 9-12 | 0.580 | | 0.376 | 2.09 |
| 9-13 | 0.329 | 5.557 | 0.191 | 3.96 |
| 9-14 | 1.639 | 5.622 | 6.47 | >20.0 |
| 10-1 | 1.367 | >20 | 1.07 | >20.0 |
| 10-2 | 0.586 | >20 | 0.760 | >20.0 |
| 11-1 | 3.996 | >20 | 19.2 | >20.0 |

Table 7 summarizes the results of some cell assays for compounds of the formula (I-1).

TABLE 7

| | | MDA-MB | MDA-MB | | | |
|---|---|---|---|---|---|---|
| | MCF7 | 436 | 468 | HCC 1937 | MiaPaca | A431 |
| Ex. | IC50 | IC50 | IC50 | IC50 | IC50 | IC50 |
| No. | [μmol/l] | [μmol/l] | [μmol/l] | [μmol/l] | [μmol/l] | [μmol/l] |
| 1-1 | 0.8 | 0.14 | 0.274 | 1.08 | 0.827 | |
| 1-2 | 0.057 | | | | | |
| 1-3 | 0.31 | | | | | |
| 1-4 | 0.054 | | | | | |
| 1-5 | 0.230 | | | | | |
| 1-6 | 0.209 | 0.313 | 0.555 | 0.873 | 1.25 | |
| 1-7 | 0.057 | | | | | |
| 1-8 | 0.191 | | | | | |
| 1-9 | 0.287 | | | | | |
| 1-10 | 1.3 | | | | | |
| 1-11 | 0.124 | | | | | |
| 1-12 | 0.322 | | | | | |
| 1-13 | 0.410 | | | | | |
| 1-14 | | | | | | |
| 1-15 | | | | | | |
| 1-16 | | | | | | |
| 1-17 | | | | | | |
| 1-18 | | | | | | |
| 1-19 | | | | | | |
| 1-20 | 0.040 | | | | | |
| 1-21 | 0.392 | | | | | |
| 1-22 | 0.436 | | | | | |
| 1-23 | 0.262 | | | | | |
| 1-24 | 0.027 | | | | | |
| 1-25 | — | | | | | |
| 1-26 | 0.188 | | | | | |
| 1-27 | | | | | | |
| 1-28 | | | | | | |
| 1-29 | 0.032 | | | | | |
| 1-30 | 0.048 | | | | | |
| 1-31 | | | | | | |
| 1-32 | 0.107 | | | | | |
| 1-33 | 0.027 | 0.07 | 0.074 | 0.108 | 0.193 | |
| 1-34 | 0.112 | | | | | |
| 1-35 | 0.094 | | | | | |
| 1-36 | | | | | | |
| 1-37 | | | | | | |
| 1-38 | | | | | | |
| 1-39 | | | | | | |
| 1-40 | | | | | | |
| 1-41 | | | | | | |
| 1-42 | 1.280 | | | | | |
| 1-43 | 1.220 | | | | | |
| 1-44 | | | | | | |
| 1-45 | 0.626 | | | | | |
| 1-46 | | | | | | |
| 1-47 | | | | | | |
| 1-48 | 0.852 | | | | | |
| 1-49 | 0.640 | | | | | |
| 1-50 | — | | | | | |
| 1-51 | 0.253 | | | | | |
| 1-52 | 0.739 | | | | | |
| 1-53 | 0.070 | | | | | |
| 1-54 | 0.088 | | | | | |
| 1-55 | | | | | | |
| 1-56 | 0.030 | 0.084 | 0.138 | 0.171 | 0.316 | 0.214 |
| 1-57 | 0.250 | | | | | |
| 1-58 | | | | | | |
| 1-59 | | | | | | |
| 1-93 | 0.078 | | | | | |
| 1-94 | 0.085 | | | | | |
| 1-95 | 0.088 | | | | | |
| 1-96 | 0.108 | | | | | |
| 1-97 | 0.155 | | | | | |
| 1-98 | 0.099 | | | | | |
| 1-99 | 0.120 | | | | | |
| 1-100 | 0.127 | | | | | |
| 1-101 | 0.272 | | | | | |
| 1-102 | 0.079 | | | | | |
| 1-103 | 0.119 | | | | | |
| 1-104 | 0.455 | | | | | |
| 1-105 | 0.106 | | | | | |
| 1-106 | 0.155 | | | | | |
| 1-107 | 0.267 | | | | | |
| 1-108 | 0.076 | | | | | |
| 1-109 | 0.037 | | | | | |
| 1-110 | 0.045 | | | | | |
| 1-111 | 0.194 | | | | | |
| 1-112 | 0.079 | | | | | |
| 1-113 | 0.046 | | | | | |
| 1-114 | 0.043 | | | | | |
| 1-115 | 0.098 | | | | | |
| 1-116 | 0.031 | | | | | |
| 1-117 | 0.064 | | | | | |
| 1-118 | 0.037 | | | | | |
| 1-119 | 0.037 | | | | | |
| 1-120 | 0.070 | | | | | |
| 1-121 | 0.233 | | | | | |
| 1-122 | 0.288 | | | | | |
| 1-123 | 0.309 | | | | | |
| 1-124 | 1.70 | | | | | |
| 1-125 | 0.322 | | | | | |
| 1-126 | 0.594 | | | | | |
| 1-127 | 1.40 | | | | | |
| 1-128 | 0.033 | | | | | |
| 1-129 | 0.172 | | | | | |
| 1-130 | 0.061 | | | | | |
| 1-131 | 0.060 | | | | | |
| 1-132 | 0.070 | | | | | |
| 1-133 | 0.075 | | | | | |
| 1-134 | 0.114 | | | | | |
| 1-135 | 0.171 | | | | | |
| 1-136 | 0.156 | | | | | |
| 1-137 | 0.439 | | | | | |
| 1-138 | 0.054 | | | | | |
| 1-139 | 0.070 | | | | | |
| 1-140 | 0.073 | | | | | |
| 1-141 | 0.079 | | | | | |
| 1-142 | 0.106 | | | | | |
| 1-143 | 0.111 | | | | | |
| 1-144 | 0.149 | | | | | |
| 1-145 | 0.273 | | | | | |
| 1-146 | 0.047 | | | | | |
| 1-147 | 0.174 | | | | | |

For some exemplary compounds, further cell line data were obtained:

| | MDA- | | PLC/ | NCLH | | | | | | |
|---|---|---|---|---|---|---|---|---|---|---|
| | MB-435 | 786O | PRF5 | 2135 | DLD1 | PC 3 | Du145 | ECC1 | KM12 | HEC-1A |
| Ex. | IC50 | IC50 | IC50 | IC50 | IC50 | IC50 | IC50 | IC50 | IC50 | IC50 |
| No. | [μmol/l] | [μmol/l] | [μmol/l] | [μmol/l] | [μmol/l] | [μmol/l] | [μmol/l] | [μmol/l] | [μmol/l] | [μmol/l] |
| 1-29 | | | | | | | | | 0.132 | |
| 1-77 | 0.14 | 0.404 | 3.89 | 0.033 | 0.875 | 0.031 | 0.051 | 0.257 | 0.08 | |
| 1-112 | | | | | | | | | 0.174 | |
| 1-118 | | | | | | 0.025 | 0.039 | 0.221 | 0.275 | 1.76 |
| 1-120 | | | | | | | | | 0.714 | |
| 1-128 | | | | | | 0.020 | 0.029 | 0.137 | 0.074 | 0.328 |

Table 8 summarizes the results of the cell assays for the compounds of the formula (I-2).

TABLE 8

| | MCF7 | MDA-MB | MDA-MB | HCC | MiaPaca |
|---|---|---|---|---|---|
| Ex. | IC50 | 436 IC50 | 468 IC50 | 1937 IC50 | IC50 |
| No. | [μmol/l] | [μmol/l] | [μmol/l] | [μmol/l] | [μmol/l] |
| 2-1 | | | | | |
| 2-2 | | | | | |
| 2-3 | 1.060 | | | | |
| 2-4 | 0.624 | | | | |
| 2-5 | | | | | |
| 2-6 | 0.724 | | | | |
| 2-7 | | | | | |
| 2-8 | | | | | |
| 2-9 | | | | | |
| 2-10 | | | | | |
| 2-11 | | | | | |
| 2-12 | 1.240 | | | | |
| 2-13 | | | | | |
| 2-14 | | | | | |
| 2-15 | | | | | |
| 2-16 | | | | | |
| 2-17 | 0.400 | | | | |
| 2-18 | 0.935 | | | | |
| 2-19 | | | | | |
| 2-20 | 0.214 | | | | |
| 2-21 | | | | | |
| 2-22 | | | | | |
| 2-23 | | | | | |
| 2-24 | | | | | |
| 2-25 | | | | | |
| 2-26 | 5.520 | | | | |
| 2-27 | | | | | |
| 2-28 | 0.296 | | | | |
| 2-29 | | | | | |
| 2-30 | 0.064 | 0.115 | 0.153 | 1.5 | 0.255 |
| 2-31 | 0.490 | | | | |
| 2-32 | 0.112 | | | | |
| 2-33 | 0.246 | | | | |
| 2-34 | 0.429 | | | | |
| 2-35 | | | | | |
| 2-36 | | | | | |
| 2-37 | | | | | |
| 2-38 | | | | | |
| 2-39 | | | | | |
| 2-40 | | | | | |
| 2-41 | | | | | |
| 2-42 | 0.466 | | | | |
| 2-43 | 0.263 | | | | |
| 2-44 | 0.121 | | | | |
| 2-45 | 6.7 | | | | |
| 2-46 | | | | | |
| 2-47 | 0.302 | | | | |
| 2-48 | 0.762 | | | | |
| 2-49 | | | | | |
| 2-50 | | | | | |

Tables 9a and 9b summarize the results of the biological assays for the compounds of the formulae (I-3), (I-6), (I-7), (I-8), (I-9), (I-10) and (I-11).

TABLE 9a

| Example | MCF7 |
|---|---|
| No. | IC50 [μmol/l] |
| 3-1 | |
| 3-2 | 0.461 |
| 3-3 | 0.575 |
| 3-4 | 1.550 |
| 6-1 | 0.545 |
| 6-2 | 0.194 |
| 6-3 | |
| 6-4 | |
| 6-5 | 0.605 |
| 6-6 | 0.582 |
| 6-7 | 0.338 |
| 6-8 | 0.254 |
| 6-9 | 1.520 |
| 7-1 | |
| 7-2 | 1.210 |
| 7-3 | 0.974 |
| 7-4 | 2.630 |
| 7-5 | 0.992 |
| 8-1 | |
| 8-2 | 0.878 |
| 8-3 | |
| 8-4 | |
| 8-5 | |
| 8-6 | |
| 8-7 | |
| 8-8 | |

TABLE 9b

| Example | MCF7 |
|---|---|
| No. | IC50 [μmol/l] |
| 8-9 | |
| 8-10 | |
| 8-11 | |
| 8-12 | |
| 8-13 | |
| 8-14 | |
| 8-15 | |
| 8-16 | |
| 8-17 | |
| 9-1 | 1.260 |
| 9-2 | 1.720 |
| 9-3 | 1.530 |
| 9-4 | 3.440 |
| 9-5 | |
| 9-6 | |
| 9-7 | 1.100 |
| 9-8 | |
| 9-9 | |
| 9-10 | |
| 9-11 | 0.299 |
| 9-12 | |
| 9-13 | |
| 9-14 | 1.460 |
| 10-1 | |
| 10-2 | |
| 11-1 | 3.330 |

Table V.2 summarizes the results of the enzyme assays for the comparative compounds.

TABLE V.2

| Example | ACC 1 | ACC 2 | ACC-non human |
|---|---|---|---|
| No. | IC50 [μmol/l] | IC50 [μmol/l] | IC50 [μmol/l] |
| V-1 | >20 | >20 | 0.0005 |
| V-2 | >20 | >20 | 0.04 |
| V-3 | >20 | >20 | 0.002 |
| V-4 | 5.4 | >20 | 0.02 |

The results show very clearly that, in spite of close structural relationship, it is not possible to predict whether structures inhibiting non-human ACC are also inhibitors of human ACCs.

### Expression of ACC1 in Tumour Tissue and Normal Tissue

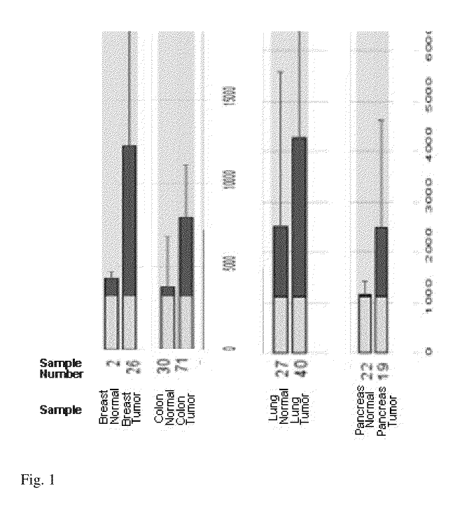

The expression of ACC1 in tumour tissue and corresponding normal tissue was determined by microarray (FIG. 1). In breast carcinoma, colorectal carcinoma, bronchial carcinoma and pancreas carcinoma, the expression of ACC1 was significantly upregulated compared to normal tissue.

1. Compounds of the formula (I) 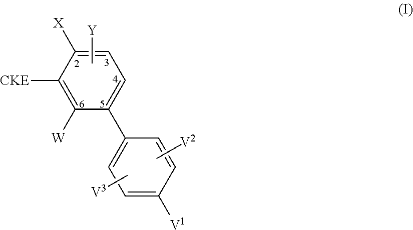
in which
X represents halogen, nitro or cyano or
represents an optionally monohalogen- or polyhalogen-substituted C₁-C₆-alkyl, C₁-C₆-alkoxy, C₁-C₆-alkoxy-C₁-C₆-alkoxy, C₃-C₇-cycloalkyl or a C₃-C₇-cycloalkyl-C₁-C₆-alkoxy radical, and
W and Y independently of one another represent hydrogen, nitro, cyano or halogen or represent an optionally monohalogen- or polyhalogen-substituted C₁-C₆-alkyl, C₁-C₆-alkoxy or C₃-C₇-cycloalkyl radical, and
V¹, V² and V³ independently of one another represent hydrogen, halogen, nitro or cyano or represent a C₁-C₆-alkyl, halo-C₁-C₆-alkyl, C₁-C₆-alkoxy, halo-C₁-C₆-alkoxy, C₁-C₆-alkylthio, C₁-C₆-alkylsulphinyl, C₁-C₆-alkylsulphonyl, C₁-C₆-alkoxy-C₁-C₆-alkyl, C₃-C₁₀-cycloalkyl radical or represent a monocyclic heterocycloalkyl radical, and/or
V¹ and V² together with the carbon atoms to which they are attached form a saturated or unsaturated cycle T¹ which optionally contains at least one further heteroatom and has 4 to 7 ring atoms and whose ring-forming atoms may be mono- or polysubstituted by identical or different substituents selected from the group consisting of halogen and a C₁-C₆-alkyl radical,
CKE represents one of the groups 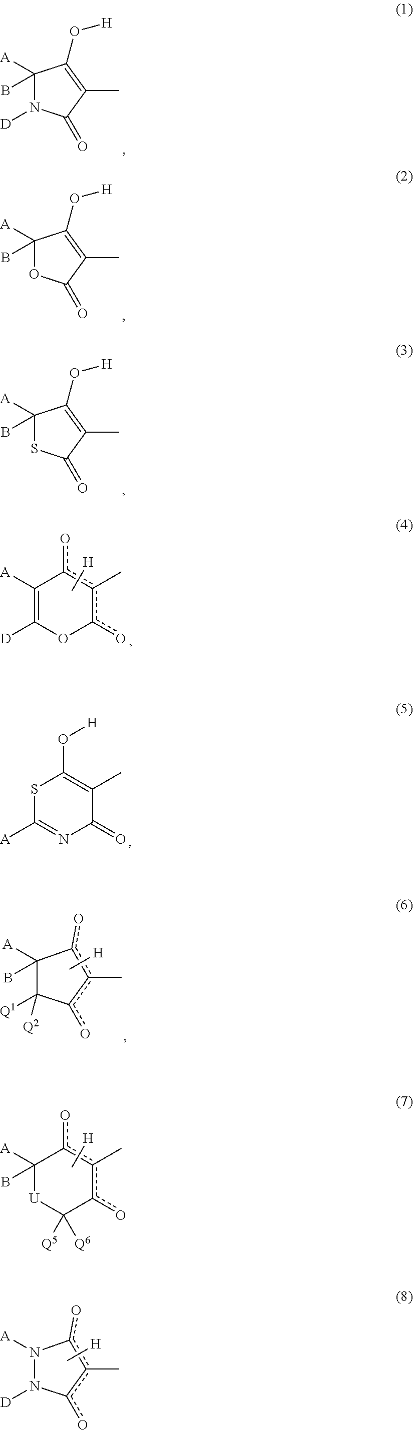 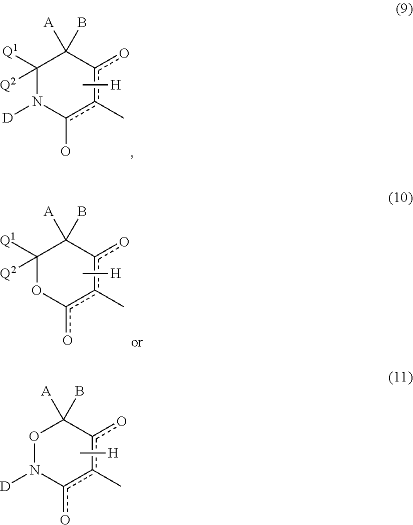
in which
U represents —S—, —S(O)—, —S(O)₂—, —O—, 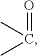
a substituted 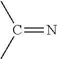
group
or represents a C₁-C₄-alkylene group which is optionally substituted by Q³ and Q⁴, and
A represents hydrogen or
represents an optionally monohalogen- or polyhalogen-substituted C₁-C₆-alkyl, C₂-C₆-alkenyl, C₁-C₆-alkoxy-C₁-C₆-alkyl or C₁-C₆-alkylthio-C₁-C₆-alkyl radical or
represents a C₃-C₇-cycloalkyl, C₃-C₇-cycloalkyl-C₁-C₄-alkyl or monocyclic heterocyclyl or heterocyclyl-C₁-C₄-alkyl radical,
each of which may be mono- or polysubstituted by identical or different substituents selected from the group consisting of halogen and a C₁-C₆-alkyl radical or
represents an aryl, aryl-C₁-C₆-alkyl or heteroaryl radical, each of which may optionally be mono- or polysubstituted by identical or different substituents selected from the group consisting of halogen, cyano, nitro and C₁-C₆-alkyl, halo-C₁-C₆-alkyl, C₁-C₆-alkoxy and halo-C₁-C₆-alkoxy radicals and
B represents hydrogen or represents a C₁-C₆-alkyl or C₁-C₆-alkoxy-C₁-C₆-alkyl radical, or
A and B together with the carbon atom to which they are attached form a saturated or unsaturated cycle T² which optionally contains at least one heteroatom and has 3 to 8 ring atoms and whose ring-forming atoms may be mono- or polysubstituted by identical or different substituents selected from the group consisting of the radicals R¹, R² and R³,
where R¹, R² and R³ independently of one another
a) represent halogen, hydroxyl or cyano or
b) represent C₁-C₆-alkyl, C₁-C₆-alkoxy, C₁-C₆-alkoxy-C₁-C₆-alkyl, C₁-C₆-alkylcarbonyl,
C₁-C₆-alkoxycarbonyl, C₁-C₆-alkylaminocarbonyl, C₁-C₆-alkylthio, C₁-C₆-alkylsulphinyl, C₁-C₆-alkylsulphonyl, C₁-C₆-alkylaminosulphonyl, C₁-C₆-alkoxy-C₁-C₆-alkoxy, halo-C₁-C₆-alkyl or halo-C₁-C₆-alkoxy radical which is optionally hydroxyl-substituted in the alkyl moiety, or
c) represent an aryl, arylcarbonyl, arylsulphonyl, arylamino, heteroaryl, heteroarylcarbonyl, heteroarylsulphonyl or heteroarylamino radical, or
d) represent a C₃-C₇-cycloalkyl, C₃-C₇-cycloalkylcarbonyl, C₃-C₇-cycloalkylsulphonyl, heterocyclyl, heterocyclylcarbonyl or heterocyclylsulphonyl radical,
where the radicals mentioned under c) and d) may optionally be mono- or polysubstituted at the ring system by identical or different substituents selected from the group consisting of halogen, hydroxyl, cyano, nitro and C₁-C₆-alkyl, C₁-C₆-alkoxy, halo-C₁-C₆-alkyl, halo-C₁-C₆-alkoxy, C₁-C₆-alkoxy-C₁-C₆-alkyl, C₃-C₁₀-cycloalkyl and 3- to 6-membered heterocycloalkyl radicals, and/or
e) two of the radicals R¹, R² and R³ together with the ring atom(s) of the cycle T² to which they are attached may form a further saturated or unsaturated cycle T³ which optionally contains at least one heteroatom and has 3 to 7 ring atoms and may be mono- or polysubstituted by identical or different substituents selected from the group consisting of the radicals R⁴, R⁵ and R⁶,
where R⁴, R⁵ and R⁶ independently of one another represent a C₁-C₆-alkyl or C₁-C₆-alkoxy radical, and
D represents hydrogen or
represents a C₁-C₆-alkyl, C₂-C₆-alkenyl, C₂-C₆-alkynyl or C₁-C₆-alkoxy-C₁-C₆-alkyl radical or
represents a C₃-C₇-cycloalkyl, C₃-C₇-cycloalkyl-C₁-C₄-alkyl or monocyclic heterocyclyl or heterocyclyl-C₁-C₄-alkyl radical or
represents an aryl, aryl-C₁-C₆-alkyl, heteroaryl or heteroaryl-C₁-C₆-alkyl radical,
where the radicals mentioned may optionally be mono- or polysubstituted by identical or different substituents selected from the group consisting of halogen, hydroxyl, cyano, nitro and C₁-C₆-alkyl, halo-C₁-C₆-alkyl, C₁-C₆-alkoxy, halo-C₁-C₆-alkoxy, C₁-C₆-alkoxy-C₁-C₆-alkyl, C₃-C₁₀-cycloalkyl and monocyclic heterocycloalkyl radicals, or
A and D together with the atoms to which they are attached form a saturated or unsaturated cycle T⁴ which optionally contains at least one further heteroatom and has 3 to 7 ring atoms, which may be bridged and whose ring-forming atoms may be mono- or polysubstituted by identical or different substituents selected from the group consisting of the radicals R⁷, R⁸ and R⁹,
where R⁷, R⁸ and R⁹ independently of one another represent hydroxyl, halogen or represent a C₁-C₆-alkyl or C₁-C₆-alkoxy radical, and
A and Q¹ together with the atoms to which they are attached form a saturated or unsaturated cycle T⁵ which optionally contains at least one further heteroatom and has 5 to 7 ring atoms and whose ring-forming atoms may be mono- or polysubstituted by identical or different substituents selected from the group consisting of halogen, hydroxyl, cyano, nitro and C₁-C₆-alkyl, halo-C₁-C₆-alkyl, C₁-C₆-alkoxy, halo-C₁-C₆-alkoxy, C₁-C₆-alkoxy-C₁-C₆-alkyl and C₃-C₁₀-cycloalkyl radicals,
with the proviso that B and Q² represent a bond if the cycle T⁵ formed by A and Q¹ is aromatic,
Q¹ represents hydrogen or
represents a C₁-C₆-alkyl or C₁-C₆-alkoxy radical which is optionally mono- or polysubstituted by identical or different substituents selected from the group consisting of halogen, hydroxyl and a C₁-C₆-alkoxy radical or
represents a C₃-C₇-cycloalkyl, C₃-C₇-cycloalkyl-C₁-C₄-alkyl or monocyclic heterocyclyl or heterocyclyl-C₁-C₄-alkyl radical,
each of which may optionally be mono- or polysubstituted by identical or different substituents selected from the group consisting of halogen, hydroxyl and C₁-C₆-alkyl, halo-C₁-C₆-alkyl, C₁-C₆-alkoxy, C₁-C₆-alkoxy-C₁-C₆-alkoxy and halo-C₁-C₆-alkoxy radicals or
represents a phenyl radical which may optionally be mono- or polysubstituted by identical or different substituents selected from the group consisting of halogen, hydroxyl, cyano, nitro and C₁-C₆-alkyl, halo-C₁-C₆-alkyl, C₁-C₆-alkoxy, halo-C₁-C₆-alkoxy, C₁-C₆-alkoxy-C₁-C₆-alkyl and C₃-C₁₀-cycloalkyl radicals, and
Q², Q⁴, Q⁵ and Q⁶ independently of one another represent hydrogen or represent a C₁-C₆-alkyl radical, and
Q³ represents hydrogen or
represents a C₁-C₆-alkyl or C₁-C₆-alkoxy radical which is optionally mono- or polysubstituted by identical or different substituents selected from the group consisting of halogen, hydroxyl and a C₁-C₆-alkoxy radical or
represents a C₃-C₇-cycloalkyl, C₃-C₇-cycloalkyl-C₁-C₄-alkyl or a monocyclic heterocyclyl or heterocyclyl-C₁-C₄-alkyl radical,
each of which may optionally be mono- or polysubstituted by identical or different substituents selected from the group consisting of halogen, hydroxyl and C₁-C₆-alkyl, halo-C₁-C₆-alkyl, C₁-C₆-alkoxy, C₁-C₆-alkoxy-C₁-C₆-alkoxy and halo-C₁-C₆-alkoxy radicals or
represents a phenyl radical which may optionally be mono- or polysubstituted by identical or different substituents selected from the group consisting of halogen, hydroxyl, cyano, nitro and C₁-C₆-alkyl, halo-C₁-C₆-alkyl, C₁-C₆-alkoxy, halo-C₁-C₆-alkoxy, C₁-C₆-alkoxy-C₁-C₆-alkyl and C₃-C₁₀-cycloalkyl radicals, or
Q¹ and Q² together with the carbon atom to which they are attached form a saturated or unsaturated cycle T⁶ which optionally contains at least one further heteroatom having 3 to 7 ring atoms,
whose ring-forming atoms may be mono- or polysubstituted by identical or different substituents selected from the group consisting of halogen, hydroxyl, cyano, nitro and C₁-C₆-alkyl, halo-C₁-C₆-alkyl, C₁-C₆-alkoxy, halo-C₁-C₆-alkoxy, C₁-C₆-alkoxy-C₁-C₆-alkyl and C₃-C₁₀-cycloalkyl radicals, or
Q³ and Q⁴ together with the carbon atom to which they are attached form a saturated or unsaturated cycle T⁷ which optionally contains at least one heteroatom and has 3 to 7 ring atoms and whose ring-forming atoms may be mono- or polysubstituted by identical or different substituents selected from the group consisting of halogen, hydroxyl, cyano, nitro and C₁-C₆-alkyl, halo-C₁-C₆-alkyl, C₁-C₆-alkoxy, halo-C₁-C₆-alkoxy, C₁-C₆-alkoxy-C₁-C₆-alkyl and C₃-C₁₀-cycloalkyl radicals,
for use as medicaments.

2. Compounds according to claim 1 of the formula (I) in which
X represents halogen or an optionally monohalogen- or polyhalogen-substituted C₁-C₃-alkyl or C₁-C₃-alkoxy radical,
for use as medicaments.

3. Compounds according to claim 1 of the formula (I) in which
W and Y independently of one another represent hydrogen or represent an optionally monohalogen- or polyhalogen-substituted C₁-C₃-alkyl radical,
for use as medicaments.

4. Compounds according to claim 1 of the formula (I) in which
V¹, V² and V³ independently of one another represent hydrogen, halogen or a C₁-C₃-alkyl or C₁-C₃-haloalkyl radical,
for use as medicaments.

5. Compounds according to claim 1 of the formula (I) in which
A represents hydrogen or
represents an optionally monohalogen- or polyhalogen-substituted C₁-C₆-alkyl or C₁-C₆-alkoxy-C₁-C₆-alkyl radical or
represents a C₃-C₆-cycloalkyl radical which may be mono- or polysubstituted by identical or different substituents selected from the group consisting of halogen and a C₁-C₃-alkyl radical
for use as medicaments.

6. Compounds according to claim 1 of the formula (I) in which
A and B together with the carbon atom to which they are attached form a saturated cycle T² which optionally contains one or two heteroatoms and has 3 to 8 ring atoms and whose ring-forming atoms may be mono- or polysubstituted by identical or different substituents selected from the group consisting of the radicals R¹, R² and R³,
where R¹, R² and R³ independently of one another
a) represent a C₁-C₃-alkyl, C₁-C₃-alkoxy, C₁-C₃-alkoxy-C₁-C₃-alkyl, C₁-C₃-alkoxy-C₁-C₃-alkoxy or halo-C₁-C₃-alkoxy radical which is optionally hydroxyl-substituted in the alkyl moiety, and/or
b) two of the radicals R¹, R² and R³ together with the ring atom(s) of the cycle T² to which they are attached may form a further saturated or aromatic cycle T³ which optionally contains at least one oxygen atom and has 5 to 7 ring atoms and which may be mono- or polysubstituted by a C₁-C₃-alkyl radical,
for use as medicaments.

7. Compounds according to claim 1 of the formula (I) in which
B represents hydrogen or represents a C₁-C₆-alkyl or C₁-C₆-alkoxy-C₁-C₆-alkyl radical,
for use as medicaments.

8. Compounds according to claim 1 in which CKE represents the group 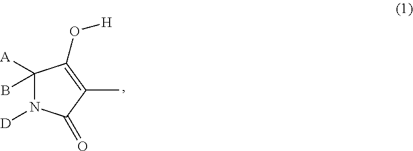
defined as compounds of the formula (I-1) 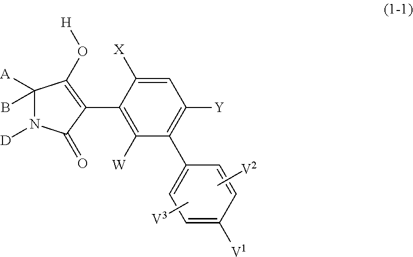
in which
X represents halogen or
represents an optionally monohalogen- or polyhalogen-substituted C₁-C₃-alkyl or C₁-C₃-alkoxy radical, and
W and Y independently of one another represent hydrogen or halogen or represent an optionally monohalogen- or polyhalogen-substituted C₁-C₃-alkyl radical, and
V¹, V² and V³ independently of one another represent hydrogen or halogen or represent a
C₁-C₃-alkyl, C₁-C₃-haloalkyl, C₁-C₃-alkoxy, C₁-C₃-haloalkoxy or C₁-C₃-alkoxy-C₁-C₃-alkyl radical, and/or
V¹ and V² together with the carbon atoms to which they are attached form a saturated or unsaturated cycle T¹ which optionally contains at least one further heteroatom and has 5 or 6 ring atoms and whose ring-forming atoms may be mono- or polysubstituted by identical or different substituents selected from the group consisting of halogen and a C₁-C₃-alkyl radical,
A represents hydrogen or
represents an optionally monohalogen- or polyhalogen-substituted C₁-C₆-alkyl or C₁-C₆-alkoxy-C₁-C₆-alkyl radical or
represents a C₃-C₇-cycloalkyl radical or 4- to 7-membered monocyclic heterocyclyl radical, each of which may be mono- or polysubstituted by identical or different substituents selected from the group consisting of halogen and a C₁-C₃-alkyl radical, and
B represents hydrogen or represents a C₁-C₆-alkyl or
C₁-C₃-alkoxy-C₁-C₃-alkyl radical, or
A and B together with the carbon atom to which they are attached form a saturated or unsaturated cycle T² which optionally contains one or two heteroatoms and has 3 to 8 ring atoms and whose ring-forming atoms may be mono- or polysubstituted by identical or different substituents selected from the group consisting of the radicals R¹, R² and R³,
where R¹, R² and R³ independently of one another
a) represent halogen or hydroxyl or
b) represent a C₁-C₄-alkyl, C₁-C₄-alkoxy, C₁-C₃-alkoxy-C₁-C₃-alkyl, C₁-C₃-alkoxy-C₁-C₃-alkoxy, halo-C₁-C₃-alkyl or halo-C₁-C₃-alkoxy radical which is optionally hydroxyl-substituted in the alkyl moiety and/or
c) two of the radicals R¹, R² and R³ together with the ring atom(s) of the cycle T² to which they are attached may form a further saturated or aromatic cycle T³ which optionally contains one or two heteroatoms and has 5 to 7 ring atoms and which may be mono- or polysubstituted by identical or different substituents selected from the group consisting of the radicals R⁴, R⁵ and R⁶, where R⁴, R⁵ and R⁶ independently of one another represent a C₁-C₃-alkyl or C₁-C₃-alkoxy radical, and
D represents hydrogen or
represents a C₁-C₆-alkyl or C₁-C₆-alkoxy-C₁-C₆-alkyl radical or
represents a C₃-C₇-cycloalkyl or 4- to 7-membered monocyclic heterocyclyl radical,
where the radicals mentioned may optionally be mono- or polysubstituted by identical or different substituents selected from the group consisting of halogen and hydroxyl and C₁-C₃-alkyl, halo-C₁-C₃-alkyl, C₁-C₃-alkoxy, halo-C₁-C₃-alkoxy and C₁-C₃-alkoxy-C₁-C₃-alkyl radicals, or
A and D together with the atoms to which they are attached form a saturated or unsaturated cycle T⁴ which optionally contains a further heteroatom and has 5 to 7 ring atoms and whose ring-forming atoms may be mono- or polysubstituted by identical or different substituents selected from the group consisting of the radicals R⁷, R⁸ and R⁹,
where R⁷, R⁸ and R⁹ independently of one another represent halogen or a C₁-C₃-alkyl or C₁-C₃-alkoxy radical,
for use as medicaments

9. Compounds according to claim 8 of the formula (I-1) in which
A and B together with the carbon atom to which they are attached form a saturated cycle T² which optionally contains a heteroatom and has 3 to 8 ring atoms and whose ring-forming atoms may be mono- or disubstituted by identical or different substituents selected from the group consisting of the radicals R¹ and R², where R¹ and R² independently of one another
a) represent hydroxyl or
b) represent a C₁-C₄-alkyl, C₁-C₄-alkoxy, C₁-C₃-alkoxy-C₁-C₃-alkyl, C₁-C₃-alkoxy-C₁-C₃-alkoxy, halo-C₁-C₃-alkyl or halo-C₁-C₃-alkoxy radical which is optionally substituted in the alkyl moiety by hydroxyl,
for use as medicaments.

10. Compounds according to claim 8 of the formula (I-1) in which A and B together with the carbon atom to which they are attached form a cyclohexane ring or tetrahydropyran ring,
for use as medicaments.

11. Compounds according to claim 8 of the general formula (I-1) in which
X represents chlorine or represents a methyl radical, and
W and Y independently of one another represent hydrogen or represent a methyl radical,
V¹ represents chlorine, fluorine or a methyl radical, and
V² and V³ independently of one another represent hydrogen, chlorine or fluorine,
A and B together with the carbon atom to which they are attached form a saturated cycle T² which optionally contains one oxygen atom and has 6 ring atoms and whose ring-forming atoms may be mono- or disubstituted by identical or different substituents selected from the group consisting of the radicals R¹ and R², where R¹ and R² independently of one another represent hydroxyl or represent a C₁-C₃-alkyl, hydroxymethyl, C₁-C₂-alkoxy, methoxy-C₁-C₂-alkyl, trifluoromethyl, pentafluoroethyl or 2,2,2-trifluoroethoxy radical, and
D represents hydrogen,
for use as medicaments

12. Compounds
(5s,8s)-3-(4′-chloro-3′-fluoro-4-methylbiphenyl-3-yl)-4-hydroxy-8-(trifluoromethyl)-1-azaspiro[4.5]dec-3-en-2-one
(5s,8s)-3-(4′-chloro-5-fluoro-4-methylbiphenyl-3-yl)-4-hydroxy-8-(trifluoromethyl)-1-azaspiro[4.5]dec-3-en-2-one
(5s,8s)-3-(4,4′-dichloro-3′-fluorobiphenyl-3-yl)-4-hydroxy-8-(trifluoromethyl)-1-azaspiro[4.5]dec-3-en-2-one
(5s,8s)-3-(4′-chloro-3′-fluoro-2,4-dimethylbiphenyl-3-yl)-4-hydroxy-8-(trifluoromethyl)-1-azaspiro[4.5]dec-3-en-2-one
3-(4′-chloro-3′,6-difluoro-4-methylbiphenyl-3-yl)-4-hydroxy-8-(trifluoromethyl)-1-azaspiro[4.5]dec-3-en-2-one
(5s,8s)-3-(4′-chloro-2,4-dimethylbiphenyl-3-yl)-4-hydroxy-8-(trifluoromethyl)-1-azaspiro[4.5]dec-3-en-2-one
(5s,8s)-4-hydroxy-8-(trifluoromethyl)-3-(3′,4′,5-trifluoro-4-methylbiphenyl-3-yl)-1-azaspiro[4.5]dec-3-en-2-one
3-(4′-chloro-4,6-dimethylbiphenyl-3-yl)-4-hydroxy-8-(trifluoromethyl)-1-azaspiro[4.5]dec-3-en-2-one
(5s,8s)-3-(4′-chloro-4,6-dimethylbiphenyl-3-yl)-4-hydroxy-8-(trifluoromethyl)-1-azaspiro[4.5]dec-3-en-2-one
(5s,8s)-3-(4-chloro-3′,4′-difluorobiphenyl-3-yl)-4-hydroxy-8-(trifluoromethyl)-1-azaspiro[4.5]dec-3-en-2-one
3-(4′-chloro-6-fluoro-4-methylbiphenyl-3-yl)-4-hydroxy-8-(trifluoromethyl)-1-azaspiro[4.5]dec-3-en-2-one
(5s,8s)-3-(3′,4′-difluoro-2,4-dimethylbiphenyl-3-yl)-4-hydroxy-8-(trifluoromethyl)-1-azaspiro[4.5]dec-3-en-2-one
(5s,8s)-3-(4′-chloro-3′,5-difluoro-4-methylbiphenyl-3-yl)-4-hydroxy-8-(trifluoromethyl)-1-azaspiro[4.5]dec-3-en-2-one
(5s,8s)-3-(4′-chloro-3′,5-difluoro-4-methylbiphenyl-3-yl)-4-hydroxy-8-methoxy-1-azaspiro[4.5]dec-3-en-2-one
(5s,8s)-3-(4′-chloro-5-fluoro-4-methylbiphenyl-3-yl)-4-hydroxy-8-methoxy-1-azaspiro[4.5]dec-3-en-2-one
(5s,8s)-4-hydroxy-8-methoxy-3-(3′,4′,5-trifluoro-4-methylbiphenyl-3-yl)-1-azaspiro[4.5]dec-3-en-2-one
(5s,8s)-3-(4′,6-dichloro-3′-fluoro-4-methylbiphenyl-3-yl)-4-hydroxy-8-methoxy-1-azaspiro[4.5]dec-3-en-2-one
(5s,8s)-3-(4′,6-dichloro-4-methylbiphenyl-3-yl)-4-hydroxy-8-methoxy-1-azaspiro[4.5]dec-3-en-2-one
(5s,8s)-3-(4′-chloro-4-methylbiphenyl-3-yl)-4-hydroxy-8-(hydroxymethyl)-1-azaspiro[4.5]dec-3-en-2-one
(5s,8s)-3-(4′-chloro-4-methylbiphenyl-3-yl)-4-hydroxy-8-(trifluoromethyl)-1-azaspiro[4.5]dec-3-en-2-one
(5s,8s)-3-(4,4′-dichlorobiphenyl-3-yl)-4-hydroxy-8-methoxy-1-azaspiro[4.5]dec-3-en-2-one
(5s,8s)-3-(4′-chloro-2,4-dimethylbiphenyl-3-yl)-4-hydroxy-8-methoxy-1-azaspiro[4.5]dec-3-en-2-one
(5r,8r)-3-(4′-chloro-4-methylbiphenyl-3-yl)-4,8-dihydroxy-8-(trifluoromethyl)-1-azaspiro[4.5]dec-3-en-2-one
(5r,8r)-3-(4′-chloro-3′-fluoro-4-methylbiphenyl-3-yl)-4,8-dihydroxy-8-(trifluoromethyl)-1-azaspiro[4.5]dec-3-en-2-one
(5r,8r)-3-(3′,4′-difluoro-4-methylbiphenyl-3-yl)-4,8-dihydroxy-8-(trifluoromethyl)-1-azaspiro[4.5]dec-3-en-2-one
(5r,8r)-3-(4′-chloro-2,4-dimethylbiphenyl-3-yl)-4,8-dihydroxy-8-(trifluoromethyl)-1-azaspiro[4.5]dec-3-en-2-one
(5r,8r)-3-(4′-chloro-3′-fluoro-2,4-dimethylbiphenyl-3-yl)-4,8-dihydroxy-8-(trifluoromethyl)-1-azaspiro[4.5]dec-3-en-2-one
(5r,8r)-3-(4′-chloro-6-fluoro-4-methylbiphenyl-3-yl)-4,8-dihydroxy-8-(trifluoromethyl)-1-azaspiro[4.5]dec-3-en-2-one
(5s,8s)-3-(3′,4′-difluoro-4-methylbiphenyl-3-yl)-4-hydroxy-8-(trifluoromethyl)-1-azaspiro[4.5]dec-3-en-2-one
(5r,8r)-3-(3′,4′-difluoro-4-methylbiphenyl-3-yl)-4-hydroxy-8-(trifluoromethyl)-1-azaspiro[4.5]dec-3-en-2-one
(5r,8r)-3-(4′-chloro-3′-fluoro-4-methylbiphenyl-3-yl)-4,8-dihydroxy-8-(pentafluoroethyl)-1-azaspiro[4.5]dec-3-en-2-one)
(5r,8r)-3-(4′-chloro-6-fluoro-4-methylbiphenyl-3-yl)-4,8-dihydroxy-8-(pentafluoroethyl)-1-azaspiro[4.5]dec-3-en-2-one
(5S,7S)-3-(4′-chloro-4-methylbiphenyl-3-yl)-4-hydroxy-7-(trifluoromethyl)-1-azaspiro[4.5]dec-3-en-2-one
(5s,8s)-3-(4,4′-dichlorobiphenyl-3-yl)-4-hydroxy-8-(trifluoromethyl)-1-azaspiro[4.5]dec-3-en-2-one
(5s,8s)-3-(4′-chloro-6-fluoro-4-methylbiphenyl-3-yl)-4-hydroxy-8-methoxy-1-azaspiro[4.5]dec-3-en-2-one
(5S,7S)-3-(4,4′-dichlorobiphenyl-3-yl)-4-hydroxy-7-(trifluoromethyl)-1-azaspiro[4.5]dec-3-en-2-one
3-(4,4′-dichlorobiphenyl-3-yl)-4-hydroxy-8,8-dimethyl-1-azaspiro[4.5]dec-3-en-2-one
3-(4,4′-dichlorobiphenyl-3-yl)-4-hydroxy-8-methyl-1-azaspiro[4.5]dec-3-en-2-one
3-(4,4′-dichlorobiphenyl-3-yl)-4-hydroxy-1-azaspiro[4.5]dec-3-en-2-one
(5s,8s)-3-(4,4′-dichlorobiphenyl-3-yl)-4-hydroxy-8-isopropyl-1-azaspiro[4.5]dec-3-en-2-one
(5s,8s)-3-(2′-chloro-4′-fluoro-4-methylbiphenyl-3-yl)-4-hydroxy-8-methoxy-1-azaspiro[4.5]dec-3-en-2-one

13. A pharmaceutical formulation comprising a compound of the formula (I) according to claim 1 and a pharmaceutically acceptable excipient.

14. A method of treating tumor disorders comprising administering a compound of the formula (I) according to claim 1 to a patient in need thereof.

15. A method of treating breast carcinomas, pancreas carcinomas, kidney cell carcinomas, hepatocellular carcinomas, malignant melanomas and other skin tumours, non-small-cell bronchial carcinomas, endometrial carcinomas, colorectal carcinomas or prostate carcinomas comprising administering a compound of the formula (I) according to claim 1 to a patient in need thereof.

16. Compound of the formula (I) according to claim 1 for the prophylaxis and/or therapy of tumour disorders.

17. Compound of the formula (I) according to claim 1 for the prophylaxis and/or therapy of breast carcinomas, pancreas carcinomas, kidney cell carcinomas, hepatocellular carcinomas, malignant melanomas and other skin tumours, non-small-cell bronchial carcinomas, endometrial carcinomas, colorectal carcinomas or prostate carcinomas.

18. Pharmaceutical formulation the form of a tablet comprising a compound of the formula (I) of claim 1 for the prophylaxis and/or therapy of breast carcinomas, pancreas carcinomas, kidney cell carcinomas, hepatocellular carcinomas, malignant melanomas and other skin tumours, non-small-cell bronchial carcinomas, endometrial carcinomas, colorectal carcinomas or prostate carcinomas.
